(19) United States
(12) Patent Application Publication
(10) Pub. No.: US 2026/0103461 A1
(43) Pub. Date: Apr. 16, 2026

(54) ESTROGEN RECEPTOR MODULATORS AND USES THEREOF

(71) Applicant: Olema Pharmaceuticals, Inc., San Francisco, CA (US)

(72) Inventors: Brian R. Hearn, Moraga, CA (US); David C. Myles, Berkeley, CA (US); Dirk A. Heerding, Bend, OR (US)

(21) Appl. No.: 19/104,880

(22) PCT Filed: Aug. 18, 2023
(86) PCT No.: PCT/US23/30595

§ 371 Date: Feb. 19, 2025

Related U.S. Application Data
(60) Provisional application No. 63/399,269, filed on Aug. 19, 2022.

(51) Int. Cl. C07D 471/04; A61K 31/4196; A61K 31/436; A61K 31/437; A61K 31/4439; A61K 31/444; A61K 31/4545; A61K 31/4709; A61K 31/506; A61K 31/517; A61K 31/519; A61K 31/553; A61K 31/566; A61K 31/675; A61K 45/06; C07K 16/32

(52) U.S. Cl. CPC C07D 471/04; A61K 31/4196; A61K 31/436; A61K 31/437; A61K 31/4439; A61K 31/444; A61K 31/4545; A61K 31/4709; A61K 31/506; A61K 31/517; A61K 31/519; A61K 31/553; A61K 31/566; A61K 31/675; A61K 45/06; C07K 16/32

(57) ABSTRACT

The present disclosure provides compounds that are estrogen receptor modulators (e.g., estrogen receptor agonists, estrogen receptor antagonists, and/or complete estrogen receptor antagonists), and uses thereof.

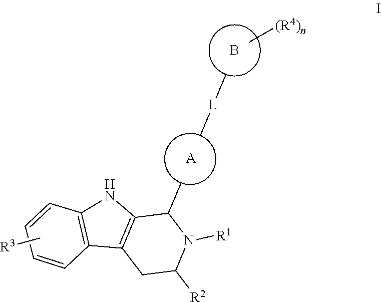

### RELATED APPLICATIONS

This application claims priority to and benefit of U.S. Application No. 63/399,269, filed Aug. 19, 2022, the entire contents of which are hereby incorporated by reference.

### BACKGROUND

The estrogen receptor (ER) plays important roles in various diseases, disorders, and conditions, such as cancers, including breast cancers, menopause-related conditions or symptoms, and osteoporosis. An, K-C. Asian Spine J. 10(4); 787-91 (2016 August). About 70% of human breast cancers are hormone dependent and ER-positive. Lumachi, et al., Curr. Med Chem., 20(5):596-604 (2013). A variety of treatments have been developed to target the estrogen receptor and/or its activities.

### SUMMARY

Selective estrogen receptor modulators (SERMs) or degraders (SERDs) are a particularly useful or promising tools for such therapy. For example, an estrogen receptor modulator that acts as an agonist (or partial agonist) in bone tissue may be useful for treating osteoporosis, e.g., in post-menopausal women. Further, an estrogen receptor modulator that acts as an antagonist in breast tissue may be useful for treating breast cancer. In some instances, the same estrogen receptor modulator may be used in both scenarios.

In some embodiments, the present disclosure provides compounds that are estrogen receptor modulators. In some embodiments, provided compounds are estrogen receptor agonists, e.g., as defined herein. In some embodiments, provided compounds are estrogen receptor antagonists, e.g., as defined herein.

Additionally, there remains a need for anti-estrogen agents that can completely inhibit estrogen receptors, including those coded for by both wild-type and mutant versions (e.g., those containing activating mutations) of the gene encoding Estrogen Receptor-alpha (ERα), Estrogen Receptor 1 (ESR1). The estrogen receptor is a tripartite protein comprising two distinct transcriptional activation functions (AF1 and AF2). Complete anti-estrogen activity requires inactivation of both AF1 and AF2. Activating mutations in the gene that codes for estrogen receptor 1 allows for activation of both AF1 and AF2 even in the absence of estrogen.

Many patients develop resistance to certain therapies that target the estrogen receptor (ER) over time. Certain first line therapies for treating ER-associated diseases, disorders, or conditions, are found to exhibit agonistic activity in conjunction with their antagonistic properties. Fulvestrant, in contrast, is the only approved therapy that exhibits complete anti-estrogenic activity, but is not orally bioavailable, and must be administered parenterally.

In some embodiments, the present disclosure provides certain compounds and compositions that are complete estrogen receptor antagonists, and therefore do not suffer from the deficiencies found in previous therapies.

Additionally, in some embodiments, provided compounds may be orally bioavailable.

In some embodiments, the present disclosure provides an estrogen receptor modulator (e.g., an estrogen receptor agonist, an estrogen receptor antagonist, and/or a complete estrogen receptor antagonist) that is a compound of Formula I:

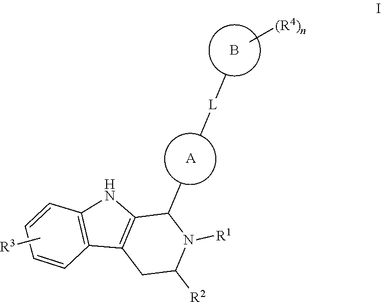

or a pharmaceutically acceptable salt thereof, wherein A, B, L, n, R¹, R², R³, and R⁴ are as defined herein.

In some embodiments, the present disclosure provides methods of treating a disease, disorder, or condition associated with an estrogen receptor. In some embodiments, the present disclosure provides a methods of treating a disease, disorder, or condition associated with a mutation of an estrogen receptor.

In some embodiments, the present disclosure provides methods of treating a cancer. In some embodiments, the present disclosure provides methods of treating a cancer comprising administering a compound of Formula I or a pharmaceutically acceptable salt thereof, optionally in combination with an anti-cancer agent.

In some embodiments, the present disclosure provides methods of preventing recurrence of a cancer. In some embodiments, the present disclosure provides methods of preventing recurrence of a cancer comprising administering a compound of Formula I or a pharmaceutically acceptable salt thereof, optionally in combination with an anti-cancer agent.

In some embodiments, the present disclosure provides methods of treating osteoporosis, e.g., in post-menopausal women. In some embodiments, the present disclosure provides methods of treating osteoporosis comprising administering a compound of Formula I or a pharmaceutically acceptable salt thereof.

In some embodiments, the present disclosure provides methods of treating one or more menopausal symptoms or conditions. In some embodiments, the present disclosure provides methods of treating one or more menopausal symptoms comprising administering a compound of Formula I or a pharmaceutically acceptable salt thereof.

### DETAILED DESCRIPTION OF CERTAIN EMBODIMENTS

The present disclosure provides compounds and compositions useful as estrogen receptor modulators (e.g., estrogen receptor agonists, estrogen receptor antagonists, and/or complete estrogen receptor antagonists). In some embodiments, such compounds include those of the formulae described herein, or a pharmaceutically acceptable salt thereof, wherein each variable is as defined and described herein.

### Compounds and Definitions

Compounds of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. For example, in some cases, Table 1 shows one or more stereoisomers of a compound, and unless otherwise indicated, represents each stereoisomer alone and/or as a mixture. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition followed by, after an interval of time, administration of a second or subsequent dose of a pharmaceutical composition.

Agonist: As used herein, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkylene: The term “alkylene” is refers to a bivalent alkyl group. In some embodiments, “alkylene” is a bivalent straight or branched alkyl group. In some embodiments, an “alkylene chain” is a polymethylene group, i.e., —(CH₂)ₙ—, wherein n is a positive integer, e.g., from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3- to 7-membered ring. The substituents can be on the same or different atoms. The suffix “-ene” when appended to certain groups herein are intended to refer to a bifunctional moiety of said group. For example, “-ene”, when appended to “cyclopropyl” becomes “cyclopropylene” and is intended to refer to a bifunctional cyclopropyl group, e.g.,

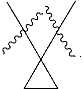 [SMILES: *C1(*)CC1]

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Aryl: The term “aryl” refers to monocyclic and bicyclic ring systems having a total of six to fourteen ring members (e.g., C₆-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

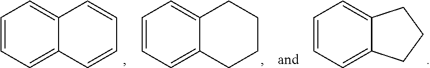

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₅₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated monocyclic or polycyclic ring system of about 3 to about 10 ring carbon atoms. Exemplary monocyclic cycloalkyl rings include cyclopropyl, cyclobutyl, cyclopentyl, cyclohexyl, and cycloheptyl.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 10 ring atoms (e.g., 5- to 6-membered monocyclic heteroaryl or 9- to 10-membered bicyclic heteroaryl); having 6, 10, or 14 π-electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. The term “heteroaryl” may be used interchangeably with the terms “heteroaryl ring”, “heteroaryl group”, or “heteroaromatic”, any of which terms include rings that are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 6- to 10-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, and tetrahydroquinolinyl. A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 6- to 11-membered bridged heterocyclic ring having one, two, or three bridging atoms).

Modulator: The term “modulator,” as used herein, refers to a compound (e.g., a small molecule) that can alter the activity of another molecule (e.g., a protein). For example, in some embodiments, a modulator can cause an increase or decrease in the magnitude of a certain activity of a type of molecule as compared to the magnitude of the activity in the absence of the modulator. For example, a modulator can be an agonist or an antagonist of a particular target, as those terms are defined herein. For example, in some embodiments, a modulator is an agonist. In some embodiments, a modulator is an antagonist.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure refers

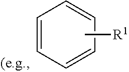

to at least

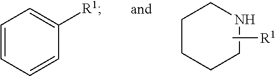

refers to at least

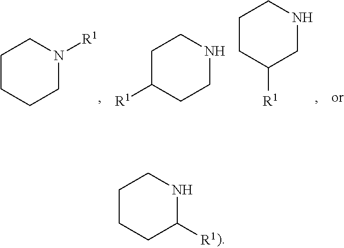

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}, —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘}S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (KD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid.

In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent.

Those of ordinary skill in the art, reading the present disclosure, will appreciate that certain small molecule compounds described herein may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more steroisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms. Additionally or alternatively, the symbol [symbol] refers to a point of attachment ring in a spirocyclic manner.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Estrogen Receptor Modulators

In some embodiments, the present disclosure provides compounds that are estrogen receptor modulators. In some embodiments, provided compounds are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by having (i) at least 80% increase in the E2-normalized signal in the AP assay (agonist mode) of Example 139 and (ii) no more than 80% reduction in the E2-normalized signal in the AP assay (antagonist mode) of Example 139. In some embodiments, provided compounds are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 139 and (ii) between 10% and 80% reduction in the E2-normalized signal in the AP assay (antagonist mode) of Example 139; or
2. (i) a pIC₅₀ greater than 6.4 and at least a 10% reduction in the E2-normalized signal in the AP assay (antagonist mode) of Example 139; and (ii) no more than 10% increase in E2-normalized signal in the AP assay (agonist mode) of Example 139; or
3. (i) a pIC₅₀ greater than 7.5 and at least a 10% reduction in the E2-normalized signal in the AP assay (antagonist mode) of Example 139; and (ii) no more than 10% increase in E2-normalized signal in the AP assay (agonist mode) of Example 139.

In some embodiments, the present disclosure provides compounds that are complete estrogen receptor (ER) antagonists. As used herein, a “complete estrogen receptor antagonist” refers to a compound or composition that produces an antagonistic effect when contacting the estrogen receptor of a subject or biological sample, with minimal agonistic effect (e.g., with no or substantially no agonistic effect). Complete estrogen receptor antagonism is determined according to methods described herein, for example in Example 139. In some embodiments, a complete estrogen receptor antagonist is characterized by having (i) a pIC₅₀ greater than 6.4 and at least a 10% reduction in the E2-normalized signal in the AP assay (antagonist mode) of Example 139; and (ii) no more than 10% increase in E2-normalized signal in the AP assay (agonist mode) of Example 139. In some embodiments, a complete estrogen receptor antagonist is characterized by having (i) a pIC₅₀ greater than 7.5 and at least a 10% reduction in the E2-normalized signal in the AP assay (antagonist mode) of Example 139; and (ii) no more than 10% increase in E2-normalized signal in the AP assay (agonist mode) of Example 139. In some embodiments, a complete estrogen receptor antagonist is an agent (e.g., a small molecule compound) that shows ER antagonism and no or substantially no ER agonism in one or more of ERα protein level assays, MCF-7 cell line assays, Ishikawa cell line assays (measuring wild type ER and certain mutants including mutants lacking AF1 and/or AF2 domains), and rodent uterine weight gain assays. See, generally, WO 2017/059139. Alternatively or additionally, in some embodiments, a complete estrogen receptor antagonist has three characteristics: it (1) inhibits both activating function 1 (AF1) and activating function 2 (AF2), as complete anti-estrogen activity requires inactivation of both AF1 and AF2; (2) promotes ER degradation; and (3) lacks the partial ER agonist activity observed with certain other agents. Without being bound by theory, it is understood that complete inhibition of both AF1 and AF2 is required for complete estrogen receptor activity, activating mutations in the gene that codes for estrogen receptor 1 allows for activation of both AF1 and AF2 even in the absence of estrogen.

In some embodiments, the present disclosure provides a compound of Formula I:

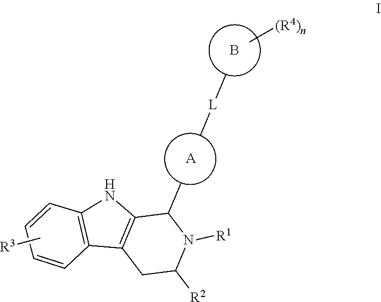

or a pharmaceutically acceptable salt thereof, wherein:
A is selected from optionally substituted 5-membered heteroaryl comprising 1-3 heteroatoms selected from N, O, and S, wherein at least 1 heteroatom is S or O, optionally substituted 7- to 10-membered bicyclic heteroaryl comprising 1-4 heteroatoms selected from N, O, and S, and optionally substituted 3- to 6-membered heterocyclyl comprising 1-3 heteroatoms selected from N, O, and S;
L is an optionally substituted bivalent group selected from —O—C₀-C₅ aliphatic- and —C₁-C₅ aliphatic-O—;
B is selected from 3- to 12-membered heterocyclyl comprising 1-3 heteroatoms selected from N, O, and S and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)OR^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)OR^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group;
each R⁴ is independently halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and
n is 0 to 5.

As defined generally above, A is selected from optionally substituted 5-membered heteroaryl comprising 1-3 heteroatoms selected from N, O, and S, wherein at least 1 heteroatom is S or O, optionally substituted 7- to 10-membered bicyclic heteroaryl comprising 1-4 heteroatoms selected from N, O, and S, and optionally substituted 3- to 6-membered heterocyclyl comprising 1-3 heteroatoms selected from N, O, and S.

In some embodiments, A is optionally substituted 5-membered heteroaryl comprising 1-3 heteroatoms selected from N, O, and S, wherein at least 1 heteroatom is S or O, or optionally substituted 7- to 10-membered bicyclic heteroaryl comprising 1-4 heteroatoms selected from N, O, and S.

In some embodiments, A is optionally substituted 5-membered heteroaryl comprising 1-3 heteroatoms selected from N, O, and S, wherein at least 1 heteroatom is S or O. In some embodiments, A is optionally substituted 5-membered heteroaryl comprising 1-3 heteroatoms selected from N, O, and S, and wherein at least 1 heteroatom is S. In some embodiments, A is 5-membered heteroaryl comprising 1-3 heteroatoms selected from N, O, and S, wherein at least 1 heteroatom is S or O, optionally substituted with halogen, —(CH₂)₀₋₄R^{∘}, or —(CH₂)₀₋₄OR^{∘}. In some embodiments, A is 5-membered heteroaryl comprising 1-3 heteroatoms selected from N, O, and S, wherein at least 1 heteroatom is S or O, optionally substituted with halogen or C₁-C₆ aliphatic. In some embodiments, A is optionally substituted thiophenyl or optionally substituted thiazolyl. In some embodiments, A is unsubstituted thiophenyl or thiazolyl. In some embodiments, A is thiophenyl or thiazolyl optionally substituted with halogen, —(CH₂)₀₋₄R^{∘}, or —(CH₂)₀₋₄OR^{∘}. In some embodiments, A is thiophenyl or thiazolyl optionally substituted with halogen. In some embodiment, A is optionally substituted thiophenyl. In some embodiments, A is unsubstituted thiophenyl. In some embodiments, A is thiophenyl optionally substituted with halogen, —(CH₂)₀₋₄R^{∘}, or —(CH₂)₀₋₄OR^{∘}. In some embodiments, A is thiophenyl optionally substituted with halogen. In some embodiments, A is optionally substituted thiazolyl. In some embodiments, A is unsubstituted thiazolyl. In some embodiments, A is thiazolyl substituted with halogen, —(CH₂)₀₋₄R^{∘}, or —(CH₂)₀₋₄OR^{∘}. In some embodiments, A is thiazolyl substituted with halogen.

In some embodiments, A is selected from:

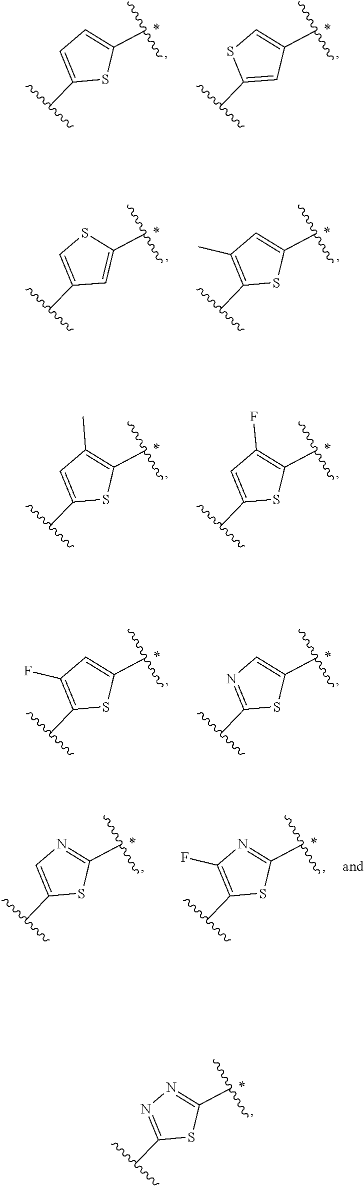

wherein * represents a point of attachment to moiety L.

In some embodiments, A is selected from:

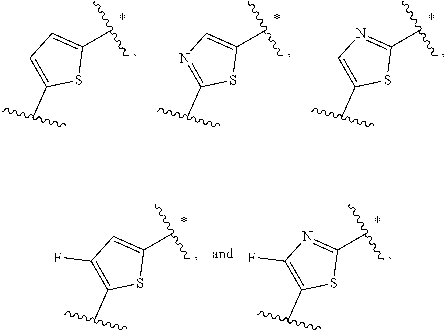 [SMILES: *c1ccc(*)s1 ; *c1cnc(*)s1 ; *c1cnc(*)s1 ; *c1cc(F)c(*)s1 ; *c1nc(F)c(*)s1]

wherein * represents a point of attachment to moiety L. In some embodiments, A is

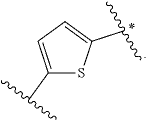 [SMILES: *c1ccc(*)s1]

In some embodiments, A is

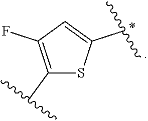 [SMILES: *c1cc(F)c(*)s1]

In some embodiments, A is

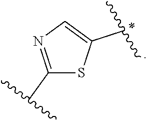 [SMILES: *c1cnc(*)s1]

In some embodiments, A is

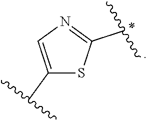 [SMILES: *c1cnc(*)s1]

In some embodiments, A is optionally substituted 7- to 10-membered bicyclic heteroaryl comprising 1-4 heteroatoms selected from N, O, and S. In some embodiments, A is 7- to 10-membered bicyclic heteroaryl comprising 1-4 heteroatoms selected from N, O, and S optionally substituted with halogen, —(CH₂)₀₋₄R^{∘}, or —(CH₂)₀₋₄OR^{∘}. In some embodiments, A is 7- to 10-membered bicyclic heteroaryl comprising 1-4 heteroatoms selected from N, O, and S, optionally substituted with halogen or C₁-C₆ aliphatic. In some embodiments, A is optionally substituted 7-membered bicyclic heteroaryl comprising 1-4 heteroatoms selected from N, O, and S. In some embodiments, A is optionally substituted 8-membered bicyclic heteroaryl comprising 1-4 heteroatoms selected from N, O, and S. In some embodiments, A is optionally substituted 9-membered bicyclic heteroaryl comprising 1-4 heteroatoms selected from N, O, and S. In some embodiments, A is optionally substituted 10-membered bicyclic heteroaryl comprising 1-4 heteroatoms selected from N, O, and S. In some embodiments, A is optionally substituted thienopyrrolyl, thienopyrazolyl, thienoimidazolyl, pyrroloisothiazolyl, or pyrrolothiazolyl.

In some embodiments, A is

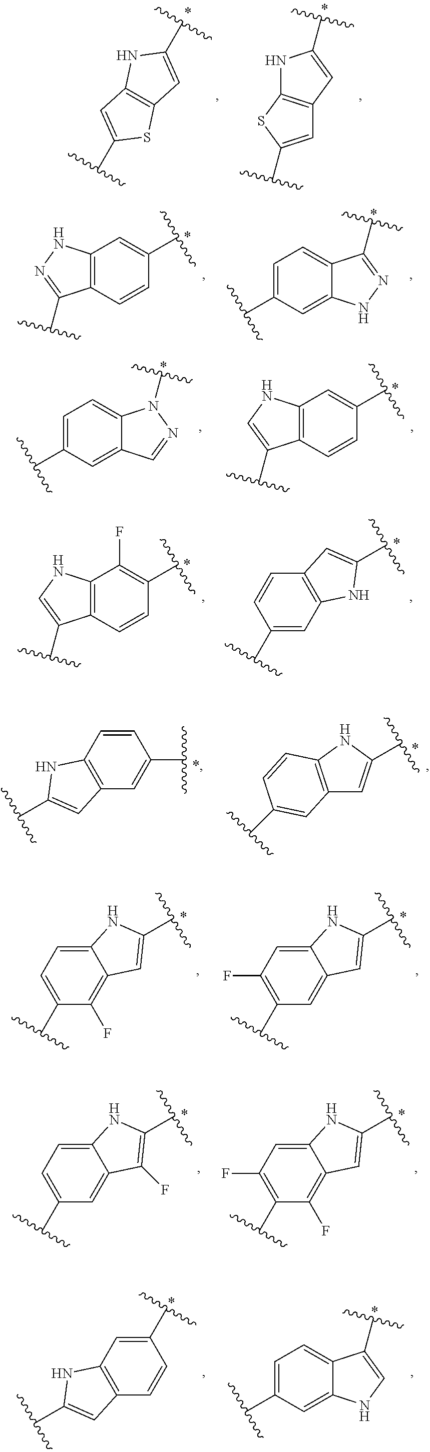

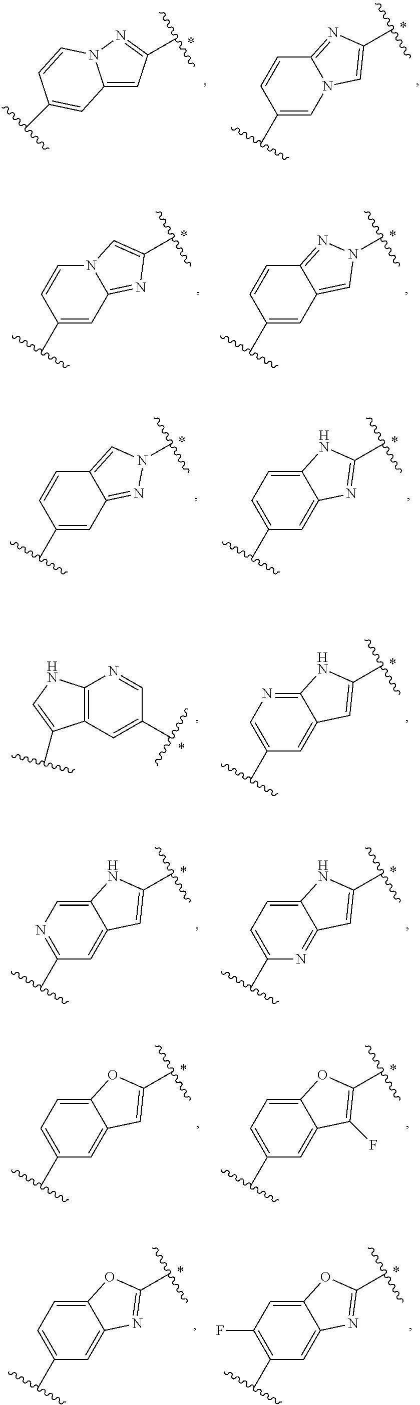

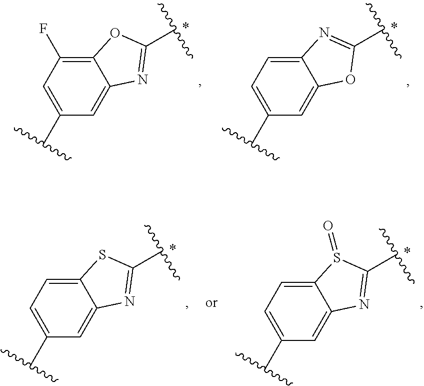 [SMILES: *c1cc(F)c2oc(*)nc2c1 ; *c1ccc2nc(*)oc2c1 ; *c1ccc2sc(*)nc2c1 ; *C1=Nc2cc(*)ccc2S1=O]

wherein * represents a point of attachment to moiety L.

In some embodiments, A is optionally substituted 3- to 6-membered heterocyclyl comprising 1-3 heteroatoms selected from N, O, and S. In some embodiments, A is optionally substituted 3-membered heterocylyl comprising 1 heteroatom selected from N, O, and S. In some embodiments, A is optionally substituted 4-membered heterocyclyl comprising 1-2 heteroatoms selected from N, O and S. In some embodiments, A is optionally substituted 5-membered heterocyclyl comprising 1-3 heteroatoms selected from N, O and S. In some embodiments, A is optionally substituted 6-membered heterocyclyl comprising 1-3 heteroatoms selected from N, O and S. In some embodiments, A is optionally substituted piperidinyl. In some embodiments, A is

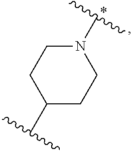 [SMILES: *C1CCN(*)CC1]

wherein * represents a point of attachment to moiety L.

In some embodiments, A is selected from:

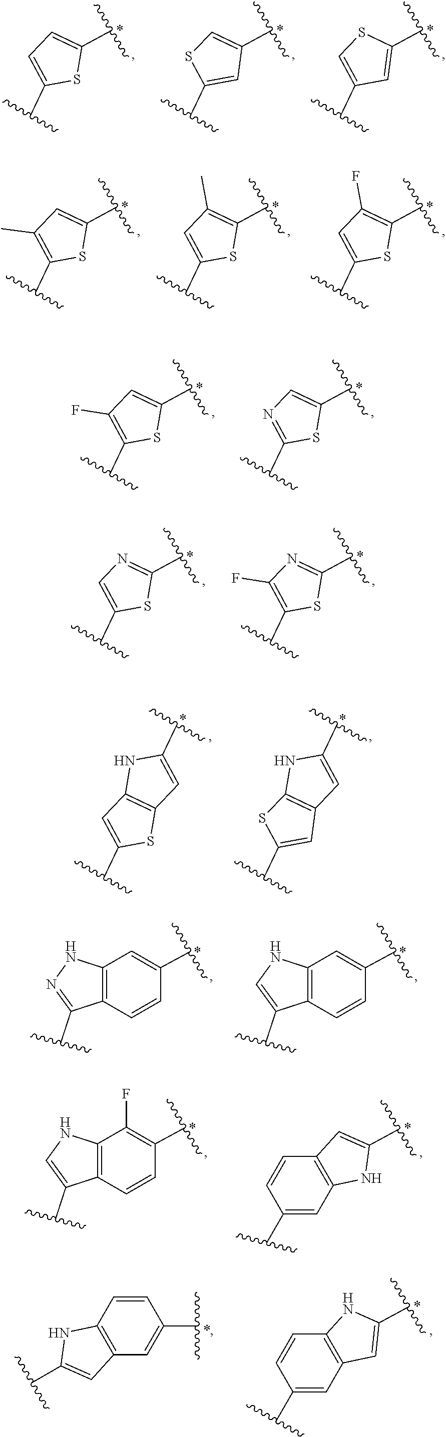

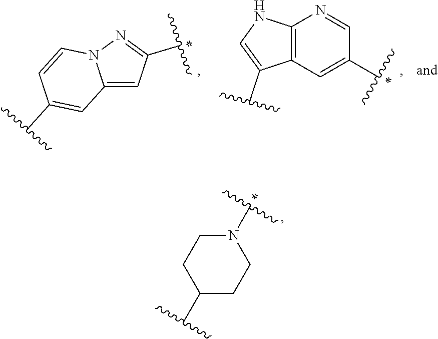 [SMILES: *c1ccn2nc(*)cc2c1 ; *c1cnc2[nH]cc(*)c2c1 ; *C1CCN(*)CC1]

wherein * represents a point of attachment to moiety L.

As described generally above, L is an optionally substituted bivalent group selected from —O—C₀-C₅ aliphatic- and —C₁-C₅ aliphatic-O—. It will be appreciated that the point of attachment of L is indicated by the direction in which its definition is written, such that the leftmost atom is attached to A and the rightmost atom is attached to B. For example, when L is —O—CH₂—, L is attached to A via the oxygen atom and to B via the carbon atom.

In some embodiments, L is optionally substituted —O—C₀-C₅ aliphatic-. In some embodiments, L is —O—. In some embodiments, L is optionally substituted —O—C₁-C₅ aliphatic-. In some embodiments, L is —C₁-C₅ aliphatic-O—.

In some embodiments, L is selected from —O—,

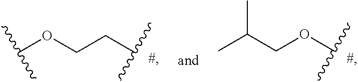

wherein # represents a point of attachment to moiety B.

As described generally above, B is selected from 3- to 12-membered heterocyclyl comprising 1-3 heteroatoms selected from N, O, and S, and C₃-C₆ cycloaliphatic.

In some embodiments, B is selected from 3- to 6-membered heterocyclyl comprising 1-3 heteroatoms selected from N, O, and S and C₃₋₆ cycloaliphatic. In some embodiments, B is selected from 4- to 6-membered heterocyclyl comprising 1-3 heteroatoms selected from N, O, and S and C₃₋₆ cycloaliphatic.

In some embodiments, B is 3- to 7-membered heterocyclyl comprising 1-3 heteroatoms selected from N, O, and S. In some embodiments, B is 4- to 5-membered heterocyclyl comprising 1-3 heteroatoms selected from N, O, and S. In some embodiments, B is 3-membered heterocyclyl comprising 1 heteroatom selected from N, O, and S. In some embodiments, B is 4-membered heterocyclyl comprising 1-2 heteroatoms selected from N, O, and S. In some embodiments, B is 5-membered heterocyclyl comprising 1-3 heteroatoms selected from N, O, and S. In some embodiments, B is 6-membered heterocyclyl comprising 1-3 heteroatoms selected from N, O, and S. In some embodiments, B is a 6-membered monocyclic heterocyclyl comprising 1-3 heteroatoms selected from N, O, and S. In some embodiments, B is a 6-membered bridged bicyclic heterocyclyl comprising 1-3 heteroatoms selected from N, O, and S. In some embodiments, B is 7-membered heterocyclyl comprising 1-3 heteroatoms selected from N, O, and S. In some embodiments, B is 7-membered monocyclic heterocyclyl comprising 1-3 heteroatoms selected from N, O, and S. In some embodiments, B is 7-membered spirocyclic fused bicyclic heterocyclyl comprising 1-3 heteroatoms selected from N, O, and S. In some embodiments, B is azetidinyl, pyrrolidinyl, isoxazolidinyl, piperidinyl, 2-azabicyclo[2.1.1]hexanyl, or 5-azaspiro[2.4]heptanyl. In some embodiments, B is azetidinyl or pyrrolidinyl. In some embodiments, B is azetidinyl. In some embodiments, B is pyrrolidinyl. In some embodiments, B is selected from:

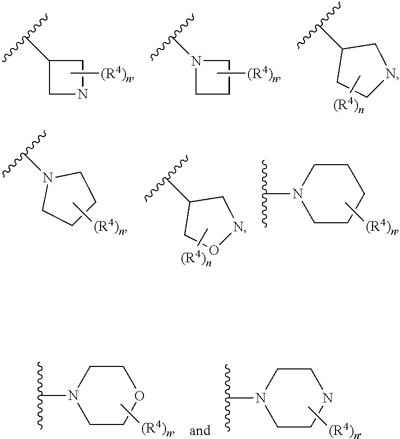

In some embodiments, B is 6- to 12-membered bicyclic fused or spirocyclic heterocyclyl. In some embodiments, B is 6- to 8-membered bicyclic fused or spirocyclic heterocyclyl. In some embodiments, B is 6- to 12-membered bicyclic fused heterocyclyl. In some embodiments, B is 6- to 8-membered bicyclic fused heterocyclyl. In some embodiments, B is 6- to 12-membered bicyclic spirocyclic heterocyclyl. In some embodiments, B is 6- to 8-membered bicyclic spirocyclic heterocyclyl. In some embodiments, B is selected from:

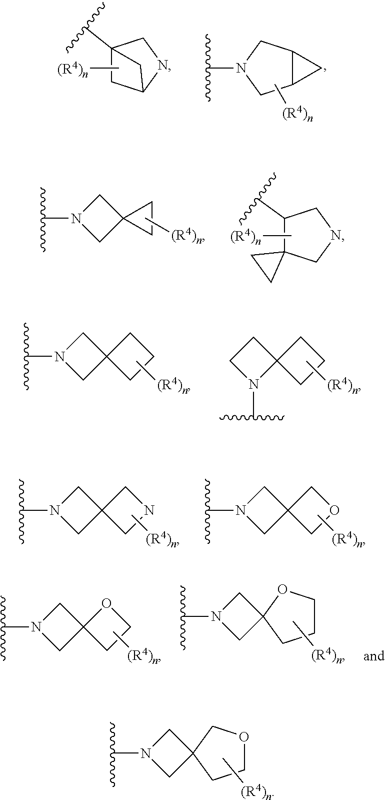

In some embodiments, B is C₃-C₆ cycloaliphatic. In some embodiments, B is cyclopropyl, cyclobutyl, cyclopentyl, or cyclohexyl.

As described generally above, each R⁴ is independently oxo, halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S.

In some embodiments, each R⁴ is independently halogen or optionally substituted C₁₋₆ aliphatic.

In some embodiments, R⁴ is oxo.

In some embodiments, R⁴ is halogen. In some embodiments, R⁴ is flouro.

In some embodiments, R⁴ is —CN.

In some embodiments, R⁴ is —OR^{a}. In some embodiments, R⁴ is —OH. In some embodiments, R⁴ is —O—C₁-C₆ aliphatic.

In some embodiments, R⁴ is —N(R^{a})₂. In some embodiments, R⁴ is —N(H)(R^{a}). In some embodiments, R⁴ is —NH₂. In some embodiments, R⁴ is —N(H)C₁-C₆ aliphatic. In some embodiments, R⁴ is —N(C₁-C₆ aliphatic)₂. In some embodiments, R⁴ is —N(H)CH₃, —N(H)CH₂CH₃, —N(CH₂CH₃)₂, —N(H)CH₂CH₂CH₃, —N(CH₃)₂, —N(CH₃)CH₂CH₃, or —N(CH₃)CH₂CH₂CH₃.

In some embodiments, R⁴ is —C(O)R^{a}. In some embodiments, R⁴ is —C(O)—C₁-C₆ aliphatic optionally substituted with —(CH₂)₀₋₄OR^{∘}. In some embodiments, R⁴ is —C(O)CH₃, —C(O)CH₂OCH₃, or —C(O)CH₂CH₂OCH₃.

In some embodiments, R⁴ is —OC(O)R^{a}. In some embodiments, R⁴ is —OC(O)—C₁-C₆ aliphatic.

In some embodiments, R⁴ is —C(O)₂R^{a}. In some embodiments, R⁴ is —C(O)OH. In some embodiments, R⁴ is —C(O)₂—C₁-C₆ aliphatic.

In some embodiments, R⁴ is —C(O)N(R^{a})₂. In some embodiments, R⁴ is —C(O)N(H)R^{a}. In some embodiments, R⁴ is —C(O) NH₂. In some embodiments, R⁴ is —C(O)N(H)C₁-C₆ aliphatic.

In some embodiments, R⁴ is —N(R^{a})C(O)R^{a}. In some embodiments, R⁴ is —N(H)C(O)R^{a}. In some embodiments, R⁴ is —N(H)C(O)C₁-C₆ aliphatic optionally substituted with —(CH₂)₀₋₄OR^{∘}. In some embodiments, R⁴ is —N(H)C(O)CH₂OH or —N(H)C(O)CH₂OCH₃.

In some embodiments, R⁴ is an optionally substituted C₁-C₆ aliphatic. In some embodiments, R⁴ is C₁-C₆ aliphatic optionally substituted with halogen, —(CH₂)₀₋₄R^{∘}, —(CH₂)₀₋₄ OR^{∘}, —O(CH₂)₀₋₄R^{∘}, —CN, —(CH₂)₀₋₄N(R^{∘})₂, or phenyl. In some embodiments, R⁴ is C₁-C₆ aliphatic substituted with halogen, —OH, —OCH₃, —CN, or 3- to 6-membered saturated, partially unsaturated, or aryl ring having 0-4 heteroatoms independently selected from nitrogen, oxygen, or sulfur. In some embodiments, R⁴ is C₁-C₆ aliphatic optionally substituted with halogen. In some embodiments, R⁴ is C₁-C₃ alkyl optionally substituted with halogen. In some embodiments, R⁴ is methyl, ethyl, propyl, butyl, pentyl, hexyl, —CH₂F, —CH₂CH₂F, —CH₂CHF₂, —CH₂CH₂CHF₂, —CH₂CH₂CF₃, —CH₂CH₂CH₂OH, —CH₂CH₂CH₂F, —CH₂CH₂CH(CH₃)₂, —CH₂CH₂-Ph, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃,

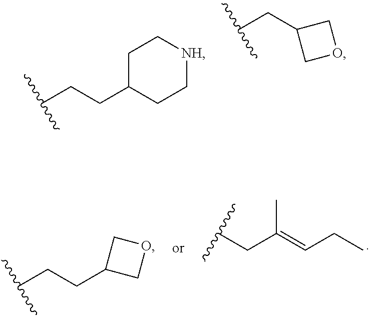

In some embodiments, R⁴ is an optionally substituted 3- to 12-membered heterocyclyl comprising 1 to 3 heteroatoms selected from N, O, and S. In some embodiments, R⁴ is optionally substituted 3- to 6-membered heterocyclyl comprising 1 to 3 heteroatoms selected from N, O, and S. In some embodiments, R⁴ is optionally substituted azetidinyl.

In some embodiments, R⁴ is oxo, —OH, —OCH₃, fluoro, methyl, ethyl, propyl, isopropyl, butyl, pentyl, hexyl, —CH₂F, —CH₂CH₂F, —CH₂CHF₂, —CH₂CH₂CHF₂, —CH₂CH₂CF₃, —CH₂CH₂CH₂OH, —CH₂CH₂CH₂F, —CH₂CH₂CH(CH₃)₂, —CH₂CH₂-Ph, —C(CH₃)₂—OH, —CH₂OCH₃, —CH₂CH₂OCH₃, —CH₂CH₂OCH₂CH₃, —CH₂CH₂CH₂OCH₂CH₃, —CH₂C≡CH, —CH₂C≡CHCH₃, —CH₂CN, —CH₂CH₂CN, —CH₂CH₂CH₂OCF₃, —NHCH₂CH₃, —NHCH₂CH₂CH₃, —NHCH₂CH₂CH₃, —NHCH₃, —N(CH₃)CH₂CH₃, —N(CH₃)CH₂CH₂CH₃, —N(CH₃)₂, —NHC(O)CH₂OCH₃, —NHC(O)CH₂CH₂OCH₃, —NHC(O)CH₂OH, —C(O)CF₃,

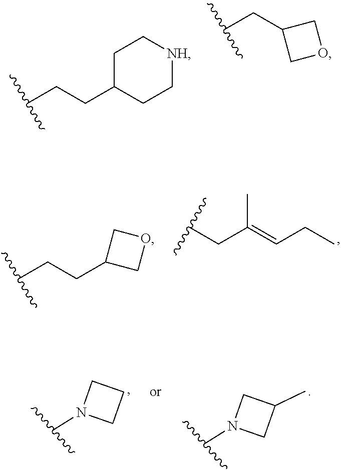

In some embodiments, each R⁴ is independently selected from fluoro, methyl, ethyl, propyl, —CH₂CH₂F, —CH₂CH₂CHF₂, and —CH₂CH₂CH₂F. In some embodiments, each R⁴ is independently selected from propyl, —CH₂CH₂CHF₂, and —CH₂CH₂CH₂F. In some embodiments, each R⁴ is independently selected from fluoro and —CH₂CH₂CH₂F.

In some embodiments, a moiety:

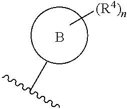

is a moiety selected from:

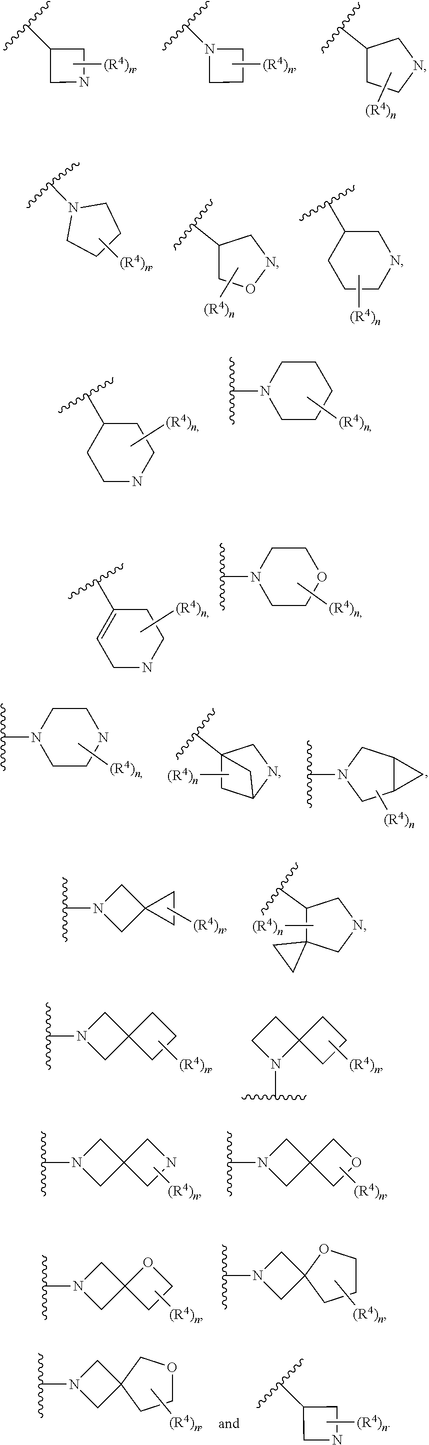

In some embodiments, a moiety:

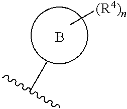

is a moiety selected from:

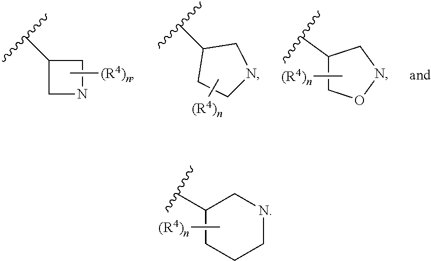

In some embodiments, a moiety:

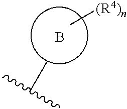

is a moiety selected from:

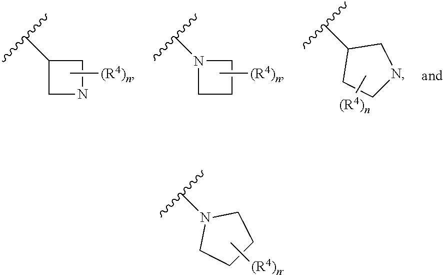

In some embodiments, a moiety:

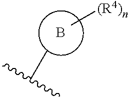

is a moiety selected from:

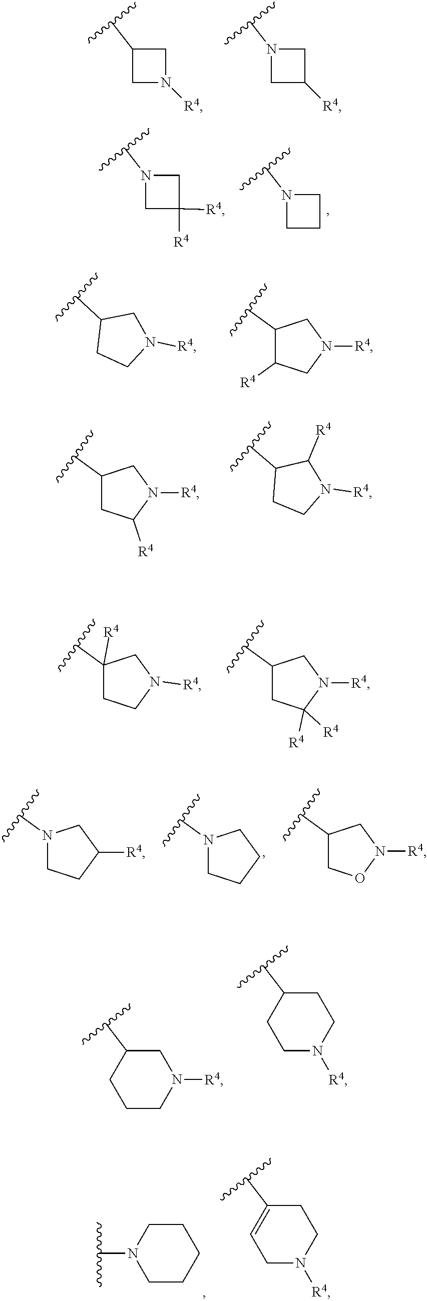

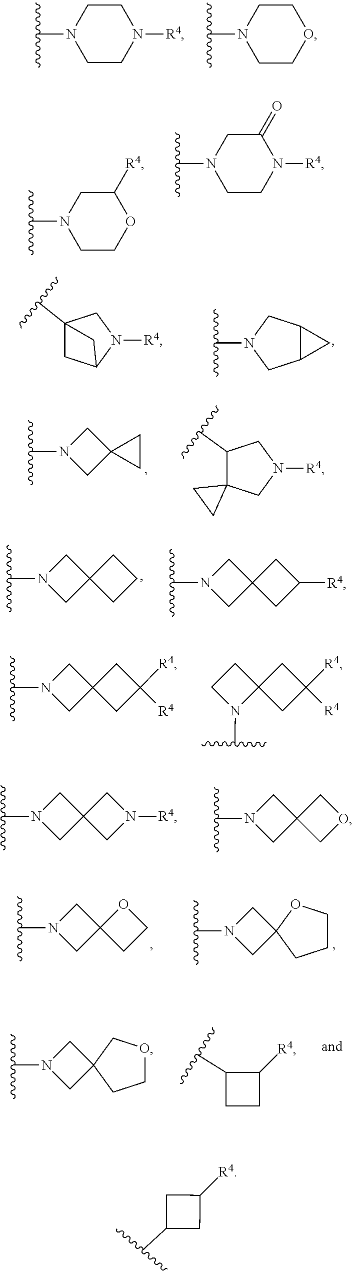

In some embodiments, a moiety:

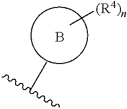

is a moiety selected from:

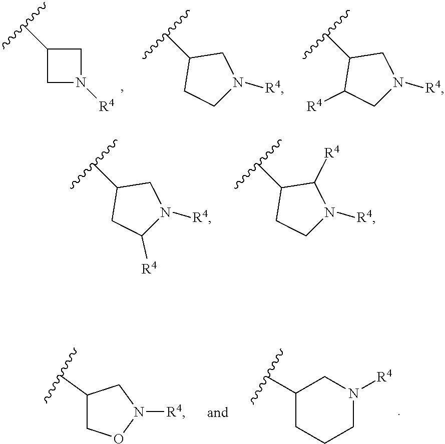

In some embodiments, a moiety:

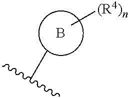

is a moiety selected from:

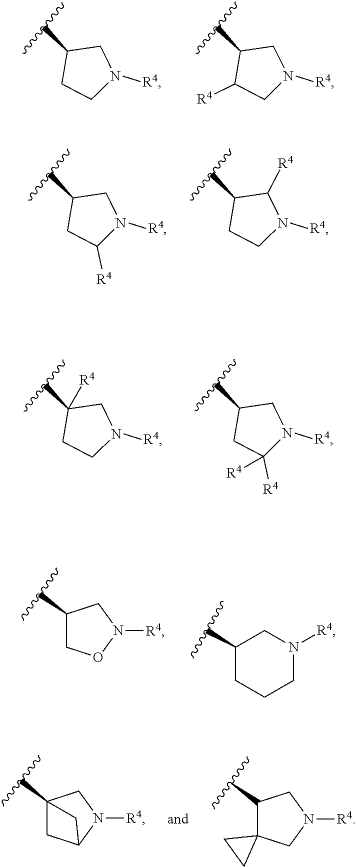

In some embodiments, a moiety:

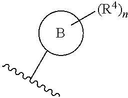

is a moiety selected from:

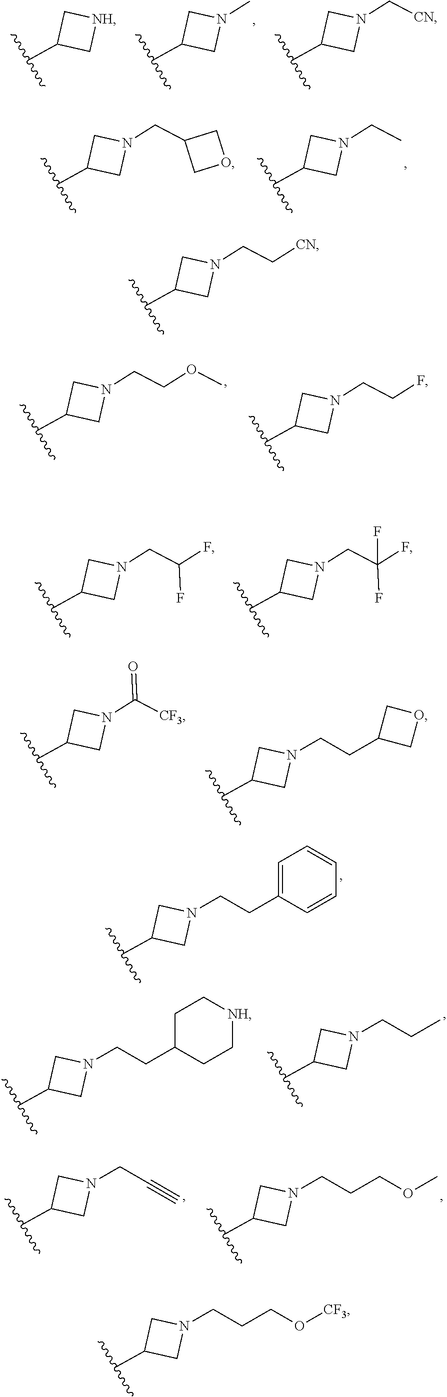

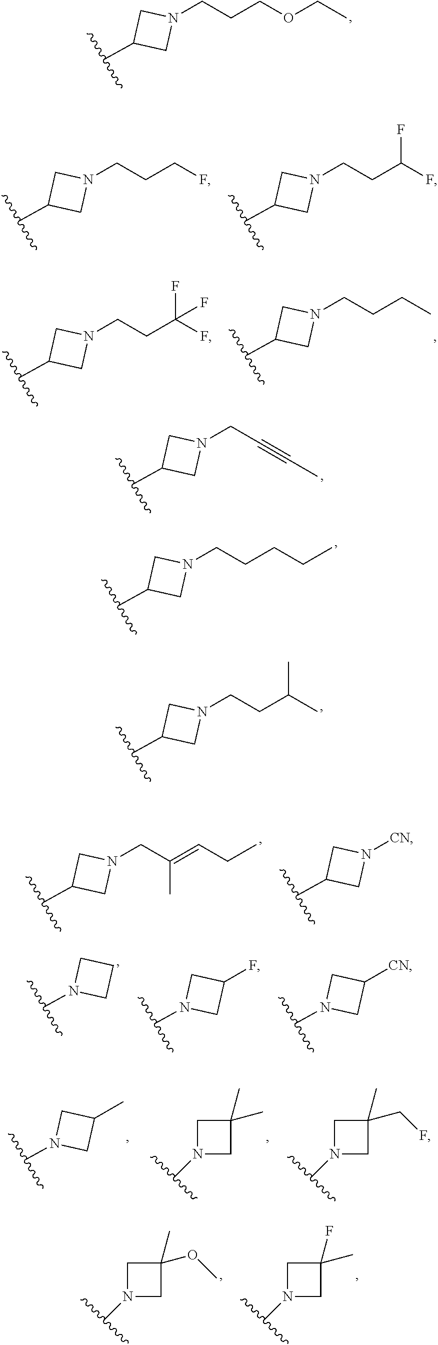

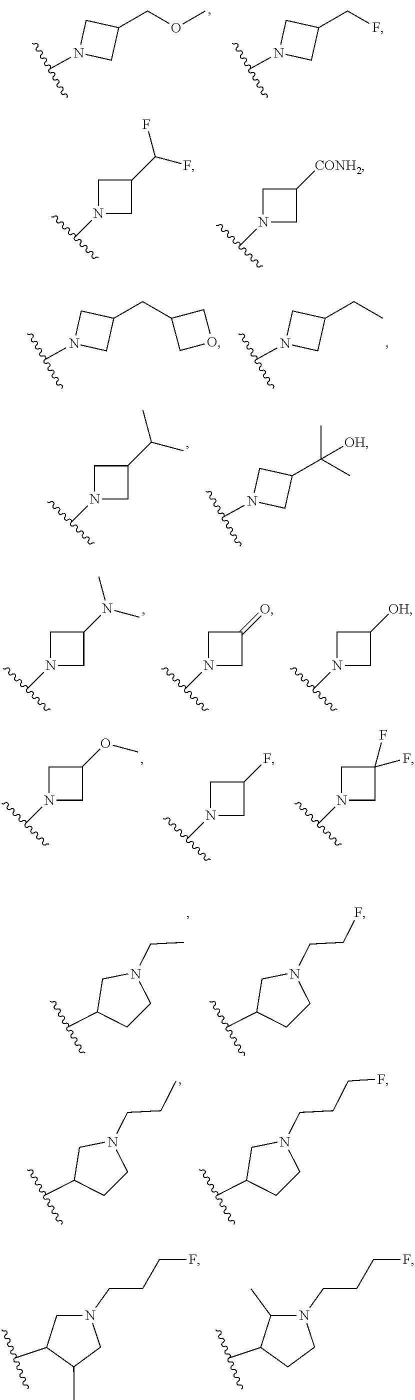

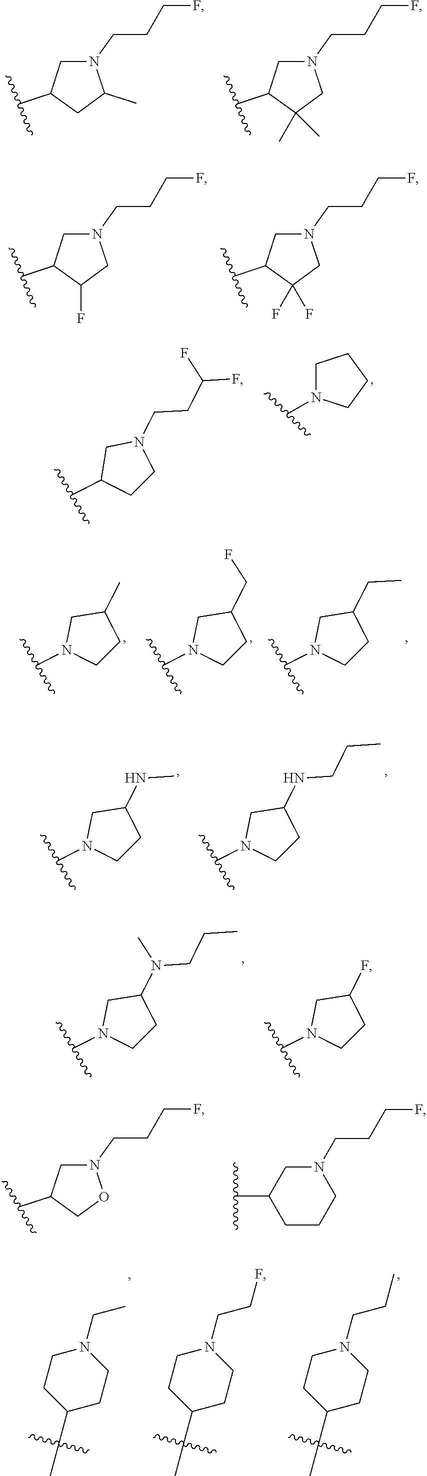

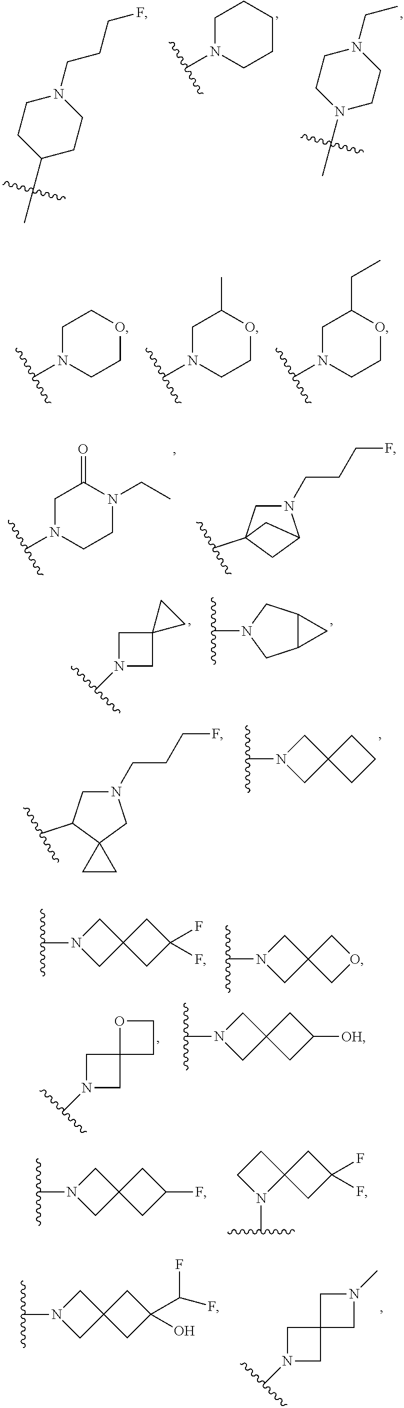

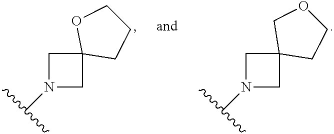 [SMILES: *N1CC2(CCCO2)C1 ; *N1CC2(CCOC2)C1]

In some embodiments, a moiety:

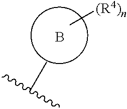

is a moiety selected from:

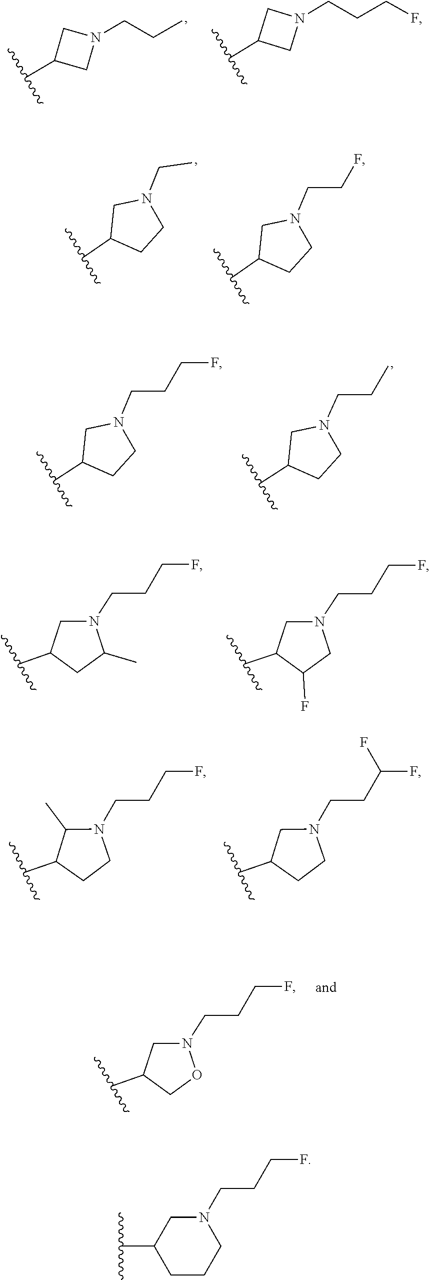

In some embodiments, a moiety:

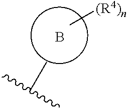

is a moiety selected from:

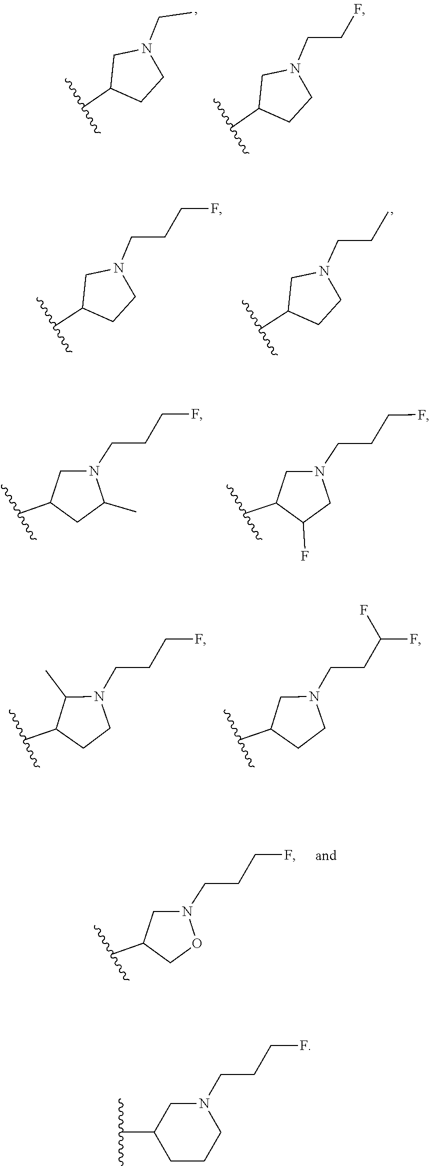

In some embodiments, a moiety:

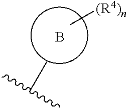

is a moiety selected from:

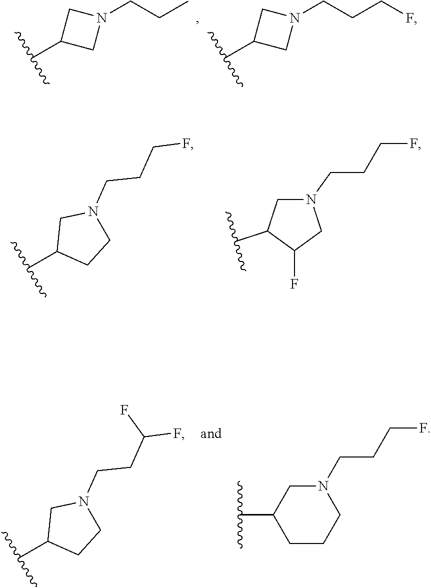

In some embodiments, a moiety:

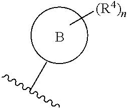

is a moiety selected from:

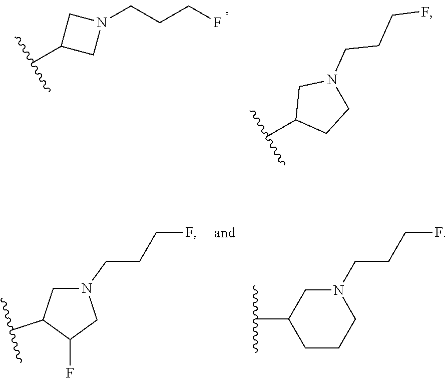 [SMILES: *C1CN(CCCF)C1 ; *C1CCN(CCCF)C1 ; *C1CN(CCCF)CC1F ; *C1CCCN(CCCF)C1]

As described generally above, R¹ is selected from hydrogen and optionally substituted C₁-C₆ aliphatic. In some embodiments, R¹ is hydrogen. In some embodiments, R¹ is C₁-C₆ aliphatic optionally substituted with halogen; —(CH₂)₀₋₄R^{∘}, —(CH₂)₀₋₄OR^{∘}, or —(CH₂)₀₋₄Ph. In some embodiments, R¹ is C₁-C₆ aliphatic optionally substituted with halogen or —OH.

In some embodiments, R¹ is selected from:

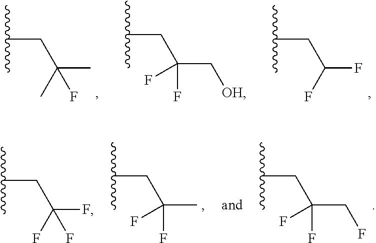

In some embodiments, R¹ is selected from:

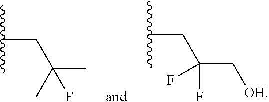

In some embodiments, R¹ is selected from:

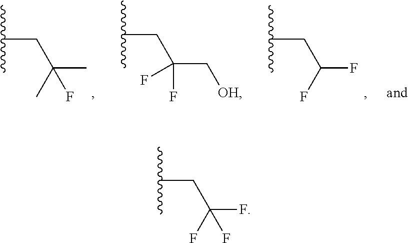

In some embodiments, R¹ is selected from:

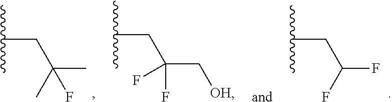

As described generally above, R² is selected from hydrogen and optionally substituted C₁-C₆ aliphatic. In some embodiments, R² is hydrogen. In some embodiments, R² is C₁-C₆ aliphatic. In some embodiments, R² is methyl, ethyl, propyl, butyl, pentyl, or hexyl. In some embodiments, R² is methyl.

As described generally above, R³ is selected from hydrogen, halogen, —CN, —OR^{a}, —C(O)R^{a}, —C(O)₂R^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)₂R^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group. In some embodiments, R³ is hydrogen.

As described generally above, n is 0-5. In some embodiments, n is 0. In some embodiments, n is 1, 2, 3, 4, or 5. In some embodiments, n is 1. In some embodiments, n is 2. In some embodiments, n is 3. In some embodiments, n is 4. In some embodiments, n is 5.

In some embodiments, the present disclosure provides a compound of Formula II

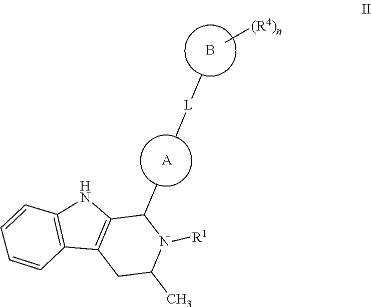

or a pharmaceutically acceptable salt thereof, wherein A, B, L, n, R¹, and R⁴ are as defined above for Formula I and described in classes and subclasses herein, both singly and in combination.

In some embodiments, the present disclosure provides a compound of Formula II-a:

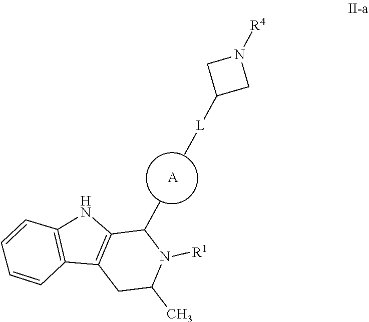

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-b:

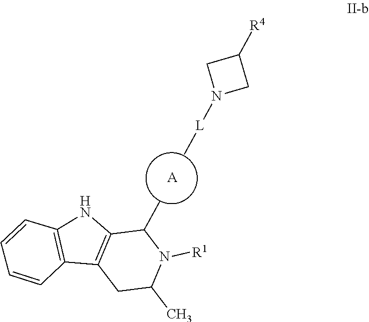

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-c:

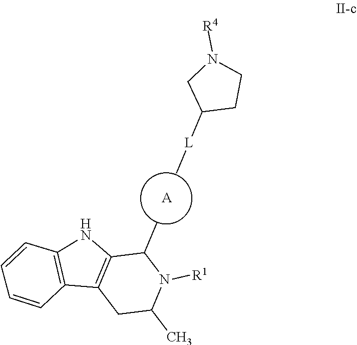

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-d:

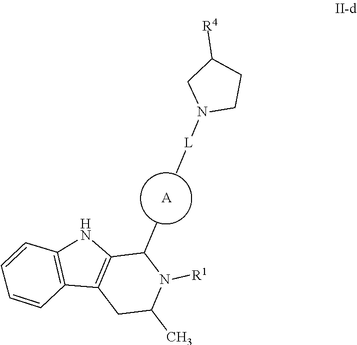

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-e:

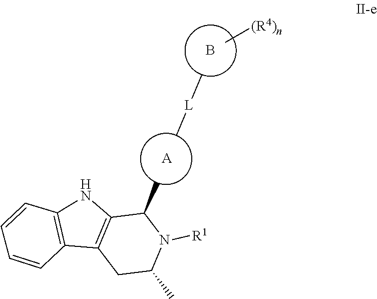

or a pharmaceutically acceptable salt thereof, wherein A, B, L, n, R¹, and R⁴ are as defined above for Formula I and described in classes and subclasses herein, both singly and in combination.

In some embodiments, the present disclosure provides a compound of Formula II-f:

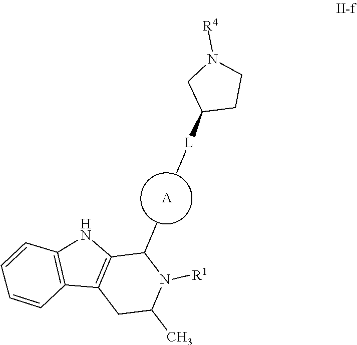

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-g:

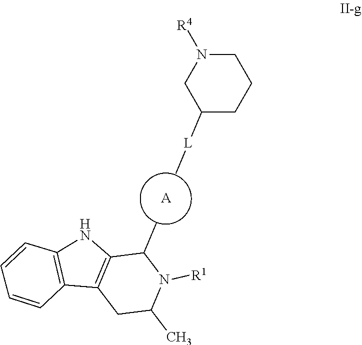

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula II-h:

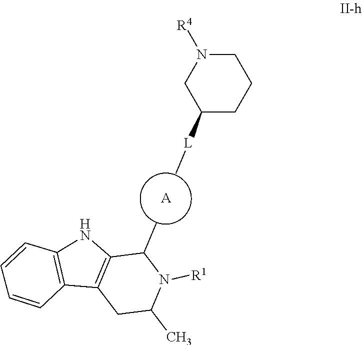

or a pharmaceutically acceptable salt thereof, wherein A, L, R¹, and R⁴ are as defined above for Formula I and described in classes and subclasses herein, both singly and in combination.

In some embodiments, the present disclosure provides a compound of Formula III:

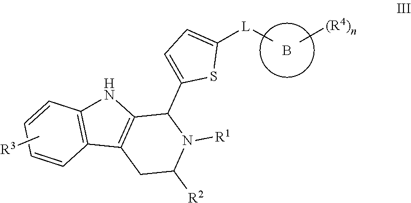

or a pharmaceutically acceptable salt thereof, wherein B, L, n, R¹, R², R³, and R⁴ are as defined above for Formula I and described in classes and subclasses herein, both singly and in combination.

In some embodiments, the present disclosure provides a compound of Formula IV:

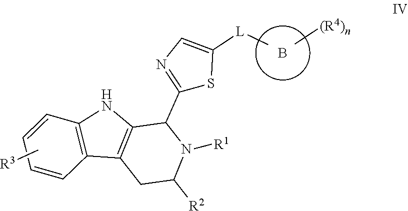

or a pharmaceutically acceptable salt thereof, wherein B, L, n, R¹, R², R³, and R⁴ are as defined above for Formula I and described in classes and subclasses herein, both singly and in combination.

In some embodiments, the present disclosure provides a compound of Formula V:

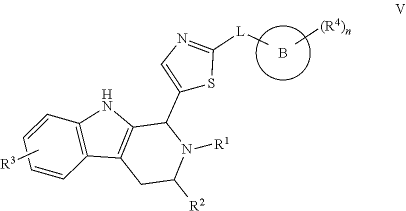

or a pharmaceutically acceptable salt thereof, wherein B, L, n, R¹, R², R³, and R⁴ are as defined above for Formula I and described in classes and subclasses herein, both singly and in combination.

In some embodiments, the present disclosure provides a compound of Formula VI:

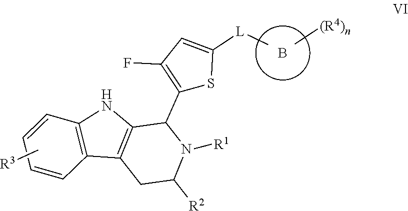

or a pharmaceutically acceptable salt thereof, wherein B, L, n, R¹, R², R³, and R⁴ are as defined above for Formula I and described in classes and subclasses herein, both singly and in combination.

In some embodiments, the present disclosure provides a compound of Formula VII:

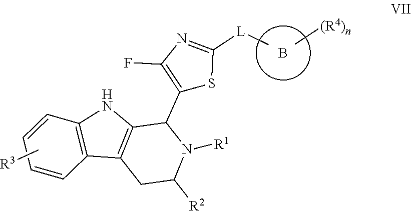

or a pharmaceutically acceptable salt thereof, wherein B, L, n, R¹, R², R³, and R⁴ are as defined above for Formula I and described in classes and subclasses herein, both singly and in combination.

In some embodiments, the present disclosure provides a compound of Formula VIII:

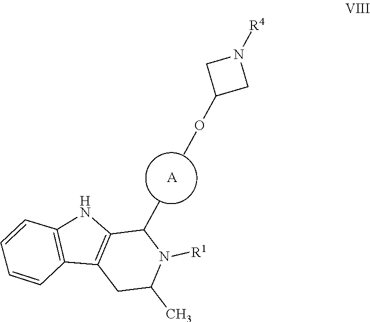

a pharmaceutically acceptable salt thereof, wherein A, R¹, and R⁴ are as defined above for Formula I and described in classes and subclasses herein, both singly and in combination.

In some embodiments, the present disclosure provides a compound of Formula IX:

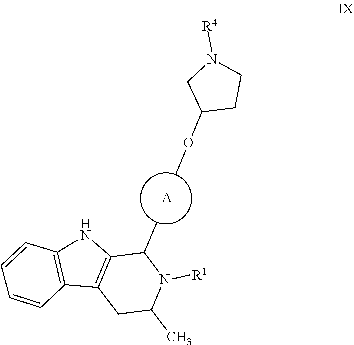

or a pharmaceutically acceptable salt thereof, wherein A, R¹, and R⁴ are as defined above for Formula I and described in classes and subclasses herein, both singly and in combination.

In some embodiments, the present disclosure provides a compound of Formula IX-a:

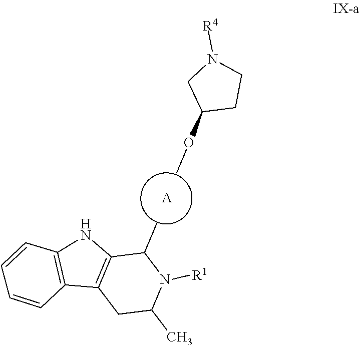

or a pharmaceutically acceptable salt thereof, wherein A, R¹, and R⁴ are as defined above for Formula I and described in classes and subclasses herein, both singly and in combination.

In some embodiments, the present disclosure provides a compound of Formula X:

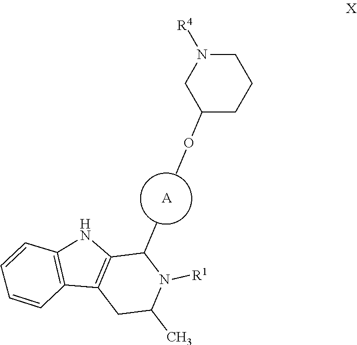

or a pharmaceutically acceptable salt thereof, wherein A, R¹, and R⁴ are as defined above for Formula I and described in classes and subclasses herein, both singly and in combination.

In some embodiments, the present disclosure provides a compound of Formula X-a:

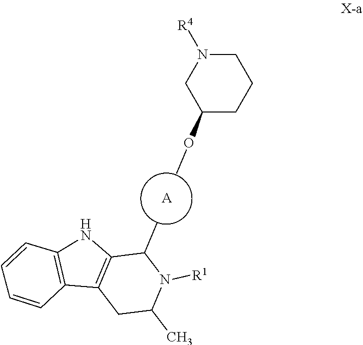

or a pharmaceutically acceptable salt thereof, wherein A, R¹, and R⁴ are as defined above for Formula I and described in classes and subclasses herein, both singly and in combination.

In some embodiments, the present disclosure provides a compound of Table 1.

TABLE 1

| | Com- |
|---|---|
| | pound |
| Structure | No. |
| 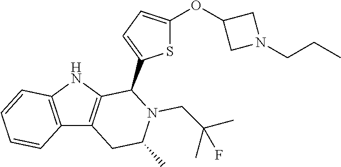 [SMILES: CCCN1CC(Oc2ccc([C@@H]3c4[nH]c5ccccc5c4C[C@@H](C)N3CC(C)(C)F)s2)C1] | I-1 |
| 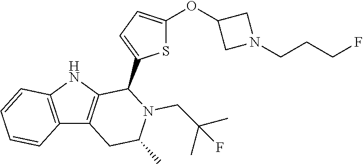 [SMILES: C[C@@H]1Cc2c([nH]c3ccccc23)[C@@H](c2ccc(OC3CN(CCCF)C3)s2)N1CC(C)(C)F] | I-2 |
| 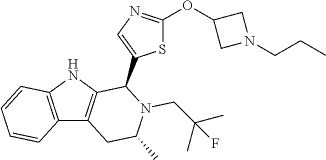 [SMILES: CCCN1CC(Oc2ncc([C@@H]3c4[nH]c5ccccc5c4C[C@@H](C)N3CC(C)(C)F)s2)C1] | I-3 |
| 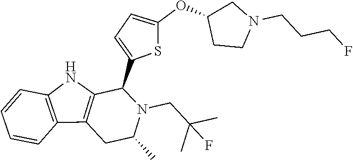 [SMILES: C[C@@H]1Cc2c([nH]c3ccccc23)[C@@H](c2ccc(O[C@H]3CCN(CCCF)C3)s2)N1CC(C)(C)F] | I-4 |
| 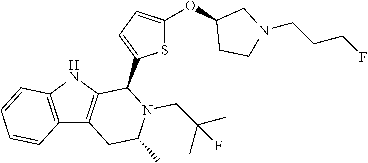 [SMILES: C[C@@H]1Cc2c([nH]c3ccccc23)[C@@H](c2ccc(O[C@@H]3CCN(CCCF)C3)s2)N1CC(C)(C)F] | I-5 |
| 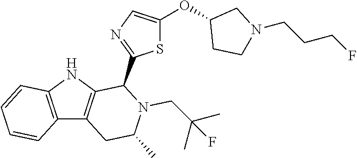 [SMILES: C[C@@H]1Cc2c([nH]c3ccccc23)[C@@H](c2ncc(O[C@H]3CCN(CCCF)C3)s2)N1CC(C)(C)F] | I-6 |
| 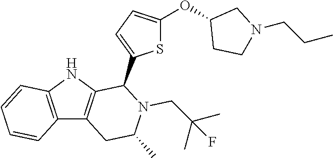 [SMILES: CCCN1CC[C@H](Oc2ccc([C@@H]3c4[nH]c5ccccc5c4C[C@@H](C)N3CC(C)(C)F)s2)C1] | I-7 |
| 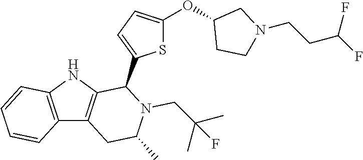 [SMILES: C[C@@H]1Cc2c([nH]c3ccccc23)[C@@H](c2ccc(O[C@H]3CCN(CCC(F)F)C3)s2)N1CC(C)(C)F] | I-8 |
| 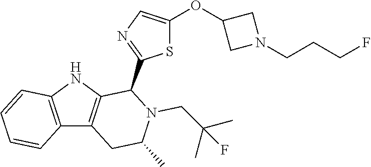 [SMILES: C[C@@H]1Cc2c([nH]c3ccccc23)[C@@H](c2ncc(OC3CN(CCCF)C3)s2)N1CC(C)(C)F] | I-9 |
| 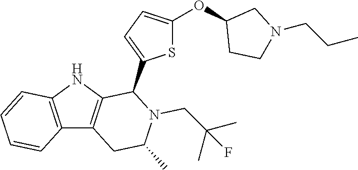 [SMILES: CCCN1CC[C@@H](Oc2ccc([C@@H]3c4[nH]c5ccccc5c4C[C@@H](C)N3CC(C)(C)F)s2)C1] | I-10 |
| 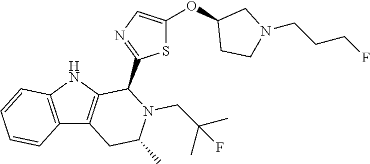 [SMILES: C[C@@H]1Cc2c([nH]c3ccccc23)[C@@H](c2ncc(O[C@@H]3CCN(CCCF)C3)s2)N1CC(C)(C)F] | I-11 |
| 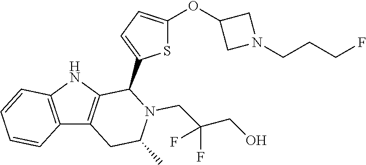 [SMILES: C[C@@H]1Cc2c([nH]c3ccccc23)[C@@H](c2ccc(OC3CN(CCCF)C3)s2)N1CC(F)(F)CO] | I-12 |
| 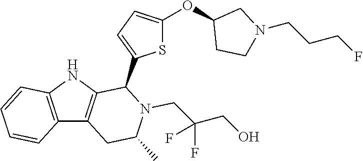 [SMILES: C[C@@H]1Cc2c([nH]c3ccccc23)[C@@H](c2ccc(O[C@@H]3CCN(CCCF)C3)s2)N1CC(F)(F)CO] | I-13 |
| 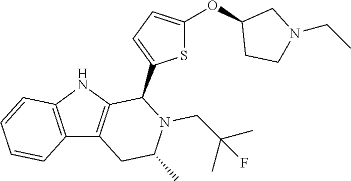 [SMILES: CCN1CC[C@@H](Oc2ccc([C@@H]3c4[nH]c5ccccc5c4C[C@@H](C)N3CC(C)(C)F)s2)C1] | I-14 |
| 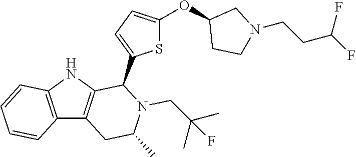 [SMILES: C[C@@H]1Cc2c([nH]c3ccccc23)[C@@H](c2ccc(O[C@@H]3CCN(CCC(F)F)C3)s2)N1CC(C)(C)F] | I-15 |
| 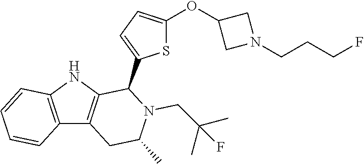 [SMILES: C[C@@H]1Cc2c([nH]c3ccccc23)[C@@H](c2ccc(OC3CN(CCCF)C3)s2)N1CC(C)(C)F] | I-16 |
| 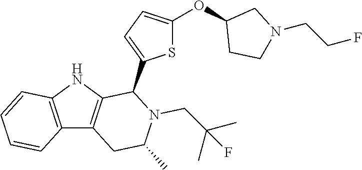 [SMILES: C[C@@H]1Cc2c([nH]c3ccccc23)[C@@H](c2ccc(O[C@@H]3CCN(CCF)C3)s2)N1CC(C)(C)F] | I-17 |
| 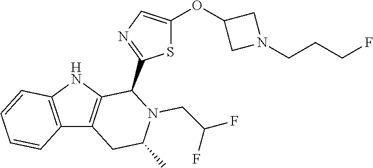 [SMILES: C[C@@H]1Cc2c([nH]c3ccccc23)[C@@H](c2ncc(OC3CN(CCCF)C3)s2)N1CC(F)F] | I-18 |
| 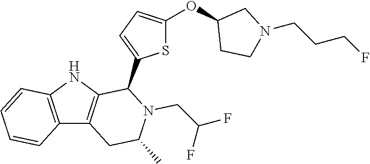 [SMILES: C[C@@H]1Cc2c([nH]c3ccccc23)[C@@H](c2ccc(O[C@@H]3CCN(CCCF)C3)s2)N1CC(F)F] | I-19 |
| 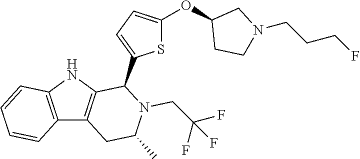 [SMILES: C[C@@H]1Cc2c([nH]c3ccccc23)[C@@H](c2ccc(O[C@@H]3CCN(CCCF)C3)s2)N1CC(F)(F)F] | I-20 |
| 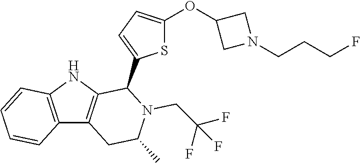 [SMILES: C[C@@H]1Cc2c([nH]c3ccccc23)[C@@H](c2ccc(OC3CN(CCCF)C3)s2)N1CC(F)(F)F] | I-21 |
| 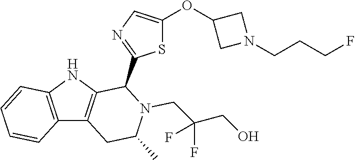 [SMILES: C[C@@H]1Cc2c([nH]c3ccccc23)[C@@H](c2ncc(OC3CN(CCCF)C3)s2)N1CC(F)(F)CO] | I-22 |
| 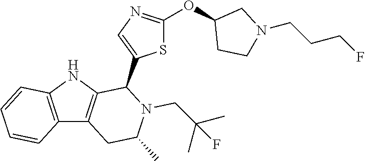 [SMILES: C[C@@H]1Cc2c([nH]c3ccccc23)[C@@H](c2cnc(O[C@@H]3CCN(CCCF)C3)s2)N1CC(C)(C)F] | I-23 |
| 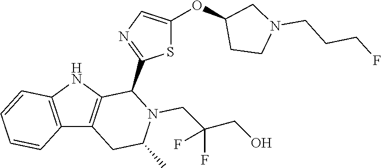 [SMILES: C[C@@H]1Cc2c([nH]c3ccccc23)[C@@H](c2ncc(O[C@@H]3CCN(CCCF)C3)s2)N1CC(F)(F)CO] | I-24 |
| 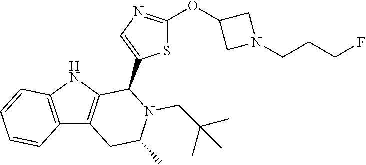 [SMILES: C[C@@H]1Cc2c([nH]c3ccccc23)[C@@H](c2cnc(OC3CN(CCCF)C3)s2)N1CC(C)(C)C] | I-25 |
| 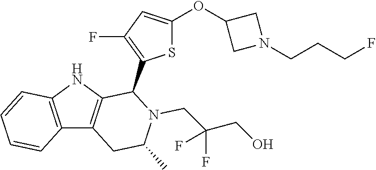 [SMILES: C[C@@H]1Cc2c([nH]c3ccccc23)[C@@H](c2sc(OC3CN(CCCF)C3)cc2F)N1CC(F)(F)CO] | I-26 |
| 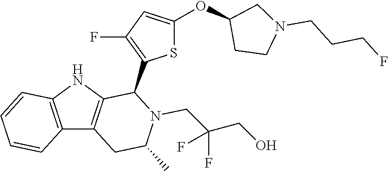 [SMILES: C[C@@H]1Cc2c([nH]c3ccccc23)[C@@H](c2sc(O[C@@H]3CCN(CCCF)C3)cc2F)N1CC(F)(F)CO] | I-27 |
| 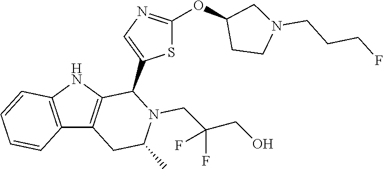 [SMILES: C[C@@H]1Cc2c([nH]c3ccccc23)[C@@H](c2cnc(O[C@@H]3CCN(CCCF)C3)s2)N1CC(F)(F)CO] | I-28 |
| 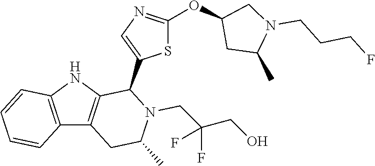 [SMILES: C[C@@H]1Cc2c([nH]c3ccccc23)[C@@H](c2cnc(O[C@@H]3C[C@H](C)N(CCCF)C3)s2)N1CC(F)(F)CO] | I-29 |
| 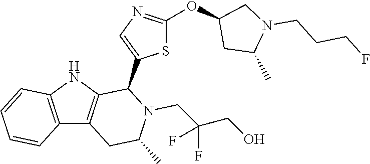 [SMILES: C[C@@H]1C[C@@H](Oc2ncc([C@@H]3c4[nH]c5ccccc5c4C[C@@H](C)N3CC(F)(F)CO)s2)CN1CCCF] | I-30 |
| 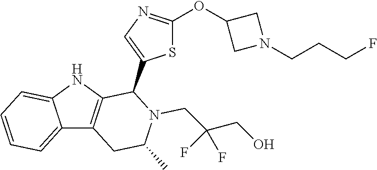 [SMILES: C[C@@H]1Cc2c([nH]c3ccccc23)[C@@H](c2cnc(OC3CN(CCCF)C3)s2)N1CC(F)(F)CO] | I-31 |
| 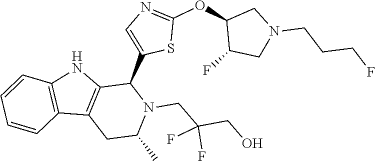 [SMILES: C[C@@H]1Cc2c([nH]c3ccccc23)[C@@H](c2cnc(O[C@H]3CN(CCCF)C[C@@H]3F)s2)N1CC(F)(F)CO] | I-32 |
| 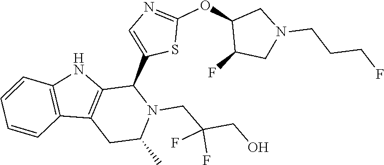 [SMILES: C[C@@H]1Cc2c([nH]c3ccccc23)[C@@H](c2cnc(O[C@H]3CN(CCCF)C[C@H]3F)s2)N1CC(F)(F)CO] | I-33 |
| 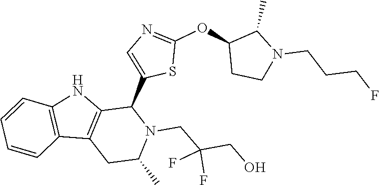 [SMILES: C[C@@H]1Cc2c([nH]c3ccccc23)[C@@H](c2cnc(O[C@@H]3CCN(CCCF)[C@H]3C)s2)N1CC(F)(F)CO] | I-34 |
| 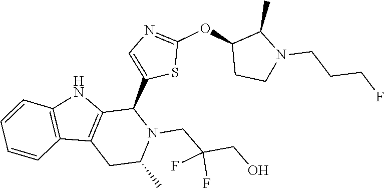 [SMILES: C[C@@H]1[C@H](Oc2ncc([C@@H]3c4[nH]c5ccccc5c4C[C@@H](C)N3CC(F)(F)CO)s2)CCN1CCCF] | I-35 |
| 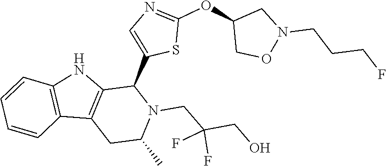 [SMILES: C[C@@H]1Cc2c([nH]c3ccccc23)[C@@H](c2cnc(O[C@@H]3CON(CCCF)C3)s2)N1CC(F)(F)CO] | I-36 |
| 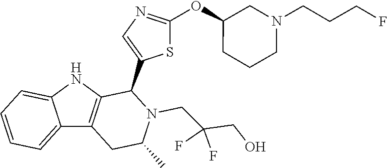 [SMILES: C[C@@H]1Cc2c([nH]c3ccccc23)[C@@H](c2cnc(O[C@@H]3CCCN(CCCF)C3)s2)N1CC(F)(F)CO] | I-37 |
| 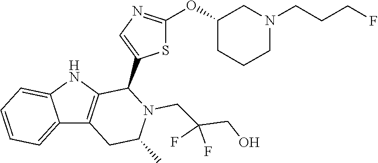 [SMILES: C[C@@H]1Cc2c([nH]c3ccccc23)[C@@H](c2cnc(O[C@H]3CCCN(CCCF)C3)s2)N1CC(F)(F)CO] | I-38 |

In some embodiments, the present disclosure provides a compound of Table 2.

TABLE 2

| Compound No. | Compound Name |
|---|---|
| I-1 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}oxy)-1-propylazetidine |
| I-2 | 3-({5-[(1S,3R)-2-(2-fluoro-2-methylpropyl)-3-methyl-1H,2H,3H,4H,9H- |
| | pyrido[3,4-b]indol-1-yl]thiophen-2-yl}oxy)-1-(3-fluoropropyl)azetidine |
| I-3 | 5-[(1S,3R)-2-(2-fluoro-2-methylpropyl)-3-methyl-1H,2H,3H,4H,9H- |
| | pyrido[3,4-b]indol-1-yl]-2-[(1-propylazetidin-3-yl)oxy]-1,3-thiazole |
| I-4 | (3S)-3-({5-[(1S,3R)-2-(2-fluoro-2-methylpropyl)-3-methyl- |
| | 1H,2H,3H,4H,9H-pyrido[3,4-b]indol-1-yl]thiophen-2-yl}oxy)-1-(3- |
| | fluoropropyl)pyrrolidine |
| I-5 | (3R)-3-({5-[(1S,3R)-2-(2-fluoro-2-methylpropyl)-3-methyl- |
| | 1H,2H,3H,4H,9H-pyrido[3,4-b]indol-1-yl]thiophen-2-yl}oxy)-1-(3- |
| | fluoropropyl)pyrrolidine |
| I-6 | 2-[(1S,3R)-2-(2-fluoro-2-methylpropyl)-3-methyl-1H,2H,3H,4H,9H- |
| | pyrido[3,4-b]indol-1-yl]-5-{[(3S)-1-(3-fluoropropyl)pyrrolidin-3-yl]oxy}- |
| | 1,3-thiazole |
| I-7 | (1S,3R)-2-(2-fluoro-2-methyl-propyl)-3-methyl-1-[5-[(3S)-1- |
| | propylpyrrolidin-3-yl]oxy-2-thienyl]-1,3,4,9-tetrahydropyrido[3,4-b]indole |
| I-8 | (1S,3R)-1-(5-(((S)-1-(3,3-difluoropropyl)pyrrolidin-3-yl)oxy)thiophen-2- |
| | yl)-2-(2-fluoro-2-methylpropyl)-3-methyl-2,3,4,9-tetrahydro-1H- |
| | pyrido[3,4-b]indole |
| I-9 | 2-[(1S,3R)-2-(2-fluoro-2-methyl-propyl)-3-methyl-1,3,4,9- |
| | tetrahydropyrido[3,4-b]indol-1-yl]-5-[1-(3-fluoropropyl)azetidin-3-yl]oxy- |
| | thiazole |
| I-10 | (1S,3R)-2-(2-fluoro-2-methyl-propyl)-3-methyl-1-[5-[(3R)-1- |
| | propylpyrrolidin-3-yl]oxy-2-thienyl]-1,3,4,9-tetrahydropyrido[3,4-b]indole |
| I-11 | 2-[(1S,3R)-2-(2-fluoro-2-methyl-propyl)-3-methyl-1,3,4,9- |
| | tetrahydropyrido[3,4-b]indol-1-yl]-5-[(3R)-1-(3-fluoropropyl)pyrrolidin-3- |
| | yl]oxy-thiazole |
| I-12 | 2,2-difluoro-3-[(1S,3R)-1-[5-[1-(3-fluoropropyl)azetidin-3-yl]oxy-2- |
| | thienyl]-3-methyl-1,3,4,9-tetrahydropyrido[3,4-b]indol-2-yl]propan-1-ol |
| I-13 | 2,2-difluoro-3-[(1S,3R)-1-[5-[(3R)-1-(3-fluoropropyl)pyrrolidin-3-yl]oxy- |
| | 2-thienyl]-3-methyl-1,3,4,9-tetrahydropyrido[3,4-b]indol-2-yl]propan-1-ol |
| I-14 | (1S,3R)-1-[5-[(3R)-1-ethylpyrrolidin-3-yl]oxy-2-thienyl]-2-(2-fluoro-2- |
| | methyl-propyl)-3-methyl-1,3,4,9-tetrahydropyrido[3,4-b]indole |
| I-15 | (1S,3R)-1-[5-[(3R)-1-(3,3-difluoropropyl)pyrrolidin-3-yl]oxy-2-thienyl]-2- |
| | (2-fluoro-2-methyl-propyl)-3-methyl-1,3,4,9-tetrahydropyrido[3,4-b]indole |
| I-16 | (1S,3R)-2-(2,2-difluoroethyl)-1-[5-[1-(3-fluoropropyl)azetidin-3-yl]oxy-2- |
| | thienyl]-3-methyl-1,3,4,9-tetrahydropyrido[3,4-b]indole |
| I-17 | (1S,3R)-1-[5-[(3R)-1-(2-fluoroethyl)pyrrolidin-3-yl]oxy-2-thienyl]-2-(2- |
| | fluoro-2-methyl-propyl)-3-methyl-1,3,4,9-tetrahydropyrido[3,4-b]indole |
| I-18 | 2-[(1S,3R)-2-(2,2-difluoroethyl)-3-methyl-1,3,4,9-tetrahydropyrido[3,4- |
| | b]indol-1-yl]-5-[1-(3-fluoropropyl)azetidin-3-yl]oxy-thiazole |
| I-19 | (1S,3R)-2-(2,2-difluoroethyl)-1-[5-[(3R)-1-(3-fluoropropyl)pyrrolidin-3- |
| | yl]oxy-2-thienyl]-3-methyl-1,3,4,9-tetrahydropyrido[3,4-b]indole |
| I-20 | (1S,3R)-1-[5-[(3R)-1-(3-fluoropropyl)pyrrolidin-3-yl]oxy-2-thienyl]-3- |
| | methyl-2-(2,2,2-trifluoroethyl)-1,3,4,9-tetrahydropyrido[3,4-b]indole |
| I-21 | (1S,3R)-1-[5-[1-(3-fluoropropyl)azetidin-3-yl]oxy-2-thienyl]-3-methyl-2- |
| | (2,2,2-trifluoroethyl)-1,3,4,9-tetrahydropyrido[3,4-b]indole |
| I-22 | 2,2-difluoro-3-[(1S,3R)-1-[5-[1-(3-fluoropropyl)azetidin-3-yl]oxythiazol-2- |
| | yl]-3-methyl-1,3,4,9-tetrahydropyrido[3,4-b]indol-2-yl]propan-1-ol |
| I-23 | 5-[(1S,3R)-2-(2-fluoro-2-methyl-propyl)-3-methyl-1,3,4,9- |
| | tetrahydropyrido[3,4-b]indol-1-yl]-2-[(3R)-1-(3-fluoropropyl)pyrrolidin-3- |
| | yl]oxy-thiazole |
| I-24 | 2,2-difluoro-3-[(1S,3R)-1-[5-[(3R)-1-(3-fluoropropyl)pyrrolidin-3- |
| | yl]oxythiazol-2-yl]-3-methyl-1,3,4,9-tetrahydropyrido[3,4-b]indol-2- |
| | yl]propan-1-ol |
| I-25 | 5-[(1S,3R)-2-(2-fluoro-2-methyl-propyl)-3-methyl-1,3,4,9- |
| | tetrahydropyrido[3,4-b]indol-1-yl]-2-[1-(3-fluoropropyl)azetidin-3-yl]oxy- |
| | thiazole |
| I-26 | 2,2-difluoro-3-[(1S,3R)-1-[3-fluoro-5-[1-(3-fluoropropyl)azetidin-3-yl]oxy- |
| | 2-thienyl]-3-methyl-1,3,4,9-tetrahydropyrido[3,4-b]indol-2-yl]propan-1-ol |
| I-27 | 2,2-difluoro-3-[(1S,3R)-1-[3-fluoro-5-[(3R)-1-(3-fluoropropyl)pyrrolidin-3- |
| | yl]oxy-2-thienyl]-3-methyl-1,3,4,9-tetrahydropyrido[3,4-b]indol-2- |
| | yl]propan-1-ol |
| I-28 | 2,2-difluoro-3-[(1S,3R)-1-[2-[(3R)-1-(3-fluoropropyl)pyrrolidin-3- |
| | yl]oxythiazol-5-yl]-3-methyl-1,3,4,9-tetrahydropyrido[3,4-b]indol-2- |
| | yl]propan-1-ol |
| I-29 | 2,2-difluoro-3-((1S,3R)-1-(2-(((3R,5S)-1-(3-fluoropropyl)-5- |
| | methylpyrrolidin-3-yl)oxy)thiazol-5-yl)-3-methyl-1,3,4,9-tetrahydro-2H- |
| | pyrido[3,4-b]indol-2-yl)propan-1-ol |
| I-30 | 2,2-difluoro-3-[(1S,3R)-1-[2-[(3R,5R)-1-(3-fluoropropyl)-5-methyl- |
| | pyrrolidin-3-yl]oxythiazol-5-yl]-3-methyl-1,3,4,9-tetrahydropyrido[3,4- |
| | b]indol-2-yl]propan-1-ol |
| I-31 | 2,2-difluoro-3-[(1S,3R)-1-[2-[1-(3-fluoropropyl)azetidin-3-yl]oxythiazol-5- |
| | yl]-3-methyl-1,3,4,9-tetrahydropyrido[3,4-b]indol-2-yl]propan-1-ol |
| I-32 | 2,2-difluoro-3-[(1S,3R)-1-[2-[(3S,4S)-4-fluoro-1-(3- |
| | fluoropropyl)pyrrolidin-3-yl]oxythiazol-5-yl]-3-methyl-1,3,4,9- |
| | tetrahydropyrido[3,4-b]indol-2-yl]propan-1-ol |
| I-33 | 2,2-difluoro-3-[(1S,3R)-1-[2-[(3S,4R)-4-fluoro-1-(3- |
| | fluoropropyl)pyrrolidin-3-yl]oxythiazol-5-yl]-3-methyl-1,3,4,9- |
| | tetrahydropyrido[3,4-b]indol-2-yl]propan-1-ol |
| I-34 | 2,2-difluoro-3-[(1S,3R)-1-[2-[(2S,3R)-1-(3-fluoropropyl)-2-methyl- |
| | pyrrolidin-3-yl]oxythiazol-5-yl]-3-methyl-1,3,4,9-tetrahydropyrido[3,4- |
| | b]indol-2-yl]propan-1-ol |
| I-35 | 2,2-difluoro-3-[(1S,3R)-1-[2-[(2R,3R)-1-(3-fluoropropyl)-2-methyl- |
| | pyrrolidin-3-yl]oxythiazol-5-yl]-3-methyl-1,3,4,9-tetrahydropyrido[3,4- |
| | b]indol-2-yl]propan-1-ol |
| I-36 | 2,2-difluoro-3-[(1S,3R)-1-[2-[(4S)-2-(3-fluoropropyl)isoxazolidin-4- |
| | yl]oxythiazol-5-yl]-3-methyl-1,3,4,9-tetrahydropyrido[3,4-b]indol-2- |
| | yl]propan-1-ol |
| I-37 | 2,2-difluoro-3-[(1S,3R)-1-[2-[[(3R)-1-(3-fluoropropyl)-3- |
| | piperidyl]oxy]thiazol-5-yl]-3-methyl-1,3,4,9-tetrahydropyrido[3,4-b]indol- |
| | 2-yl]propan-1-ol |
| I-38 | 2,2-difluoro-3-[(1S,3R)-1-[2-[(3S)-1-(3-fluoropropyl)-3- |
| | piperidyl]oxy]thiazol-5-yl]-3-methyl-1,3,4,9-tetrahydropyrido[3,4-b]indol- |
| | 2-yl]propan-1-ol |

In some embodiments, the present disclosure provides a compound selected from Table 3, or a pharmaceutically acceptable salt thereof:

TABLE 3

Compound No.
I-5
I-10
I-14
I-15
I-17
I-25
I-29
I-30
I-32
I-34
I-35
I-36
I-38

In some embodiments, a compound provided herein is an agonist, and is selected from Table 3.

In some embodiments, the present disclosure provides a compound selected from Table 4, or a pharmaceutically acceptable salt thereof:

TABLE 4

Compound No.
I-1
I-2
I-3
I-6
I-8
I-9
I-11
I-12
I-13
I-16
I-18
I-19
I-20
I-21
I-22
I-23
I-24
I-26
I-27
I-28
I-31
I-33
I-37

In some embodiments, a compound provided herein is an antagonist, and is selected from Table 4.

In some embodiments, the present disclosure provides a compound selected from Table 5, or a pharmaceutically acceptable salt thereof:

TABLE 5

Compound No.
I-4
I-12
I-13
I-16
I-19
I-23
I-26
I-27
I-28
I-33
I-37

In some embodiments, a compound described herein is an antagonist and is selected from Table 5.

In some embodiments, provided compounds are provided and/or utilized in a salt form (e.g., a pharmaceutically acceptable salt form). Reference to a compound provided herein is understood to include reference to salts thereof, unless otherwise indicated.

It will be appreciated that throughout the present disclosure, unless otherwise indicated, reference to a compound of Formula I is intended to also include Formulae I-X, and compound species of such formulas disclosed herein.

### Preparing Provided Compounds

Provided compounds may generally be made by the processes described in the ensuing schemes and examples. In some embodiments, provided compounds are prepared according to Schemes 1 and 2:

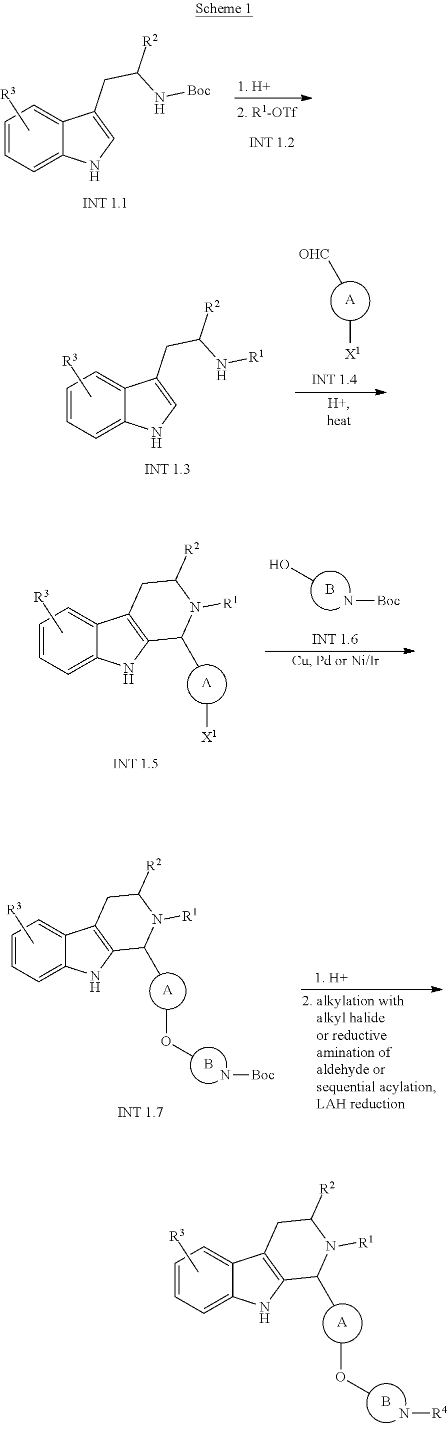

In some embodiments, preparation of target compounds is achieved by deprotection and alkylation of INT 1.1 with INT 1.2 (wherein R¹, R², and R³ are as defined in classes and subclasses herein with respect to Formula I both singly and in combination) to provide INT 1.3. In some embodiments, INT 1.3 undergoes a Pictet-Spengler reaction with a carbaldehyde INT 1.4 (wherein A is as defined in classes and subclasses herein with respect to Formula I both singly and in combination, and X¹ is halogen) to provide INT 1.5. In some embodiments, INT 1.5 is subjected to various C—O bond forming reactions, including copper-mediated, palladium-catalyzed, nickel/iridium-catalyzed-photoredox, or SNAr reactions. For example, in some embodiments, INT 1.5 is reacted with INT 1.6 (wherein B is as defined in classes and subclasses herein with respect to Formula I) in the presence of catalytic copper, palladium, or a nickel/iridium complex to provide INT 1.7. In some embodiments, when R¹ is hydrogen, the resulting free amine is protected as a Boc carbamate or other suitable protecting group prior to C—O bond formation. In some embodiments, acidic deprotection of Boc protecting group(s) (e.g., INT 1.7) provides an amine that may be modified by S_{N}2 alkylation, reductive amination or a two-step acylation-amide reduction sequence to yield target compounds (wherein R⁴ is as defined in classes and subclasses herein with respect to Formula I both singly and in combination).

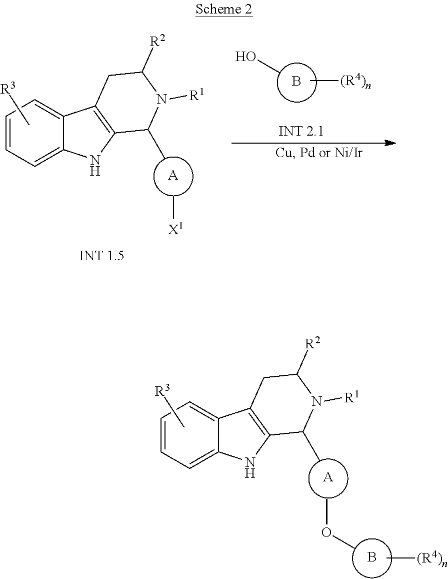

In some embodiments, INT 1.5 is coupled to an alcohol of INT 2.1 (wherein B, R⁴, and n are as defined in classes and subclasses herein with respect to Formula I both singly and in combination) via copper-mediated, palladium-catalyzed, nickel/iridium-catalyzed-photoredox, or SNAr reactions. In some embodiments, when R¹ is hydrogen, the resulting free amine is protected (e.g., with a Boc moiety) prior to the coupling. In some embodiments, a further deprotection step occurs after the coupling of INT 1.5 and INT 2.1 to yield the target compounds.

### Uses, Formulation, and Administration

The present disclosure provides uses for compounds and compositions described herein. In some embodiments, provided compounds and compositions are useful in medicine (e.g., as therapy). In some embodiments, provided compounds and compositions are useful in research as, for example, analytical tools and/or control compounds in biological assays.

### Pharmaceutically Acceptable Compositions

According to another embodiment, the present disclosure provides a composition comprising a compound described herein or a pharmaceutically acceptable salt thereof and a pharmaceutically acceptable carrier, adjuvant, or vehicle. In certain embodiments, the amount of compound in compositions described herein is such that it is effective to measurably induce degradation of a target in a biological sample or in a patient. In certain embodiments, a composition described herein is formulated for administration to a patient in need of such composition. In some embodiments, a composition described herein is formulated for oral administration to a patient.

Compounds and compositions, according to method of the present disclosure, are administered using any amount and any route of administration effective for treating or lessening the severity of a disorder provided herein. The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage.

Compositions of the present disclosure may be administered orally, parenterally, by inhalation spray, topically, rectally, nasally, buccally, vaginally, intraperitoneally, intracisternallyor via an implanted reservoir. In some embodiments, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In some embodiments, provided pharmaceutically acceptable compositions are formulated for oral administration. Such formulations may be administered with or without food. In some embodiments, pharmaceutically acceptable compositions described herein are administered without food. In other embodiments, pharmaceutically acceptable compositions described herein are administered with food. Pharmaceutically acceptable compositions described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Pharmaceutically acceptable compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Estrogen Receptor-Associated Diseases and Disorders

The estrogen receptor (“ER”) is involved in a variety of biological processes, relating, for example, to development of the female reproductive system, maintenance of bone mass, protection of cardiovascular and/or central nervous system components, etc. (see, for example, Pearce & Jordan Crit. Rev. Onc/Hem 50:3, 2004; Heldring Phys. Rev. 87:905, 2007). The ER has been implicated in a variety of cancers. In many tumors that express the estrogen receptor (i.e., ER⁺ tumors), active ERα signaling has been demonstrated to drive cell proliferation (although ERβ signaling has been reported to be able to achieve tumor suppressor effects; see, for example, Nilsson & Gustafson Clin. Pharmacol. Ther. 89:44, 2011). Typically, tumors (e.g., breast tumors) with as few as 1% of cells staining positive for ER are classified as “ER⁺”. Therapies targeting the ER are standard of care for many patients with ER⁺ tumors (see, for example, Cardoso et al Annals Onc. https://doi.org/10.1093/announc/mdmx036>, 2017; Rugo et al. J. Clin. Oncol. 34:3069, 2016; Senkus et al Annal Onc. 26: v8, 2015; Sareddy & Vadlamudi Clin. J Nat. Med, 13:801, 2015). For early stage breast cancer patients, for example, recommended therapy typically involves tumor resection, followed by ER-targeted therapy (e.g., as discussed below). For advanced breast cancer, including metastatic breast cancer, ER-targeted therapy is the mainstay.

Given the importance of ER signaling in many cancers, as well as in certain cardiovascular, inflammatory, and neurodegenerative diseases, significant effort has been invested in developing therapeutic agents and modalities that target the ER. There is some fluidity/flexibility in terminology that has been used to describe ER-targeting agents, but a variety of agents, with different mechanisms, have been developed and/or studied.

For example, some ER-targeting agents are designed and/or documented to reduce levels of estrogen (i.e., 17β estradiol) production. In other embodiments, some ER-targeting agents are designed and/or documented to increase levels of estrogen production.

Some ER-targeting agents are designed and/or documented to bind directly to the ER; in some cases, such agents compete with estrogen for binding to the ER and/or interfere with the allosteric changes that estrogen binding would naturally produce. Often, the term “antiestrogen” is used to refer to agents that bind to the ER, and sometimes is specifically used to indicate those agents that compete with estrogen for ER binding.

The term “selective estrogen receptor modulator, “SERM”, has been used to refer to compounds that are designed and/or documented to alter some aspect of ER activity. Some writings refer to “SERMs” as representing a particular type of anti-estrogens; other writings, however, use the term “SERM” more generally, to refer to a compound that specifically impacts some feature of ER (particularly ERα) expression and/or activity.

The term “selective estrogen receptor degrader” (“SERD”) has been used to refer to compounds that are designed and/or documented to trigger or enhance degradation of the ER. In many instances, if presence of a compound correlates with reduced level of ER, the compound may be referred to as a SERD. Some writings classify compounds either as SERMs or as SERDs; others refer to SERDs as a particular type, or species, of compounds that are SERMs.

Regardless of mechanism of action of a particular agent, clinical experience thus far has revealed that incomplete effects (e.g., within an individual patient and/or across patient populations) and/or development of resistance remain a problem.

Among other things, presence or development of certain ER mutations has been reported to impact effectiveness of various ER-targeted therapies (see, for example, Jeselsohn et al Nature Rev. Clin. Onc. 12, 573, 2015; Gelsomino et al. Breast Cancer Res. Treat 157:253, 2016; Toy et al. 2013). Some particularly problematic mutations are those that “activate” one or more aspects of ER expression and/or function; some activating mutations have been reported that can render the ER ligand-independent (i.e., constitutively active). For example, particular mutations in the ER ligand binding domain, including D538G and Y537S, have been demonstrated to constitutively activate the ER; other mutations including deletions and/or fusions that remove the ligand binding domain, can have similar effects (see, for example, Li et al. Cell Repts 4:1116, 2013; Veeraraghavan et al Breast Cancer Research and Treatment 158, 219-232, 2016; Veeraraghavan, et al. Nature Comms 5:4577, 2014). Some reports have indicated that as many as 50% of women with metastatic breast cancer may have activating ER mutations detectible in circulating tumor DNA.

### Estrogen Receptor Antagonists

In some embodiments, compounds provided herein are estrogen receptor antagonists. As used herein, an “estrogen receptor antagonist” refers to a compound or composition that produces an antagonistic effect when contacting the estrogen receptor of a subject or biological sample. In some embodiments, an estrogen receptor antagonist is characterized by having:
1. (i) between 10% and 80% increase in the E2-normalized signal in the AP assay (agonist mode) of Example 139 and (ii) between 10% and 80% reduction in the E2-normalized signal in the AP assay (antagonist mode) of Example 139;
2. (i) a pIC₅₀ greater than 6.4 and at least a 10% reduction in the E2-normalized signal in the AP assay (antagonist mode) of Example 139; and (ii) no more than 10% increase in E2-normalized signal in the AP assay (agonist mode) of Example 139; or
3. (i) a pIC₅₀ greater than 7.5 and at least a 10% reduction in the E2-normalized signal in the AP assay (antagonist mode) of Example 139; and (ii) no more than 10% increase in E2-normalized signal in the AP assay (agonist mode) of Example 139.

In some embodiments, compounds provided herein are complete estrogen receptor antagonists. As described herein, a complete estrogen receptor antagonist (a “CERAN”) is one that (1) inhibits both AF1 and AF2, and in particular inhibits AF1 activity that remains present in constitutively active ER mutants; (2) promotes ER degradation; and (3) lacks the partial ER agonist activity observed with certain other agents. The present disclosure further appreciates that many previous therapies, including for example, ARN-810, AZD9496, tamoxifen, and others, are less effective than CERANs at least in part because they only partially antagonize ER, and specifically because they inhibit activation of AF2 but not AF1. In some embodiments, an estrogen receptor antagonist is characterized by having (i) a pIC₅₀ greater than 6.4 and at least a 10% reduction in the E2-normalized signal in the AP assay (antagonist mode) of Example 139; and (ii) no more than 10% increase in E2-normalized signal in the AP assay (agonist mode) of Example 139. In some embodiments, an estrogen receptor antagonist is characterized by having (i) a pIC₅₀ greater than 7.5 and at least a 10% reduction in the E2-normalized signal in the AP assay (antagonist mode) of Example 139; and (ii) no more than 10% increase in E2-normalized signal in the AP assay (agonist mode) of Example 139.

### Estrogen Receptor Agonists

In some embodiments, compounds provided herein are estrogen receptor agonists. As used herein, an “estrogen receptor agonist” refers to a compound or composition that produces an agonistic effect when contacting the estrogen receptor of a subject or biological sample. In some embodiments, an estrogen receptor agonist is characterized by having (i) at least 80% increase in the E2-normalized signal in the AP assay (agonist mode) of Example 139 and (ii) no more than 80% reduction in the E2-normalized signal in the AP assay (antagonist mode) of of Example 139.

### Diseases, Disorders, and Conditions

The present disclosure encompasses the insight that provided compounds have a number of uses, including treatment of an ER-associated disorder (e.g., an ER-associated cancer, such as breast cancer, osteoporosis, or menopause symptoms), detection, and/or characterization of certain tumors. In some embodiments, a disease, disorder, or condition is a cancer. In some embodiments, a disease, disorder, or condition is associated with a mutation in an estrogen receptor.

In some embodiments, provided compounds are useful for treating a disorder associated with increased ER activity (e.g., an ER-associated cancer such as breast cancer). In some embodiments, provided estrogen receptor antagonists (e.g., complete estrogen receptor antagonists) are useful for treating such disorders.

In some embodiments, provided compounds are useful for treating a disorder associated with decreased ER activity (e.g., menopause-related conditions or symptoms, or osteoporosis). In some embodiments, provided estrogen receptor agonists are useful for treating such disorders. Other uses of estrogen receptor agonists exist; see, e.g., Harrison, R. F. and Bonnar, J., Pharmac. Ther., 1980, 11, 451-67.

In some embodiments, the present disclosure provides a method of treating a disorder mediated by an estrogen receptor in a subject, comprising administering to the subject a therapeutically effective amount of a compound described herein.

In some embodiments, a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, the present disclosure provides a method of treating a disorder associated with a mutation of an estrogen receptor in a subject, comprising administering to the subject a therapeutically effective amount of a compound described herein. In some embodiments, a disorder is selected from the group consisting of breast cancer, ovarian cancer, endometrial cancer, vaginal cancer, lung cancer, bone cancer, uterine cancer, and endometriosis. In some embodiments, a disorder is breast cancer. In some embodiments, a disorder is ovarian cancer. In some embodiments, a disorder is endometrial cancer. In some embodiments, a disorder is vaginal cancer. In some embodiments, a disorder is lung cancer. In some embodiments, a disorder is bone cancer. In some embodiments, a disorder is uterine cancer. In some embodiments, a disorder is endometriosis.

In some embodiments, a method of treating a disorder in a subject described herein comprises administering to the subject a compound described herein in combination with or alternation with an anti-cancer agent. In some embodiments, an anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

In some embodiments, an anti-cancer agent is a HER2 inhibitor. In some embodiments, a HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

In some embodiments, an anti-cancer agent is an mTOR inhibitor. In some embodiments, an mTOR inhibitor is selected from everolimus, sirolimus, temsirolimus, and LY3023414.

In some embodiments, an anti-cancer agent is a CDK4/6 inhibitor. In some embodiments, a CDK4/6 inhibitor is selected from palbociclib, abemaciclib, ribociclib, lerociclib, trilaciclib, and SHR6390.

In some embodiments, an anti-cancer agent is a PI3 kinase inhibitor. In some embodiments, a PI3 kinase inhibitor is selected from perifosine, CAL101, BEZ235, XL147, XL765, GDC-0941, and IPI-145.

In some embodiments, a PI3 kinase inhibitor is a PIK3CA inhibitor. In some embodiments, a PIK3CA inhibitor is selected from alpelisib, taselisib, and LY3023414.

In some embodiments, an anti-cancer agent is an aromatase inhibitor. In some embodiments, an aromatase inhibitor is selected from aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

In some embodiments, an anti-cancer agent is an antibody to or inhibitor of PD-1, PD-L1 or CTLA-4.

In some embodiments, an anti-cancer agent is an antibody to or inhibitor of EGFR, PGFR, or IGFR. In some embodiments, an anti-cancer agent is erlotinib or gefitinib.

In some embodiments, a method described herein comprises administering a compound reported herein in combination or in alternation with an estrogen receptor antagonist or a partial estrogen receptor antagonist.

In some embodiments, the present disclosure provides a method of preventing recurrence of a cancer in a subject comprising administering to the subject a compound described herein. In some embodiments, a cancer is selected from breast cancer, ovarian cancer, endometrial cancer, vaginal cancer, lung cancer, bone cancer, and uterine cancer. In some embodiments, a compound described herein is administered as an adjunctive therapy after or instead of chemotherapy, radiation, or surgery. In some embodiments, a compound is administered after surgery. In some embodiments, a compound is administered prior to surgery. In some embodiments, a cancer is a breast cancer that has progressed in the presence of endocrine or aromatase therapy.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

### Example 1: 2-Fluoro-2-methylpropan-1-ol

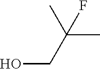

To a flask equipped with a mechanical stirrer, nitrogen inlet, and thermocouple was added methyl 2-fluoro-2-methylpropanoate (28.63 g, 238.4 mmol, 1.0 equiv) in diethyl ether (560 mL). The solution was cooled to 0° C. and treated with lithium aluminum hydride (9.05 g, 238.4 mmol, 1.0 equiv) over the course of 20 min while maintaining the temperature below 10° C. The reaction was stirred at 0° C. for one h. Water (9.1 mL), 15% sodium hydroxide solution (9.1 mL) and water (17.8 mL) were sequentially added at 0° C. The mixture was vigorously stirred for 15 min at 0° C., then allowed to warm to room temperature and stirred for additional 15 min. Magnesium sulfate (15 g) was added to the resulting white cloudy suspension which was stirred for an additional 10 min. The mixture was filtered and the solid was washed with diethyl ether (2×50 mL). The solvent was removed under reduced pressure to give the title compound as a colorless liquid (20.54 g, 94% yield). ¹H-NMR (400 MHz, CDCl₃) δ=3.59-3.52 (dd, J=6.7 Hz, J=13.6 Hz, 2H), 3.47 (s, 1H), 1.39 (s, 3H), 1.34 (s, 3H).

### Example 2: 2-Fluoro-2-methylpropyl trifluoromethanesulfonate

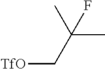

A mixture of compound 2-fluoro-2-methylpropan-1-ol (30.0 g, 325.7 mmol) and triethylamine (42.9 g, 59.1 mL, 423.8 mmol, 1.3 equiv) in MTBE (300 mL) was cooled to −20° C. Trifluoromethanesulfonic anhydride (110.3 g, 65.7 mL, 390.8 mmol, 1.2 equiv) was added via an addition funnel while maintaining the temperature below −5° C. After stirring at −10° C. for 1 h, the reaction was cooled to −20° C., diluted with MTBE (150 mL) and 1M HCl. The mixture was warmed to room temperature and stirred for additional 10 min. The layers were separated, and the organic layer was washed with saturated sodium bicarbonate solution (2×150 mL), saturated brine (250 mL), dried over magnesium sulfate (7 g), filtered, and concentrated under reduced pressure to give the title compound as a colorless liquid (36.1 g, 49% yield). ¹H-NMR (300 MHz, CDCl₃) δ=4.43-4.37 (dd, J=6.7 Hz, J=13.6 Hz, 2H), 1.51 (s, 3H), 1.46 (s, 3H).

### Example 3: (R)-1-(1H-Indol-3-yl)propan-2-amine

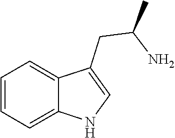

Trifluoroacetic acid (51.9 g, 34.9 mL, 455.6 mmol, 5.0 equiv) was slowly added to a stirred solution of tert-butyl (R)-(1-(1H-indol-3-yl)propan-2-yl) carbamate (25 g, 91.1 mmol, 1.0 equiv) in dichloromethane (250 mL) at room temperature. After stirring for 16 h, the pH was adjusted to 13 with 10% sodium hydroxide. The mixture was extracted with dichloromethane (3×100 mL). The combined organic layers were concentrated under reduced pressure to give the title compound as a brown solid, which was used subsequently. LCMS: m/z=175.2 [M+H]⁺

### Example 4: (R)—N-(1-(1H-indol-3-yl)propan-2-yl)-2-fluoro-2-methylpropan-1-amine

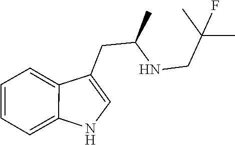 [SMILES: C[C@H](Cc1c[nH]c2ccccc12)NCC(C)(C)F]

N,N-Diisopropylethylamine (17.1 g, 22.8 mL, 132.1 mmol, 1.45 equiv) was added at room temperature to a mixture of compounds (R)-1-(1H-indol-3-yl)propan-2-amine (15.9 g, 91.1 mmol, 1.0 equiv) and 2-fluoro-2-methylpropyl trifluoromethanesulfonate (20.4 g, 91.1 mmol, 1.0 equiv) in 1,4-dioxane (180 mL). After heating at 75° C. for 16 h, the reaction was cooled to room temperature, and diluted with water (180 mL) and MTBE (70 mL). The layers were separated, and the aqueous layer was extracted with MTBE (3×70 mL). The combined organic layers were washed with water (150 mL), saturated brine (150 mL), dried over magnesium sulfate (8 g) and filtered. The filtrate was cooled to 0° C. and carefully treated with 5-6 N HCl in 2-propanol (59 mL, 3.0 equiv). After stirring for 30 min, the suspension was filtered and the solid was triturated with 10% 2-propanol in MTBE (150 mL) to give the hydrochloride salt of the title compound as a white solid. The solid was suspended in 2-propanol (15 mL) and carefully treated with 15% sodium hydroxide (180 mL). The mixture was extracted with MTBE (3×150 mL). The combined organic layers were washed with saturated brine (150 mL), dried over magnesium sulfate (5 g), filtered and concentrated under reduced pressure to give the title compound as a brown solid (13.5 g, 60% yield).

¹H NMR (400 MHz, CDCl₃) δ=8.09 (br s, 1H) 7.62-7.60 (d, 1H), 7.35-7.33 (d, 1H), 7.20-7.09 (m, 2H), 7.02 (s, 1H), 3.02-2.91 (m, 1H), 2.87-281 (m, 1H), 2.79-2.69 (m, 3H), 149 (br s, 1H), 1.37 (s, 3H), 1.30 (s, 3H), 1.11-1.09 (d, J=6.7 Hz, 3H); ¹⁹F NMR: (376 MHz, CDCl₃) δ=−144.1; LCMS: m/z=249.2 [M+H]⁺

### Example 5: (R)—N-(1-(1H-Indol-3-yl)propan-2-yl)-3-((tert-butyldiphenylsilyl)oxy)-2,2-difluoropropan-1-amine

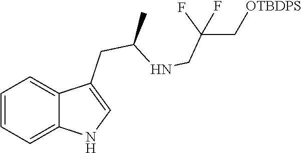

N,N-Diisopropylethylamine (3.5 mL, 20.3 mmol, 1.45 equiv) was added at room temperature to a mixture of (R)-1-(1H-indol-3-yl)propan-2-amine (2.44 g, 14.0 mmol, 1.0 equiv) and 3-((tert-butyldiphenylsilyl)oxy)-2,2-difluoropropyl trifluoromethanesulfonate (6.75 g, 14.0 mmol, 1.0 equiv) in 1,4-dioxane (27 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Biotage® Sfär Silica HC High Capacity 20 μm 350 g silica gel column), eluting with a gradient of 0 to 50% ethyl acetate in heptanes with 1% trimethylamine to give the title compound (3.9 g, 55% yield) as a brown oil. LCMS: m/z=507.3 [M+H]⁺.

### Example 6: (R)—N-(2,2-Difluoroethyl)-1-(1H-indol-3-yl)propan-2-amine

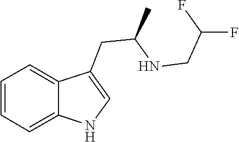 [SMILES: C[C@H](Cc1c[nH]c2ccccc12)NCC(F)F]

N,N-Diisopropylethylamine (3.75 mL, 21.8 mmol, 1.45 equiv) was added at room temperature to a mixture of (R)-1-(1H-indol-3-yl)propan-2-amine (2.61 g, 15.0 mmol, 1.0 equiv) and 2,2-difluoroethyl trifluoromethanesulfonate (3.21 g, 15.0 mmol, 1.0 equiv) in 1,4-dioxane (30 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Sorbtech 330 g silica gel column), eluting with a gradient of 0 to 70% ethyl acetate in heptanes with 1% trimethylamine to give the title compound (2.4 g, 67% yield) as a brown oil. LCMS: m/z=239.1 [M+H]⁺.

### Example 7: (R)-1-(1H-Indol-3-yl)-N-(2,2,2-trifluoroethyl)propan-2-amine

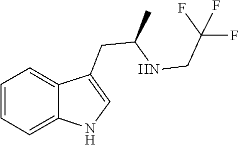 [SMILES: C[C@H](Cc1c[nH]c2ccccc12)NCC(F)(F)F]

N,N-Diisopropylethylamine (3.75 mL, 21.8 mmol, 1.45 equiv) was added at room temperature to a mixture of (R)-1-(1H-indol-3-yl)propan-2-amine (2.61 g, 15.0 mmol, 1.0 equiv) and 2,2,2-trifluoroethyl trifluoromethanesulfonate (3.48 g, 15.0 mmol, 1.0 equiv) in 1,4-dioxane (30 mL). After heating at 85° C. for 16 h, the reaction was cooled to room temperature and diluted with water (30 mL) and methyl tert-butyl ether (30 mL). The layers were separated, and the aqueous layer was extracted with methyl tert-butyl ether (2×30 mL). The combined organic layers were washed with water (50 mL), saturated brine (50 mL), dried over magnesium sulfate, filtered and concentrated under reduced pressure. The residue was purified on an Interchim automated chromatography system (Sorbtech 330 g silica gel column), eluting with a gradient of 0 to 70% ethyl acetate in heptanes with 1% trimethylamine to give the title compound (3.2 g, 83% yield) as a brown oil. LCMS: m/z=257.1 [M+H]⁺.

### Example 8: (1S,3R)-1-(5-Bromothiophen-2-yl)-2-(2-fluoro-2-methylpropyl)-3-methyl-2,3,4,9-tetrahydro-1H-pyrido[3,4-b]indole

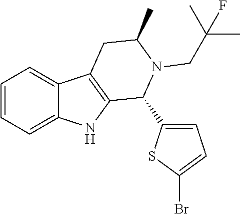 [SMILES: C[C@@H]1Cc2c([nH]c3ccccc23)[C@@H](c2ccc(Br)s2)N1CC(C)(C)F]

5-Bromothiophene-2-carbaldehyde (6.4 g, 33.5 mmol, 1.0 equiv) was added at room temperature to a solution of (R)—N-(1-(1H-indol-3-yl)propan-2-yl)-2-fluoro-2-methylpropan-1-amine (10 g, 40.2 mmol, 1.2 equiv) and acetic acid (3.8 mL, 67 mmol, 2.0 equiv) in toluene (30 mL). After heating at 80° C. for 3 h, the reaction was cooled to room temperature and diluted with saturated sodium bicarbonate (100 mL). After heating at 80° C. for 3 h, the reaction was cooled to room temperature and diluted with saturated sodium bicarbonate (100 mL). The layers were separated, and the aqueous layer was extracted with ethyl acetate (3×50 mL). The combined organic layers were washed with saturated brine (50 mL), dried over magnesium sulfate (10 g), filtered and concentrated under reduced pressure. The crude yellow oil was purified on an Interchim automated chromatography system (Biotage Sfär Silica HC 20 μm, 250 g), eluting with a gradient of 0 to 20% ethyl acetate in heptanes to give the title compound (8.07 g, 57% yield) as a yellow solid. LCMS: m/z=422.3 [M+H]⁺.

### Example 9: (1S,3R)-2-(2-Fluoro-2-methylpropyl)-3-methyl-1-(5-((1-propylazetidin-3-yl)oxy)thiophen-2-yl)-2,3,4,9-tetrahydro-1H-pyrido[3,4-b]indole

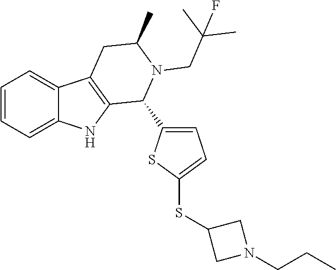 [SMILES: CCCN1CC(Sc2ccc([C@@H]3c4[nH]c5ccccc5c4C[C@@H](C)N3CC(C)(C)F)s2)C1]

(1S,3R)-1-(5-Bromothiophen-2-yl)-2-(2-fluoro-2-methylpropyl)-3-methyl-2,3,4,9-tetrahydro-1H-pyrido[3,4-b]indole (1.0 g, 2.37 mmol, 1.0 equiv) and N-propyl azetidine-3-ol (546.7 mg, 4.75 mmol, 2.0 equiv) were dissolved in anhydrous 1,4-dioxane (2.5 mL). The mixture was sparged with nitrogen under sonication for 15 min. To an oven dried 40 mL vial equipped with magnetic stirrer bar was added copper iodide, (22.6 mg, 0.119 mmol, 0.05 equiv), N,N′-bis(2-phenylethyl) ethanediamide (35.3 mg, 0.119 mmol, 0.05 equiv), sodium tert-butoxide (273 mg, 2.84 mmol, 1.2 equiv) and freshly activated 4 Å MS (400 mg). The vial was sealed and evacuated and backfilled with nitrogen three times. The solution of the starting materials was then added via syringe at room temperature and the mixture was heated at 95° C. for 24 h. An aliquot of the reaction mixture (1.25 mL) was diluted with 15% ammonium hydroxide (5 mL) at room temperature. The layers were separated, and the aqueous layer was extracted with ethyl acetate (3×10 mL). The combined organic layers were washed with saturated brine (10 mL), dried over sodium sulfate, filtered and concentrated under reduced pressure. The crude dark brown residue was purified on a Biotage automated chromatography system (Biotage Sfär Silica HC 20 μm, 25 g), eluting with a gradient of 0 to 10% methanol in dichloromethane to give the title compound (120 mg, 22% yield) as a brown solid. ¹H NMR (400 MHz, CDCl₃) δ=8.11 (br s, 1H), 7.52 (d, J=7.7 Hz, 1H), 7.31 (d, J=7.7 Hz, 1H), 7.17 (dt, J=1.3, 7.5 Hz, 1H), 7.14-7.08 (m, 1H), 6.29 (br d, J=2.3 Hz, 1H), 5.82 (d, J=3.8 Hz, 1H), 5.12 (br s, 1H), 4.67 (quin, J=5.8 Hz, 1H), 3.74 (dt, J=2.4, 5.6 Hz, 2H), 3.47 (br s, 1H), 3.09 (ddd, J=1.5, 5.7, 8.8 Hz, 2H), 2.69-2.55 (m, 3H), 2.54-2.44 (m, 3H), 1.55-1.46 (m, 3H), 1.44-1.36 (m, 2H), 1.36-1.30 (m, 3H), 1.15 (d, J=6.8 Hz, 3H), 0.90 (t, J=7.4 Hz, 3H); LCMS: m/z=456.3 [M+H]⁺.

### Example 10: 3-((tert-Butyldimethylsilyl)oxy)-1-(3-fluoropropyl)azetidine

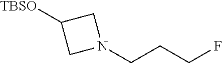 [SMILES: CC(C)(C)[Si](C)(C)OC1CN(CCCF)C1]

A suspension of 3-((tert-butyldimethylsilyl)oxy)azetidine (13.57 g, 72.4 mmol, 1.1 equiv), 1-bromo-3-fluoropropane (9.28 g, 65.8 mmol, 1.0 equiv) and potassium carbonate (26.4 g, 190.8 mmol, 2.9 equiv) in acetonitrile (300 mL) was heated at reflux for 12 h, at which time the TLC analysis indicated that the reaction was complete. After cooling to room temperature, the reaction mixture was filtered through a Celite® pad (25 g). The filtrate was concentrated under reduced pressure. The residue was purified on an Interchim automated chromatography system (350 g, Biotage® Sfär Silica HC High Capacity 20 μm), eluting with a gradient of 0 to 40% ethyl acetate in heptanes with 1% triethylamine to give the title compound as a clear oil (9.9 g, 61% yield). LCMS: m/z=248.2 [M+H]⁺.

### Example 11: 1-(3-Fluoropropyl)azetidin-3-ol

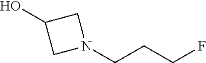

Concentrated HCl (15 mL, 180 mmol, 4.5 equiv) was slowly added to a solution of 3-((tert-butyldimethylsilyl)oxy)-1-(3-fluoropropyl)azetidine (9.9 g, 40.0 mmol, 1.0 equiv) in methanol (100 mL) at room temperature. The resulting solution was heated at 50° C. for 24 h, at which time the LCMS analysis indicated the reaction was complete. After cooling to room temperature, the reaction solution was concentrated under reduced pressure. The resulting oil was sequentially triturated with diethyl ether (2×10 mL), hexanes (2×10 mL) and acetonitrile (3×5 mL) to give 1-(3-fluoropropyl)azetidin-3-ol hydrogen chloride as a white solid (6.6 g, 97% yield). The white solid (1 g, 5.9 mmol) was dissolved in methanol (10 mL), and the solution was passed through a Biotage ISOLUTE® PE-AX SPE column (500 mg). The resulting solution was concentrated under reduced pressure to give the title compound as a yellow oil (550 mg, 70% yield). LCMS: m/z=134.2 [M+H]⁺.

### Example 12: (1S,3R)-2-(2-Fluoro-2-methylpropyl)-1-(5-((1-(3-fluoropropyl)azetidin-3-yl)oxy)thiophen-2-yl)-3-methyl-2,3,4,9-tetrahydro-1H-pyrido[3,4-b]indole

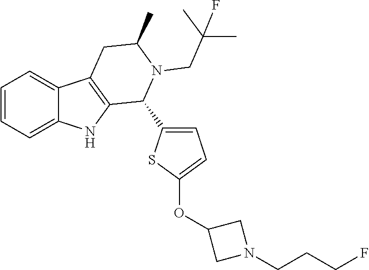 [SMILES: C[C@@H]1Cc2c([nH]c3ccccc23)[C@@H](c2ccc(OC3CN(CCCF)C3)s2)N1CC(C)(C)F]

(1S,3R)-1-(5-Bromothiophen-2-yl)-2-(2-fluoro-2-methylpropyl)-3-methyl-2,3,4,9-tetrahydro-1H-pyrido[3,4-b]indole (500 mg, 1.19 mmol, 1.0 equiv) and 1-(3-fluoropropyl)azetidin-3-ol (317 mg, 2.38 mmol, 2.0 equiv) were dissolved in anhydrous 1,4-dioxane (1.5 mL). The mixture was sparged with nitrogen under sonication for 15 min. To an oven dried 40 mL vial equipped with magnetic stirrer bar was added copper iodide, (11.4 mg, 0.06 mmol, 0.05 equiv), N,N′-bis(2-phenylethyl) ethanediamide (17.8 mg, 0.06 mmol, 0.05 equiv), sodium tert-butoxide (137.4 mg, 1.43 mmol, 1.2 equiv) and freshly activated 4 Å MS (300 mg). The vial was sealed and evacuated and backfilled with nitrogen three times. The solution of the starting materials was then added via syringe at room temperature and the mixture was heated at 95° C. for 38 h. The reaction was cooled to room temperature and diluted with 15% ammonium hydroxide (5 mL). The layers were separated, and the aqueous layer was extracted with ethyl acetate (3×10 mL). The combined organic layers were washed with saturated brine (10 mL), dried over sodium sulfate (5 g), filtered and concentrated under reduced pressure. The crude dark brown residue was purified on an Interchim automated chromatography system (Biotage Sfär Silica HC 20 μm, 25 g), eluting with a gradient of 0 to 10% methanol in dichloromethane to give the title compound (9.1 mg, 1% yield) as a brown solid. ¹H NMR (400 MHz, CDCl₃) δ=7.85 (br s, 1H), 7.51 (d, J=7.8 Hz, 1H), 7.32 (d, J=7.9 Hz, 1H), 7.18 (dt, J=1.3, 7.5 Hz, 1H), 7.14-7.09 (m, 1H), 6.30 (br d, J=2.8 Hz, 1H), 5.84 (d, J=3.9 Hz, 1H), 5.12 (br s, 1H), 4.71 (quin, J=5.7 Hz, 1H), 4.54 (t, J=6.0 Hz, 1H), 4.42 (t, J=5.9 Hz, 1H), 3.81-3.70 (m, 2H), 3.54-3.40 (m, 1H), 3.13 (ddd, J=1.7, 5.6, 8.7 Hz, 2H), 2.69-2.56 (m, 5H), 2.54-2.47 (m, 1H), 1.83-1.69 (m, 2H), 1.35 (s, 3H), 1.30 (s, 3H), 1.15 (d, J=6.8 Hz, 3H); LCMS: m/z=474.3 [M+H]⁺.

### Example 13: tert-Butyl (1S,3R)-1-(5-bromothiophen-2-yl)-2-(2-fluoro-2-methylpropyl)-3-methyl-1,2,3,4-tetrahydro-9H-pyrido[3,4-b]indole-9-carboxylate

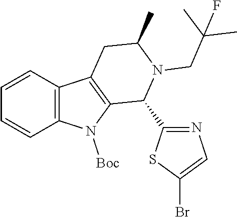 [SMILES: C[C@@H]1Cc2c(n(C(=O)OC(C)(C)C)c3ccccc23)[C@@H](c2ncc(Br)s2)N1CC(C)(C)F]

A solution of (1S,3R)-1-(5-bromothiophen-2-yl)-2-(2-fluoro-2-methylpropyl)-3-methyl-2,3,4,9-tetrahydro-1H-pyrido[3,4-b]indole (1.37 g, 3.25 mmol, 1 equiv) in anhydrous dichloromethane (30 mL) was sequentially treated with triethylamine (0.9 mL, 6.5 mmol, 2 equiv) and di-tert-butyl dicarbonate (0.82 mL, 3.58 mmol, 1.1 equiv) at room temperature for 18 h. The reaction mixture was diluted with dichloromethane (30 mL) and washed with DI water (50 mL) and saturated brine (50 mL). The organic layer was dried over sodium sulfate, filtered and concentrated under reduced pressure. The crude product was pre-absorbed on Celite® (10 g) and purified on an Interchim automated chromatography system (Sorbtech 120 g silica-gel column), eluting with a gradient of 0 to 50% ethyl acetate in heptanes to give the title compound (1.49 mg, 88% yield) as a white foam. LCMS (ESI) m/z=622 [M+H]⁺.

### Example 14: tert-Butyl (1S,3R)-1-(5-(((S)-1-(tert-butoxycarbonyl)pyrrolidin-3-yl)oxy)thio-phen-2-yl)-2-(2-fluoro-2-methylpropyl)-3-methyl-1,2,3,4-tetrahydro-9H-pyrido[3,4-b]indole-9-carboxylate

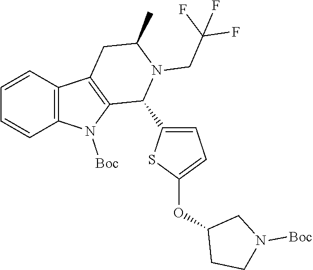 [SMILES: C[C@@H]1Cc2c(n(C(=O)OC(C)(C)C)c3ccccc23)[C@@H](c2ccc(O[C@H]3CCN(C(=O)OC(C)(C)C)C3)s2)N1CC(F)(F)F]

A solution of tert-butyl (1S,3R)-1-(5-bromothiophen-2-yl)-2-(2-fluoro-2-methylpropyl)-3-methyl-1,2,3,4-tetrahydro-9H-pyrido[3,4-b]indole-9-carboxylate (0.84 g, 1.61 mmol, 1.0 equiv) and tert-butyl (S)-3-hydroxypyrrolidine-1-carboxylate (1.0 g, 5.39 mmol, 3.34 equiv) in anhydrous acetonitrile (20 mL) in a 100-mL round bottom flask was sparged with nitrogen for 30 min. 2,2,6,6-Tetramethylpiperidine (2.0 mL, 11.75 mmol, 7.3 equiv) was added dropwise via syringe and the reaction mixture was sparged with nitrogen for an additional 10 min. The flask was briefly opened under a stream of nitrogen and NiCl₂ (glyme) (177 mg, 0.805 mmol, 0.5 equiv), 4,4′-di-tert-butyl-2,2′-dipyridyl (216 mg, 0.805 mmol, 0.5 equiv) and (Ir[dF(CF₃)ppy]₂(dtbpy))PF₆ (181 mg, 0.161 mmol, 0.1 equiv) were added sequentially. The flask was quickly closed and sparged with nitrogen for an additional 5 min. The reaction was stirred under irradiation of LED light (Kessil 40W, 1.5-2 cm distance) at room temperature for 18 h under a nitrogen atmosphere. Celite® (5 g) was added to the reaction mixture, and volatiles were removed under reduced pressure. The residue was purified on an Interchim automated chromatography system (Sorbtech 120 g silica-gel column), eluting with a gradient of 0 to 100% ethyl acetate in heptanes to give the title compound (287 mg, 28% yield) as a tan foam. LCMS (ESI) m/z=628 [M+H]⁻.

### Example 15: (1S,3R)-2-(2-Fluoro-2-methylpropyl)-3-methyl-1-(5-(((S)-pyrrolidin-3-yl)oxy)thiophen-2-yl)-2,3,4,9-tetrahydro-1H-pyrido[3,4-b]indole

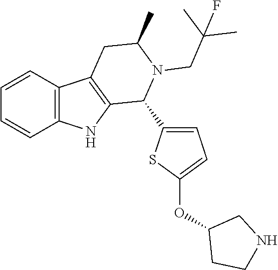 [SMILES: C[C@@H]1Cc2c([nH]c3ccccc23)[C@@H](c2ccc(O[C@H]3CCNC3)s2)N1CC(C)(C)F]

Trifluoroacetic acid (2.1 mL, 7.46 mmol, 60 equiv) was added to a stirred solution of tert-butyl (1S,3R)-1-(5-(((S)-1-(tert-butoxycarbonyl)pyrrolidin-3-yl)oxy)thio-phen-2-yl)-2-(2-fluoro-2-methylpropyl)-3-methyl-1,2,3,4-tetrahydro-9H-pyrido[3,4-b]indole-9-carboxylate (287 mg, 0.46 mmol, 1.0 equiv) in anhydrous dichloromethane (3 mL) at room temperature under a nitrogen atmosphere. The resulting mixture was stirred at room temperature for 1 h. The reaction was diluted with cold dichloromethane (50 mL) and cold saturated sodium carbonate (70 mL). The organic layer was separated, dried over sodium sulfate, filtered and concentrated under reduced pressure to give the crude title compound (181 mg, 92% yield) as a tan solid. LCMS (ESI) m/z=428 [M+H]⁺.

### Example 16: (1S,3R)-2-(2-fluoro-2-methylpropyl)-1-(5-(((S)-1-(3-fluoropropyl)pyrrolidin-3-yl)oxy)thiophen-2-yl)-3-methyl-2,3,4,9-tetrahydro-1H-pyrido[3,4-b]indole

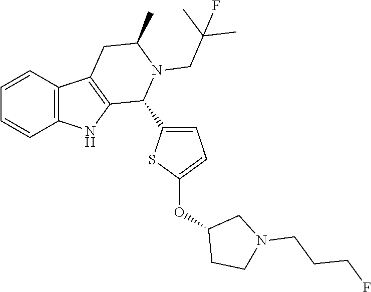 [SMILES: C[C@@H]1Cc2c([nH]c3ccccc23)[C@@H](c2ccc(O[C@H]3CCN(CCCF)C3)s2)N1CC(C)(C)F]

1-Bromo-3-fluoropropane (43 μL, 0.466 mmol, 1.1 equiv) and N,N-diisopropyl-N-ethylamine (0.37 mL, 2.12 mmol, 5 equiv) were sequentially added to a solution of crude (1S,3R)-2-(2-fluoro-2-methylpropyl)-3-methyl-1-(5-(((S)-pyrrolidin-3-yl)oxy)thiophen-2-yl)-2,3,4,9-tetrahydro-1H-pyrido[3,4-b]indole (181 mg, 0.42 mmol, 1 equiv) in anhydrous N,N-dimethylacetamide (8 mL) at room temperature. After stirring for 18 h, the reaction was diluted with ethyl acetate (30 mL) and washed with saturated brine (30 mL). The organic layer was dried over sodium sulfate, filtered and concentrated under reduced pressure. The residue was pre-absorbed on Celite® (1 g) and purified on an Interchim automated chromatography system (KP-Amino Biotage 28 g silica gel column), eluting with a gradient of 0 to 100% dichloromethane in heptanes to give the title compound (87 mg, 42% yield) as a white solid. LCMS (ESI) m/z=488.2 [M+H]⁺; ¹H NMR (400 MHz, CDCl₃) δ=7.85 (br s, 1H), 7.53 (d, J=7.7 Hz, 1H), 7.33 (d, J=7.5 Hz, 1H), 7.22-7.16 (m, 1H), 7.15-7.08 (m, 1H), 6.32 (br s, 1H), 5.91 (d, J=3.8 Hz, 1H), 5.13 (br s, 1H), 4.78-4.65 (m, 1H), 4.58 (t, J=6.0 Hz, 1H), 4.46 (t, J=6.0 Hz, 1H), 3.50 (br s, 1H), 2.87-2.76 (m, 3H), 2.73-2.43 (m, 7H), 2.29-2.18 (m, 1H), 2.10-2.02 (m, 1H), 1.99-1.85 (m, 2H), 1.65 (br s, 1H), 1.55-1.44 (m, 3H), 1.38-1.30 (m, 3H), 1.16 (d, J=6.7 Hz, 3H).

### Example 17: tert-Butyl (1S,3R)-1-(5-(((R)-1-(tert-butoxycarbonyl)pyrrolidin-3-yl)oxy)thiophen-2-yl)-2-(2-fluoro-2-methylpropyl)-3-methyl-1,2,3,4-tetrahydro-9H-pyrido[3,4-b]indole-9-carboxylate

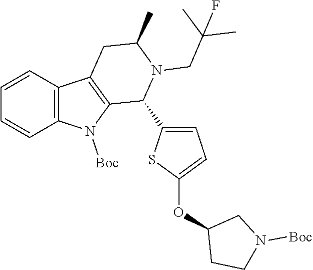 [SMILES: C[C@@H]1Cc2c(n(C(=O)OC(C)(C)C)c3ccccc23)[C@@H](c2ccc(O[C@@H]3CCN(C(=O)OC(C)(C)C)C3)s2)N1CC(C)(C)F]

A mixture of tert-butyl (1S,3R)-1-(5-bromothiophen-2-yl)-2-(2-fluoro-2-methylpropyl)-3-methyl-1,2,3,4-tetrahydro-9H-pyrido[3,4-b]indole-9-carboxylate (0.89 g, 1.709 mmol, 1 equiv), tert-butyl (R)-3-hydroxypyrrolidine-1-carboxylate (1.07 g, 5.705 mmol, 3.34 equiv), and 1,4-diazabicyclo[2.2.2]octane (0.575 g, 5.124 mmol, 3 equiv) in acetonitrile (20 mL) was sparged with nitrogen for 20 min. Nickel(II) chloride (glyme) complex (0.188 g, 0.854 mmol, 0.5 equiv), 4,4′-di-tert-butyl-2,2′-dipyridyl (0.229 g, 0.854 mmol, 0.5 equiv) and (Ir[dF(CF₃)ppy]₂ (dtbpy))PF₆ (0.192 g, 0.171 mmol, 0.1 equiv) were sequentially added at room temperature, The mixture was sparged with nitrogen for an additional 5 min. The reaction vessel was sealed, and the reaction was irradiated with Blue LED light for 20 h. The mixture was dry-loaded onto Celite® and purified on an Interchim automated chromatography system (Sorbtech 80 g silica gel column), eluting with a gradient of 0 to 100% ethyl acetate in heptanes to give the title compound (0.169 g, 16% yield) as a white foam. LCMS: m/z=628 [M+H]⁺.

### Example 18: (1S,3R)-2-(2-Fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole

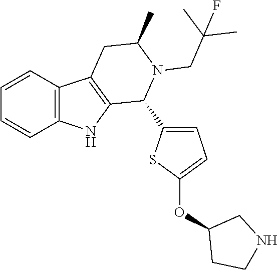 [SMILES: C[C@@H]1Cc2c([nH]c3ccccc23)[C@@H](c2ccc(O[C@@H]3CCNC3)s2)N1CC(C)(C)F]

Trifluoroacetic acid (1.0 mL, 13.238 mmol, 50 equiv) was added to a solution of tert-butyl (1S,3R)-1-(5-(((R)-1-(tert-butoxycarbonyl)pyrrolidin-3-yl)oxy)thiophen-2-yl)-2-(2-fluoro-2-methylpropyl)-3-methyl-1,2,3,4-tetrahydro-9H-pyrido[3,4-b]indole-9-carboxylate (0.166 g, 0.265 mmol, 1 equiv) in dichloromethane (5 mL) at 0° C. After stirring at room temperature for 3 h, LCMS analysis indicated that a 40 to 60 mixture of product and mono-Boc product. The mixture was warmed to room temperature and stirred at room temperature for 2 h. LC analysis indicated that the reaction was complete. Saturated sodium carbonate was added carefully at 0° C., adjusting the pH to 9-10. The mixture was extracted with dichloromethane (3×40 mL). The combined organic layers were dried over anhydrous sodium sulfate, filtered and concentrated under reduced pressure to give the crude title compound (0.107 g, 95% yield) as a brown foam. LCMS: m/z=428 [M+H]⁺

### Example 19: (1S,3R)-2-(2-Fluoro-2-methylpropyl)-3-methyl-1-(5-(((R)-1-propylpyrrolidin-3-yl)oxy)thiophen-2-yl)-2,3,4,9-tetrahydro-1H-pyrido[3,4-b]indole

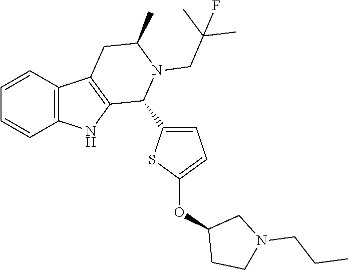 [SMILES: CCCN1CC[C@@H](Oc2ccc([C@@H]3c4[nH]c5ccccc5c4C[C@@H](C)N3CC(C)(C)F)s2)C1]

N,N-Diisopropylethylamine (0.21 mL, 1.183 mmol, 5 equiv) was added to a solution of (1S,3R)-2-(2-fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole (0.101 g, 0.236 mmol, 1 equiv) in N,N-dimethylacetamide (2.5 mL). After stirring at room temperature for 10 min, 1-bromopropane (34.9 mg, 0.284 mmol, 1.2 equiv) was added and the mixture was stirred at room temperature for 18 h at which time LC analysis indicated ˜70% conversion. Additional 1-bromopropane (10 mg, 0.081 mmol, 0.34 equiv) was added and the mixture was stirred at room temperature for 20 h. LC analysis indicated >95% conversion. The mixture was diluted with ethyl acetate (80 mL) and washed with water (40 mL). The aqueous layer was extracted with ethyl acetate (2×40 mL). The combined organic layers were washed with saturated brine (50 mL), dried over anhydrous sodium sulfate, filtered and concentrated under reduced pressure. The residue was dry-loaded onto Celite® and purified on an Interchim automated chromatography system (28 g Biotage Sfar KP-Amino D column), eluting with a gradient of 0 to 100% dichloromethane in heptanes, to give the title compound (33 mg, 30% yield) as a white solid. LCMS: m/z=470 [M+H]⁺; ¹H NMR (400 MHz, CDCl₃) δ=7.82 (br s, 1H), 7.51 (d, J=7.6 Hz, 1H), 7.35-7.29 (m, 1H), 7.21-7.08 (m, 2H), 6.31 (br s, 1H), 5.90 (d, J=3.8 Hz, 1H), 5.11 (br s, 1H), 4.70 (br dd, J=5.3, 7.6 Hz, 1H), 3.49 (br s, 1H), 2.87-2.73 (m, 3H), 2.71-2.61 (m, 2H), 2.58 (s, 1H), 2.55-2.37 (m, 4H), 2.22 (qd, J=7.1, 14.0 Hz, 1H), 2.08-2.00 (m, 1H), 1.57-1.43 (m, 5H), 1.37-1.29 (m, 3H), 1.15 (d, J=6.7 Hz, 3H), 0.92 (t, J=7.3 Hz, 3H); ¹⁹F NMR (376 MHz, CDCl₃) δ=−140.13 (br s, 1F).

### Example 20: (1S,3R)-2-(2-Fluoro-2-methylpropyl)-1-(5-(((R)-1-(3-fluoropropyl)pyrrolidin-3-yl)oxy)thiophen-2-yl)-3-methyl-2,3,4,9-tetrahydro-1H-pyrido[3,4-b]indole

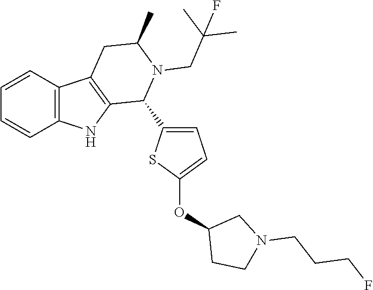 [SMILES: C[C@@H]1Cc2c([nH]c3ccccc23)[C@@H](c2ccc(O[C@@H]3CCN(CCCF)C3)s2)N1CC(C)(C)F]

1-Bromo-3-fluoropropane (39 μL, 0.423 mmol, 1.2 equiv) and N,N-diisopropylethylamine (0.31 mL, 1.76 mmol, 5 equiv) were sequentially added to a solution of (1S,3R)-2-(2-fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole (151 mg, 0.353 mmol, 1 equiv) in anhydrous N,N-dimethylacetamide (1.5 mL) at room temperature. After stirring for 18 h, the reaction was diluted with ethyl acetate (30 mL) and washed with saturated brine (30 mL). The organic layer was dried over sodium sulfate, filtered and concentrated under reduced pressure. The residue was pre-absorbed on Celite® (1 g) and purified on an Interchim automated chromatography system (11 g Biotage Sfar KP-Amino D column), eluting with a gradient of 0 to 100% dichloromethane in heptanes, to give the title compound (65.5 mg, 38% yield) as a yellow solid. LCMS (ESI) m/z=488.3 [M+H]⁺; ¹H NMR (400 MHz, CDCl₃) δ=7.84 (br s, 1H), 7.56 (s, 1H), 7.52 (d, J=7.7 Hz, 1H), 7.32 (d, J=7.9 Hz, 1H), 7.18 (dt, J=1.3, 7.5 Hz, 1H), 7.14-7.09 (m, 1H), 6.43 (dd, J=1.2, 3.9 Hz, 1H), 6.31 (br d, J=1.0 Hz, 1H), 5.93 (d, J=3.8 Hz, 1H), 5.90 (d, J=3.8 Hz, 1H), 5.12 (br s, 1H), 4.70 (dt, J=4.2, 7.2 Hz, 1H), 4.57 (t, J=5.9 Hz, 1H), 4.45 (t, J=6.0 Hz, 1H), 3.49 (br s, 1H), 2.86-2.76 (m, 3H), 2.73-2.40 (m, 7H), 2.29-2.17 (m, 1H), 2.10-2.01 (m, 1H), 1.99-1.84 (m, 2H), 1.57-1.43 (m, 2H), 1.38-1.29 (m, 3H), 1.19-1.11 (m, 3H).

### Example 21: (1S,3R)-1-(5-(((R)-1-(3,3-Difluoropropyl)pyrrolidin-3-yl)oxy)thiophen-2-yl)-2-(2-fluoro-2-methylpropyl)-3-methyl-2,3,4,9-tetrahydro-1H-pyrido[3,4-b]indole

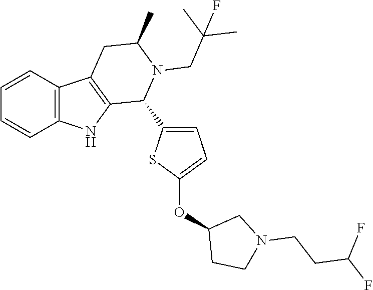 [SMILES: C[C@@H]1Cc2c([nH]c3ccccc23)[C@@H](c2ccc(O[C@@H]3CCN(CCC(F)F)C3)s2)N1CC(C)(C)F]

N,N-Diisopropylethylamine (0.31 mL, 1.754 mmol, 5 equiv) was added to a solution of (1S,3R)-2-(2-fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole (0.150 g, 0.351 mmol, 1 equiv) in N,N-dimethylacetamide (3 mL). After stirring at room temperature for 10 min, 3,3-difluoropropyl 4-methylbenzenesulfonate (132 mg, 0.526 mmol, 1.5 equiv) was added and the mixture was stirred at room temperature for 4 days, at which time LC analysis indicated ˜82% completion. The mixture was diluted with ethyl acetate (80 mL) and washed with water (40 mL). The aqueous layer was extracted with ethyl acetate (2×40 mL). The combined organic layers were washed with saturated brine (50 mL), dried over anhydrous sodium sulfate, filtered and concentrated under reduced pressure. The residue was dry-loaded onto Celite® and purified on an Interchim automated chromatography system (11 g Biotage Sfar KP-Amino D column), eluting with a gradient of 0 to 100% dichloromethane in heptanes, to give the title compound (66.9 mg, 38% yield) as a yellow solid. LCMS: m/z=506 [M+H]⁺; ¹H NMR (400 MHz, CDCl₃) δ=7.80 (d, 1H), 7.56-7.50 (m, 1H), 7.33 (d, J=7.4 Hz, 1H), 7.18 (t, J=7.0 Hz, 1H), 7.14-7.09 (m, 1H), 6.45-6.40 (m, 1H), 6.31 (br s, 1H), 6.09-5.75 (m, 2H), 5.17 (s, 1H), 5.12 (br s, 1H), 4.73-4.67 (m, 1H), 3.49 (br s, 1H), 2.88 (s, 1H), 2.88-2.77 (m, 3H), 2.72-2.47 (m, 7H), 2.29-2.16 (m, 1H), 2.11-1.97 (m, 3H), 1.60-1.54 (m, 1H), 1.50-1.41 (m, 2H), 1.37-1.29 (m, 3H), 1.18-1.11 (m, 3H); ¹⁹F NMR (376 MHz, CDCl₃) δ=−116.80 (s, 2F), −139.25-−141.33 (br s, 1F).

### Example 22: (1S,3R)-2-(2-Fluoro-2-methylpropyl)-1-(5-(((R)-1-(2-fluoroethyl)pyrrolidin-3-yl)oxy)thiophen-2-yl)-3-methyl-2,3,4,9-tetrahydro-1H-pyrido[3,4-b]indole

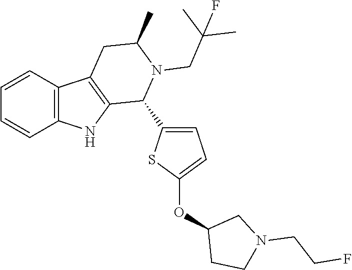 [SMILES: C[C@@H]1Cc2c([nH]c3ccccc23)[C@@H](c2ccc(O[C@@H]3CCN(CCF)C3)s2)N1CC(C)(C)F]

N,N-Diisopropylethylamine (0.31 mL, 1.754 mmol, 5 equiv) was added to a solution of (1S,3R)-2-(2-fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole (0.150 g, 0.351 mmol, 1 equiv) in N,N-dimethylacetamide (3 mL). After stirring at room temperature for 10 min, 1-fluoro-2-iodoethane (73.6 mg, 0.423 mmol, 1.2 equiv) was added and the mixture was stirred at room temperature for 20 h, at which time LC analysis indicated ˜78% completion. Additional 1-fluoro-2-iodoethane (18 mg, 0.105 mmol, 0.3 equiv) was added and the mixture was stirred at room temperature for 20 h. The mixture was diluted with ethyl acetate (80 mL) and washed with water (40 mL). The aqueous layer was extracted with ethyl acetate (2×40 mL). The combined organic layers were washed with saturated brine (50 mL), dried over anhydrous sodium sulfate, filtered and concentrated under reduced pressure. The residue was dry-loaded onto Celite® and purified on an Interchim automated chromatography system (11 g Biotage Sfar KP-Amino D column), eluting with a gradient of 0 to 100% dichloromethane in heptanes, to give the title compound (90 mg, 54% yield) as a yellow solid. LCMS: m/z=474 [M+H]⁺; ¹H NMR (400 MHz, CDCl₃) δ=7.92-7.71 (m, 1H), 7.56-7.49 (m, 1H), 7.36-7.29 (m, 1H), 7.17 (t, J=7.0 Hz, 1H), 7.14-7.08 (m, 1H), 6.31 (br s, 1H), 5.95-5.87 (m, 1H), 5.21-5.15 (m, 1H), 5.12 (br s, 1H), 4.76-4.67 (m, 1H), 4.63 (t, J=5.0 Hz, 1H), 4.51 (t, J=5.0 Hz, 1H), 3.49 (br s, 1H), 3.10-2.77 (m, 5H), 2.69-2.57 9 m, 3H), 2.55-2.45 (m, 1H), 2.30-2.19 (m, 1H), 2.12-2.02 (m, 1H), 1.53-1.43 (m, 3H), 1.37-1.30 (m, 3H), 1.17-1.12 (m, 3H); ¹⁹F NMR (376 MHz, CDCl₃) δ=−140.25 (br s, 1F), −219.34-−219.80 (m, 1F).

### Example 23: (1S,3R)-1-(5-(((R)-1-Ethylpyrrolidin-3-yl)oxy)thiophen-2-yl)-2-(2-fluoro-2-methylpropyl)-3-methyl-2,3,4,9-tetrahydro-1H-pyrido[3,4-b]indole

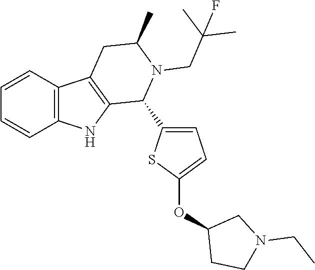 [SMILES: CCN1CC[C@@H](Oc2ccc([C@@H]3c4[nH]c5ccccc5c4C[C@@H](C)N3CC(C)(C)F)s2)C1]

N,N-Diisopropylethylamine (0.35 mL, 1.989 mmol, 5 equiv) was added to a solution of (1S,3R)-2-(2-fluoro-2-methylpropyl)-3-methyl-1-(5-(((R)-pyrrolidin-3-yl)oxy)thiophen-2-yl)-2,3,4,9-tetrahydro-1H-pyrido[3,4-b]indole (0.170 g, 0.398 mmol, 1 equiv) in N,N-dimethylacetamide (3 mL). After stirring at room temperature for 10 min, bromoethane (65 mg, 0.526 mmol, 1.5 equiv) was added and the mixture was stirred at room temperature for 24 h. The mixture was diluted with ethyl acetate (80 mL) and washed with water (40 mL). The aqueous layer was extracted with ethyl acetate (2×40 mL). The combined organic layers were washed with saturated brine (50 mL), dried over anhydrous sodium sulfate, filtered and concentrated under reduced pressure. The residue was dry-loaded onto Celite® and purified on an Interchim automated chromatography system (11 g Biotage Sfar KP-Amino D column), eluting with a gradient of 0 to 100% dichloromethane in heptanes, to give the title compound (54 mg, 30% yield) as a yellow solid. LCMS: m/z=456 [M+H]⁺; ¹H NMR (400 MHz, CDCl₃) δ=7.87 (br s, 1H), 7.56-7.49 (m, 1H), 7.32 (d, J=7.6 Hz, 1H), 7.17 (t, J=7.0 Hz, 1H), 7.13-7.10 (m, 1H), 6.30 (br s, 1H), 5.98-5.86 (m, 1H), 5.11 (br s, 1H), 4.74-4.67 (m, 1H), 3.49 (br s, 1H), 2.85-2.75 (m, 3H), 2.74-2.39 (m, 7H), 2.30-2.17 (m, 1H), 2.10-1.99 (m, 1H), 1.75 (br s, 1H), 1.53-1.43 (m, 3H), 1.37-1.30 (m, 3H), 1.17-1.10 (m, 6H); ¹⁹F NMR (376 MHz, CDCl₃) δ=−140.25 (br s, 1F).

### Example 24: (1S,3R)-1-(5-Bromothiophen-2-yl)-2-(3-((tert-butyldiphenylsilyl)oxy)-2,2-difluoropropyl)-3-methyl-2,3,4,9-tetrahydro-1H-pyrido[3,4-b]indole

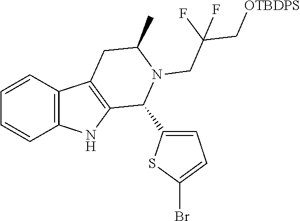

5-Bromothiophene-2-carbaldehyde (1.66 g, 8.71 mmol, 1 equiv) and acetic acid (2.0 mL, 36 mmol, 4.0 equiv) were sequentially added to a solution of (R)—N-(1-(1H-indol-3-yl)propan-2-yl)-3-((tert-butyldiphenylsilyl)oxy)-2,2-difluoropropan-1-amine (5.00 g, 10.4 mmol, 1.1 equiv) in toluene (44 mL) in a 250 mL round bottom flask. After heating at 80° C. for 32 h, the reaction was cooled to room temperature and diluted with ethyl acetate (80 mL) and saturated sodium bicarbonate (60 mL). The layers were separated, and the aqueous solution was extracted with ethyl acetate (2×80 mL). The combined organic layers were dried over sodium sulfate, filtered and concentrated under reduced pressure onto silica (38 g). The product was purified on a Biotage automated chromatography system (330 g, Sorbtech 40-75 μm silica gel column), eluting with a gradient of 0 to 25% ethyl acetate in heptanes. The solid was dried under vacuum at 40° C. for 3 h to give the title compound (5.4 g, 91% yield) as a pale-yellow foam. LCMS: m/z=679.1 [M+H]⁺.

### Example 25: tert-Butyl (1S,3R)-1-(5-bromothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

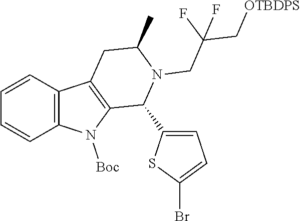

Di-tert-butyl dicarbonate (2.7 mL, 11.7 mmol, 1.5 equiv), triethylamine (2.2 mL, 15.6 mmol, 2.0 equiv) and 4-dimethylaminopyridine (0.133 g, 1.09 mmol, 0.14 equiv) were sequentially added to (1S,3R)-1-(5-bromothiophen-2-yl)-2-(3-((tert-butyldiphenylsilyl)oxy)-2,2-difluoropropyl)-3-methyl-2,3,4,9-tetrahydro-1H-pyrido[3,4-b]indole (5.29 g, 7.78 mmol, 1 equiv) in dichloromethane (97 mL) in a 250 mL round-bottom flask. After stirring at room temperature for 5 h, the reaction mixture was transferred, diluted with dichloromethane (80 mL) and washed with water (2×100 mL) and saturated brine (100 mL). The organic layer was dried over sodium sulfate, filtered and concentrated under reduced pressure onto silica (30 g). The residue was purified on a Büchi automated chromatography system (220 g, Sorbtech 40-75 μm silica gel column), eluting with a gradient of 0 to 10% ethyl acetate in heptanes. The solid was dried under vacuum at 45° C. for 1 h to give the title compound (5.28 g, 87% yield) as a white foam. LCMS: m/z=781.2 [M+H]⁺.

### Example 26: tert-Butyl (1S,3R)-1-(5-((1-(tert-butoxycarbonyl)azetidin-3-yl)oxy)thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

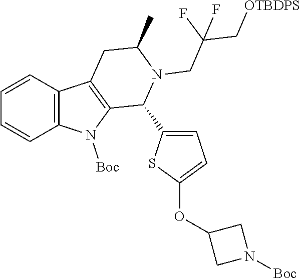

tert-Butyl (1S,3R)-1-(5-bromothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.500 g, 0.642 mmol, 1 equiv) and tert-butyl 3-hydroxyazetidine-1-carboxylate (0.367 g, 2.12 mmol, 3.3 equiv) in acetonitrile (6.4 mL) was sparged with nitrogen for 20 min in a 40 mL vial. Nickel(II) chloride ethylene glycol dimethylene ether complex (71 mg, 0.321 mmol, 0.5 equiv), 4,4′-bis(di-t-butyl)-2,2′-bipyridine (86 mg, 0.321 mmol, 0.5 equiv), Ir[dF(CF₃)PPy]₂dtbbpy]PF₆ (72 mg, 0.064 mmol, 0.10 equiv) and 1,4-diazabicyclo[2.2.2]octane (0.216 g, 1.93 mmol, 3.0 equiv) were sequentially added. The reaction mixture was sparged with nitrogen for an additional 5 min. The reaction was irradiated with blue LED light at room temperature for 24 h. This reaction was performed in duplicate on this scale and combined for isolation. The reaction mixture was filtered through Celite® and concentrated under reduced pressure onto silica (6 g). The residue was purified on a Biotage automated chromatography system (50 g, Biotage 60 μm silica gel column), eluting with a gradient of 0 to 30% ethyl acetate in heptanes. The solid was dried under vacuum at room temperature for 16 h to give the title compound (0.25 g, 23% yield) as an off-white foam. LCMS: m/z=872.4 [M+H]⁺.

### Example 27: (1S,3R)-1-(5-(Azetidin-3-yloxy)thiophen-2-yl)-2-(3-((tert-butyldiphenylsilyl)oxy)-2,2-difluoropropyl)-3-methyl-2,3,4,9-tetrahydro-1H-pyrido[3,4-b]indole

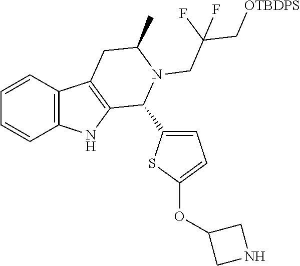

Trifluoroacetic acid (1.3 mL, 17 mmol, 60 equiv) was added to tert-butyl (1S,3R)-1-(5-((1-(tert-butoxycarbonyl)azetidin-3-yl)oxy)thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.253 g, 0.290 mmol, 1 equiv) in dichloromethane (5.8 mL) at 0° C. in a 40 mL vial. The reaction was stirred at 0° C. for 2.5 h, then warmed to room temperature and stirred for 4 h. The reaction was cooled back down to 0° C. and diluted with cold dichloromethane (50 mL), then poured into a cold saturated sodium carbonate (50 mL). The mixture was separated, and the aqueous layer was extracted with dichloromethane (2×25 mL). The combined organics were washed with saturated sodium carbonate (50 mL), dried over sodium sulfate, filtered and concentrated under reduced pressure to give the title compound (0.176 g, 90% yield) as a yellow/orange foam, which was used subsequently. LCMS: m/z=672.3 [M+H]⁺.

### Example 28: (1S,3R)-2-(3-((tert-Butyldiphenylsilyl)oxy)-2,2-difluoropropyl)-1-(5-((1-(3-fluoropropyl)azetidin-3-yl)oxy)thiophen-2-yl)-3-methyl-2,3,4,9-tetrahydro-1H-pyrido[3,4-b]indole

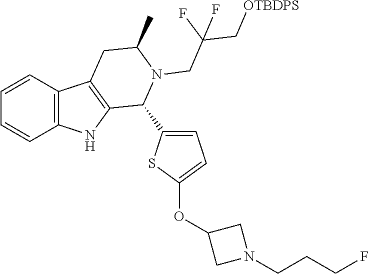

1-Bromo-3-fluoropropane (0.03 mL, 0.3 mmol, 1.1 equiv) and N,N-diisopropylethylamine (0.23 mL, 1.3 mmol, 5 equiv) were sequentially added to a solution of (1S,3R)-1-(5-(azetidin-3-yloxy)thiophen-2-yl)-2-(3-((tert-butyldiphenylsilyl)oxy)-2,2-difluoropropyl)-3-methyl-2,3,4,9-tetrahydro-1H-pyrido[3,4-b]indole (0.176 g, 0.262 mmol, 1 equiv) in N,N-dimethylacetamide (2.6 mL) in a 40 mL vial. After stirring for 28 h at room temperature, the reaction mixture was diluted with ethyl acetate (30 mL) and sequentially washed with water (3×30 mL) and saturated brine (30 mL). The organic layer was dried over sodium sulfate, filtered and concentrated under reduced pressure onto Celite® (2 g). The residue was purified on a Biotage automated chromatography system (28 g, Biotage 50 μm KP-amino-d column), eluting with a gradient of 0 to 30% ethyl acetate in heptanes to give the title compound (0.123 g, 64% yield) as an off-white foam. LCMS: m/z=732.3 [M+H]⁺.

### Example 29: 2,2-Difluoro-3-((1S,3R)-1-(5-((1-(3-fluoropropyl)azetidin-3-yl)oxy)thiophen-2-yl)-3-methyl-1,3,4,9-tetrahydro-2H-pyrido[3,4-b]indol-2-yl)propan-1-ol

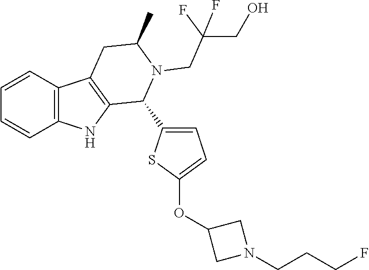 [SMILES: C[C@@H]1Cc2c([nH]c3ccccc23)[C@@H](c2ccc(OC3CN(CCCF)C3)s2)N1CC(F)(F)CO]

1 M Tetrabutylammonium fluoride (0.18 mL, 0.18 mmol, 1.1 equiv) was added to (1S,3R)-2-(3-((tert-butyldiphenylsilyl)oxy)-2,2-difluoropropyl)-1-(5-((1-(3-fluoropropyl)azetidin-3-yl)oxy)thiophen-2-yl)-3-methyl-2,3,4,9-tetrahydro-1H-pyrido[3,4-b]indole (0.121 g, 0.165 mmol, 1 equiv) in THF (1.5 mL) in a 40 mL vial at room temperature. After stirring for 1.5 h at room temperature, the reaction mixture was concentrated under reduced pressure. The residue was diluted with ethyl acetate (10 mL) and washed with saturated ammonium chloride (10 mL). The aqueous layer was extracted with ethyl acetate (10 mL). The combined organic layers were sequentially washed with water (15 mL) and saturated brine (15 mL). The organic layer was dried over sodium sulfate, filtered and concentrated under reduced pressure onto Celite® (1.5 g). The residue was purified on a Biotage automated chromatography system (11 g, Biotage 50 μm KP-amino-d column), eluting with a gradient of 0 to 50% ethyl acetate in dichloromethane, to give the title compound (48 mg, 59% yield) as an off-white foam. ¹H NMR (400 MHz, CDCl₃) δ=7.79 (s, 1H), 7.52 (d, J=7.8 Hz, 1H), 7.33-7.29 (m, 1H), 7.22-7.10 (m, 2H), 6.42 (d, J=3.4 Hz, 1H), 5.90 (d, J=3.9 Hz, 1H), 5.10 (s, 1H), 4.69 (quin, J=5.7 Hz, 1H), 4.54 (t, J=6.0 Hz, 1H), 4.42 (t, J=5.9 Hz, 1H), 4.00-3.89 (m, 2H), 3.76-3.70 (m, 2H), 3.62-3.52 (m, 1H), 3.22-3.10 (m, 3H), 2.93 (td, J=11.2, 14.8 Hz, 1H), 2.78 (dd, J=4.3, 16.0 Hz, 1H), 2.64-2.52 (m, 3H), 1.82-1.68 (m, 2H), 1.23 (d, J=6.8 Hz, 3H); LCMS: m/z=494.2 [M+H]⁺.

### Example 30: tert-Butyl (1S,3R)-1-(5-(((R)-1-(tert-butoxycarbonyl)pyrrolidin-3-yl)oxy)thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

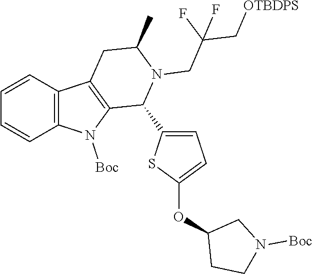

A solution of tert-butyl (1S,3R)-1-(5-bromothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (1.00 g, 1.3 mmol, 1 equiv) and tert-butyl (R)-3-hydroxypyrrolidine-1-carboxylate (0.80 g, 4.28 mmol, 3.34 equiv) in anhydrous acetonitrile (20 mL) was sparged with a nitrogen for 20 min. 1,4-Diazabicyclo[2.2.2]octane (0.44 g, 3.84 mmol, 3.0 equiv), nickel (II) chloride ethylene glycol dimethylene ether complex (0.14 g, 0.64 mmol, 0.5 equiv), 4,4′-bis(di-t-butyl)-2,2′-bipyridine (0.17 g, 0.64 mmol, 0.5 equiv), and Ir[dF(CF₃)PPy]₂dtbbpy]PF₆ catalyst (0.14 g, 0.13 mmol, 0.1 equiv) were sequentially added and the mixture was sparged with nitrogen for 20 min. The resulting clear yellow solution was stirred at room temperature under irradiation with blue LED light for 24 h. The mixture was filtered through Celite®, and the filter pad was washed with ethyl acetate (3×10 mL). The filtrate was concentrated under reduced pressure, and the residue was purified on a Büchi automated chromatography system (Sorbtech 40 g silica gel column), eluting with a gradient of 0 to 100% ethyl acetate in heptanes, to give the title compound (0.36 g, 31% yield) as a white foam. LCMS: m/z=886.4 [M+H]⁺.

### Example 31: (1S,3R)-2-(3-((tert-Butyldiphenylsilyl)oxy)-2,2-difluoropropyl)-3-methyl-1-(5-(((R)-pyrrolidin-3-yl)oxy)thiophen-2-yl)-2,3,4,9-tetrahydro-1H-pyrido[3,4-b]indole

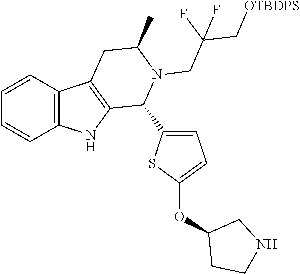

Trifluoroacetic acid (1.9 mL, 24 mmol, 60 equiv) was added dropwise over ˜2 min to tert-butyl (1S,3R)-1-(5-(((R)-1-(tert-butoxycarbonyl)pyrrolidin-3-yl)oxy)thiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.36 g, 0.41 mmol, 1.0 equiv) in dichloromethane (5.0 mL) in a 40 mL vial at 0° C. The reaction was stirred at 0° C. for 18 h and warmed to room temperature for 2 h. Cold saturated sodium carbonate was added to the mixture adjusting the pH to 11. The solution was transferred to a separatory funnel and was extracted with dichloromethane (3×15 mL). The combined organic layers were washed with saturated brine (1×20 mL). The organic layer was dried over sodium sulfate, filtered and concentrated under reduced pressure and dried under vacuum at 25° C. for 1.5 h to give the crude title compound (0.276 g, 100% yield) as a green foam. LCMS: m/z=686.3 [M+H]⁺.

### Example 32: (1S,3R)-2-(3-((tert-Butyldiphenylsilyl)oxy)-2,2-difluoropropyl)-1-(5-(((R)-1-(3-fluoropropyl)pyrrolidin-3-yl)oxy)thiophen-2-yl)-3-methyl-2,3,4,9-tetrahydro-1H-pyrido[3,4-b]indole

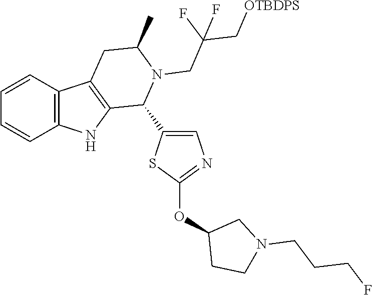

1-Bromo-3-fluoropropane (55 μL, 0.6 mmol, 1.5 equiv) and diisopropylethylamine (0.35 mL, 2.0 mmol, 5 equiv) were added to (1S,3R)-2-(3-((tert-butyldiphenylsilyl)oxy)-2,2-difluoropropyl)-3-methyl-1-(5-(((R)-pyrrolidin-3-yl)oxy)thiophen-2-yl)-2,3,4,9-tetrahydro-1H-pyrido[3,4-b]indole (0.276 g, 0.40 mmol, 1.0 equiv) in N,N-dimethylacetamide (2.0 mL) in a 40 mL vial and stirred at room temperature for 32 h. The reaction mixture was transferred to a separatory funnel with ethyl acetate (20 mL) and washed with saturated brine (2×15 mL). The organic layers were dried over sodium sulfate, filtered and concentrated under reduced pressure onto silica (12 g). The material was purified on a Büchi automated chromatography system (28 g, Biotage 50 μm KP-amino-d column), eluting with a gradient of 0 to 100% ethyl acetate in heptanes. The oil was dried under vacuum at 25° C. for 2 h to give the title compound (0.22 g, 73% yield). LCMS: m/z=747.3 [M+H]⁺.

### Example 33: 2,2-Difluoro-3-((1S,3R)-1-(5-(((R)-1-(3-fluoropropyl)pyrrolidin-3-yl)oxy)thiophen-2-yl)-3-methyl-1,3,4,9-tetrahydro-2H-pyrido[3,4-b]indol-2-yl)propan-1-ol

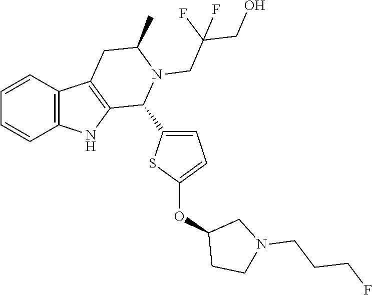 [SMILES: C[C@@H]1Cc2c([nH]c3ccccc23)[C@@H](c2ccc(O[C@@H]3CCN(CCCF)C3)s2)N1CC(F)(F)CO]

1M Tetra-n-butyl ammonium fluoride in tetrahydrofuran (0.31 mL, 0.31 mmol, 1.10 equiv) was added dropwise to (1S,3R)-2-(3-((tert-butyldiphenylsilyl)oxy)-2,2-difluoropropyl)-1-(5-(((R)-1-(3-fluoropropyl)pyrrolidin-3-yl)oxy)thiophen-2-yl)-3-methyl-2,3,4,9-tetrahydro-1H-pyrido[3,4-b]indole (0.22 g, 0.28 mmol, 1.0 equiv) in tetrahydrofuran (2.0 mL) in a 40 mL vial and stirred at room temperature for 1.5 h. Solvent was concentrated under reduced pressure and saturated ammonium chloride solution (5 mL) was added. The solution was transferred to a separatory funnel and was extracted with ethyl acetate (3×10 mL). The combined organic layers were washed with saturated brine (20 mL), dried over sodium sulfate, filtered and concentrated under reduced pressure onto silica (12 g). The material was purified on a Büchi automated chromatography system (11 g, Biotage 50 μm KP-amino-d column), eluting with a gradient of 0 to 100% ethyl acetate in dichloromethane. The solid was dried under vacuum at 25° C. for 16 h to give the title compound (43 mg, 30% yield) as a yellow solid. ¹H NMR (400 MHz, CDCl₃) δ=7.79 (s, 1H), 7.52 (d, J=7.8 Hz, 1H), 7.33-7.29 (m, 1H), 7.19 (dt, J=1.3, 7.5 Hz, 1H), 7.15-7.10 (m, 1H), 6.43 (d, J=3.4 Hz, 1H), 5.95 (d, J=3.8 Hz, 1H), 5.10 (s, 1H), 4.69 (tdd, J=2.8, 5.2, 7.7 Hz, 1H), 4.57 (t, J=5.9 Hz, 1H), 4.45 (t, J=5.9 Hz, 1H), 3.99-3.88 (m, 2H), 3.59 (qd, J=6.8, 13.2 Hz, 1H), 3.22-3.10 (m, 1H), 3.01-2.90 (m, 1H), 2.86-2.75 (m, 4H), 2.64-2.45 (m, 4H), 2.28-2.18 (m, 1H), 2.08-1.99 (m, 1H), 1.97-1.83 (m, 2H), 1.23 (d, J=6.7 Hz, 3H); LCMS: m/z=508.2 [M+H]⁺.

### Example 34: (1S,3R)-1-(5-Bromothiophen-2-yl)-2-(2,2-difluoroethyl)-3-methyl-2,3,4,9-tetrahydro-1H-pyrido[3,4-b]indole

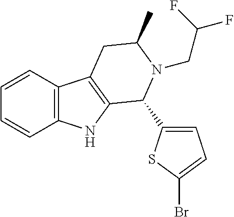 [SMILES: C[C@@H]1Cc2c([nH]c3ccccc23)[C@@H](c2ccc(Br)s2)N1CC(F)F]

5-Bromothiophene-2-carbaldehyde (3.00 g, 15.7 mmol, 1 equiv) and acetic acid (1.8 mL, 36 mmol, 2.0 equiv) were sequentially added to a solution of (R)—N-(2,2-difluoroethyl)-1-(1H-indol-3-yl)propan-2-amine (4.48 g, 18.8 mmol, 1.2 equiv) in toluene (79 mL) in a 250 ml round bottom flask. After heating at 80° C. for 21 h, the reaction was cooled to room temperature and diluted with ethyl acetate (80 mL) and saturated sodium bicarbonate (60 mL). The layers were separated, and the aqueous layer was extracted with ethyl acetate (2×80 mL). The combined organic layers were dried over sodium sulfate, filtered and concentrated under reduced pressure onto silica (38 g). The product was purified on a Büchi automated chromatography system (330 g, Sorbtech 40-75 μm silica gel column), eluting with a gradient of 0 to 20% methyl tert-butyl ether in heptanes. The solid was dried under vacuum at 40° C. for 16 h to give the title compound (3.9 g, 61% yield) as a pale-yellow semi-solid. LCMS: m/z=411.0 [M+H]⁺.

### Example 35: tert-Butyl (1S,3R)-1-(5-bromothiophen-2-yl)-2-(2,2-difluoroethyl)-3-methyl-1,2,3,4-tetrahydro-9H-pyrido[3,4-b]indole-9-carboxylate

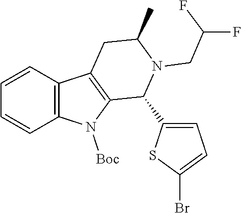 [SMILES: C[C@@H]1Cc2c(n(C(=O)OC(C)(C)C)c3ccccc23)[C@@H](c2ccc(Br)s2)N1CC(F)F]

Di-tert-butyl dicarbonate (3.3 mL, 14 mmol, 1.5 equiv), triethylamine (2.7 mL, 19 mmol, 2.0 equiv) and 4-dimethylaminopyridine (0.16 g, 1.3 mmol, 0.14 equiv) were added to a solution of (1S,3R)-1-(5-bromothiophen-2-yl)-2-(2,2-difluoroethyl)-3-methyl-2,3,4,9-tetrahydro-1H-pyrido[3,4-b]indole (3.93 g, 9.56 mmol, 1 equiv) in dichloromethane (120 mL) in a 250 mL round-bottom flask. After stirring at room temperature for 3 h, the reaction mixture was diluted with dichloromethane (100 mL) and washed with water (2×100 mL) and saturated brine (100 mL). The organic layer was dried over sodium sulfate, filtered and concentrated under reduced pressure onto silica gel (30 g). The residue was purified on a Büchi automated chromatography system (100 g, Sorbtech 40-75 μm silica gel column), eluting with a gradient of 0 to 10% ethyl acetate in heptanes. The solid was dried under vacuum at 45° C. for 2 h to give the title compound (4.04 g, 83% yield) as a white foam. LCMS: m/z=511.1 [M+H]⁺.

### Example 36: tert-Butyl (1S,3R)-1-(5-((1-(tert-butoxycarbonyl)azetidin-3-yl)oxy)thiophen-2-yl)-2-(2,2-difluoroethyl)-3-methyl-1,2,3,4-tetrahydro-9H-pyrido[3,4-b]indole-9-carboxylate

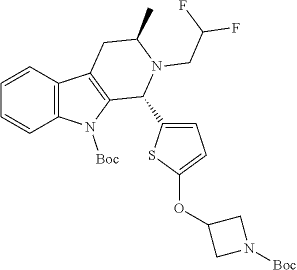 [SMILES: C[C@@H]1Cc2c(n(C(=O)OC(C)(C)C)c3ccccc23)[C@@H](c2ccc(OC3CN(C(=O)OC(C)(C)C)C3)s2)N1CC(F)F]

A solution of tert-butyl (1S,3R)-1-(5-bromothiophen-2-yl)-2-(2,2-difluoroethyl)-3-methyl-1,2,3,4-tetrahydro-9H-pyrido[3,4-b]indole-9-carboxylate (1.00 g, 1.96 mmol, 1 equiv) and tert-butyl 3-hydroxyazetidine-1-carboxylate (1.12 g, 6.46 mmol, 3.3 equiv) in acetonitrile (20 mL) was sparged with nitrogen for 20 min in a 40 mL vial. Nickel (II) chloride ethylene glycol dimethylene ether complex (0.220 g, 0.980 mmol, 0.5 equiv), 4,4′-bis(di-t-butyl)-2,2′-bipyridine (0.263 g, 0.980 mmol, 0.5 equiv), Ir[dF(CF₃)PPy]₂dtbbpy]PF₆ (0.220 g, 0.200 mmol, 0.10 equiv) and 1,4-diazabicyclo[2.2.2]octane (0.660 g, 5.88 mmol, 3.0 equiv) were added. The reaction mixture was sparged with nitrogen for an additional 5 min. The reaction was irradiated with blue LED light at room temperature for 5 days. The reaction mixture was filtered through Celite® and concentrated under reduced pressure onto silica gel (6 g). The residue was purified on a Biotage automated chromatography system (50 g, Biotage 60 μm silica gel column), eluting with a gradient of 0 to 30% ethyl acetate in heptanes. The solid was dried under vacuum at 40° C. for 3 h to give the title compound (0.23 g, 19% yield) as a white foam. LCMS: m/z=604.3 [M+H]⁺.

### Example 37: (1S,3R)-1-(5-(Azetidin-3-yloxy)thiophen-2-yl)-2-(2,2-difluoroethyl)-3-methyl-2,3,4,9-tetrahydro-1H-pyrido[3,4-b]indole

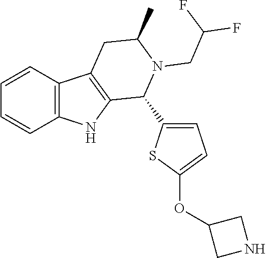 [SMILES: C[C@@H]1Cc2c([nH]c3ccccc23)[C@@H](c2ccc(OC3CNC3)s2)N1CC(F)F]

Trifluoroacetic acid (1.7 mL, 22 mmol, 60 equiv) was added to tert-butyl (1S,3R)-1-(5-((1-(tert-butoxycarbonyl)azetidin-3-yl)oxy)thiophen-2-yl)-2-(2,2-difluoroethyl)-3-methyl-1,2,3,4-tetrahydro-9H-pyrido[3,4-6]indole-9-carboxylate (0.222 g, 0.368 mmol, 1 equiv) in dichloromethane (7.4 mL) at 0° C. in a 40 mL vial. The reaction was stirred at 0° C. for 2.5 h, then warmed to room temperature and stirred for 1.5 h. The reaction was cooled to 0° C. and diluted with cold dichloromethane (60 mL) then poured into a solution of cold saturated sodium carbonate (60 mL). The layers were separated and the aqueous was extracted with dichloromethane (2×30 mL). The combined organic layers were washed with saturated sodium carbonate (60 mL), dried over sodium sulfate, filtered and concentrated under reduced pressure to give the title compound (0.162 g, >95% yield, 3:1 dr) as a yellow/orange solid, which was used subsequently. LCMS: m/z=404.2 [M+H]⁻.

### Example 38: (1S,3R)-2-(2,2-Difluoroethyl)-1-(5-((1-(3-fluoropropyl)azetidin-3-yl)oxy)thiophen-2-yl)-3-methyl-2,3,4,9-tetrahydro-1H-pyrido[3,4-b]indole

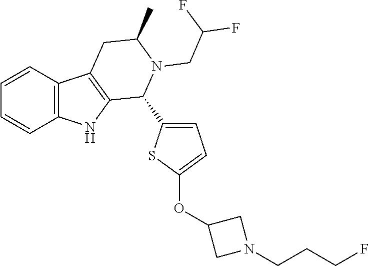 [SMILES: C[C@@H]1Cc2c([nH]c3ccccc23)[C@@H](c2ccc(OC3CN(CCCF)C3)s2)N1CC(F)F]

1-Bromo-3-fluoropropane (0.04 mL, 0.4 mmol, 1.2 equiv) and N,N-diisopropylethylamine (0.32 mL, 1.8 mmol, 5 equiv) were sequentially added to a solution of (1S,3R)-1-(5-(azetidin-3-yloxy)thiophen-2-yl)-2-(2,2-difluoroethyl)-3-methyl-2,3,4,9-tetrahydro-1H-pyrido[3,4-b]indole (0.148 g, 0.367 mmol, 1 equiv) in N,N-dimethylacetamide (3.6 mL) in a 40 mL vial. After stirring for 24 h at room temperature, the reaction mixture was diluted with ethyl acetate (20 mL) and sequentially washed with water (2×20 mL) and saturated brine (20 mL). The organic layer was dried over sodium sulfate, filtered and concentrated under reduced pressure onto Celite® (1 g). The residue was purified on a Biotage automated chromatography system (28 g, Biotage 50 μm KP-amino-d column), eluting with a gradient of 0 to 50% ethyl acetate in heptanes, to give partially purified title compound (64 mg, 38% yield, 3:1 dr). The crude mixture was purified by SFC with an IC column (25×0.46 cm) eluting with an isocratic method 85:15 carbon dioxide/methanol with 0.1% diethylamine to give the title compound (28 mg, 16% yield) as an off-white solid. ¹H NMR (400 MHz, CDCl₃) δ=7.68 (s, 1H), 7.51 (d, J=7.2 Hz, 1H), 7.30 (d, J=7.9 Hz, 1H), 7.21-7.08 (m, 2H), 6.48 (dd, J=1.0, 3.8 Hz, 1H), 5.96-5.58 (m, 2H), 4.96 (d, J=0.9 Hz, 1H), 4.69 (quin, J=5.7 Hz, 1H), 4.54 (t, J=5.9 Hz, 1H), 4.42 (t, J=5.9 Hz, 1H), 3.77-3.70 (m, 2H), 3.54-3.45 (m, 1H), 3.15-3.09 (m, 2H), 3.00 (dq, J=4.5, 14.9 Hz, 1H), 2.88-2.74 (m, 2H), 2.62 (t, J=7.1 Hz, 2H), 2.55 (ddd, J=1.2, 7.4, 15.7 Hz, 1H), 1.82-1.68 (m, 2H), 1.17 (d, J=6.7 Hz, 3H); LCMS: m/z=464.2 [M+H]⁺.

### Example 39: tert-Butyl (1S,3R)-1-(5-(((R)-1-(tert-butoxycarbonyl)pyrrolidin-3-yl)oxy)thiophen-2-yl)-2-(2,2-difluoroethyl)-3-methyl-1,2,3,4-tetrahydro-9H-pyrido[3,4-b]indole-9-carboxylate

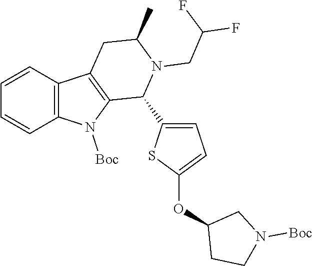 [SMILES: C[C@@H]1Cc2c(n(C(=O)OC(C)(C)C)c3ccccc23)[C@@H](c2ccc(O[C@@H]3CCN(C(=O)OC(C)(C)C)C3)s2)N1CC(F)F]

A solution of tert-butyl (1S,3R)-1-(5-bromothiophen-2-yl)-2-(2,2-difluoroethyl)-3-methyl-1,2,3,4-tetrahydro-9H-pyrido[3,4-b]indole-9-carboxylate (1.00 g, 2.0 mmol, 1 equiv) and tert-butyl (R)-3-hydroxypyrrolidine-1-carboxylate (1.22 g, 6.54 mmol, 3.34 equiv) in anhydrous acetonitrile (40 mL) was sparged with nitrogen for 20 min. 1,4-Diazabicyclo[2.2.2]octane (0.67 g, 5.88 mmol, 3.0 equiv), nickel (II) chloride ethylene glycol dimethylene ether complex (0.22 g, 0.98 mmol, 0.5 equiv), 4,4′-bis(di-t-butyl)-2,2′-bipyridine (0.26 g, 0.98 mmol, 0.5 equiv) and Ir[dF(CF₃)PPy]₂dtbbpy]PF₆ catalyst (0.22 g, 0.20 mmol, 0.1 equiv) were sequentially added, and the mixture was sparged with a stream of nitrogen for 20 min. The resulting clear yellow solution was stirred at room temperature under irradiation with blue LED light for 3 days. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 100% ethyl acetate in heptanes, to give the title compound (0.25 g, 20% yield) as a white solid. LCMS: m/z=618.3 [M+H]⁺.

### Example 40: (1S,3R)-2-(2,2-Difluoroethyl)-3-methyl-1-(5-(((R)-pyrrolidin-3-yl)oxy)thiophen-2-yl)-2,3,4,9-tetrahydro-1H-pyrido[3,4-b]indole

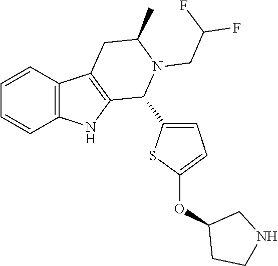 [SMILES: C[C@@H]1Cc2c([nH]c3ccccc23)[C@@H](c2ccc(O[C@@H]3CCNC3)s2)N1CC(F)F]

Trifluoroacetic acid (1.9 mL, 24.2 mmol, 60 equiv) was added dropwise over ˜2 min to tert-butyl (1S,3R)-1-(5-(((R)-1-(tert-butoxycarbonyl)pyrrolidin-3-yl)oxy)thiophen-2-yl)-2-(2,2-difluoroethyl)-3-methyl-1,2,3,4-tetrahydro-9H-pyrido[3,4-b]indole-9-carboxylate (0.25 g, 0.40 mmol, 1.0 equiv) in dichloromethane (5.0 mL) in a 40 mL vial at 0° C. The reaction was stirred at 0° C. for 18 h. Cold saturated sodium carbonate was added to the mixture adjusting the pH to 10. The solution was transferred to a separatory funnel and was extracted with dichloromethane (3×15 mL). The combined organic layers were washed with saturated brine (20 mL), dried over sodium sulfate, filtered and concentrated under reduced pressure. The product was dried under vacuum at 25° C. for 1.5 h to give the crude title compound (0.17 g, 100% yield) as an orange foam. LCMS: m/z=418.2 [M+H]⁺.

### Example 41: (1S,3R)-2-(2,2-Difluoroethyl)-1-(5-(((R)-1-(3-fluoropropyl)pyrrolidin-3-yl)oxy)thiophen-2-yl)-3-methyl-2,3,4,9-tetrahydro-1H-pyrido[3,4-b]indole

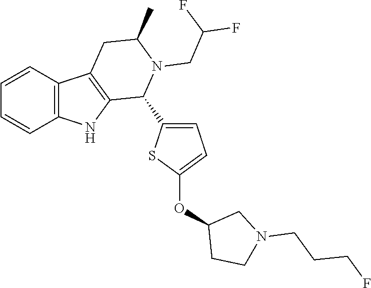 [SMILES: C[C@@H]1Cc2c([nH]c3ccccc23)[C@@H](c2ccc(O[C@@H]3CCN(CCCF)C3)s2)N1CC(F)F]

1-Bromo-3-fluoropropane (44 μL, 0.48 mmol, and N,N,-diisopropylethylamine (0.35 mL, 2.00 mmol, 5.0 equiv) were added to (1S,3R)-2-(2,2-difluoroethyl)-3-methyl-1-(5-(((R)-pyrrolidin-3-yl)oxy)thiophen-2-yl)-2,3,4,9-tetrahydro-1H-pyrido[3,4-b]indole (0.17 g, 0.40 mmol, 1.0 equiv) in N,N-dimethylacetamide (2.0 mL) in a 40 mL vial and stirred at room temperature for 36 h. The reaction mixture was diluted with ethyl acetate (20 mL) and washed with saturated brine (2×15 mL). The organic layer was dried over sodium sulfate, filtered and concentrated under reduced pressure onto silica (12 g). The material was purified on a Büchi automated chromatography system (28 g, Biotage 50 μm KP-amino-d column), eluting with a gradient of 0 to 100% dichloromethane in heptanes, to give the title compound (50 mg, 26% yield, ˜2:1 dr). It was further purified by SFC (Wavelength: 220, 254 and 280 nm; Column: OJ-H, 25×0.46 cm; Isocratic method: 80:20 CO₂/MeOH (0.1% DEA); Flow rate: 3.0 mL/min; Retention time: 5.17 min) to give the title compound (30 mg) as a yellow solid. ¹H NMR (400 MHz, CDCl₃) δ=7.69 (s, 1H), 7.51 (d, J=7.7 Hz, 1H), 7.30 (d, J=7.7 Hz, 1H), 7.17 (dt, J=1.2, 7.5 Hz, 1H), 7.14-7.08 (m, 1H), 6.49 (dd, J=0.9, 3.8 Hz, 1H), 5.95 (d, J=3.9 Hz, 1H), 5.77 (tt, J=4.5, 56.7 Hz, 1H), 4.96 (s, 1H), 4.68 (dt, J=3.2, 7.6 Hz, 1H), 4.57 (t, J=5.9 Hz, 1H), 4.45 (t, J=5.9 Hz, 1H), 3.56-3.46 (m, 1H), 3.07-2.93 (m, 1H), 2.89-2.74 (m, 5H), 2.66-2.45 (m, 4H), 2.29-2.17 (m, 1H), 2.08-1.84 (m, 3H), 1.17 (d, J=6.7 Hz, 3H); LCMS: m/z=478.2 [M+H]⁺.

### Example 42: (1S,3R)-1-(5-Bromothiophen-2-yl)-3-methyl-2-(2,2,2-trifluoroethyl)-2,3,4,9-tetrahydro-1H-pyrido[3,4-b]indole

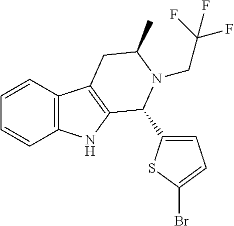 [SMILES: C[C@@H]1Cc2c([nH]c3ccccc23)[C@@H](c2ccc(Br)s2)N1CC(F)(F)F]

5-Bromothiophene-2-carbaldehyde (3.0 g, 15.7 mmol, 1 equiv) and acetic acid (4.1 mL, 42.4 mmol, 2.7 equiv) were sequentially added to a solution of (R)-1-(1H-indol-3-yl)-N-(2,2,2-trifluoroethyl)propan-2-amine (4.83 g, 18.84 mmol, 1.2 equiv) in toluene (80 mL) in a 250 mL round bottom flask. After heating at 80° C. for 40 h, the reaction was cooled to room temperature and diluted with ethyl acetate (80 mL) and saturated sodium bicarbonate (60 mL). The layers were separated, and the aqueous solution was extracted with ethyl acetate (2×80 mL). The combined organic layers were dried over sodium sulfate, filtered and concentrated under reduced pressure onto silica gel (38 g). The product was purified on a Büchi automated chromatography system (120 g, Sorbtech 40-75 μm silica gel column), eluting with a gradient of 0 to 25% ethyl acetate in heptanes. The solid was dried under vacuum at 25° C. for 16 h to give the title compound (4.70 g, 70% yield) as a yellow solid. LCMS: m/z=430.3 [M+H]⁺.

### Example 43: tert-Butyl (1S,3R)-1-(5-bromothiophen-2-yl)-3-methyl-2-(2,2,2-trifluoroethyl)-1,2,3,4-tetrahydro-9H-pyrido[3,4-b]indole-9-carboxylate

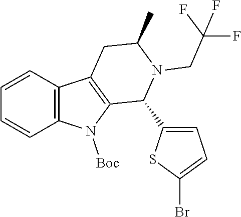 [SMILES: C[C@@H]1Cc2c(n(C(=O)OC(C)(C)C)c3ccccc23)[C@@H](c2ccc(Br)s2)N1CC(F)(F)F]

Di-tert-butyl dicarbonate (2.87 g, 13.1 mmol, 1.2 equiv), triethylamine (3.1 mL, 21.9 mmol, 2.0 equiv) and 4-dimethylaminopyridine (0.188 g, 1.53 mmol, 0.14 equiv) were added to a solution of (1S,3R)-1-(5-bromothiophen-2-yl)-3-methyl-2-(2,2,2-trifluoroethyl)-2,3,4,9-tetrahydro-1H-pyrido[3,4-b]indole (4.70 g, 11.0 mmol, 1 equiv) in dichloromethane (150 mL) in a 250 mL round-bottom flask. After stirring at room temperature for 2 h, the reaction mixture diluted with dichloromethane (80 mL) and was washed with water (2×100 mL) and saturated brine (100 mL). The organic layer was dried over sodium sulfate, filtered and concentrated under reduced pressure onto silica gel (40 g). The residue was purified on a Büchi automated chromatography system (120 g, Sorbtech 40-75 μm silica gel column), eluting with a gradient of 0 to 100% ethyl acetate in heptanes. The solid was dried under vacuum at 25° C. for 16 h to give the title compound (5.20 g, 90% yield) as a white foam. LCMS: m/z=530.1 [M+H]⁺.

### Example 44: tert-Butyl (1S,3R)-1-(5-((1-(tert-butoxycarbonyl)azetidin-3-yl)oxy)thiophen-2-yl)-3-methyl-2-(2,2,2-trifluoroethyl)-1,2,3,4-tetrahydro-9H-pyrido[3,4-b]indole-9-carboxylate

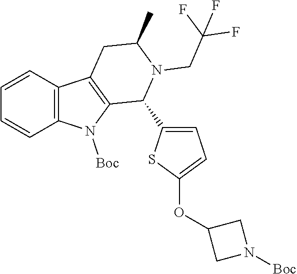 [SMILES: C[C@@H]1Cc2c(n(C(=O)OC(C)(C)C)c3ccccc23)[C@@H](c2ccc(OC3CN(C(=O)OC(C)(C)C)C3)s2)N1CC(F)(F)F]

A solution of tert-butyl (1S,3R)-1-(5-bromothiophen-2-yl)-3-methyl-2-(2,2,2-trifluoroethyl)-1,2,3,4-tetrahydro-9H-pyrido[3,4-b]indole-9-carboxylate (1.00 g, 1.89 mmol, 1 equiv) and tert-butyl 3-hydroxyazetidine-1-carboxylate (1.08 g, 6.23 mmol, 3.3 equiv) in acetonitrile (19 mL) was sparged with nitrogen for 20 min in a 40 mL vial. Nickel (II) chloride ethylene glycol dimethylene ether complex (0.220 g, 0.980 mmol, 0.5 equiv), 4,4′-bis(di-t-butyl)-2,2′-bipyridine (0.263 g, 0.980 mmol, 0.5 equiv), Ir[dF(CF₃)PPy]₂dtbbpy]PF₆ (0.220 g, 0.200 mmol, 0.10 equiv) and 1,4-diazabicyclo[2.2.2]octane (0.660 g, 5.88 mmol, 3.0 equiv) were sequentially added. The reaction mixture was sparged with nitrogen for an additional 5 min. The reaction was irradiated with blue LED light at room temperature for 5 days. The reaction mixture was filtered through Celite® and concentrated under reduced pressure onto silica gel (6 g). The residue was purified on a Biotage automated chromatography system (50 g, Biotage 60 μm silica gel column), eluting with a gradient of 0 to 30% ethyl acetate in heptane. The solid was dried under vacuum at 40° C. for 3 h to give the title compound (0.23 g, 19% yield) as a white foam. LCMS: m/z=622.3 [M+H]⁺.

### Example 45: (1S,3R)-1-(5-(Azetidin-3-yloxy)thiophen-2-yl)-3-methyl-2-(2,2,2-trifluoroethyl)-2,3,4,9-tetrahydro-1H-pyrido[3,4-b]indole

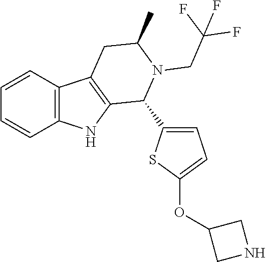 [SMILES: C[C@@H]1Cc2c([nH]c3ccccc23)[C@@H](c2ccc(OC3CNC3)s2)N1CC(F)(F)F]

Trifluoroacetic acid (1.6 mL, 21 mmol, 60 equiv) was added to tert-butyl (1S,3R)-1-(5-((1-(tert-butoxycarbonyl)azetidin-3-yl)oxy)thiophen-2-yl)-3-methyl-2-(2,2,2-trifluoroethyl)-1,2,3,4-tetrahydro-9H-pyrido[3,4-6]indole-9-carboxylate (0.218 g, 0.351 mmol, 1 equiv) in dichloromethane (7.0 mL) at 0° C. in a 40 mL vial. The reaction was stirred at 0° C. for 20 h. The reaction was diluted with cold dichloromethane (60 mL). Cold saturated sodium carbonate (20 mL) was added adjusting the pH to 10. The mixture washed with saturated sodium carbonate (60 mL). The organic layer was dried over sodium sulfate, filtered and concentrated under reduced pressure to give the title compound (0.115 g, 78% yield) as a gold solid. LCMS: m/z=422.1 [M+H]⁺.

### Example 46: (1S,3R)-1-(5-((1-(3-Fluoropropyl)azetidin-3-yl)oxy)thiophen-2-yl)-3-methyl-2-(2,2,2-trifluoroethyl)-2,3,4,9-tetrahydro-1H-pyrido[3,4-b]indole

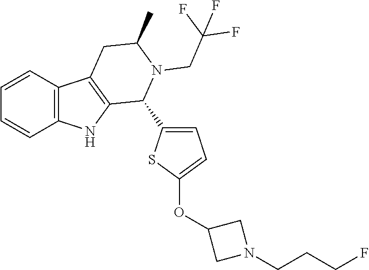 [SMILES: C[C@@H]1Cc2c([nH]c3ccccc23)[C@@H](c2ccc(OC3CN(CCCF)C3)s2)N1CC(F)(F)F]

1-Bromo-3-fluoropropane (0.04 mL, 0.4 mmol, 1.2 equiv) and N,N-diisopropylethylamine (0.23 mL, 1.2 mmol, 5 equiv) were sequentially added to a solution of (1S,3R)-1-(5-(azetidin-3-yloxy)thiophen-2-yl)-3-methyl-2-(2,2,2-trifluoroethyl)-2,3,4,9-tetrahydro-1H-pyrido[3,4-b]indole (0.109 g, 0.260 mmol, 1 equiv) in N,N-dimethylacetamide (2.6 mL) in a 40 mL vial. After stirring for 32 h at room temperature, the reaction mixture was transferred, diluted with ethyl acetate (20 mL) and sequentially washed with water (3×20 mL) and saturated brine (20 mL). The organic layer was dried over sodium sulfate, filtered and concentrated under reduced pressure onto Celite® (1 g). The residue was purified on a Biotage automated chromatography system (28 g, Biotage 50 μm KP-amino-d column), eluting with a gradient of 0 to 50% ethyl acetate in heptanes to give the title compound (48 mg, 38% yield) a yellow solid. ¹H NMR (400 MHz, CDCl₃) δ=7.82 (s, 1H), 7.52 (d, J=7.8 Hz, 1H), 7.33 (d, J=8.1 Hz, 1H), 7.20 (dt, J=1.2, 7.6 Hz, 1H), 7.15-7.10 (m, 1H), 6.36 (dd, J=1.2, 3.9 Hz, 1H), 5.88 (d, J=3.8 Hz, 1H), 5.03 (s, 1H), 4.70 (quin, J=5.7 Hz, 1H), 4.54 (t, J=5.9 Hz, 1H), 4.42 (t, J=6.0 Hz, 1H), 3.77-3.71 (m, 2H), 3.54-3.45 (m, 1H), 3.28-3.09 (m, 3H), 3.02-2.90 (m, 1H), 2.75 (dd, J=4.3, 16.1 Hz, 1H), 2.62 (t, J=7.1 Hz, 2H), 2.56-2.47 (m, 1H), 1.82-1.69 (m, 2H), 1.21 (d, J=6.7 Hz, 3H); LCMS: m/z=482.2 [M+H]⁺.

### Example 47: tert-Butyl (1S,3R)-1-(5-(((R)-1-(tert-butoxycarbonyl)pyrrolidin-3-yl)oxy)thiophen-2-yl)-3-methyl-2-(2,2,2-trifluoroethyl)-1,2,3,4-tetrahydro-9H-pyrido[3,4-b]indole-9-carboxylate

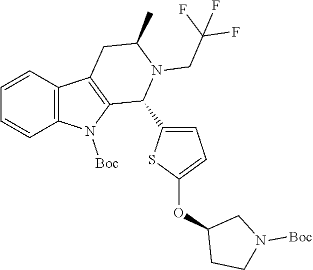 [SMILES: C[C@@H]1Cc2c(n(C(=O)OC(C)(C)C)c3ccccc23)[C@@H](c2ccc(O[C@@H]3CCN(C(=O)OC(C)(C)C)C3)s2)N1CC(F)(F)F]

A solution of tert-butyl (1S,3R)-1-(5-bromothiophen-2-yl)-3-methyl-2-(2,2,2-trifluoroethyl)-1,2,3,4-tetrahydro-9H-pyrido[3,4-b]indole-9-carboxylate (1.00 g, 1.9 mmol, 1 equiv) and tert-butyl (R)-3-hydroxypyrrolidine-1-carboxylate (1.2 g, 6.32 mmol, 3.34 equiv) in anhydrous acetonitrile (40 mL) was sparged with nitrogen for 20 min. 1,4-Diazabicyclo[2.2.2]octane (0.64 g, 5.64 mmol, 3.0 equiv), nickel (II) chloride ethylene glycol dimethylene ether complex (0.21 g, 0.94 mmol, 0.5 equiv), 4,4′-bis(di-t-butyl)-2,2′-bipyridine (0.25 g, 0.94 mmol, 0.5 equiv) and Ir[dF(CF₃)PPy]₂dtbbpy]PF₆ catalyst (0.21 g, 0.19 mmol, 0.1 equiv) were sequentially added, and the mixture was sparged with nitrogen for an additional 20 min. The resulting clear yellow solution was stirred at room temperature under irradiation with blue LED light for 3 days. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 20% ethyl acetate in heptanes, to give the title compound (0.22 g, 18% yield) as a white solid. LCMS: m/z=636.3 [M+H]⁺.

### Example 48: (1S,3R)-3-Methyl-1-(5-(((R)-pyrrolidin-3-yl)oxy)thiophen-2-yl)-2-(2,2,2-trifluoroethyl)-2,3,4,9-tetrahydro-1H-pyrido[3,4-b]indole

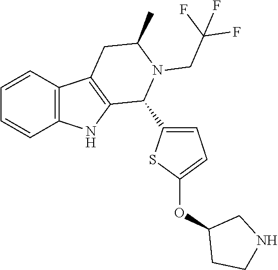 [SMILES: C[C@@H]1Cc2c([nH]c3ccccc23)[C@@H](c2ccc(O[C@@H]3CCNC3)s2)N1CC(F)(F)F]

Trifluoroacetic acid (1.6 mL, 20.3 mmol, 60 equiv) was added dropwise over ˜2 min to tert-butyl (1S,3R)-1-(5-(((R)-1-(tert-butoxycarbonyl)pyrrolidin-3-yl)oxy)thiophen-2-yl)-3-methyl-2-(2,2,2-trifluoroethyl)-1,2,3,4-tetrahydro-9H-pyrido[3,4-b]indole-9-carboxylate (0.22 g, 0.34 mmol, 1.0 equiv) in dichloromethane (5.0 mL) in a 40 mL vial at 0° C. The reaction was stirred at 0° C. for 18 h. Cold saturated sodium carbonate was added to the mixture adjusting the pH to 10. The solution was extracted with dichloromethane (3×15 mL). The combined organic layers were washed with saturated brine (20 mL), dried over sodium sulfate, filtered and concentrated under reduced pressure. The product was dried under vacuum at 25° C. for 1.5 h to give crude compound (0.15 g, 100% yield) as a green foam. LCMS: m/z=436.1 [M+H]⁺.

### Example 49: (1S,3R)-1-(5-(((R)-1-(3-Fluoropropyl)pyrrolidin-3-yl)oxy)thiophen-2-yl)-3-methyl-2-(2,2,2-trifluoroethyl)-2,3,4,9-tetrahydro-1H-pyrido[3,4-b]indole

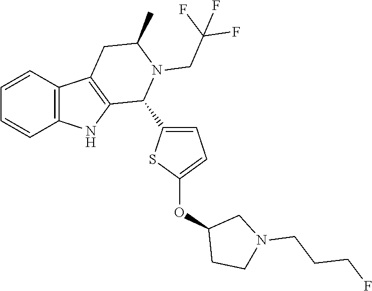 [SMILES: C[C@@H]1Cc2c([nH]c3ccccc23)[C@@H](c2ccc(O[C@@H]3CCN(CCCF)C3)s2)N1CC(F)(F)F]

1-Bromo-3-fluoropropane (48 μL, 0.44 mmol, 1.5 equiv) and N,N-diisopropylethylamine (0.30 mL, 1.70 mmol, 5.0 equiv) were added to a solution of (1S,3R)-3-methyl-1-(5-(((R)-pyrrolidin-3-yl)oxy)thiophen-2-yl)-2-(2,2,2-trifluoroethyl)-2,3,4,9-tetrahydro-1H-pyrido[3,4-b]indole (0.15 g, 0.34 mmol, 1.0 equiv) in N,N-dimethylacetamide (2.0 mL) in a 40 mL vial. After stirring at room temperature for 36 h, the reaction mixture was diluted with ethyl acetate (20 mL) and washed with saturated brine (2×15 mL). The organic layer was dried over sodium sulfate, filtered and concentrated under reduced pressure onto silica (12 g). The material was purified on a Büchi automated chromatography system (28 g, Biotage 50 μm KP-amino-d column), eluting with a gradient of 0 to 100% dichloromethane in heptanes. The solid was dried under vacuum at 25° C. for 4 h to give the title compound (47 mg, 28% yield) as an orange solid. ¹H NMR (400 MHz, CDCl₃) δ=7.82 (s, 1H), 7.52 (d, J=7.8 Hz, 1H), 7.32 (d, J=8.3 Hz, 1H), 7.20 (dt, J=1.2, 7.6 Hz, 1H), 7.15-7.10 (m, 1H), 6.37 (dd, J=1.2, 3.8 Hz, 1H), 5.93 (d, J=3.8 Hz, 1H), 5.04 (s, 1H), 4.75-4.66 (m, 1H), 4.57 (t, J=6.0 Hz, 1H), 4.45 (t, J=6.0 Hz, 1H), 3.56-3.46 (m, 1H), 3.22 (qd, J=9.5, 15.5 Hz, 1H), 2.96 (qd, J=9.1, 15.5 Hz, 1H), 2.90-2.69 (m, 4H), 2.69-2.43 (m, 4H), 2.30-2.17 (m, 1H), 2.09-1.84 (m, 3H), 1.23-1.20 (m, 3H); LCMS: m/z=496.2 [M+H]⁺.

### Example 50: 5-Bromo-3-fluorothiophen-2-yl)methanol

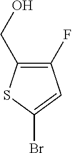

Methyl 5-bromo-3-fluorothiophene-2-carboxylate (1.0 g, 4.2 mmol, 1 equiv) was reacted with lithium borohydride (0.27 g, 8.4 mmol, 2 equiv) in diethyl ether (20 mL) at room temperature for 28 h. The reaction mixture was poured into saturated ammonium chloride (40 mL) and stirred for 90 min. The mixture was extracted with methyl t-butyl ether (3×20 mL). The combined organic layers were dried over magnesium sulfate (5 g), filtered and concentrated under reduced pressure to give the title compound (0.85 g, 96% yield) as a light-yellow oil. GCMS: m/z=210, 212 [M].

### Example 51: 5-Bromo-3-fluorothiophene-2-carbaldehyde

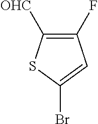

Manganese dioxide (8.7 g, 100 mmol, 25 equiv) was added to a solution of 5-bromo-3-fluorothiophen-2-yl)methanol (0.85 g, 4.0 mmol, 1 equiv) in dioxane (110 mL) at room temperature. After stirring for 5 h, GCMS analysis of a filtered aliquot indicated the reaction was complete. The reaction mixture was filtered through Celite® (20 g) and the filter pad was washed with dioxane (3×25 mL). The filtrate was concentrated under reduced pressure to give an oil. The oil was dissolved in dichloromethane (10 mL), absorbed onto silica gel (3 g) and concentrated under reduced pressure. Purification on a Biotage automated system (Biotage silica gel column 25 g, 20 μm), eluting with a gradient of 0 to 20% methyl 1-butyl ether in heptanes gave the title compound (0.46 g, 55% yield) as a pale-yellow oil. GCMS: m/z=208, 210 [M].

### Example 52: (1S,3R)-1-(5-Bromo-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole

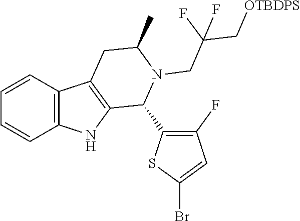

A solution of 5-bromo-3-fluorothiophene-2-carbaldehyde (1.0 g, 4.7 mmol, 1.0 equiv), (R)—N-(1-(1H-indol-3-yl)propan-2-yl)-3-((tert-butyldiphenylsilyl)oxy)-2,2-difluoropropan-1-amine (2.9 g, 5.7 mmol, 1.2 equiv) and acetic acid (1.3 mL, 23.7 mmol, 5.0 equiv) in toluene (25 mL) was heated at 80° C. under nitrogen for 20 hours. After cooling to room temperature, the reaction mixture was diluted with ethyl acetate (25 mL), washed with a saturated sodium carbonate (3×20 mL), dried over magnesium sulfate (5 g), filtered and concentrated under reduced pressure to give a red oil. The crude product was absorbed onto silica gel (40 g) with dichloromethane (100 mL) and then concentrated under reduced pressure. The material was purified on a Biotage automated chromatography system (200 g, 20 μm column), eluting with a gradient of 0 to 100% methyl t-butyl ether in heptanes, to give the title compound (2.9 g, 65% yield) as a yellow solid. LCMS: m/z=697, 699 [M+H]⁺.

### Example 53: tert-Butyl (1S,3R)-1-(5-bromo-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

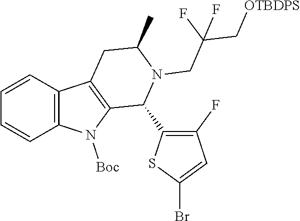

Di-tert-butyl dicarbonate (0.74 mL, 3.2 mmol, 1.5 equiv), triethylamine (0.60 mL, 4.3 mmol, 2.0 equiv) and 4-dimethylaminopyridine (0.039 g, 0.32 mmol, 0.15 equiv) were added to (1S,3R)-1-(5-bromo-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole (1.49 g, 2.13 mmol, 1 equiv) in dichloromethane (27 mL) in a 100 mL round-bottom flask. The reaction was stirred at room temperature for 20 hours. The reaction mixture was diluted with dichloromethane (20 mL) and was washed with water (2×27 mL) and saturated brine (27 mL). The organic layer was dried over sodium sulfate, filtered and concentrated under reduced pressure onto silica (10 g). The residue was purified on a Biotage automated chromatography system (50 g, Biotage 60 μm silica gel column), eluting with a gradient of 0 to 10% ethyl acetate in heptanes. The solid was dried under vacuum at room temperature for 16 hours to give the title compound (1.43 g, 84% yield) as a white foam. LCMS: m/z=797.2 [M+H]⁺.

### Example 54: tert-Butyl (1S,3R)-1-(5-((1-(tert-butoxycarbonyl)azetidin-3-yl)oxy)-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

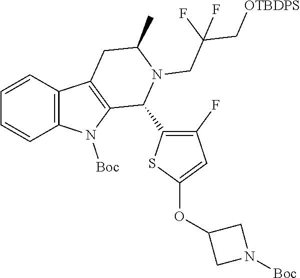

Two batches of tert-butyl (1S,3R)-1-(5-bromo-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.625 mmol, 1 equiv), tert-butyl 3-hydroxyazetidine-1-carboxylate (242 mg, 2.1 mmol, 3.34 equiv) and 1,4-diazabicyclo[2.2.2]octane (211 mg, 1.88 mmol, 3 equiv) in acetonitrile (10 mL) in 40 mL vials were sparged with nitrogen for 20 minutes. Two batches of nickel (II) chloride (glyme) complex (69 mg, 0.313 mmol, 0.5 equiv), 4,4′-di-tert-butyl-2,2′-dipyridyl (84 mg, 0.313 mmol, 0.5 equiv), and (Ir[dF(CF₃)ppy]₂(dtbpy))PF₆ (70 mg, 0.063 mmol, 0.1 equiv) were added to each solution at room temperature, and the mixture was sparged with nitrogen for an additional 5 minutes. The reaction vessels were sealed and both reactions were irradiated with blue LED light for 72 hours. The two batches were combined and filtered through Celite®, which was rinsed with ethyl acetate (2×30 mL). The filtrate was concentrated under reduced pressure. The residue was purified on a Biotage automated chromatography system (Biotage 50 g HC column), eluting with gradient of 0 to 40% methyl-tert-butyl ether in hexanes, to give title compound (274 mg, 25% yield) as a white solid. LCMS: m/z=890.4 [M+H]⁻.

### Example 55: (1S,3R)-1-(5-(Azetidin-3-yloxy)-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole

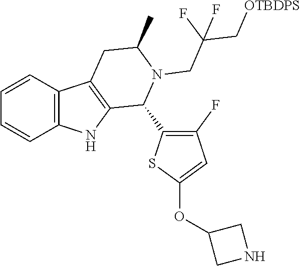

Trifluoroacetic acid (1.41 mL, 18.4 mmol, 60 equiv) was added at 0° C. to a solution of tert-butyl (1S,3R)-1-(5-((1-(tert-butoxycarbonyl)azetidin-3-yl)oxy)-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (273 mg, 0.307 mmol, 1 equiv) in dichloromethane (3 mL). After stirring at 0° C. for 24 hours, the reaction was diluted with dichloromethane (20 mL). The pH of the solution was carefully adjusted to pH 10 with saturated sodium carbonate at 0° C. The mixture was warmed to room temperature. The layers were separated and the aqueous layer was extracted with dichloromethane (3×5 mL). The combined organic layers were washed with saturated brine (5 mL), dried over sodium sulfate, filtered and concentrated under reduced pressure to give title compound (216 mg, >95% yield) as an orange solid. LCMS: m/z=690.3 [M+H]⁺.

### Example 56: (1S,3R)-2-(3-((tert-Butyldiphenylsilyl)oxy)-2,2-difluoropropyl)-1-(3-fluoro-5-((1-(3-fluoropropyl)azetidin-3-yl)oxy)thiophen-2-yl)-3-methyl-2,3,4,9-tetrahydro-1H-pyrido[3,4-b]indole

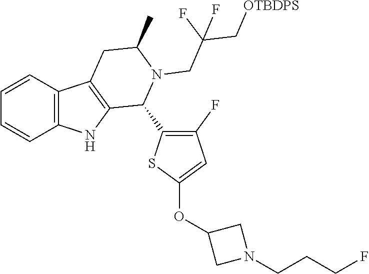

N,N-Diisopropylethylamine (0.080 mL, 0.461 mmol, 1.5 equiv) and 1-bromo-3-fluoropropane (52 mg, 0.37 mmol, 1.2 equiv) were sequentially added to a solution of (1S,3R)-1-(5-(azetidin-3-yloxy)-3-fluorothiophen-2-yl)-2-(3-((tert-butyldiphenylsilyl)oxy)-2,2-difluoropropyl)-3-methyl-2,3,4,9-tetrahydro-1H-pyrido[3,4-b]indole (216 mg, 0.307 mmol, 1 equiv) in N,N-dimethylacetamide (0.9 mL). After stirring for 16 hours at room temperature, saturated sodium bicarbonate (7 mL) and ethyl acetate (7 mL) were sequentially added. The layers were separated and the aqueous layer was extracted with ethyl acetate (3×10 mL). The combined organic layers were washed with saturated brine (10 mL), dried over sodium sulfate, filtered and concentrated under reduced pressure. The resulting yellow oil was purified on a Biotage automated chromatography system (Biotage 28 g KPNH column), eluting with a gradient of 5 to 50% ethyl acetate in hexanes to give title compound (121 mg, 52% yield) as a white solid. LCMS: m/z=750.3 [M+H]⁺.

### Example 57: 2,2-Difluoro-3-((1S,3R)-1-(3-fluoro-5-((1-(3-fluoropropyl)azetidin-3-yl)oxy)thiophen-2-yl)-3-methyl-1,3,4,9-tetrahydro-2H-pyrido[3,4-b]indol-2-yl)propan-1-ol

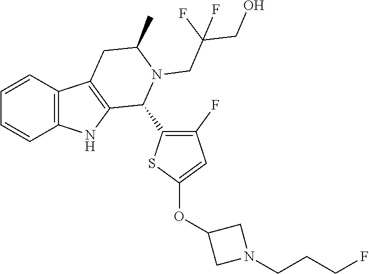 [SMILES: C[C@@H]1Cc2c([nH]c3ccccc23)[C@@H](c2sc(OC3CN(CCCF)C3)cc2F)N1CC(F)(F)CO]

1M Tetrabutylammonium fluoride in THF (0.14 mL, 0.14 mmol, 1.1 equiv) was added dropwise at room temperature to a solution of (1S,3R)-2-(3-((tert-butyldiphenylsilyl)oxy)-2,2-difluoropropyl)-1-(3-fluoro-5-((1-(3-fluoropropyl)azetidin-3-yl)oxy)thiophen-2-yl)-3-methyl-2,3,4,9-tetrahydro-1H-pyrido[3,4-b]indole (96 mg, 0.127 mmol, 1 equiv) in THF (2 mL). After stirring for 1 hour, the reaction was concentrated under reduced pressure. The residue was diluted with saturated ammonium chloride (10 mL). The suspension was extracted with ethyl acetate (3×10 mL). The combined organic layers were dried over sodium sulfate, filtered and concentrated under reduced pressure. The residue was combined with a batch from front run (used 24 mg of (1S,3R)-2-(3-((tert-butyldiphenylsilyl)oxy)-2,2-difluoropropyl)-1-(3-fluoro-5-((1-(3-fluoropropyl)azetidin-3-yl)oxy)thiophen-2-yl)-3-methyl-2,3,4,9-tetrahydro-1H-pyrido[3,4-b]indole) and purified on a Biotage automated chromatography system (Biotage 28 g KPNH column), eluting with gradient of 5 to 95% ethyl acetate in hexanes, to give title compound (59.9 mg, 92% yield) as a white solid. ¹H NMR (400 MHz, CDCl₃) δ=7.80 (s, 1H), 7.51 (d, J=7.8 Hz, 1H), 7.30 (d, J=7.7 Hz, 1H), 7.18 (dt, J=1.3, 7.5 Hz, 1H), 7.14-7.09 (m, 1H), 5.85 (s, 1H), 5.24 (s, 1H), 4.65 (quin, J=5.6 Hz, 1H), 4.53 (t, J=5.9 Hz, 1H), 4.41 (t, J=5.9 Hz, 1H), 3.87 (t, J=12.3 Hz, 2H), 3.73-3.65 (m, 3H), 3.23-3.08 (m, 3H), 3.00-2.85 (m, 2H), 2.63-2.55 (m, 3H), 1.80-1.67 (m, 2H), 1.23 (d, J=6.7 Hz, 3H): LCMS: m/z=512.2 [M+H]⁺.

### Example 58: tert-Butyl (1S,3R)-1-(5-(((R)-1-(tert-butoxycarbonyl)pyrrolidin-3-yl)oxy)-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

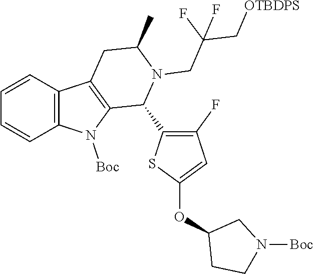

tert-Butyl (1S,3R)-1-(5-bromo-3-fluorothiophen-2-yl)-2-(3-((tert-butyldiphenylilyl)oxy)-2,2-difluoropropyl)-3-methyl-1,2,3,4-tetrahydro-9H-pyrido[3,4-b]indole-9-carboxylate (0.422 g, 0.529 mmol, 1 equiv) and tert-butyl (R)-3-hydroxypyrrolidine-1-carboxylate (0.328 g, 1.75 mmol, 3.3 equiv) in acetonitrile (5.3 mL) was sparged with nitrogen for 20 minutes in a 40 mL vial. Nickel(II) chloride ethylene glycol dimethylene ether complex (58 mg, 0.265 mmol, 0.5 equiv), 4,4′-bis(di-t-butyl)-2,2′-bipyridine (71 mg, 0.265 mmol, 0.5 equiv), Ir[dF(CF₃)PPy]₂dtbbpy]PF₆ (59 mg, 0.053 mmol, 0.10 equiv), and 1,4-diazabicyclo[2.2.2]octane (0.178 g, 1.59 mmol, 3.0 equiv) were added. The reaction mixture was sparged with nitrogen for an additional 10 minutes. The reaction was irradiated with blue LED light at room temperature for 40 hours. The reaction mixture was filtered through Celite® and concentrated under reduced pressure onto silica (4 g). The residue was purified on a Biotage automated chromatography system (25 g, Biotage 60 μm silica gel column), eluting with a gradient of 0 to 30% ethyl acetate in heptanes. The solid was dried under vacuum at room temperature for 16 hours to give the title compound (82 mg, 17% yield) as a white semi-solid. LCMS: m/z=904.3 [M+H]⁺.

### Example 59: (1S,3R)-2-(3-((tert-Butyldiphenylsilyl)oxy)-2,2-difluoropropyl)-1-(3-fluoro-5-(((R)-pyrrolidin-3-yl)oxy)thiophen-2-yl)-3-methyl-2,3,4,9-tetrahydro-1H-pyrido[3,4-b]indole

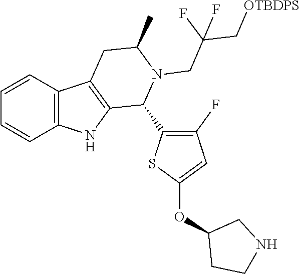

Trifluoroacetic acid (1.0 mL, 12.9 mmol, 60 equiv) was added to tert-butyl (1S,3R)-1-(5-(((R)-1-(tert-butoxycarbonyl)pyrrolidin-3-yl)oxy)-3-fluorothiophen-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.194 g, 0.215 mmol, 1 equiv) in dichloromethane (4.3 mL) at 0° C. in a 40 mL vial. The reaction was stirred at 0° C. for 24 hours. The reaction was diluted with cold dichloromethane (50 mL), then saturated sodium carbonate (25 mL) until pH ˜10. The layers were separated and the organic layer was washed with saturated sodium carbonate (2×50 mL) and saturated brine (50 mL). The organic layer was dried over sodium sulfate, filtered and concentrated under reduced pressure to give the title compound (0.180 g, >95% yield) as a dark yellow solid, which was used subsequently. LCMS: m/z=704.3 [M+H]⁻.

### Example 60: (1S,3R)-2-(3-((tert-Butyldiphenylsilyl)oxy)-2,2-difluoropropyl)-1-(3-fluoro-5-(((R)-1-(3-fluoropropyl)pyrrolidin-3-yl)oxy)thiophen-2-yl)-3-methyl-2,3,4,9-tetrahydro-1H-pyrido[3,4-b]indole

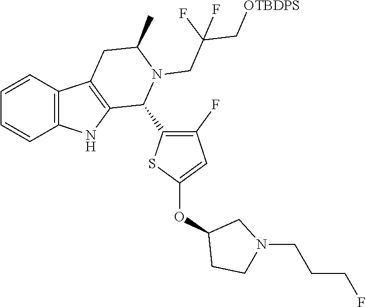

1-Bromo-3-fluoropropane (0.03 mL, 0.3 mmol, 1.5 equiv) and N,N-diisopropylethylamine (0.19 mL, 1.08 mmol, 5 equiv) were sequentially added to (1S,3R)-2-(3-((tert-butyldiphenylsilyl)oxy)-2,2-difluoropropyl)-1-(3-fluoro-5-(((R)-pyrrolidin-3-yl)oxy)thiophen-2-yl)-3-methyl-2,3,4,9-tetrahydro-1H-pyrido[3,4-b]indole (0.151 g, 0.215 mmol, 1 equiv) in N,N-dimethylacetamide (2.2 mL) in a 40 mL vial. After stirring for 28 hours at room temperature, the reaction mixture was transferred to a separatory funnel with ethyl acetate (20 mL) and sequentially washed with water (3×20 mL) and saturated brine (20 mL). The organic layer was dried over sodium sulfate, filtered and concentrated under reduced pressure onto Celite® (1.5 g). The residue was purified on a Biotage automated chromatography system (28 g, Biotage 50 μm KP-amino-d column), eluting with a gradient of 0 to 30% ethyl acetate in heptanes to give the title compound (71 mg, 43% yield) as a white semi-solid. LCMS: m/z=764.3 [M+H]⁺.

### Example 61: 2,2-Difluoro-3-((1S,3R)-1-(3-fluoro-5-(((R)-1-(3-fluoropropyl)pyrrolidin-3-yl)oxy)thiophen-2-yl)-3-methyl-1,3,4,9-tetrahydro-2H-pyrido[3,4-b]indol-2-yl)propan-1-ol

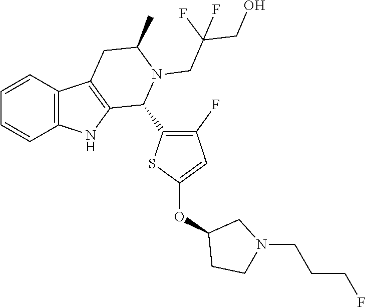 [SMILES: C[C@@H]1Cc2c([nH]c3ccccc23)[C@@H](c2sc(O[C@@H]3CCN(CCCF)C3)cc2F)N1CC(F)(F)CO]

1M Tetrabutylammonium fluoride (0.10 mL, 0.099 mmol, 1.1 equiv) was added to (1S,3R)-2-(3-((tert-butyldiphenylsilyl)oxy)-2,2-difluoropropyl)-1-(3-fluoro-5-(((R)-1-(3-fluoropropyl)pyrrolidin-3-yl)oxy)thiophen-2-yl)-3-methyl-2,3,4,9-tetrahydro-1H-pyrido[3,4-b]indole (0.069 g, 0.090 mmol, 1 equiv) in THF (0.90 mL) in a 40 mL vial at room temperature. After stirring for 2 hours at room temperature, the reaction mixture was concentrated under reduced pressure. The residue was transferred to a separatory funnel with ethyl acetate (10 mL) and washed with saturated ammonium chloride (10 mL). The aqueous layer was extracted with ethyl acetate (10 mL). The combined organic layers were sequentially washed with water (15 mL) and saturated brine (15 mL). The organic layer was dried over sodium sulfate, filtered and concentrated under reduced pressure onto Celite® (1.5 g). The residue was purified on a Biotage automated chromatography system (11 g, Biotage 50 μm KP-amino-d column), eluting with a gradient of 0 to 50% ethyl acetate in dichloromethane. The solid was dried under vacuum at room temperature for 16 hours to give the title compound (25.7 mg, 54% yield) as a yellow solid. ¹H NMR (400 MHz, CDCl₃) δ=7.73 (s, 1H), 7.51 (d, J=8.1 Hz, 1H), 7.32-7.28 (m, 1H), 7.18 (dt, J=1.3, 7.5 Hz, 1H), 7.14-7.09 (m, 1H), 5.91 (s, 1H), 5.24 (s, 1H), 4.64 (tdd, J=2.8, 5.1, 7.7 Hz, 1H), 4.56 (t, J=5.9 Hz, 1H), 4.44 (t, J=6.0 Hz, 1H), 3.87 (t, J=12.3 Hz, 2H), 3.75-3.66 (m, 1H), 3.39-3.12 (m, 2H), 3.03-2.87 (m, 2H), 2.84-2.76 (m, 3H), 2.64-2.54 (m, 3H), 2.45 (dt, J=6.3, 8.3 Hz, 1H), 2.26-2.16 (m, 1H), 2.03-1.82 (m, 3H), 1.23 (d, J=6.6 Hz, 3H). LCMS: m/z=526.2.2 [M+H]⁺.

### Example 62: 5-Bromo-2-((1S,3R)-2-(2-fluoro-2-methylpropyl)-3-methyl-2,3,4,9-tetrahydro-1H-pyrido[3,4-b]indol-1-yl)thiazole

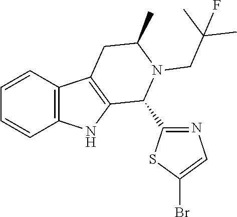 [SMILES: C[C@@H]1Cc2c([nH]c3ccccc23)[C@@H](c2ncc(Br)s2)N1CC(C)(C)F]

A mixture of 5-bromothiazole-2-carbaldehyde (4.95 g, 25.8 mmol, 1 equiv), (R)—N-(1-(1H-indol-3-yl)propan-2-yl)-2-fluoro-2-methylpropan-1-amine (7.68 g, 30.9 mmol, 1.2 equiv) and acetic acid (2.95 mL, 51.6 mmol, 2.0 equiv) in toluene (120 mL) was heated at 80° C. for 6 hours. After cooling to room temperature, the mixture was diluted with ethyl acetate (100 mL) and washed with saturated sodium bicarbonate (200 mL). The aqueous layer was extracted with ethyl acetate (3×100 mL). The combined organic layers were washed with saturated brine (200 mL), dried over sodium sulfate, filtered and concentrated under reduced pressure. The residue was purified on a Biotage automated chromatography system (Sorbtech 220 g column), eluting with a gradient of 0 to 30% methyl-t-butyl ether in heptanes to give the title compound (10.3 g, 94% yield) as a white solid. LCMS: m/z=423.1 [M+H]⁺.

### Example 63: tert-Butyl (1S,3R)-1-(5-bromothiazol-2-yl)-2-(2-fluoro-2-methylpropyl)-3-methyl-1,2,3,4-tetrahydro-9H-pyrido[3,4-b]indole-9-carboxylate

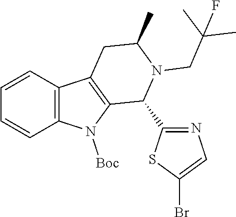 [SMILES: C[C@@H]1Cc2c(n(C(=O)OC(C)(C)C)c3ccccc23)[C@@H](c2ncc(Br)s2)N1CC(C)(C)F]

Di-tert-butyl dicarbonate (2.49 g, 11.4 mmol, 1.2 equiv) was added to a solution of 5-bromo-2-((1S,3R)-2-(2-fluoro-2-methylpropyl)-3-methyl-2,3,4,9-tetrahydro-1H-pyrido[3,4-b]indol-1-yl)thiazole (4 g, 9.49 mmol, 1.0 equiv), triethylamine (2.64 mL, 18.98 mmol, 2.0 equiv) and 4-dimethylaminopyridine (162 mg, 1.33 mmol, 0.14 equiv) in dichloromethane (60 mL). After stirring at room temperature for 16 hours, the reaction was diluted with dichloromethane (30 mL) and washed with water (50 mL) and saturated brine (50 mL). The organic layer was dried over sodium sulfate, filtered and concentrated under reduced pressure. The residue was purified on a Biotage automated chromatography system (Sorbtech 220 g column), eluting with a gradient of 0 to 30% methyl-t-butyl ether in heptanes, to give the title compound (4.7 g, 95% yield) as a white solid. LCMS: m/z=524.1 [M+H]⁺.

### Example 64: tert-Butyl (1S,3R)-1-(5-((1-(tert-butoxycarbonyl)azetidin-3-yl)oxy)thiazol-2-yl)-2-(2-fluoro-2-methylpropyl)-3-methyl-1,2,3,4-tetrahydro-9H-pyrido[3,4-b]indole-9-carboxylate

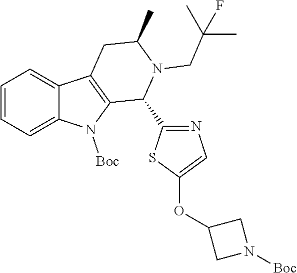 [SMILES: C[C@@H]1Cc2c(n(C(=O)OC(C)(C)C)c3ccccc23)[C@@H](c2ncc(OC3CN(C(=O)OC(C)(C)C)C3)s2)N1CC(C)(C)F]

tert-Butyl (1S,3R)-1-(5-bromothiazol-2-yl)-2-(2-fluoro-2-methylpropyl)-3-methyl-1,2,3,4-tetrahydro-9H-pyrido[3,4-b]indole-9-carboxylate (520 mg, 0.996 mmol, 1.0 equiv), tert-butyl 3-hydroxyazetidine-1-carboxylate (569 mg, 3.29 mmol, 3.34 equiv) and 1,4-diazabicyclo[2.2.2]octane (335.2 mg, 2.99 mmol, 3.0 equiv) in acetonitrile (10 mL) in a 40 mL vial was sparged with nitrogen for 20 minutes. Nickel (II) chloride (glyme) complex (109 mg, 0.498 mmol, 0.5 equiv), 4,4′-di-tert-butyl-2,2′-dipyridyl (134 mg, 0.498 mmol, 0.5 equiv) and (Ir[dF(CF3)ppy]2(dtbpy))PF6 (112 mg, 0.0996 mmol, 0.1 equiv) were added at room temperature and the mixture was sparged for an additional 5 minutes with nitrogen. The reaction vessel was sealed, and the reaction was irradiated with blue LED light for 72 hours. The mixture was filtered through Celite®, which was rinsed with ethyl acetate (30 mL). The filtrate was concentrated under reduced pressure. The residue was purified on a Biotage automated chromatography system, eluting with gradient of 0 to 30% ethyl acetate in hexanes, to give the title compound (123 mg, 20% yield) as an orange solid. LCMS: m/z=615.3 [M+H]⁺.

### Example 65: 5-(Azetidin-3-yloxy)-2-((1S,3R)-2-(2-fluoro-2-methylpropyl)-3-methyl-2,3,4,9-tetrahydro-1H-pyrido[3,4-b]indol-1-yl)thiazole

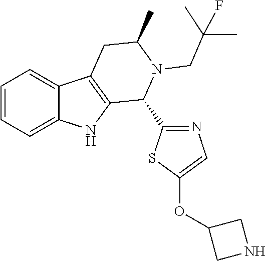 [SMILES: C[C@@H]1Cc2c([nH]c3ccccc23)[C@@H](c2ncc(OC3CNC3)s2)N1CC(C)(C)F]

Trifluoroacetic acid (0.66 mL, 8.65 mmol, 60 equiv) was added at 0° C. to a solution of tert-butyl (1S,3R)-1-(5-((1-(tert-butoxycarbonyl)azetidin-3-yl)oxy)thiazol-2-yl)-2-(2-fluoro-2-methylpropyl)-3-methyl-1,2,3,4-tetrahydro-9H-pyrido[3,4-b]indole-9-carboxylate (90 mg, 0.144 mmol, 1.0 equiv) in dichloromethane (3.3 mL). After stirring at 0° C. for 24 hours, the reaction was diluted with dichloromethane (18 mL). The pH of the solution was carefully adjusted to pH 10 with saturated sodium carbonate at 0° C. The mixture was warmed to room temperature. The layers were separated, and the aqueous layer was extracted with dichloromethane (3×5 mL). The combined organic layers were washed with saturated brine (5 mL), dried over sodium sulfate, filtered and concentrated under reduced pressure to give the title compound (53.2 mg, 89% yield) as an orange solid. LCMS: m/z=415.2 [M+H]⁺.

### Example 66: 2-((1S,3R)-2-(2-Fluoro-2-methylpropyl)-3-methyl-2,3,4,9-tetrahydro-1H-pyrido[3,4-b]indol-1-yl)-5-((1-(3-fluoropropyl)azetidin-3-yl)oxy)thiazole

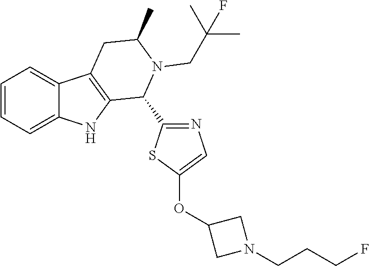 [SMILES: C[C@@H]1Cc2c([nH]c3ccccc23)[C@@H](c2ncc(OC3CN(CCCF)C3)s2)N1CC(C)(C)F]

N,N-Diisopropylethylamine (0.033 mL, 0.19 mmol, 1.5 equiv) and 1-bromo-3-fluoropropane (22 mg, 0.154 mmol, 1.2 equiv) were sequentially added to a solution of 5-(azetidin-3-yloxy)-2-((1S,3R)-2-(2-fluoro-2-methylpropyl)-3-methyl-2,3,4,9-tetrahydro-1H-pyrido[3,4-b]indol-1-yl)thiazole (53 mg, 0.128 mmol, 1.0 equiv) in N,N-dimethylacetamide (0.4 mL). After stirring for 16 hours at room temperature, saturated sodium bicarbonate (5 mL) and ethyl acetate (5 mL) were sequentially added. The layers were separated, and the aqueous layer was extracted with ethyl acetate (3×5 mL). The combined organic layers were washed with saturated brine (5 mL), dried over sodium sulfate, filtered and concentrated under reduced pressure. The residue was purified on Biotage automated chromatography system (Biotage 11 g KPNH column), eluting with a gradient of 10 to 50% ethyl acetate in hexanes to give the title compound (17.3 mg, 29% yield) as a white solid. 1H NMR (400 MHz, CDCl₃) δ=8.44 (br s, 1H), 7.48 (d, J=7.7 Hz, 1H), 7.35 (d, J=8.1 Hz, 1H), 7.15 (dt, J=1.2, 7.6 Hz, 1H), 7.10-7.05 (m, 1H), 6.83 (s, 1H), 5.17 (d, J=3.2 Hz, 1H), 4.68 (quin, J=5.6 Hz, 1H), 4.54 (t, J=6.0 Hz, 1H), 4.42 (t, J=6.0 Hz, 1H), 3.76-3.68 (m, 2H), 3.45-3.33 (m, 1H), 3.13 (dd, J=5.4, 7.5, 8.5 Hz, 2H), 2.75-2.56 (m, 6H), 1.82-1.68 (m, 2H), 1.56-1.50 (m, 3H), 1.38-1.31 (m, 3H), 1.27 (d, J=6.8 Hz, 3H); LCMS: m/z=475.3 [M+H]⁺.

### Example 67: tert-Butyl (1S,3R)-1-(5-(((S)-1-(tert-butoxycarbonyl)pyrrolidin-3-yl)oxy)thiazol-2-yl)-2-(2-fluoro-2-methylpropyl)-3-methyl-1,2,3,4-tetrahydro-9H-pyrido[3,4-b]indole-9-carboxylate

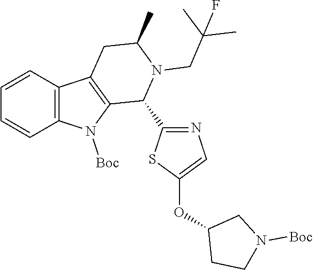 [SMILES: C[C@@H]1Cc2c(n(C(=O)OC(C)(C)C)c3ccccc23)[C@@H](c2ncc(O[C@H]3CCN(C(=O)OC(C)(C)C)C3)s2)N1CC(C)(C)F]

A mixture of tert-butyl (1S,3R)-1-(5-bromothiazol-2-yl)-2-(2-fluoro-2-methylpropyl)-3-methyl-1,2,3,4-tetrahydro-9H-pyrido[3,4-b]indole-9-carboxylate (0.860 g, 1.65 mmol, 1 equiv), tert-butyl (S)-3-hydroxypyrrolidine-1-carboxylate (1.03 g, 5.50 mmol, 3.34 equiv), and 1,4-diazabicyclo[2.2.2]octane (0.555 g, 4.95 mmol, 3 equiv) in acetonitrile (30 mL) was sparged with nitrogen for 20 minutes. Nickel(II) chloride (glyme) complex (0.181 g, 0.825 mmol, 0.5 equiv), 4,4′-di-tert-butyl-2,2′-dipyridyl (0.221 g, 0.825 mmol, 0.5 equiv) and (Ir[dF(CF₃)ppy]₂(dtbpy))PF₆ (0.185 g, 0.165 mmol, 0.1 equiv) were sequentially added at room temperature, The mixture was sparged with nitrogen for an additional 5 minutes. The reaction vessel was sealed and the reaction was irradiated with Blue LED light for 18 hours. Additional batch at 100 mg scale were processed in same manner, and two batches were combined for purification. The mixture was dry-loaded onto Celite® and purified on an Interchim automated chromatography system (Sorbtech 80 g silica gel column), eluting with a gradient of 0 to 100% ethyl acetate in heptanes, to give the title compound (87 mg, 6.9% combined yield) as a white foam. LCMS: m/z=629 [M+H]⁺.

### Example 68: 2-((1S,3R)-2-(2-Fluoro-2-methylpropyl)-3-methyl-2,3,4,9-tetrahydro-1H-pyrido[3,4-b]indol-1-yl)-5-(((S)-pyrrolidin-3-yl)oxy)thiazole

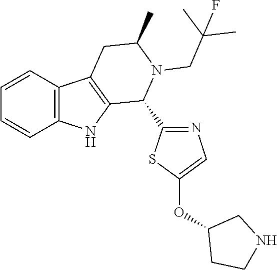 [SMILES: C[C@@H]1Cc2c([nH]c3ccccc23)[C@@H](c2ncc(O[C@H]3CCNC3)s2)N1CC(C)(C)F]

Trifluoroacetic acid (0.64 mL, 8.31 mmol, 60 equiv) was added to a solution of tert-butyl (1S,3R)-1-(5-(((R)-1-(tert-butoxycarbonyl)pyrrolidin-3-yl)oxy)thiazol-2-yl)-2-(2-fluoro-2-methylpropyl)-3-methyl-1,2,3,4-tetrahydro-9H-pyrido[3,4-b]indole-9-carboxylate (87 mg, 0.139 mmol, 1 equiv) in dichloromethane (2 mL) at room temperature. After stirring for 1 hour, LC analysis indicated that the reaction was complete. Saturated sodium carbonate was added carefully at 0° C., adjusting the pH to 9-10. The mixture was extracted with dichloromethane (3×50 mL). The combined organic layers were dried over anhydrous sodium sulfate, filtered and concentrated under reduced pressure to give the title compound (46.9 mg, 79% yield) as a brown foam. LCMS: m/z=429 [M+H]⁺.

### Example 69: 2-((1S,3R)-2-(2-Fluoro-2-methylpropyl)-3-methyl-2,3,4,9-tetrahydro-1H-pyrido[3,4-b]indol-1-yl)-5-(((R)-1-(3-fluoropropyl)pyrrolidin-3-yl)oxy)thiazole

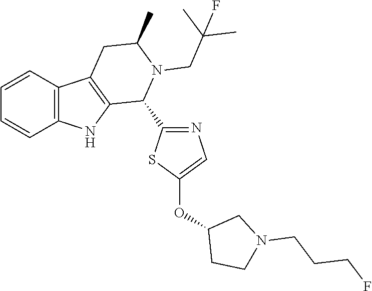 [SMILES: C[C@@H]1Cc2c([nH]c3ccccc23)[C@@H](c2ncc(O[C@H]3CCN(CCCF)C3)s2)N1CC(C)(C)F]

N,N-Diisopropylethylamine (0.095 mL, 0.55 mmol, 5 equiv) was added to a solution of 2-((1S,3R)-2-(2-fluoro-2-methylpropyl)-3-methyl-2,3,4,9-tetrahydro-1H-pyrido[3,4-b]indol-1-yl)-5-(((S)-pyrrolidin-3-yl)oxy)thiazole (46.9 mg, 0.11 mmol, 1 equiv) in N,N-dimethylacetamide (2 mL). After stirring at room temperature for 10 minutes, 1-bromo-3-fluoropropane (11.6 μL, 0.126 mmol, 1.15 equiv) was added and the mixture was stirred at room temperature for 18 hours, at which time LC analysis indicated ˜80% conversion. Additional 1-bromo-3-fluoropropane (30 μL, 0.029 mmol, 0.23 equiv) was added and the mixture was stirred at room temperature for 20 hours. LC analysis indicated >90% conversion. The mixture was diluted with ethyl acetate (30 mL) and washed with water (30 mL). The aqueous layer was extracted with ethyl acetate (3×20 mL). The combined organic layers were washed with saturated brine (20 mL), dried over anhydrous sodium sulfate, filtered and concentrated under reduced pressure. The residue was purified on an Interchim automated chromatography system (Biotage 11 g KPNH column), eluting with a gradient of 0 to 100% dichloromethane in heptanes to give the title compound (11.8 mg, 24% yield) as an off-white solid. LCMS: m/z=489 [M+H]⁺; ¹H NMR (400 MHz, CDCl₃) δ=8.43 (br s, 1H), 7.52 (br d, J=8.2 Hz, 1H), 7.48 (d, J=7.9 Hz, 1H), 7.40 (d, J=7.9 Hz, 1H), 7.36 (d, J=7.9 Hz, 1H), 7.15 (dt, J=1.2, 7.6 Hz, 1H), 7.10-7.05 (m, 1H), 6.88 (s, 1H), 5.18 (d, J=3.1 Hz, 1H), 5.05 (s, 1H), 4.70-4.62 (m, 1H), 4.57 (t, J=5.9 Hz, 1H), 4.45 (t, J=5.9 Hz, 1H), 3.49-3.33 (m, 1H), 2.89-2.72 (m, 4H), 2.72-2.53 (m, 5H), 2.52-2.44 (m, 1H), 2.28-2.17 (m, 1H), 2.09-1.99 (m, 1H), 1.98-1.83 (m, 2H), 1.56-1.49 (m, 3H), 1.38-1.32 (m, 3H), 1.27 (d, J=6.7 Hz, 3H).

### Example 70: tert-Butyl (1S,3R)-1-(5-(((R)-1-(tert-butoxycarbonyl)pyrrolidin-3-yl)oxy)thiazol-2-yl)-2-(2-fluoro-2-methylpropyl)-3-methyl-1,2,3,4-tetrahydro-9H-pyrido[3,4-b]indole-9-carboxylate

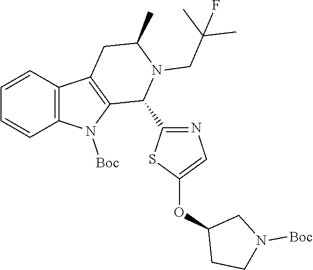 [SMILES: C[C@@H]1Cc2c(n(C(=O)OC(C)(C)C)c3ccccc23)[C@@H](c2ncc(O[C@@H]3CCN(C(=O)OC(C)(C)C)C3)s2)N1CC(C)(C)F]

A mixture of tert-butyl (1S,3R)-1-(5-bromothiazol-2-yl)-2-(2-fluoro-2-methylpropyl)-3-methyl-1,2,3,4-tetrahydro-9H-pyrido[3,4-b]indole-9-carboxylate (0.522 g, 1.0 mmol, 1 equiv), tert-butyl (R)-3-hydroxypyrrolidine-1-carboxylate (0.625 g, 3.34 mmol, 3.34 equiv), and 1,4-diazabicyclo[2.2.2]octane (0.337 g, 3.0 mmol, 3 equiv) in acetonitrile (20 mL) was sparged with nitrogen for 20 min. Nickel(II) chloride (glyme) complex (0.110 g, 0.50 mmol, 0.5 equiv), 4,4′-di-tert-butyl-2,2′-dipyridyl (0.134 g, 0.50 mmol, 0.5 equiv) and (Ir[dF(CF₃)ppy]₂(dtbpy))PF₆ (0.112 g, 0.10 mmol, 0.1 equiv) were sequentially added at room temperature. The mixture was sparged with nitrogen for an additional 5 min. The reaction vessel was sealed, and the reaction was irradiated with Blue LED light for 40 h. An additional two batches at same scale were processed in parallel and three batches were combined for purification. The mixture was dry-loaded onto Celite® and purified on an Interchim automated chromatography system (Sorbtech 80 g silica gel column), eluting with a gradient of 0 to 100% ethyl acetate in heptanes, to give the title compound (0.185 g, 10% yield) as a white foam. LCMS: m/z=629 [M+H]⁺.

### Example 71: 2-((1S,3R)-2-(2-Fluoro-2-methylpropyl)-3-methyl-2,3,4,9-tetrahydro-1H-pyrido[3,4-b]indol-1-yl)-5-(((R)-pyrrolidin-3-yl)oxy)thiazole

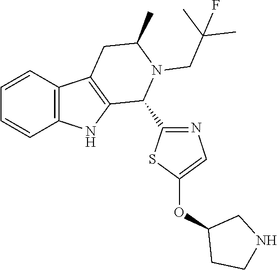 [SMILES: C[C@@H]1Cc2c([nH]c3ccccc23)[C@@H](c2ncc(O[C@@H]3CCNC3)s2)N1CC(C)(C)F]

Trifluoroacetic acid (1.1 mL, 14.33 mmol, 50 equiv) was added to a solution of tert-butyl (1S,3R)-1-(5-(((R)-1-(tert-butoxycarbonyl)pyrrolidin-3-yl)oxy)thiazol-2-yl)-2-(2-fluoro-2-methylpropyl)-3-methyl-1,2,3,4-tetrahydro-9H-pyrido[3,4-b]indole-9-carboxylate (0.18 g, 0.287 mmol, 1 equiv) in dichloromethane (6 mL) at 0° C. After stirring at 0° C. for 48 h, LC analysis indicated that the reaction was complete. Saturated sodium carbonate was added carefully at 0° C., adjusting the pH to 9-10. The mixture was extracted with dichloromethane (3×50 mL). The combined organic layers were dried over anhydrous sodium sulfate, filtered and concentrated under reduced pressure to give the title compound (0.108 g, 88% yield) as a brown foam. LCMS: m/z=429 [M+H]⁺.

### Example 72: 2-((1S,3R)-2-(2-Fluoro-2-methylpropyl)-3-methyl-2,3,4,9-tetrahydro-1H-pyrido[3,4-b]indol-1-yl)-5-(((R)-1-(3-fluoropropyl)pyrrolidin-3-yl)oxy)thiazole

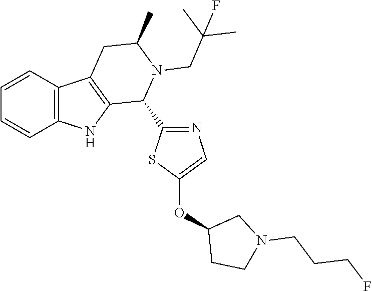 [SMILES: C[C@@H]1Cc2c([nH]c3ccccc23)[C@@H](c2ncc(O[C@@H]3CCN(CCCF)C3)s2)N1CC(C)(C)F]

N,N-Diisopropylethylamine (0.21 mL, 1.211 mmol, 5 equiv) was added to a solution of 2-((1S,3R)-2-(2-Fluoro-2-methylpropyl)-3-methyl-2,3,4,9-tetrahydro-1H-pyrido[3,4-b]indol-1-yl)-5-(((R)-pyrrolidin-3-yl)oxy)thiazole (0.104 g, 0.242 mmol, 1 equiv) in N,N-dimethylacetamide (3 mL). After stirring at room temperature for 10 min, 1-bromo-3-fluoropropane (41 mg, 0.291 mmol, 1.2 equiv) was added and the mixture was stirred at room temperature for 18 h, at which time LC analysis indicated ˜82% conversion. Additional 1-bromo-3-fluoropropane (10 mg, 0.08 mmol, 0.5 equiv) was added and the mixture was stirred at room temperature for 20 h. LC analysis indicated >90% conversion. The mixture was diluted with ethyl acetate (50 mL) and washed with water (20 mL). The aqueous layer was extracted with ethyl acetate (3×20 mL). The combined organic layers were washed with saturated brine (50 mL), dried over anhydrous sodium sulfate, filtered and concentrated under reduced pressure. The residue was purified on an Interchim automated chromatography system (Biotage 11 g KPNH column), eluting with a gradient of 0 to 100% dichloromethane in heptanes, to give the title compound (25 mg, 21% yield) as an off-white solid. LCMS: m/z=489 [M+H]⁺; ¹H NMR (400 MHz, CDCl₃) δ=8.51 (br s, 1H), 7.48 (d, J=7.8 Hz, 1H), 7.34 (d, J=7.7 Hz, 1H), 7.14 (dt, J=1.3, 7.5 Hz, 1H), 7.10-7.05 (m, 1H), 6.88 (s, 1H), 5.18 (d, J=3.2 Hz, 1H), 4.70-4.63 (m, 1H), 4.57 (t, J=5.9 Hz, 1H), 4.45 (t, J=5.9 Hz, 1H), 3.46-3.36 (m, 1H), 2.88-2.72 (m, 4H), 2.69-2.54 (m, 5H), 2.48 (dt, J=6.3, 8.3 Hz, 1H), 2.29-2.19 (m, 1H), 2.10-2.02 (m, 1H), 1.97-1.83 (m, 2H), 1.57-1.50 (m, 3H), 1.38-1.32 (m, 3H), 1.27 (d, J=6.8 Hz, 3H).

### Example 73: 5-Bromo-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole

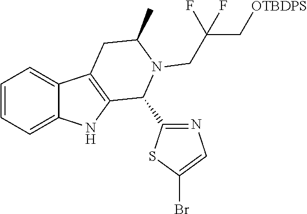

A mixture of 5-bromothiazole-2-carbaldehyde (0.9 g, 4.7 mmol, 1 equiv), (R)—N-(1-(1H-indol-3-yl)propan-2-yl)-3-((tert-butyldiphenylsilyl)oxy)-2,2-difluoropropan-1-amine (2.85 g, 5.6 mmol, 1.2 equiv) and acetic acid (0.54 mL, 9.4 mmol, 2 equiv) in toluene (16 mL) was heated at 80° C. in a 100 mL round bottom flask for 6 h. After cooling to room temperature, the mixture was diluted with ethyl acetate (30 mL) and washed with saturated sodium bicarbonate (50 mL). The aqueous layer was extracted with ethyl acetate (3×20 mL). The combined organic layers were washed with saturated brine (50 mL), dried over sodium sulfate, filtered and concentrated under reduced pressure. The residue was purified on an Biotage automated chromatography system (Biotage 100 g HC column), eluting with a gradient of 0 to 30% methyl-t-butyl ether in heptanes, to give the title compound (2.69 g, 84% yield) as a white solid. LCMS: m/z=681.2 [M+H]⁺.

### Example 74: tert-Butyl (1S,3R)-1-(5-bromothiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

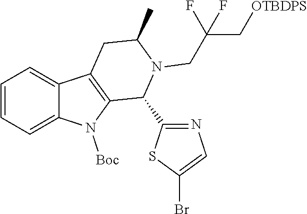

Di-tert-butyl dicarbonate (0.94 g, 4.3 mmol, 1.1 equiv), 4-dimethylaminopyridine (0.067 g, 0.55 mmol, 0.14 equiv) and triethylamine (1.1 mL, 7.88 mmol, 2 equiv) were added to a solution of 5-bromo-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (2.68 g, 3.94 mmol, 1 equiv) in dichloromethane (30 mL) at room temperature. After stirring at room temperature for 16 h, the reaction was diluted with dichloromethane (30 mL) and washed with water (50 mL) and saturated brine (50 mL). The organic layer was dried over sodium sulfate, filtered and concentrated under reduced pressure. The residue was purified on a Biotage automated chromatography system (Biotage 100 g HC column), eluting with gradient of 0 to 30% methyl-tert-butyl ether in heptanes, to give the title compound (2.67 g, 80% yield) as white solid. LCMS: m/z=781.2 [M+H]⁺.

### Example 75: tert-Butyl (1S,3R)-1-(5-((1-(tert-butoxycarbonyl)azetidin-3-yl)oxy)thiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

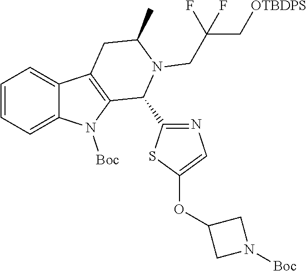

Two batches of tert-butyl (1S,3R)-1-(5-bromothiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (620 mg, 0.79 mmol, 1 equiv), tert-butyl 3-hydroxyazetidine-1-carboxylate (302 mg, 2.62 mmol, 3.34 equiv) and 1,4-diazabicyclo[2.2.2]octane (266 mg, 2.37 mmol, 3 equiv) in acetonitrile (10 mL) in 40 mL vials were sparged with nitrogen for 20 min. Two batches of nickel (II) chloride (glyme) complex (87 mg, 0.395 mmol, 0.5 equiv), 4,4′-di-tert-butyl-2,2′-dipyridyl (106 mg, 0.395 mmol, 0.5 equiv), and (Ir[dF(CF₃)ppy]₂(dtbpy))PF₆ (89 mg, 0.079 mmol, 0.1 equiv) were added to each solution at room temperature and the mixture was sparged with nitrogen for an additional 5 min. The reaction vessels were sealed and both reactions were irradiated with blue LED light for 72 h. The two batches were combined and filtered through Celite®, which was rinsed with ethyl acetate (2×30 mL). The filtrate was concentrated under reduced pressure. The residue was purified on a Biotage automated chromatography system (Biotage 50 g HC column), eluting with gradient of 0 to 30% ethyl acetate in hexanes to give the title compound (253 mg, 18% yield) as a white solid. LCMS: m/z=874.4 [M+H]⁺.

### Example 76: 5-(Azetidin-3-yloxy)-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole

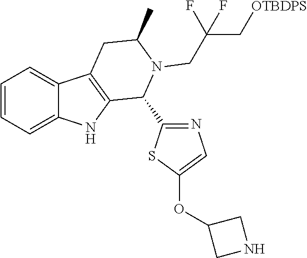

Trifluoroacetic acid (0.64 mL, 8.35 mmol, 60 equiv) was added at 0° C. to a solution of tert-butyl (1S,3R)-1-(5-((1-(tert-butoxycarbonyl)azetidin-3-yl)oxy)thiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (121 mg, 0.139 mmol, 1 equiv) in dichloromethane (3.2 mL). After stirring at 0° C. for 24 h, the reaction was diluted with dichloromethane (15 mL). The pH of the solution was carefully adjusted to pH 10 with saturated sodium carbonate at 0° C. The mixture was warmed to room temperature. The layers were separated, and the aqueous layer was extracted with dichloromethane (3×5 mL). The combined organic layers were washed with saturated brine (5 mL), dried over sodium sulfate, filtered and concentrated under reduced pressure to give the title compound (82.4 mg, 88% yield) as an orange solid. LCMS: m/z=674.3 [M+H]⁺.

### Example 77: 2-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-5-((1-(3-fluoropropyl)azetidin-3-yl)oxy)thiazole

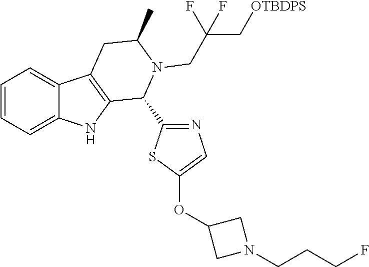

N,N-Diisopropylethylamine (0.031 mL, 0.146 mmol, 1.5 equiv) and 1-bromo-3-fluoropropane (21 mg, 0.146 mmol, 1.2 equiv) were sequentially added to a solution of 5-(azetidin-3-yloxy)-2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (82 mg, 0.122 mmol, 1 equiv) in N,N-dimethylacetamide (0.35 mL). After stirring for 16 h at room temperature, saturated sodium bicarbonate (5 mL) and ethyl acetate (5 mL) were sequentially added. The layers were separated, and the aqueous layer was extracted with ethyl acetate (3×5 mL). The combined organic layers were washed with saturated brine (5 mL), dried over sodium sulfate, filtered and concentrated under reduced pressure. The resulting yellow oil was purified on Biotage automated chromatography system (Biotage 11 g KPNH column), eluting with a gradient of 5 to 50% ethyl acetate in hexanes, to give the title compound (45.9 mg, 51% yield) as a white solid. LCMS: m/z=733.3 [M+H]⁺.

### Example 78: 2,2-Difluoro-3-((1S,3R)-1-(5-((1-(3-fluoropropyl)azetidin-3-yl)oxy)thiazol-2-yl)-3-methyl-1,3,4,9-tetrahydro-2H-pyrido[3,4-b]indol-2-yl)propan-1-ol

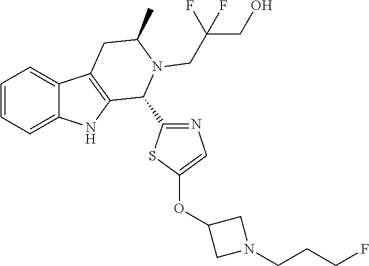 [SMILES: C[C@@H]1Cc2c([nH]c3ccccc23)[C@@H](c2ncc(OC3CN(CCCF)C3)s2)N1CC(F)(F)CO]

A 1M solution of tetra-N-butylammonium fluoride in THF (0.045 mL, 0.045 mmol, 1.1 equiv) was added dropwise at room temperature to a solution of 2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-5-((1-(3-fluoropropyl)azetidin-3-yl)oxy)thiazole (30 mg, 0.041 mmol, 1 equiv) in THF (1 mL). After stirring for 1 h, the reaction was concentrated under reduced pressure. The residue was diluted with saturated ammonium chloride (5 mL). The suspension was extracted with ethyl acetate (3×5 mL). The combined organic layers were dried over sodium sulfate, filtered and concentrated under reduced pressure. The residue was combined with a batch from front run (used 7 mg of the starting TBDPS ether) and purified on a Biotage automated chromatography system (Biotage 11 g KPNH column), eluting with 50% ethyl acetate in dichloromethane, to give the title compound (16.4 mg, 65% yield) as a yellow solid. ¹H NMR (400 MHz, DMSO-d₆) δ=10.94 (s, 1H), 7.41 (d, J=7.7 Hz, 1H), 7.33 (d, J=8.1 Hz, 1H), 7.06 (t, J=7.6 Hz, 1H), 6.97 (t, J=7.0 Hz, 2H), 5.53 (t, J=5.9 Hz, 1H), 5.12 (s, 1H), 4.74 (quin, J=5.5 Hz, 1H), 4.50 (t, J=6.1 Hz, 1H), 4.38 (t, J=6.1 Hz, 1H), 3.76 (dt, J=5.8, 13.6 Hz, 2H), 3.66-3.58 (m, 2H), 3.41-3.32 (m, 1H), 3.27-3.15 (m, 2H), 3.06-2.98 (m, 2H), 2.76-2.64 (m, 1H), 2.60-2.51 (m, 3H), 1.72-1.58 (m, 2H), 1.23-1.20 (m, 3H); LCMS: m/z=495.2 [M+H]⁺.

### Example 79: tert-Butyl (1S,3R)-1-(5-(((R)-1-(tert-butoxycarbonyl)pyrrolidin-3-yl)oxy)thiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

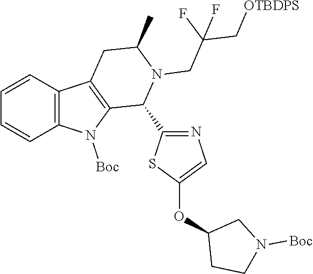

A mixture of tert-butyl (1S,3R)-1-(5-bromothiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (1.303 g, 1.669 mmol, 1 equiv), tert-butyl (R)-3-hydroxypyrrolidine-1-carboxylate (1.04 g, 5.573 mmol, 3.34 equiv), and 1,4-diazabicyclo[2.2.2]octane (0.561 g, 5.006 mmol, 3 equiv) in acetonitrile (17 mL) was sparged with nitrogen for 20 min. Nickel(II) chloride (glyme) complex (0.183 g, 0.834 mmol, 0.5 equiv), 4,4′-di-tert-butyl-2,2′-dipyridyl (0.224 g, 0.834 mmol, 0.5 equiv) and (Ir[dF(CF₃)ppy]₂(dtbpy))PF₆ (0.187 g, 0.167 mmol, 0.1 equiv) were sequentially added at room temperature, The mixture was sparged with nitrogen for an additional 5 min. The reaction vessel was sealed, and the reaction was irradiated with Blue LED light for 48 h. The mixture was dry-loaded onto Celite® and purified on an Interchim automated chromatography system (Sorbtech 80 g silica gel column), eluting with a gradient of 0 to 100% ethyl acetate in heptanes, to give the title compound (0.165 g, 11% yield) as a colorless oil. LCMS: m/z=888 [M+H]⁺.

### Example 80: 2-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-5-(((R)-pyrrolidin-3-yl)oxy)thiazole

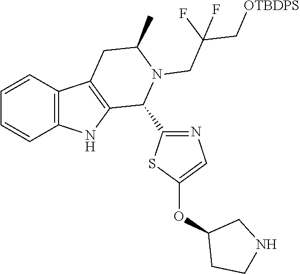

Trifluoroacetic acid (0.62 mL, 8.071 mmol, 50 equiv) was added to a solution of tert-butyl (1S,3R)-1-(5-(((R)-1-(tert-butoxycarbonyl)pyrrolidin-3-yl)oxy)thiazol-2-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.143 g, 0.161 mmol, 1 equiv) in dichloromethane (5 mL) at 0° C. After stirring for 43 h, LCMS analysis indicated that a 6 to 3 to 1 mixture of desired product and mono-Boc product and unknown by-product. Additional trifluoroacetic acid (0.25 mL, 3.228 mmol, 20 equiv) was added, and the mixture was stirred at 0° C. for 48 h. LC analysis indicated that the reaction was complete. Saturated sodium carbonate was added carefully at 0° C., adjusting the pH to 9-10. The mixture was extracted with dichloromethane (3×50 mL). The combined organic layers were dried over anhydrous sodium sulfate, filtered and concentrated under reduced pressure to give the title compound (0.110 g, 99% yield) as a brown oil. LCMS: m/z=688 [M+H]⁺.

### Example 81: 2-((1S,3R)-2-(3-((tert-Butyldiphenylsilyl)oxy)-2,2-difluoropropyl)-3-methyl-2,3,4,9-tetrahydro-1H-pyrido[3,4-6]indol-1-yl)-5-(((R)-1-(3-fluoropropyl)pyrrolidin-3-yl)oxy)thiazole

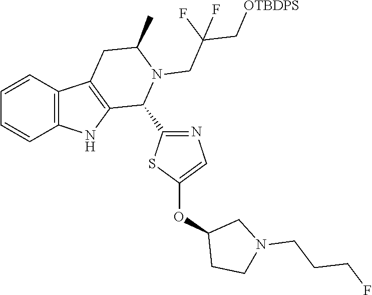

N,N-Diisopropylethylamine (0.14 mL, 0.80 mmol, 5 equiv) was added to a solution of 2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-5-(((R)-pyrrolidin-3-yl)oxy)thiazole (0.110 g, 0.16 mmol, 1 equiv) in N,N-dimethylacetamide (3 mL). After stirring at room temperature for 10 min, 1-bromo-3-fluoropropane (33.8 mg, 0.24 mmol, 1.5 equiv) was added and the mixture was stirred at room temperature for 18 h at which time LC analysis indicated ˜75% conversion. Additional 1-bromo-3-fluoropropane (11 mg, 0.08 mmol, 0.5 equiv) was added and the mixture was stirred at room temperature for 20 h. LC analysis indicated >95% conversion. The mixture was diluted with ethyl acetate (50 mL) and washed with water (20 mL). The aqueous layer was extracted with ethyl acetate (3×20 mL). The combined organic layers were washed with saturated brine (50 mL), dried over anhydrous sodium sulfate, filtered and concentrated under reduced pressure. The residue was purified on an Interchim automated chromatography system (Sorbtech 25 g silica gel column), eluting with a gradient of 0 to 100% ethyl acetate in heptanes, to give the title compound (45 mg, 38% yield) as a brown oil. LCMS: m/z=748 [M+H]⁺.

### Example 82: 2,2-Difluoro-3-((1S,3R)-1-(5-(((R)-1-(3-fluoropropyl)pyrrolidin-3-yl)oxy)thiazol-2-yl)-3-methyl-1,3,4,9-tetrahydro-2H-pyrido[3,4-b]indol-2-yl)propan-1-ol

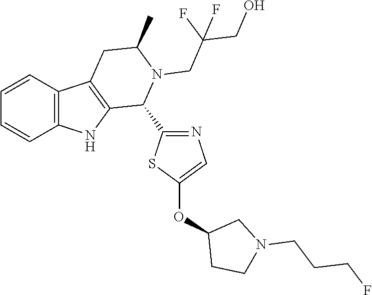 [SMILES: C[C@@H]1Cc2c([nH]c3ccccc23)[C@@H](c2ncc(O[C@@H]3CCN(CCCF)C3)s2)N1CC(F)(F)CO]

1M tetra-N-Butylammonium fluoride in THF (0.055 mL, 0.055 mmol, 1.1 equiv) was added dropwise at room temperature to a solution of 2-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-5-(((R)-1-(3-fluoropropyl)pyrrolidin-3-yl)oxy)thiazole (0.037 g, 0.0495 mmol, 1 equiv) in THF (2 mL). After stirring for 2 h, LC analysis indicated >99% conversion. The residue was diluted with saturated ammonium chloride (10 mL). The suspension was extracted with ethyl acetate (3×20 mL). The combined organic layers were dried over sodium sulfate, filtered and concentrated under reduced pressure. The residue was purified on an Interchim automated chromatography system (Biotage 11 g KPNH column), eluting with a gradient of 0 to 80% ethyl acetate in dichloromethane, to give the title compound (7 mg, 28% yield) as an off-white solid. LCMS: m/z=509 [M+H]⁺; ¹H NMR (400 MHz, CDCl₃) δ=8.48 (s, 1H), 7.49 (d, J=7.8 Hz, 1H), 7.34 (d, J=8.0 Hz, 1H), 7.17 (dt, J=1.2, 7.5 Hz, 1H), 7.13-7.07 (m, 1H), 6.91 (s, 1H), 5.17 (s, 1H), 4.65 (tdd, J=2.6, 5.3, 7.6 Hz, 1H), 4.56 (t, J=5.9 Hz, 1H), 4.45 (q, J=5.9 Hz, 1H), 4.06-3.91 (m, 2H), 3.49-3.38 (m, 1H), 3.34-3.16 (m, 2H), 2.96-2.75 (m, 4H), 2.72-2.55 (m, 4H), 2.50-2.43 (m, 1H), 2.29-2.19 (m, 1H), 2.09-2.02 (m, 1H), 2.00-1.75 (m, 3H), 1.75-1.54 (m, 2H), 1.45 (qd, J=7.3, 14.8 Hz, 1H), 1.32 (d, J=6.8 Hz, 3H).

### Example 83: 5-Bromo-2-((1S,3R)-2-(2,2-difluoroethyl)-3-methyl-2,3,4,9-tetrahydro-1H-pyrido[3,4-b]indol-1-yl)thiazole

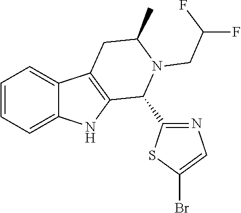 [SMILES: C[C@@H]1Cc2c([nH]c3ccccc23)[C@@H](c2ncc(Br)s2)N1CC(F)F]

A mixture of 5-bromothiazole-2-carbaldehyde (1 g, 5.21 mmol, 1 equiv), (R)—N-(2,2-difluoroethyl)-1-(1H-indol-3-yl)propan-2-amine (1.49 g, 6.25 mmol, 1.2 equiv) and acetic acid (0.66 mL, 10.4 mmol, 2 equiv) in toluene (21 mL) was heated at 80° C. in a 100 mL round bottom flask for 1 h. After cooling to room temperature, the mixture was diluted with ethyl acetate (30 mL) and washed with saturated sodium bicarbonate (50 mL). The aqueous layer was extracted with ethyl acetate (3×20 mL). The combined organic layers were washed with saturated brine (50 mL), dried over sodium sulfate, filtered and concentrated under reduced pressure. The residue was purified on an Biotage automated chromatography system (Biotage 100 g HC column), eluting with a gradient of 0 to 30% methyl-t-butyl ether in heptanes, to give the title compound (1.38 g, 64% yield) as a white solid. LCMS: m/z=412.0 [M+H]⁺.

### Example 84: tert-Butyl (1S,3R)-1-(5-bromothiazol-2-yl)-2-(2,2-difluoroethyl)-3-methyl-1,2,3,4-tetrahydro-9H-pyrido[3,4-b]indole-9-carboxylate

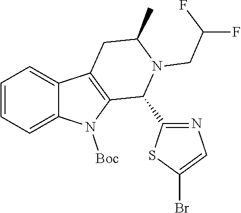 [SMILES: C[C@@H]1Cc2c(n(C(=O)OC(C)(C)C)c3ccccc23)[C@@H](c2ncc(Br)s2)N1CC(F)F]

Di-tert-butyl dicarbonate (1.43 g, 6.53 mmol, 1.5 equiv), 4-dimethylaminopyridine (0.074 g, 0.61 mmol, 0.14 equiv) and triethylamine (1.22 mL, 8.72 mmol, 2 equiv) were added to a solution of 5-bromo-2-((1S,3R)-2-(2,2-difluoroethyl)-3-methyl-2,3,4,9-tetrahydro-1H-pyrido[3,4-b]indol-1-yl)thiazole (1.79 g, 4.36 mmol, 1 equiv) in dichloromethane (30 mL) at room temperature. After stirring at room temperature for 16 h, the reaction was diluted with dichloromethane (30 mL) and washed with water (50 mL) and saturated brine (50 mL). The organic layer was dried over sodium sulfate, filtered and concentrated under reduced pressure. The residue was purified on a Biotage automated chromatography system (Biotage 100 g HC column), eluting with gradient of 0 to 30% methyl-tert-butyl ether in heptane, to give the title compound (2.0 g, 90% yield) as white solid. LCMS: m/z=512.1 [M+H]⁺.

### Example 85: tert-Butyl (1S,3R)-1-(5-((1-(tert-butoxycarbonyl)azetidin-3-yl)oxy)thiazol-2-yl)-2-(2,2-difluoroethyl)-3-methyl-1,2,3,4-tetrahydro-9H-pyrido[3,4-b]indole-9-carboxylate)

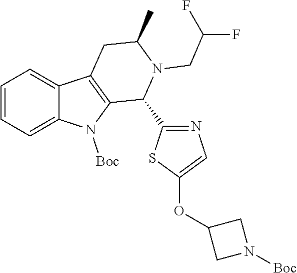 [SMILES: C[C@@H]1Cc2c(n(C(=O)OC(C)(C)C)c3ccccc23)[C@@H](c2ncc(OC3CN(C(=O)OC(C)(C)C)C3)s2)N1CC(F)F]

Two batches of tert-butyl (1S,3R)-1-(5-bromothiazol-2-yl)-2-(2,2-difluoroethyl)-3-methyl-1,2,3,4-tetrahydro-9H-pyrido[3,4-b]indole-9-carboxylate (500 mg, 0.976 mmol, 1 equiv), tert-butyl 3-hydroxyazetidine-1-carboxylate (376 mg, 3.26 mmol, 3.34 equiv) and 1,4-diazabicyclo[2.2.2]octane (328 mg, 2.93 mmol, 3 equiv) in acetonitrile (10 mL) in 40 ml vials were sparged with nitrogen for 20 min. Two batches of nickel (II) chloride (glyme) complex (107 mg, 0.488 mmol, 0.5 equiv), 4,4′-di-tert-butyl-2,2′-dipyridyl (131 mg, 0.488 mmol, 0.5 equiv), and (Ir[dF(CF₃)ppy]₂(dtbpy))PF₆ (109 mg, 0.0976 mmol, 0.1 equiv) were added to each solution at room temperature, and the mixture was sparged with nitrogen for an additional 5 min. The reaction vessels were sealed and both reactions were irradiated with blue LED light for 72 h. The two batches were combined and filtered through Celite® which was rinsed with ethyl acetate (2×30 mL). The filtrate was concentrated under reduced pressure. The residue was purified on a Biotage automated chromatography system (Biotage 50 g HC column), eluting with gradient of 0 to 30% ethyl acetate in hexanes, to give the title compound (112 mg, 10% yield) as a white solid. LCMS: m/z=605.3 [M+H]⁻.

### Example 86: 5-(Azetidin-3-yloxy)-2-((1S,3R)-2-(2,2-difluoroethyl)-3-methyl-2,3,4,9-tetrahydro-1H-pyrido[3,4-b]indol-1-yl)thiazole

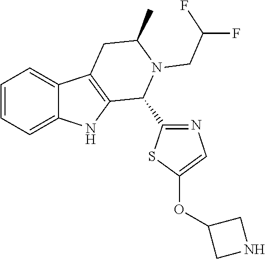 [SMILES: C[C@@H]1Cc2c([nH]c3ccccc23)[C@@H](c2ncc(OC3CNC3)s2)N1CC(F)F]

Trifluoroacetic acid (0.85 mL, 11.1 mmol, 60 equiv) was added at 0° C. to a solution of tert-butyl (1S,3R)-1-(5-((1-(tert-butoxycarbonyl)azetidin-3-yl)oxy)thiazol-2-yl)-2-(2,2-difluoroethyl)-3-methyl-1,2,3,4-tetrahydro-9H-pyrido[3,4-b]indole-9-carboxylate) (112 mg, 0.185 mmol, 1.0 equiv) in dichloromethane (4.2 mL). After stirring at 0° C. for 24 h, the reaction was diluted with dichloromethane (16 mL). The pH of the solution was carefully adjusted to pH 10 with saturated sodium carbonate at 0° C. The mixture was warmed to room temperature. The layers were separated, and the aqueous layer was extracted with dichloromethane (3×5 mL). The combined organic layers were washed with saturated brine (5 mL), dried over sodium sulfate, filtered and concentrated under reduced pressure to give the title compound (69.4 mg, 93% yield) as an orange solid. LCMS: m/z=405.1 [M+H]⁺.

### Example 87: 2-((1S,3R)-2-(2,2-Difluoroethyl)-3-methyl-2,3,4,9-tetrahydro-1H-pyrido[3,4-b]indol-1-yl)-5-((1-(3-fluoropropyl)azetidin-3-yl)oxy)thiazole

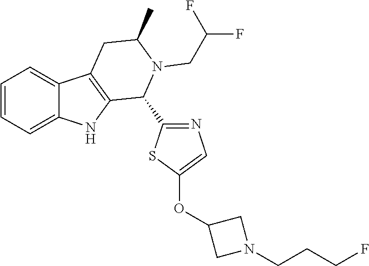 [SMILES: C[C@@H]1Cc2c([nH]c3ccccc23)[C@@H](c2ncc(OC3CN(CCCF)C3)s2)N1CC(F)F]

N,N-Diisopropylethylamine (0.064 mL, 0.257 mmol, 1.5 equiv) and 1-bromo-3-fluoropropane (27 mg, 0.189 mmol, 1.2 equiv) were sequentially added to a solution of 5-(azetidin-3-yloxy)-2-((1S,3R)-2-(2,2-difluoroethyl)-3-methyl-2,3,4,9-tetrahydro-1H-pyrido[3,4-b]indol-1-yl)thiazole (69 mg, 0.171 mmol, 1.0 equiv) in N,N-dimethylacetamide (0.5 mL). After stirring for 16 h at room temperature, saturated sodium bicarbonate (5 mL) and ethyl acetate (5 mL) were sequentially added. The layers were separated, and the aqueous layer was extracted with ethyl acetate (3×5 mL). The combined organic layers were washed with saturated brine (5 mL), dried over sodium sulfate, filtered and concentrated under reduced pressure. The resulting yellow oil was purified on Biotage automated chromatography system (Biotage 25 g KPNH), eluting with a gradient of 0 to 50% ethyl acetate in hexanes to give the title compound (15 mg, 19% yield) as a peach solid. ¹H NMR (400 MHz, CDCl₃) δ=8.38 (s, 1H), 7.49 (d, J=7.8 Hz, 1H), 7.34 (d, J=8.1 Hz, 1H), 7.17 (dt, J=1.2, 7.6 Hz, 1H), 7.12-7.07 (m, 1H), 6.86 (s, 1H), 6.05-5.72 (m, 1H), 5.04 (s, 1H), 4.67 (quin, J=5.6 Hz, 1H), 4.54 (t, J=5.9 Hz, 1H), 4.42 (t, J=6.0 Hz, 1H), 3.72 (ddd, J=1.9, 4.8, 10.4 Hz, 2H), 3.47-3.37 (m, 1H), 3.23-3.09 (m, 3H), 2.90-2.71 (m, 2H), 2.67-2.56 (m, 3H), 1.82-1.68 (m, 2H), 1.29 (d, J=6.8 Hz, 3H); LCMS: m/z=465.2 [M+H]⁺.

### Example 88: 2-Bromo-5-((1S,3R)-2-(2-fluoro-2-methylpropyl)-3-methyl-2,3,4,9-tetrahydro-1H-pyrido[3,4-b]indol-1-yl)thiazole

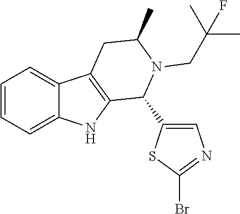 [SMILES: C[C@@H]1Cc2c([nH]c3ccccc23)[C@@H](c2cnc(Br)s2)N1CC(C)(C)F]

A mixture 2-bromothiazole-5-carbaldehyde (2 g, 10.4 mmol, 1 equiv), (R)—N-(1-(1H-indol-3-yl)propan-2-yl)-2-fluoro-2-methylpropan-1-amine (3.1 g, 12.5 mmol, 1.2 equiv) and acetic acid (1.2 mL, 20.8 mmol, 2.0 equiv) in toluene (35 mL) was heated at 80° C. in a 100 mL round bottom flask for 12 h. After cooling to room temperature, the mixture was diluted with ethyl acetate (50 mL) and washed with saturated sodium bicarbonate (100 mL). The aqueous layer was extracted with ethyl acetate (3×50 mL). The combined organic layers were washed with saturated brine (100 mL), dried over sodium sulfate, filtered and concentrated under reduced pressure. The residue was purified on an Biotage automated chromatography system (Biotage 100 g HC column), eluting with a gradient of 0 to 30% methyl-t-butyl ether in heptanes, to give the title compound (3.65 g, 83% yield) as a white solid. LCMS: m/z=423.1 [M+H]⁺.

### Example 89: tert-Butyl (1S,3R)-1-(2-bromothiazol-5-yl)-2-(2-fluoro-2-methylpropyl)-3-methyl-1,2,3,4-tetrahydro-9H-pyrido[3,4-b]indole-9-carboxylate

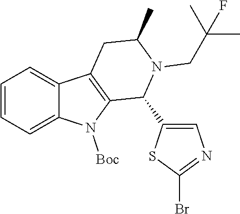 [SMILES: C[C@@H]1Cc2c(n(C(=O)OC(C)(C)C)c3ccccc23)[C@@H](c2cnc(Br)s2)N1CC(C)(C)F]

Di-tert-butyl dicarbonate (2.84 g, 13.0 mmol, 1.5 equiv) was added to a solution of 2-bromo-5-((1S,3R)-2-(2-fluoro-2-methylpropyl)-3-methyl-2,3,4,9-tetrahydro-1H-pyrido[3,4-b]indol-1-yl)thiazole (3.65 g, 8.64 mmol, 1.0 equiv), triethylamine (2.41 mL, 17.3 mmol, 2.0 equiv) and 4-dimethylaminopyridine (148 mg, 1.21 mmol, 0.14 equiv) in dichloromethane (60 mL). After stirring at room temperature for 16 h, the reaction was diluted with dichloromethane (30 mL) and washed with water (50 mL) and saturated brine (50 mL). The organic layer was dried over sodium sulfate, filtered and concentrated under reduced pressure. The residue was purified on a Biotage automated chromatography system (Biotage 100 g HC column), eluting with a gradient of 0 to 30% methyl-t-butyl ether in heptanes, to give the title compound (4.22 g, 93% yield) as a white solid. LCMS: m/z=524.1 [M+H]⁺.

### Example 90: tert-Butyl (1S,3R)-1-(2-((1-(tert-butoxycarbonyl)azetidin-3-yl)oxy)thiazol-5-yl)-2-(2-fluoro-2-methylpropyl)-3-methyl-1,2,3,4-tetrahydro-9H-pyrido[3,4-b]indole-9-carboxylate

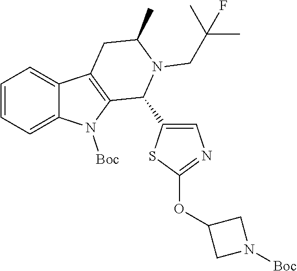 [SMILES: C[C@@H]1Cc2c(n(C(=O)OC(C)(C)C)c3ccccc23)[C@@H](c2cnc(OC3CN(C(=O)OC(C)(C)C)C3)s2)N1CC(C)(C)F]

Two batches of tert-butyl (1S,3R)-1-(2-bromothiazol-5-yl)-2-(2-fluoro-2-methylpropyl)-3-methyl-1,2,3,4-tetrahydro-9H-pyrido[3,4-b]indole-9-carboxylate (500 mg, 0.958 mmol, 1.0 equiv), tert-butyl 3-hydroxyazetidine-1-carboxylate (369 mg, 3.26 mmol, 3.34 equiv) and 1,4-diazabicyclo[2.2.2]octane (323 mg, 2.87 mmol, 3.0 equiv) in acetonitrile (10 mL) in 40 mL vials were sparged with nitrogen for 20 min. Two batches of nickel (II) chloride (glyme) complex (105 mg, 0.479 mmol, 0.5 equiv), 4,4′-di-tert-butyl-2,2′-dipyridyl (129 mg, 0.479 mmol, 0.5 equiv) and (Ir[dF(CF₃)ppy]₂(dtbpy))PF₆ (108 mg, 0.0958 mmol, 0.1 equiv) were added to each solution at room temperature and the mixture was sparged for an additional 5 min with nitrogen. The reaction vessels were sealed and both reactions were irradiated with blue LED light for 72 h. The two batches were combined and filtered through Celite®, which was rinsed with ethyl acetate (2×30 mL). The filtrate was concentrated under reduced pressure. The residue was purified on a Biotage automated chromatography system, eluting with gradient of 0 to 30% ethyl acetate in hexanes, to give the title compound (800 mg, 68% yield). LCMS: m/z=615.3 [M+H]⁺.

### Example 91: 2-(Azetidin-3-yloxy)-5-((1S,3R)-2-(2-fluoro-2-methylpropyl)-3-methyl-2,3,4,9-tetrahydro-1H-pyrido[3,4-b]indol-1-yl)thiazole

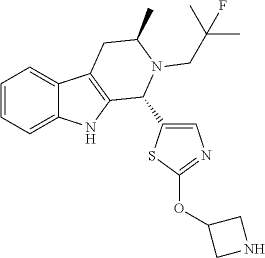 [SMILES: C[C@@H]1Cc2c([nH]c3ccccc23)[C@@H](c2cnc(OC3CNC3)s2)N1CC(C)(C)F]

Trifluoroacetic acid (0.9 mL, 11.7 mmol, 60 equiv) was added at 0° C. to a solution of tert-butyl (1S,3R)-1-(2-((1-(tert-butoxycarbonyl)azetidin-3-yl)oxy)thiazol-5-yl)-2-(2-fluoro-2-methylpropyl)-3-methyl-1,2,3,4-tetrahydro-9H-pyrido[3,4-b]indole-9-carboxylate (120 mg, 0.195 mmol, 1.0 equiv) in dichloromethane (1.8 mL). After stirring at 0° C. for 24 h, the reaction was diluted with dichloromethane (18 mL). The pH of the solution was carefully adjusted to pH 10 with saturated sodium carbonate at 0° C. The mixture was warmed to room temperature. The layers were separated, and the aqueous layer was extracted with dichloromethane (3×5 mL). The combined organic layers were washed with saturated brine (5 mL), dried over sodium sulfate, filtered and concentrated under reduced pressure to give the title compound (94.8 mg, >95% yield) as an orange. LCMS: m/z=415.2 [M+H]⁻.

### Example 92: 5-((1S,3R)-2-(2-Fluoro-2-methylpropyl)-3-methyl-2,3,4,9-tetrahydro-1H-pyrido[3,4-b]indol-1-yl)-2-((1-propylazetidin-3-yl)oxy)thiazole

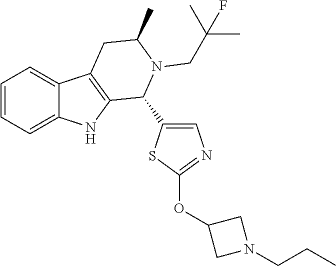 [SMILES: CCCN1CC(Oc2ncc([C@@H]3c4[nH]c5ccccc5c4C[C@@H](C)N3CC(C)(C)F)s2)C1]

2-Bromo-5-((1S,3R)-2-(2-fluoro-2-methylpropyl)-3-methyl-2,3,4,9-tetrahydro-1H-pyrido[3,4-b]indol-1-yl)thiazole (500 mg, 1.19 mmol, 1.0 equiv) and 1-propylazetidin-3-ol (301 mg, 2.62 mmol, 2.2 equiv) were dissolved in anhydrous 1,4-dioxane (3 mL). The mixture was sparged with nitrogen under sonication for 15 min. To an oven dried 40 mL vial equipped with magnetic stirrer bar was added copper iodide (17 mg, 0.09 mmol, 0.075 equiv), N,N-bis(2-phenylethyl) ethanediamide (27 mg, 0.09 mmol, 0.075 equiv), sodium tert-butoxide (137 mg, 1.43 mmol, 1.2 equiv) and freshly activated 4 Å MS (200 mg). The vial was sealed and evacuated and backfilled with nitrogen three times. The solution of the starting materials was then added via syringe at room temperature and the mixture was heated at 80° C. for 5 days. After cooling to room temperature, the reaction was diluted with ethyl acetate (30 mL) and 15% ammonium hydroxide (30 mL). The layers were separated, and the aqueous layer was extracted with ethyl acetate (3×30 mL). The combined organic layers were washed with 15% ammonium hydroxide (30 mL) and saturated brine (30 mL), dried over sodium sulfate, filtered and concentrated under reduced pressure. The residue was purified on a Biotage automated chromatography system (Biotage Sfär 50 g HC column), eluting with a gradient of 0 to 10% methanol in dichloromethane to give the title compound (74 mg, 14% yield) as a brown solid. ¹H NMR (400 MHz, CDCl₃) δ=8.60 (br s, 1H), 7.51 (d, J=7.7 Hz, 1H), 7.31 (d, J=7.9 Hz, 1H), 7.17 (dt, J=1.3, 7.5 Hz, 1H), 7.13-7.08 (m, 1H), 6.63 (s, 1H), 5.17 (quin, J=5.7 Hz, 1H), 5.12 (br s, 1H), 3.81-3.70 (m, 2H), 3.44 (br s, 1H), 3.15 (ddd, J=0.9, 5.3, 8.1 Hz, 2H), 2.68-2.43 (m, 6H), 1.48 (d, J=21.8 Hz, 3H), 1.38 (sxt, J=7.5 Hz, 2H), 1.32 (d, J=21.4 Hz, 3H), 1.16 (d, J=6.7 Hz, 3H), 0.89 (t, J=7.4 Hz, 3H); LCMS: m/z=457.2 [M+H]⁺.

### Example 93: 5-((1S,3R)-2-(2-Fluoro-2-methylpropyl)-3-methyl-2,3,4,9-tetrahydro-1H-pyrido[3,4-b]indol-1-yl)-2-((1-(3-fluoropropyl)azetidin-3-yl)oxy)thiazole

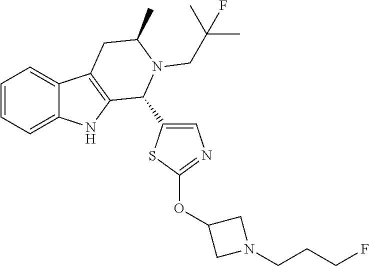 [SMILES: C[C@@H]1Cc2c([nH]c3ccccc23)[C@@H](c2cnc(OC3CN(CCCF)C3)s2)N1CC(C)(C)F]

N,N-Diisopropylethylamine (0.050 mL, 0.292 mmol, 1.5 equiv) and 1-bromo-3-fluoropropane (33 mg, 0.234 mmol, 1.2 equiv) were sequentially added to a solution of 2-(azetidin-3-yloxy)-5-((1S,3R)-2-(2-fluoro-2-methylpropyl)-3-methyl-2,3,4,9-tetrahydro-1H-pyrido[3,4-b]indol-1-yl)thiazole (81 mg, 0.195 mmol, 1.0 equiv) in N,N-dimethylacetamide (0.6 mL). After stirring for 16 h at room temperature, saturated sodium bicarbonate (5 mL) and ethyl acetate (5 mL) were sequentially added. The layers were separated, and the aqueous layer was extracted with ethyl acetate (3×5 mL). The combined organic layers were washed with saturated brine (5 mL), dried over sodium sulfate, filtered and concentrated under reduced pressure. The residue was purified on Biotage automated chromatography system (Biotage 11 g KPNH column), eluting with a gradient of 10 to 50% ethyl acetate in hexanes, to give the title compound (52.7 mg, 57% yield) as a white solid. ¹H NMR (400 MHz, CDCl₃) δ=7.95 (br s, 1H), 7.51 (d, J=7.8 Hz, 1H), 7.32 (d, J=8.1 Hz, 1H), 7.21-7.09 (m, 2H), 6.67 (s, 1H), 5.19 (quin, J=5.7 Hz, 1H), 5.13 (br s, 1H), 4.54 (t, J=6.0 Hz, 1H), 4.42 (t, J=6.0 Hz, 1H), 3.79-3.73 (m, 2H), 3.45 (br s, 1H), 3.16 (dd, J=5.3, 8.6 Hz, 2H), 2.69-2.48 (m, 6H), 1.82-1.68 (m, 2H), 1.51-1.43 (m, 3H), 1.34-1.28 (m, 3H), 1.15 (d, J=6.8 Hz, 3H); LCMS: m/z=475.3 [M+H]⁺.

### Example 94: tert-Butyl (1S,3R)-1-(2-(((R)-1-(tert-butoxycarbonyl)pyrrolidin-3-yl)oxy)thiazol-5-yl)-2-(2-fluoro-2-methylpropyl)-3-methyl-1,2,3,4-tetrahydro-9H-pyrido[3,4-b]indole-9-carboxylate

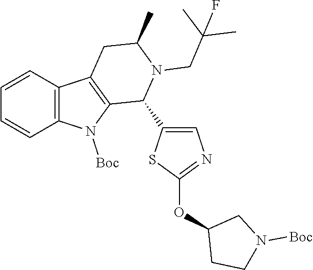 [SMILES: C[C@@H]1Cc2c(n(C(=O)OC(C)(C)C)c3ccccc23)[C@@H](c2cnc(O[C@@H]3CCN(C(=O)OC(C)(C)C)C3)s2)N1CC(C)(C)F]

Two batches of tert-butyl (1S,3R)-1-(2-bromothiazol-5-yl)-2-(2-fluoro-2-methylpropyl)-3-methyl-1,2,3,4-tetrahydro-9H-pyrido[3,4-b]indole-9-carboxylate (500 mg, 0.958 mmol, 1.0 equiv), tert-butyl (R)-3-hydroxypyrrolidine-1-carboxylate (599 mg, 3.2 mmol, 3.34 equiv) and 1,4-diazabicyclo[2.2.2]octane (323 mg, 2.87 mmol, 3.0 equiv) in acetonitrile (10 mL) in 40 mL vials were sparged with nitrogen for 20 min. Two batches of nickel (II) chloride (glyme) complex (105 mg, 0.479 mmol, 0.5 equiv), 4,4′-di-tert-butyl-2,2′-dipyridyl (129 mg, 0.479 mmol, 0.5 equiv) and (Ir[dF(CF₃)ppy]₂(dtbpy))PF₆ (108 mg, 0.0958 mmol, 0.1 equiv) were added to each solution at room temperature and the mixture was sparged with nitrogen for an additional 5 min. The reaction vessels were sealed and both reactions were irradiated with blue LED light for 72 h. The two batches were combined and filtered through Celite®, which was rinsed with ethyl acetate (2×30 mL). The filtrate was concentrated under reduced pressure. The residue was purified on a Biotage automated chromatography system (Biotage 50 g HC column), eluting with gradient of 0 to 60% methyl-t-butyl ether in heptanes, to give the title compound (535 mg, 44% yield) as a white solid. LCMS: m/z=629.3 [M+H]⁺.

### Example 95: 5-((1S,3R)-2-(2-Fluoro-2-methylpropyl)-3-methyl-2,3,4,9-tetrahydro-1H-pyrido[3,4-b]indol-1-yl)-2-(((R)-pyrrolidin-3-yl)oxy)thiazole

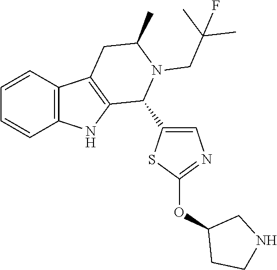 [SMILES: C[C@@H]1Cc2c([nH]c3ccccc23)[C@@H](c2cnc(O[C@@H]3CCNC3)s2)N1CC(C)(C)F]

Trifluoroacetic acid (0.73 mL, 9.55 mmol, 60 equiv) was added at 0° C. to a solution of tert-butyl (1S,3R)-1-(2-(((R)-1-(tert-butoxycarbonyl)pyrrolidin-3-yl)oxy)thiazol-5-yl)-2-(2-fluoro-2-methylpropyl)-3-methyl-1,2,3,4-tetrahydro-9H-pyrido[3,4-b]indole-9-carboxylate (100 mg, 0.159 mmol, 1 equiv) in dichloromethane (1.5 mL). After stirring at 0° C. for 24 h, the reaction was diluted with dichloromethane (10 mL). The pH of the solution was carefully adjusted to pH 10 with saturated sodium carbonate at 0° C. The mixture was warmed to room temperature. The layers were separated, and the aqueous layer was extracted with dichloromethane (3×5 mL). The combined organic layers were washed with saturated brine (5 mL), dried over sodium sulfate, filtered and concentrated under reduced pressure to give the title compound (78.5 mg, >95% yield) as an orange solid. LCMS: m/z=429.2 [M+H]⁺.

### Example 96: 5-((1S,3R)-2-(2-Fluoro-2-methylpropyl)-3-methyl-2,3,4,9-tetrahydro-1H-pyrido[3,4-b]indol-1-yl)-2-(((R)-1-(3-fluoropropyl)pyrrolidin-3-yl)oxy)thiazole

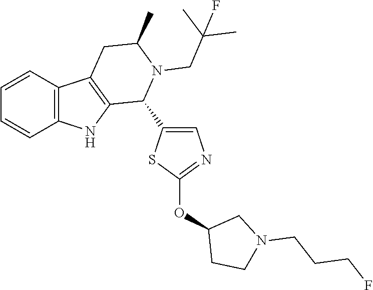 [SMILES: C[C@@H]1Cc2c([nH]c3ccccc23)[C@@H](c2cnc(O[C@@H]3CCN(CCCF)C3)s2)N1CC(C)(C)F]

N,N-Diisopropylethylamine (0.041 mL, 0.239 mmol, 1.5 equiv) and 1-bromo-3-fluoropropane (27 mg, 0.191 mmol, 1.2 equiv) were sequentially added to a solution of 5-((1S,3R)-2-(2-fluoro-2-methylpropyl)-3-methyl-2,3,4,9-tetrahydro-1H-pyrido[3,4-b]indol-1-yl)-2-(((R)-pyrrolidin-3-yl)oxy)thiazole (78 mg, 0.159 mmol, 1 equiv) in N,N-dimethylacetamide (0.5 mL). After stirring for 16 h at room temperature, saturated sodium bicarbonate (5 mL) and ethyl acetate (5 mL) were sequentially added. The layers were separated, and the aqueous layer was extracted with ethyl acetate (3×5 mL). The combined organic layers were washed with saturated brine (5 mL), dried over sodium sulfate, filtered and concentrated under reduced pressure. The residue was purified on Biotage automated chromatography system (Biotage 11 g KPNH column), eluting with a gradient of 10 to 60% ethyl acetate in hexanes, to give the title compound (65.9 mg, 85% yield) as a white solid. ¹H NMR (400 MHz, CDCl₃) δ=7.96 (br s, 1H), 7.51 (d, J=7.8 Hz, 1H), 7.33 (d, J=8.3 Hz, 1H), 7.18 (dt, J=1.3, 7.5 Hz, 1H), 7.14-7.09 (m, 1H), 6.68 (br s, 1H), 5.33 (dt, J=4.2, 7.2 Hz, 1H), 5.12 (br s, 1H), 4.56 (t, J=6.0 Hz, 1H), 4.45 (t, J=6.0 Hz, 1H), 3.46 (br s, 1H), 2.88-2.79 (m, 3H), 2.69-2.42 (m, 7H), 2.37-2.26 (m, 1H), 2.07-2.00 (m, 1H), 1.97-1.84 (m, 2H), 1.50-1.42 (m, 3H), 1.34-1.28 (m, 3H), 1.15 (d, J=6.7 Hz, 3H); LCMS: m/z=489.3 [M+H]⁺.

### Example 97: 2-Bromo-5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole

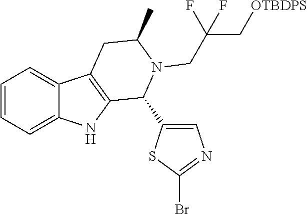

A solution of 2-bromothiazole-5-carbaldehyde (0.63 g, 3.3 mmol, 1.0 equiv), (R)—N-(1-(1H-indol-3-yl)propan-2-yl)-3-((tert-butyldiphenylsilyl)oxy)-2,2-difluoropropan-1-amine (2.0 g, 3.9 mmol, 1.2 equiv) and acetic acid (0.9 mL, 16.5 mmol, 5.0 equiv) in toluene (11 mL) was stirred at 95° C. under nitrogen for 24 hours. After cooling to room temperature, the reaction mixture was diluted with ethyl acetate (40 mL), washed with a saturated sodium carbonate solution (3×20 mL), dried over magnesium sulfate (5 g), filtered and concentrated under reduced pressure to give a red brown oil. The crude product was absorbed onto silica gel (20 g) with dichloromethane (100 mL) and then concentrated under reduced pressure. The material was purified on a Biotage automated chromatography system (50 g, 20 μm column), eluting with a gradient of 0 to 50% methyl t-butyl ether in heptanes, to give the title compound (2.0 g, 88% yield) as a yellow solid. LCMS: m/z=680, 682 [M+H]⁺.

### Example 98: tert-Butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

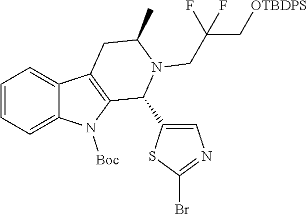

A mixture of 2-bromo-5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazole (2.0 g, 2.9 mmol, 1.0 equiv), di-tert-butyldicarbonate (0.95 g, 4.4 mmol, 1.5 equiv), triethylamine (0.59 g, 0.81 mL, 5.5 mmol, 2.0 equiv) and 4-dimethylaminopyridine in dichloromethane (20 mL) was stirred for 24 hours. The reaction mixture was diluted twofold with dichloromethane, washed with water (2×10 mL), dried over magnesium sulfate (5 g), filtered and concentrated under reduced pressure. The residue was absorbed onto silica gel (20 g) with dichloromethane (30 mL), then solvent was removed under reduced pressure. The material was purified on a Biotage automated chromatography system (50 g, 20 μm column), eluting with a gradient of 0 to 50% ethyl acetate in heptanes, to give the title compound (1.9 g, 83% yield) as a white foam. LCMS: m/z=780, 782 [M+H]⁺.

### Example 99: tert-Butyl (1S,3R)-1-(2-(((R)-1-(tert-butoxycarbonyl)pyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

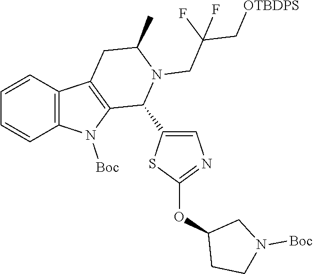

Two batches of tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (500 mg, 0.64 mmol, 1.0 equiv), tert-butyl (R)-3-hydroxypyrrolidine-1-carboxylate (400 mg, 2.1 mmol, 3.34 equiv) and 1,4-diazabicyclo[2.2.2]octane (215 mg, 1.9 mmol, 3.0 equiv) in acetonitrile (7 mL) in 40 mL vials were sparged with nitrogen for 25 minutes. Nickel (II) chloride (glyme) complex (70 mg, 0.32 mmol, 0.5 equiv), 4,4′-di-tert-butyl-2,2′-dipyridyl (86 mg, 0.32 mmol, 0.5 equiv) and (Ir[dF(CF₃)ppy]₂(dtbpy))PF₆ (72 mg, 0.06 mmol, 0.1 equiv) were added to each solution at room temperature and the mixture was sparged for an additional 5 minutes with nitrogen. The reaction vessels were sealed and both reactions were irradiated with blue LED light for 48 hours. The two batches were combined and filtered through Celite®, which was rinsed with ethyl acetate (3×30 mL). The filtrate was concentrated under reduced pressure. The material was purified on a Biotage automated chromatography system (50 g, 20 μm column), eluting with a gradient of 0 to 50% ethyl acetate to heptanes, to give the title compound (626 mg, 55% yield) as a white solid. LCMS: m/z=888 [M+H]⁺

### Example 100: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((R)-pyrrolidin-3-yl)oxy)thiazole

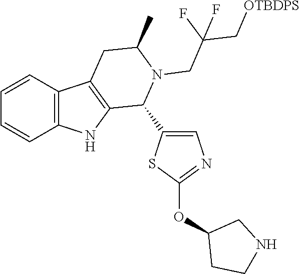

A solution of tert-butyl (1S,3R)-1-(2-(((R)-1-(tert-butoxycarbonyl)pyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (313 mg, 0.35 mmol, 1.0 equiv), trifluoroacetic acid (0.7 mL, 8.8 mmol, 25 equiv) and dichloromethane (3 mL) was stirred at 0 to 5° C. for 48 hours at which time thin layer chromatography and ICMS analysis indicated most of the starting material was consumed. The reaction mixture was added to saturated sodium carbonate (60 mL) such that the internal temperature did not exceed 5° C. The aqueous mixture (pH=10 by indicator paper) was extracted with dichloromethane (3×20 mL). The combined organic layers were washed with a saturated sodium carbonate solution (2×20 mL), dried over magnesium sulfate, filtered and concentrated under reduced pressure to give the title compound (195 mg, 80% yield) as a tan solid, which was used subsequently. LCMS: m/z=687 [M+H]⁺.

### Example 101: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)pyrrolidin-3-yl)oxy)thiazole

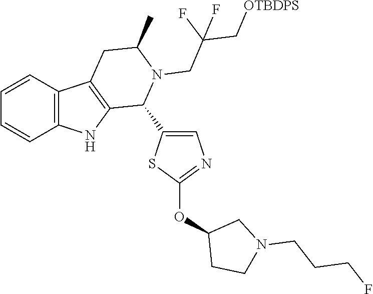

N,N-Diisopropylethylamine (0.18 g, 0.25 mL, 1.42 mmol, 5.0 equiv) was added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-pyrrolidin-3-yl)oxy)thiazole (195 mg, 0.28 mmol, 1.0 equiv) and 1-bromo-3-fluoropropane (44 mg, 0.31 mmol, 1.1 equiv) in N,N-dimethylacetamide (3 mL). After stirring for 18 hours, the reaction mixture was diluted with ethyl acetate (10 mL), washed with water (4×10 mL), dried over magnesium sulfate (1 g), filtered and concentrated under reduced pressure. The crude product was absorbed onto Celite® (1 g) with dichloromethane (1 mL) and solvent was removed under reduced pressure. The material was purified on a Biotage automated chromatography system (Amino Duo column, 11 g, 50 μm), eluting with a gradient from 0 to 50% ethyl acetate in hexanes to give the title compound (103 mg, 49% yield) as white solid. LCMS: m/z=748 [M+H]⁺.

### Example 102: 2,2-Difluoro-3-((1S,3R)-1-(2-(((R)-1-(3-fluoropropyl)pyrrolidin-3-yl)oxy)thiazol-5-yl)-3-methyl-1,3,4,9-tetrahydro-2H-pyrido[3,4-b]indol-2-yl)propan-1-ol

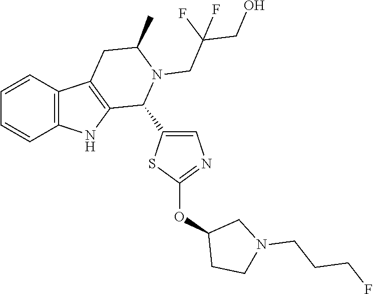 [SMILES: C[C@@H]1Cc2c([nH]c3ccccc23)[C@@H](c2cnc(O[C@@H]3CCN(CCCF)C3)s2)N1CC(F)(F)CO]

1 M Tetrabutylammonium fluoride in tetrahydrofuran (0.140 mL, 0.14 mmol, 1.1 equiv) was added to a solution of 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)pyrrolidin-3-yl)oxy)thiazole (103 mg, 0.04 mmol, 1.0 equiv) in anhydrous tetrahydrofuran (1.3 mL). The reaction mixture was stirred for 1.3 hours, at which time thin layer chromatography and LCMS analysis indicated that the starting material was mostly consumed. The reaction was concentrated under reduced pressure. The residue was treated with saturated ammonium chloride solution (7 mL) for 15 minutes, then extracted with ethyl acetate (3×5 mL). The combined organic layers were washed with saturated ammonium chloride (3×1 mL), dried over magnesium sulfate, filtered and concentrated under reduced pressure. The material was purified on a Biotage automated chromatography system (Amino Duo column, 5 g, 50 μm), eluting with a gradient of 0 to 100% ethyl acetate in hexanes, to give the title compound (41 mg, 58% yield) as white solid after lyophilization. ¹H NMR (400 MHz, CDCl₃) ¿=7.92 (s, 1H), 7.52 (d, J=7.8 Hz, 1H), 7.35-7.30 (m, 1H), 7.20 (dt, J=1.2, 7.6 Hz, 1H), 7.16-7.11 (m, 1H), 6.74 (s, 1H), 5.36-5.30 (m, 1H), 5.12 (s, 1H), 4.57 (t, J=5.9 Hz, 1H), 4.45 (t, J=5.9 Hz, 1H), 3.99-3.84 (m, 2H), 3.78-3.56 (m, 1H), 3.56-3.48 (m, 1H), 3.23-3.11 (m, 1H), 2.95-2.81 (m, 4H), 2.76 (dd, J=4.3, 16.1 Hz, 1H), 2.63-2.52 (m, 3H), 2.50-2.42 (m, 1H), 2.37-2.28 (m, 1H), 2.08-1.84 (m, 4H), 1.23 (d, J=6.7 Hz, 3H); LCMS: m/z=509.2 [M+H]⁺.

### Example 103: tert-Butyl (1S,3R)-1-(2-(((3R,5S)-1-(tert-butoxycarbonyl)-5-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

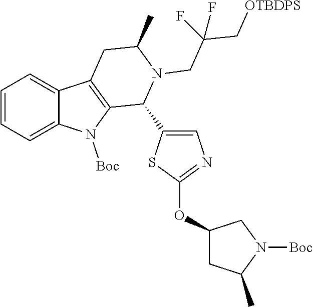

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.5 g, 0.64 mmol, 1 equiv) and tert-butyl (2S,4R)-4-hydroxy-2-methylpyrrolidine-1-carboxylate (0.43 g, 2.14 mmol, 3.34 equiv) in anhydrous acetonitrile (15 mL) was sparged with nitrogen for 20 minutes. 1,4-Diazabicyclo[2.2.2]octane (0.216 g, 1.92 mmol, 3.0 equiv), nickel (II) chloride ethylene glycol dimethylene ether complex (0.073 g, 0.32 mmol, 0.5 equiv), 4,4′-bis(di-t-butyl)-2,2′-bipyridine (0.086 g, 0.32 mmol, 0.5 equiv) and Ir[dF(CF₃)PPy]₂dtbbpy]PF₆ catalyst (0.072 g, 0.064 mmol, 0.1 equiv) were sequentially added and the mixture was sparged with a stream of nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 100% ethyl acetate in heptanes, to give the title compound (0.35 g, 61% yield) as a white solid. LCMS: m/z=902.2 [M+H]⁺.

### Example 104: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((3R,5S)-5-methylpyrrolidin-3-yl)oxy)thiazole

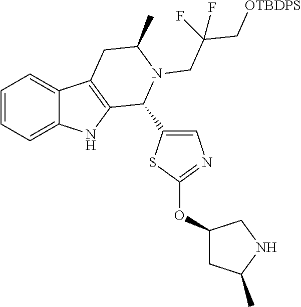

Trifluoroacetic acid (1.78 mL, 23.3 mmol, 60 equiv) was added dropwise over ˜2 minutes to tert-butyl (1S,3R)-1-(2-(((3R,5S)-1-(tert-butoxycarbonyl)-5-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.35 g, 0.39 mmol, 1.0 equiv) in dichloromethane (3.0 mL) in a 20 mL vial at 0° C. The reaction was stirred at 0° C. for 2 days. The pH was adjusted to 10 with cold saturated sodium carbonate and the solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.237 g, 86% yield) as a yellow solid, which was used subsequently. LCMS: m/z=701.2 [M+H]⁺.

### Example 105: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((3R,5S)-1-(3-fluoropropyl)-5-methylpyrrolidin-3-yl)oxy)thiazole

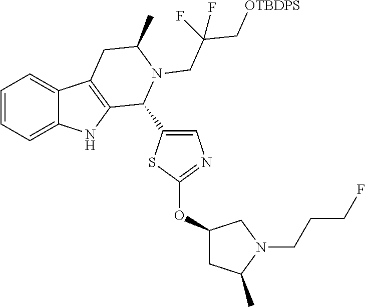

1-Bromo-3-fluoropropane (47 μL, 0.34 mmol, 1.5 equiv) and N,N-diisopropylethylamine (0.3 mL, 1.7 mmol, 5.0 equiv) were added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5S)-5-methylpyrrolidin-3-yl)oxy)thiazole (0.237 g, 0.34 mmol, 1.0 equiv) in N,N-dimethylacetamide (2.0 mL) in a 40 mL vial and stirred at room temperature for 4 days. The reaction mixture was diluted with ethyl acetate (20 mL) and washed with saturated brine (2×15 mL). The organic layer was dried over sodium sulfate, filtered and concentrated under reduced pressure. The residue was dried under vacuum at 25° C. for 1.5 hours to give the title compound (0.259 g, 100% yield) as an orange oil, which was used subsequently. LCMS: m/z=762.3 [M+H]⁺.

### Example 106: 2,2-Difluoro-3-((1S,3R)-1-(2-(((3R,5S)-1-(3-fluoropropyl)-5-methylpyrrolidin-3-yl)oxy)thiazol-5-yl)-3-methyl-1,3,4,9-tetrahydro-2H-pyrido[3,4-b]indol-2-yl)propan-1-ol

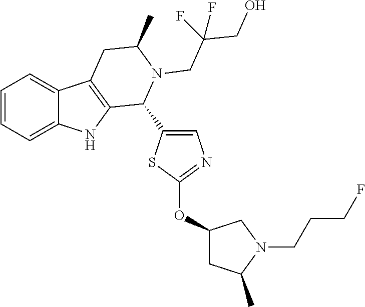 [SMILES: C[C@@H]1Cc2c([nH]c3ccccc23)[C@@H](c2cnc(O[C@@H]3C[C@H](C)N(CCCF)C3)s2)N1CC(F)(F)CO]

1M Tetra-n-butyl ammonium fluoride in tetrahydrofuran (0.37 mL, 0.37 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5S)-1-(3-fluoropropyl)-5-methylpyrrolidin-3-yl)oxy)thiazole (0.26 g, 0.34 mmol, 1.0 equiv) in tetrahydrofuran (2.0 mL) in a 40 mL vial and stirred at room temperature for 1.5 hours. The solvent was concentrated under reduced pressure. The residue was diluted with saturated ammonium chloride solution (5 mL) and extracted with ethyl acetate (3×10 mL). The combined organic layers were washed with saturated brine (1×20 mL), dried over sodium sulfate, filtered and concentrated under reduced pressure onto silica (12 g). The material was purified on a Büchi automated chromatography system (28 g, Biotage 50 μm KP-amino-d column), eluting with a gradient of 0 to 100% ethyl acetate in hexanes. The solid was dried under vacuum at 25° C. for 16 hours to give title compound (120 mg, 67% yield) as a white solid. ¹H NMR (400 MHz, CDCl₃) δ=7.92 (s, 1H), 7.51 (d, J=7.8 Hz, 1H), 7.32 (d, J=7.8 Hz, 1H), 7.20 (dt, J=1.2, 7.6 Hz, 1H), 7.15-7.10 (m, 1H), 6.73 (s, 1H), 5.31-5.23 (m, 1H), 5.11 (s, 1H), 4.61-4.53 (m, 1H), 4.50-4.41 (m, 1H), 4.00-3.84 (m, 2H), 3.57-3.48 (m, 1H), 3.36 (d, J=11.4 Hz, 1H), 3.24-3.06 (m, 1H), 3.04-2.84 (m, 3H), 2.75 (dd, J=4.3, 16.1 Hz, 1H), 2.61-2.48 (m, 2H), 2.44-2.29 (m, 2H), 2.16-2.00 (m, 1H), 1.95-1.81 (m, 2H), 1.73-1.62 (m, 1H), 1.28-1.13 (m, 6H). LCMS: m/z=523.3 [M+H]⁺.

### Example 107: tert-Butyl (1S,3R)-1-(2-(((3R,5R)-1-(tert-butoxycarbonyl)-5-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

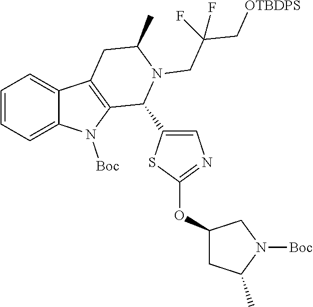

A solution of tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylatebutyl 4-(5-(hydroxymethyl)thiophen-2-yl)piperidine-1-carboxylate (0.78 g, 1.0 mmol, 1 equiv) and tert-butyl (2R,4R)-4-hydroxy-2-methylpyrrolidine-1-carboxylate (0.67 g, 3.34 mmol, 3.34 equiv) in anhydrous acetonitrile (15 mL) was sparged with nitrogen for 20 minutes. 1,4-Diazabicyclo[2.2.2]octane (0.336 g, 3.0 mmol, 3.0 equiv), nickel (II) chloride ethylene glycol dimethylene ether complex (0.11 g, 0.5 mmol, 0.5 equiv), 4,4′-bis(di-t-butyl)-2,2′-bipyridine (0.134 g, 0.5 mmol, 0.5 equiv) and Ir[dF(CF₃)PPy]₂dtbbpy]PF₆ catalyst (0.112 g, 0.1 mmol, 0.1 equiv) were sequentially added and the mixture was sparged with nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 100% ethyl acetate in hexanes, to give title compound (0.51 g, 57% yield) as a white solid. LCMS: m/z=902.2 [M+H]⁺.

### Example 108: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5R)-5-methylpyrrolidin-3-yl)oxy)thiazole

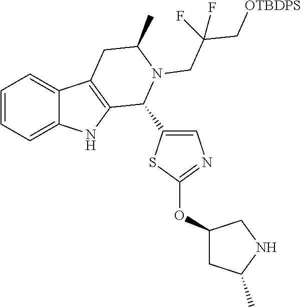

Trifluoroacetic acid (2.60 mL, 34 mmol, 60 equiv) was added dropwise over ˜2 minutes to tert-butyl (1S,3R)-1-(2-(((3R,5R)-1-(tert-butoxycarbonyl)-5-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.51 g, 0.56 mmol, 1.0 equiv) in dichloromethane (2.60 mL) in a 20 mL vial at 0° C. After stirring at 0° C. for 24 hours the pH was adjusted to 10 with cold saturated sodium carbonate and extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.368 g, 86% yield) as a yellow solid, which was used subsequently. LCMS: m/z=701.2 [M+H]⁺.

### Example 109: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5R)-1-(3-fluoropropyl)-5-methylpyrrolidin-3-yl)oxy)thiazole

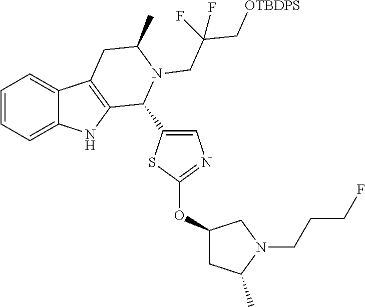

1-Bromo-3-fluoropropane (72 μL, 0.74 mmol, 1.5 equiv) and N,N-diisopropylethylamine (0.46 mL, 2.6 mmol, 5.0 equiv) were added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5R)-5-methylpyrrolidin-3-yl)oxy)thiazole (0.368 g, 0.52 mmol, 1.0 equiv) in N,N-dimethylacetamide (4.0 mL) in a 40 mL vial and stirred at room temperature for 2 days. The reaction mixture was diluted with ethyl acetate (20 mL) and washed with saturated brine (2×15 mL). The organic layer was dried over sodium sulfate, filtered and concentrated under reduced pressure and dried under vacuum at 25° C. for 1.5 hours to give the title compound (0.377 g, 95% yield) as an orange oil, which was used subsequently. LCMS: m/z=762.3 [M+H]⁺.

### Example 110: 2,2-Difluoro-3-((1S,3R)-1-(2-(((3R,5R)-1-(3-fluoropropyl)-5-methylpyrrolidin-3-yl)oxy)thiazol-5-yl)-3-methyl-1,3,4,9-tetrahydro-2H-pyrido[3,4-b]indol-2-yl)propan-1-ol

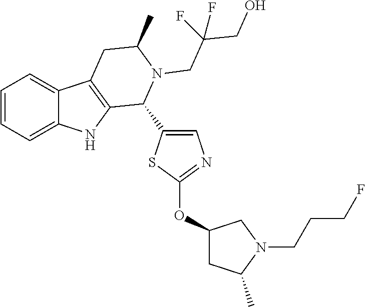 [SMILES: C[C@@H]1C[C@@H](Oc2ncc([C@@H]3c4[nH]c5ccccc5c4C[C@@H](C)N3CC(F)(F)CO)s2)CN1CCCF]

1M Tetra-n-butyl ammonium fluoride in tetrahydrofuran (0.54 mL, 0.54 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3R,5R)-1-(3-fluoropropyl)-5-methylpyrrolidin-3-yl)oxy)thiazole (0.377 g, 0.5 mmol, 1.0 equiv) in tetrahydrofuran (5.0 mL) in a 40 mL vial and stirred at room temperature for 1.5 hours. The solvent was concentrated under reduced pressure. The residue was diluted with saturated ammonium chloride (5 mL) and extracted with ethyl acetate (3×10 mL). The combined organic layers were washed with saturated brine (1×20 mL), dried over sodium sulfate, filtered and concentrated under reduced pressure onto silica (12 g). The material was purified on a Büchi automated chromatography system (28 g, Biotage 50 μm KP-amino-d column), eluting with a gradient of 0 to 100% ethyl acetate in hexanes. The solid was dried under vacuum at 25° C. for 16 hours to give title compound (100 mg, 38% yield) as a white solid. ¹H NMR (400 MHz, CDCl₃) δ=7.88 (s, 1H), 7.52 (d, J=7.8 Hz, 1H), 7.32 (d, J=7.8 Hz, 1H), 7.20 (dt, J=1.3, 7.5 Hz, 1H), 7.15-7.10 (m, 1H), 6.75 (s, 1H), 5.28-5.22 (m, 1H), 5.12 (s, 1H), 4.60-4.51 (m, 1H), 4.48-4.39 (m, 1H), 4.00-3.85 (m, 2H), 3.68 (dd, J=6.5, 11.0 Hz, 1H), 3.58-3.47 (m, 1H), 3.23-3.07 (m, 1H), 3.06-2.85 (m, 3H), 2.80-2.72 (m, 1H), 2.72-2.64 (m, 1H), 2.56 (dd, J=8.9, 15.7 Hz, 1H), 2.39 (dd, J=4.1, 10.9 Hz, 1H), 2.32-2.23 (m, 1H), 2.13 (ddd, J=1.5, 6.0, 13.9 Hz, 1H), 1.94-1.76 (m, 3H), 1.24 (d, J=6.7 Hz, 3H), 1.09 (d, J=6.0 Hz, 3H); LCMS: m/z=523.3 [M+H]⁺.

### Example 111: tert-Butyl (1S,3R)-1-(2-(((3S,4S)-1-(tert-butoxycarbonyl)-4-fluoropyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

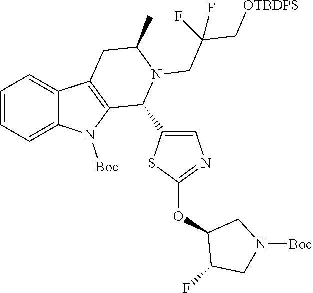

tert-Butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.200 g, 0.256 mmol, 1 equiv) and tert-butyl (3S,4S)-3-fluoro-4-hydroxypyrrolidine-1-carboxylate (0.176 g, 0.855 mmol, 3.3 equiv) in acetonitrile (6 mL) were sparged with nitrogen for 20 minutes in a 40 mL vial. Nickel (II) chloride ethylene glycol dimethylene ether complex (28 mg, 0.13 mmol, 0.5 equiv), 4,4′-bis(di-t-butyl)-2,2′-bipyridine (34 mg, 0.13 mmol, 0.5 equiv), Ir[dF(CF₃)PPy]₂dtbbpy]PF₆ (29 mg, 0.03 mmol, 0.10 equiv) and 1,4-diazabicyclo[2.2.2]octane (86 mg, 0.77 mmol, 3.0 equiv) were added. The reaction mixture was sparged with nitrogen for an additional 20 minutes. The reaction was irradiated with blue LED light at room temperature for 16 hours. The reaction mixture was concentrated under reduced pressure directly onto silica (4 g). The residue was purified on a Biotage automated chromatography system (40 g, Sorbtech silica gel column), eluting with a gradient of 0 to 20% ethyl acetate in hexanes. The solid was dried under vacuum at 40° C. for 1 hour to give title compound (0.155 g, 78% yield) as a white foam. LCMS: m/z=905.4 [M+H]⁺.

### Example 112: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((3S,4S)-4-fluoropyrrolidin-3-yl)oxy)thiazole

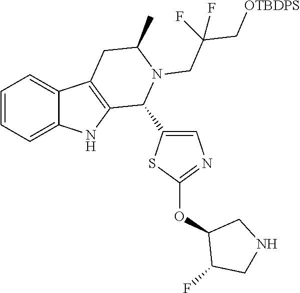

Trifluoroacetic acid (0.75 mL, 9.8 mmol, 60 equiv) was added to tert-butyl (1S,3R)-1-(2-(((3S,4S)-1-(tert-butoxycarbonyl)-4-fluoropyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.149 g, 0.164 mmol, 1 equiv) in dichloromethane (1.3 mL) at 0° C. in a 40 mL vial. The reaction was stirred at 0° C. for 32 hours. The reaction was diluted with cold dichloromethane (100 mL) and adjusted to pH 10 with saturated sodium carbonate (6 mL). The mixture was washed with saturated sodium carbonate (70 mL). The organic layer was dried over sodium sulfate, filtered, and concentrated under reduced pressure to give title compound (0.116 g, >95% yield) as a tan foam, which was used subsequently. LCMS: m/z=705.3 [M+H]⁺.

### Example 113: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((3S,4S)-4-fluoro-1-(3-fluoropropyl)pyrrolidin-3-yl)oxy)thiazole

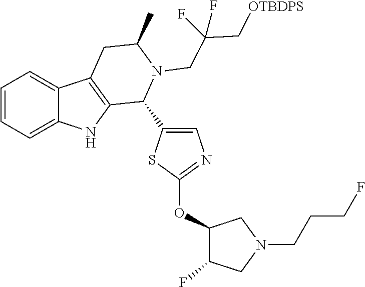

1-Bromo-3-fluoropropane (0.02 mL, 0.2 mmol, 1.5 equiv) and N,N-diisopropylethylamine (0.14 mL, 0.80 mmol, 5 equiv) were sequentially added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3S,4S)-4-fluoropyrrolidin-3-yl)oxy)thiazole (0.113 g, 0.160 mmol, 1 equiv) in N,N-dimethylacetamide (1.6 mL) in a 40 mL vial. After stirring for 6 days at room temperature, the reaction mixture was diluted with ethyl acetate (30 mL) and sequentially washed with water (3×30 mL) and saturated brine (30 mL). The organic layer was dried over sodium sulfate, filtered and concentrated under reduced pressure to give the title compound (0.110 g, 90% yield, 60% pure), which was used subsequently. LCMS: m/z=765.4 [M+H]⁺.

### Example 114: 2,2-Difluoro-3-((1S,3R)-1-(2-(((3S,4S)-4-fluoro-1-(3-fluoropropyl)pyrrolidin-3-yl)oxy)thiazol-5-yl)-3-methyl-1,3,4,9-tetrahydro-2H-pyrido[3,4-b]indol-2-yl)propan-1-ol

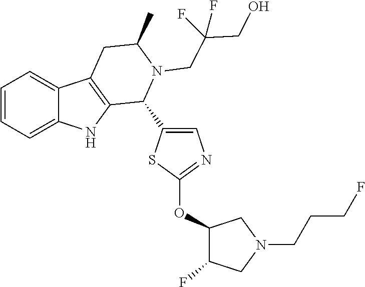 [SMILES: C[C@@H]1Cc2c([nH]c3ccccc23)[C@@H](c2cnc(O[C@H]3CN(CCCF)C[C@@H]3F)s2)N1CC(F)(F)CO]

1M Tetrabutylammonium fluoride (0.16 mL, 0.16 mmol, 1.1 equiv) was added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3S,4S)-4-fluoro-1-(3-fluoropropyl)pyrrolidin-3-yl)oxy)thiazole (0.110 g, 0.144 mmol, 1 equiv) in THF (1.5 mL) in a 40 mL vial at room temperature. After stirring for 1 hour at room temperature, the reaction mixture was concentrated under reduced pressure. The residue was diluted with ethyl acetate (20 mL) and washed with saturated ammonium chloride (10 mL). The aqueous layer was extracted with ethyl acetate (20 mL). The combined organic layers were sequentially washed with water (20 mL) and saturated brine (20 mL). The organic layer was dried over sodium sulfate, filtered and concentrated under reduced pressure onto Celite® (1 g). The residue was purified on a Biotage automated chromatography system (11 g, Biotage 50 μm KP-amino-d column), eluting with a gradient of 0 to 100% ethyl acetate in hexanes. The resulting solid was dried in a vacuum oven at 40° C. for 1.5 hours to give title compound (13.8 mg, 18% yield) as a white foam. LCMS: m/z=527.2 [M+H]⁺; ¹H NMR (400 MHz, CDCl₃) δ=7.82 (s, 1H), 7.52 (d, J=7.8 Hz, 1H), 7.33 (d, J=8.1 Hz, 1H), 7.20 (dt, J=1.2, 7.6 Hz, 1H), 7.15-7.10 (m, 1H), 6.36 (dd, J=1.2, 3.9 Hz, 1H), 5.88 (d, J=3.8 Hz, 1H), 5.03 (s, 1H), 4.70 (quin, J=5.7 Hz, 1H), 4.54 (t, J=5.9 Hz, 1H), 4.42 (t, J=6.0 Hz, 1H), 3.77-3.71 (m, 2H), 3.54-3.45 (m, 1H), 3.28-3.09 (m, 3H), 3.02-2.90 (m, 1H), 2.75 (dd, J=4.3, 16.1 Hz, 1H), 2.62 (t, J=7.1 Hz, 2H), 2.56-2.47 (m, 1H), 1.82-1.69 (m, 2H), 1.21 (d, J=6.7 Hz, 3H).

### Example 115: tert-Butyl (1S,3R)-1-(2-(((3S,4R)-1-(tert-butoxycarbonyl)-4-fluoropyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

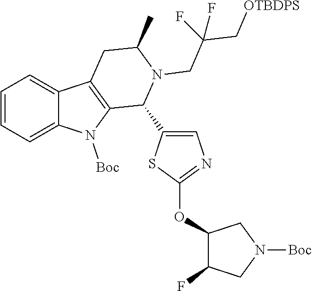

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.2 g, 0.26 mmol, 1 equiv) and tert-butyl (3R,4S)-3-fluoro-4-hydroxypyrrolidine-1-carboxylate (0.176 g, 0.86 mmol, 3.34 equiv) in anhydrous acetonitrile (15 mL) was sparged with a nitrogen for 20 minutes. 1,4-Diazabicyclo[2.2.2]octane (0.086 g, 0.77 mmol, 3.0 equiv), nickel (II) chloride ethylene glycol dimethylene ether complex (0.028 g, 0.13 mmol, 0.5 equiv), 4,4′-bis(di-t-butyl)-2,2′-bipyridine (0.034 g, 0.13 mmol, 0.5 equiv) and Ir[dF(CF₃)PPy]₂dtbbpy]PF₆ catalyst (0.029 g, 0.026 mmol, 0.1 equiv) were sequentially added and the mixture was sparged with a nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 100% ethyl acetate in heptanes, to give title compound (0.17 g, 73% yield) as a white solid. LCMS: m/z=906.2 [M+H]⁻.

### Example 116: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3S,4R)-4-fluoropyrrolidin-3-yl)oxy)thiazole

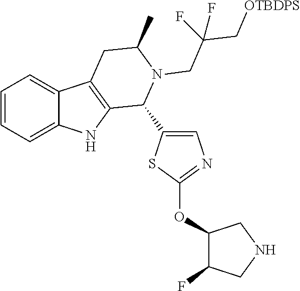

Trifluoroacetic acid (0.9 mL, 11.3 mmol, 60 equiv) was added dropwise over ˜2 minutes to tert-butyl (1S,3R)-1-(2-(((3S,4R)-1-(tert-butoxycarbonyl)-4-fluoropyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.17 g, 0.19 mmol, 1.0 equiv) in dichloromethane (0.9 mL) in a 20 mL vial at 0° C. The reaction was stirred at 0° C. for 3 days. The pH was adjusted to 10 with cold saturated sodium carbonate. The solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.134 g, 100% yield) as a yellow foam, which was used subsequently. LCMS: m/z=705.2 [M+H]⁺.

### Example 117: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((3S,4R)-4-fluoro-1-(3-fluoropropyl)pyrrolidin-3-yl)oxy)thiazole

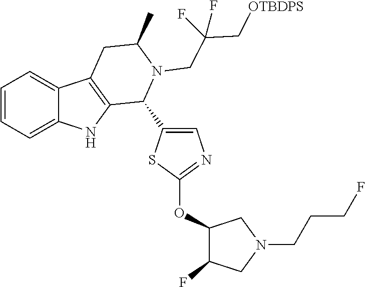

1-Bromo-3-fluoropropane (26 μL, 0.29 mmol, 1.5 equiv) and N,N-diisopropylethylamine (0.17 mL, 0.95 mmol, 5.0 equiv) was added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3S,4R)-4-fluoropyrrolidin-3-yl)oxy)thiazole (0.134 g, 0.19 mmol, 1.0 equiv) in N,N-dimethylacetamide (2.0 mL) in a 40 mL vial and stirred at room temperature for 3 days. The reaction mixture was diluted with ethyl acetate (20 mL) and washed with saturated brine (2×15 mL). The organic layer was dried over sodium sulfate, filtered and concentrated under reduced pressure and dried under vacuum at 25° C. for 1.5 hours to give the title compound (0.108 g, 100% yield) as a yellow foam, which was used subsequently. LCMS: m/z=766.2 [M+H]⁺

### Example 118: 2,2-Difluoro-3-((1S,3R)-1-(2-(((3S,4R)-4-fluoro-1-(3-fluoropropyl)pyrrolidin-3-yl)oxy)thiazol-5-yl)-3-methyl-1,3,4,9-tetrahydro-2H-pyrido[3,4-b]indol-2-yl)propan-1-ol

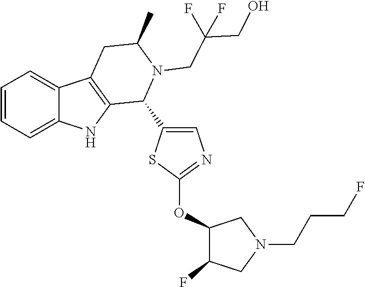 [SMILES: C[C@@H]1Cc2c([nH]c3ccccc23)[C@@H](c2cnc(O[C@H]3CN(CCCF)C[C@H]3F)s2)N1CC(F)(F)CO]

1M Tetra-n-butyl ammonium fluoride in tetrahydrofuran (0.16 mL, 0.16 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((3S,4R)-4-fluoro-1-(3-fluoropropyl)pyrrolidin-3-yl)oxy)thiazole (0.108 g, 0.14 mmol, 1.0 equiv) in tetrahydrofuran (2.0 mL) in a 40 mL vial and stirred at room temperature for 1.5 hours. The solvent was concentrated under reduced pressure. The residue was diluted with saturated ammonium chloride (5 mL) and extracted with ethyl acetate (3×10 mL). The combined organic layers were washed with saturated brine (1×20 mL), dried over sodium sulfate, filtered and concentrated under reduced pressure onto silica (12 g). The material was purified on a Büchi automated chromatography system (11 g, Biotage 50 μm KP-amino-d column), eluting with a gradient of 0 to 100% ethyl acetate in hexanes. The solid was dried under vacuum at 25° C. for 16 hours to give title compound (17 mg, 23% yield) as a white solid. ¹H NMR (400 MHz, CDCl₃) δ=7.84 (s, 1H), 7.52 (d, J=7.7 Hz, 1H), 7.33 (d, J=8.1 Hz, 1H), 7.21 (dt, J=1.2, 7.6 Hz, 1H), 7.16-7.11 (m, 1H), 6.73-6.68 (m, 1H), 5.35-5.12 (m, 3H), 4.56 (t, J=5.9 Hz, 1H), 4.44 (t, J=5.9 Hz, 1H), 4.01-3.84 (m, 2H), 3.56-3.45 (m, 1H), 3.23-3.11 (m, 1H), 3.08-2.82 (m, 6H), 2.78-2.52 (m, 4H), 1.95-1.80 (m, 2H), 1.24 (d, J=6.7 Hz, 3H). LCMS: m/z=527.2 [M+H]⁺.

### Example 119: tert-Butyl (1S,3R)-1-(2-(((2S,3R)-1-(tert-butoxycarbonyl)-2-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

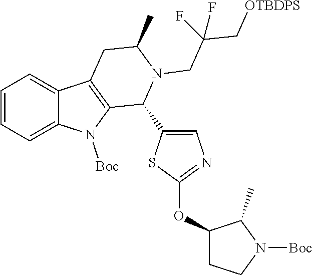

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.2 g, 0.26 mmol, 1 equiv) and tert-butyl (2S,3R)-3-hydroxy-2-methylpyrrolidine-1-carboxylate (0.175 g, 0.86 mmol, 3.34 equiv) in anhydrous acetonitrile (15 mL) was sparged with nitrogen for 20 minutes. 1,4-Diazabicyclo[2.2.2]octane (0.086 g, 0.77 mmol, 3.0 equiv), nickel (II) chloride ethylene glycol dimethylene ether complex (0.028 g, 0.13 mmol, 0.5 equiv), 4,4′-bis(di-t-butyl)-2,2′-bipyridine (0.034 g, 0.13 mmol, 0.5 equiv) and Ir[dF(CF₃)PPy]₂dtbbpy]PF₆ catalyst (0.029 g, 0.026 mmol, 0.1 equiv) were sequentially added and the mixture was sparged with stream of nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure and the residue was purified on a Büchi automated chromatography system (25 g, Sorbtech 40-75 μm silica gel column), eluting with a gradient of 0 to 100% ethyl acetate in heptanes, to give the title compound (0.135 g, 58% yield) as a white solid. LCMS: m/z=902.2 [M+H]⁻.

### Example 120: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((2S,3R)-2-methylpyrrolidin-3-yl)oxy)thiazole

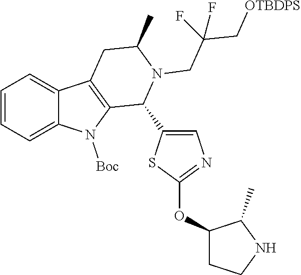

Trifluoroacetic acid (0.69 mL, 9.0 mmol, 60 equiv) was added dropwise over ˜2 minutes to tert-butyl (1S,3R)-1-(2-(((2S,3R)-1-(tert-butoxycarbonyl)-2-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.135 g, 0.15 mmol, 1.0 equiv) in dichloromethane (1.0 mL) in a 20 mL vial at 0° C. After stirring at 0° C. for 3 days, the pH was adjusted to 10 with cold saturated sodium carbonate. The solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.103 g, 98% yield) as a yellow foam, which was used subsequently. LCMS: m/z=702.2 [M+H]⁺.

### Example 121: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2S,3R)-1-(3-fluoropropyl)-2-methylpyrrolidin-3-yl)oxy)thiazole

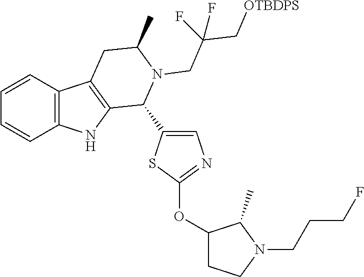

1-Bromo-3-fluoropropane (17 μL, 0.18 mmol, 1.2 equiv) and N,N-diisopropylethylamine (0.13 mL, 0.75 mmol, 5.0 equiv) were added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2S,3R)-2-methylpyrrolidin-3-yl)oxy)thiazole (0.103 g, 0.15 mmol, 1.0 equiv) in N,N-dimethylacetamide (2.0 mL) in a 40 mL vial and stirred at room temperature for 3 days. The reaction mixture was extracted with ethyl acetate (20 mL). The organic layer was washed with saturated brine (2×15 mL), dried over sodium sulfate, filtered and concentrated under reduced pressure. The residue was dried under vacuum at 25° C. for 1.5 hours to give the title compound (0.101 g, 89% yield) as an orange oil, which was used subsequently. LCMS: m/z=762.2 [M+H]⁺

### Example 122: 2,2-Difluoro-3-((1S,3R)-1-(2-(((2S,3R)-1-(3-fluoropropyl)-2-methylpyrrolidin-3-yl)oxy)thiazol-5-yl)-3-methyl-1,3,4,9-tetrahydro-2H-pyrido[3,4-b]indol-2-yl)propan-1-ol

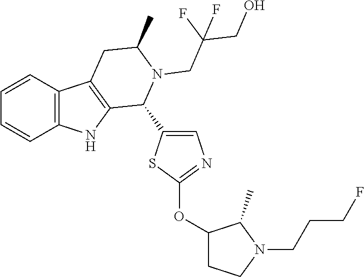 [SMILES: C[C@@H]1Cc2c([nH]c3ccccc23)[C@@H](c2cnc(OC3CCN(CCCF)[C@H]3C)s2)N1CC(F)(F)CO]

1M Tetra-n-butyl ammonium fluoride in tetrahydrofuran (0.15 mL, 0.15 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2S,3R)-1-(3-fluoropropyl)-2-methylpyrrolidin-3-yl)oxy)thiazole (0.101 g, 0.13 mmol, 1.0 equiv) in tetrahydrofuran (1.5 mL) in a 40 mL vial and stirred at room temperature for 1.5 hours. The solvent was concentrated under reduced pressure and the residue was diluted with saturated ammonium chloride (5 mL). The solution was extracted with ethyl acetate (3×10 mL). The combined organic layers were washed with saturated brine (1×20 mL), dried over sodium sulfate, filtered and concentrated under reduced pressure onto silica (12 g). The material was purified on a Büchi automated chromatography system (11 g, Biotage 50 μm KP-amino-d column), eluting with a gradient of 0 to 100% ethyl acetate in hexanes. The solid was dried under vacuum at 25° C. for 16 hours to give title compound (32 mg, 47% yield) as a white solid. ¹H NMR (400 MHz, CDCl₃) δ=7.88 (s, 1H), 7.52 (d, J=7.8 Hz, 1H), 7.32 (d, J=7.9 Hz, 1H), 7.20 (dt, J=1.2, 7.6 Hz, 1H), 7.16-7.10 (m, 1H), 6.75 (s, 1H), 5.13 (s, 1H), 4.87 (ddd, J=2.3, 4.2, 6.8 Hz, 1H), 4.61-4.40 (m, 2H), 4.01-3.85 (m, 2H), 3.64-3.48 (m, 1H), 3.24-3.00 (m, 3H), 3.00-2.84 (m, 2H), 2.76 (dd, J=4.3, 16.1 Hz, 1H), 2.68-2.52 (m, 2H), 2.48-2.33 (m, 2H), 2.29-2.17 (m, 1H), 1.99-1.76 (m, 3H), 1.33-1.17 (m, 6H); LCMS: m/z=523.2 [M+H]⁺.

### Example 123: tert-Butyl (1S,3R)-1-(2-(((2R,3R)-1-(tert-butoxycarbonyl)-2-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

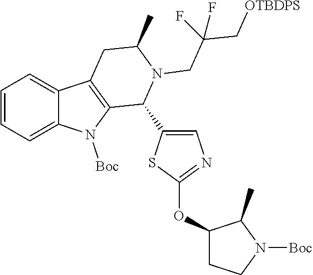

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.2 g, 0.26 mmol, 1 equiv) and tert-butyl (2R,3R)-3-hydroxy-2-methylpyrrolidine-1-carboxylate (0.175 g, 0.86 mmol, 3.34 equiv) in anhydrous acetonitrile (15 mL) was sparged with nitrogen for 20 minutes. 1,4-Diazabicyclo[2.2.2]octane (0.086 g, 0.77 mmol, 3.0 equiv), nickel (II) chloride ethylene glycol dimethylene ether complex (0.028 g, 0.13 mmol, 0.5 equiv), 4,4′-bis(di-t-butyl)-2,2′-bipyridine (0.034 g, mmol, 0.5 equiv) and 0.13 Ir[dF(CF₃)PPy]₂dtbbpy]PF₆ catalyst (0.029 g, 0.026 mmol, 0.1 equiv) were sequentially added and the mixture was sparged with nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure, and the residue was purified on a Büchi automated chromatography system (25 g, Sorbtech 40-75 μm silica gel column), eluting with a gradient of 0 to 100% ethyl acetate in heptanes, to give the title compound (0.095 g, 41% yield) as a white solid. LCMS: m/z=902.2 [M+H]⁺.

### Example 124: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2R,3R)-2-methylpyrrolidin-3-yl)oxy)thiazole

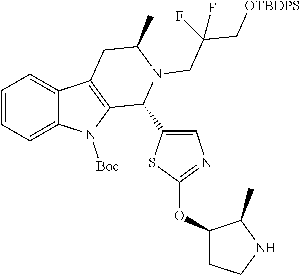

Trifluoroacetic acid (0.5 mL, 6.32 mmol, 60 equiv) was added dropwise over ˜2 minutes to tert-butyl (1S,3R)-1-(2-(((2R,3R)-1-(tert-butoxycarbonyl)-2-methylpyrrolidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.095 g, 0.11 mmol, 1.0 equiv) in dichloromethane (0.8 mL) in a 20 mL vial at 0° C. After stirring at 0° C. for 3 days. The pH was adjusted to 10 with cold saturated sodium. The solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.075 g, 97% yield) as a yellow foam, which was used subsequently. LCMS: m/z=702.2 [M+H]⁺.

### Example 125: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2R,3R)-1-(3-fluoropropyl)-2-methylpyrrolidin-3-yl)oxy)thiazole

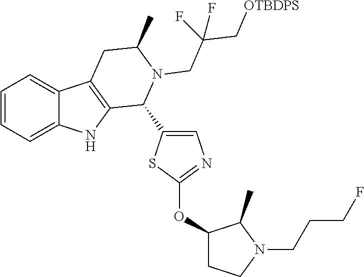

1-Bromo-3-fluoropropane (12 μL, 0.13 mmol, 1.2 equiv) and N,N-diisopropylethylamine (0.1 mL, 0.55 mmol, 5.0 equiv) was added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2R,3R)-2-methylpyrrolidin-3-yl)oxy)thiazole (0.075 g, 0.15 mmol, 1.0 equiv) in N,N-dimethylacetamide (2.0 mL) in a 40 mL vial and stirred at room temperature for 3 days. The reaction mixture was diluted with ethyl acetate (20 mL) and washed with saturated brine (2×15 mL). The organic layer was dried over sodium sulfate, filtered and concentrated under reduced pressure. The residue was dried under vacuum at 25° C. for 1.5 hours to give the title compound (0.071 g, 85% yield) as an orange oil, which was used subsequently. LCMS: m/z=762.2 [M+H]⁺.

### Example 126: 2,2-Difluoro-3-((1S,3R)-1-(2-(((2R,3R)-1-(3-fluoropropyl)-2-methylpyrrolidin-3-yl)oxy)thiazol-5-yl)-3-methyl-1,3,4,9-tetrahydro-2H-pyrido[3,4-b]indol-2-yl)propan-1-ol

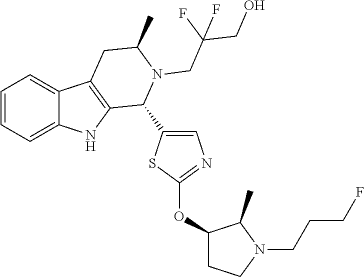 [SMILES: C[C@@H]1[C@H](Oc2ncc([C@@H]3c4[nH]c5ccccc5c4C[C@@H](C)N3CC(F)(F)CO)s2)CCN1CCCF]

1M Tetra-n-butyl ammonium fluoride in tetrahydrofuran (0.10 mL, 0.10 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((2R,3R)-1-(3-fluoropropyl)-2-methylpyrrolidin-3-yl)oxy)thiazole (0.071 g, 0.093 mmol, 1.0 equiv) in tetrahydrofuran (1.5 mL) in a 40 mL vial and stirred at room temperature for 1.5 hours. The solvent was concentrated under reduced pressure and the residue was diluted with saturated ammonium chloride (5 mL). The solution was extracted with ethyl acetate (3×10 mL). The combined organic layers were washed with saturated brine (1×20 mL), dried over sodium sulfate, filtered and concentrated under reduced pressure onto silica gel (12 g). The material was purified on a Büchi automated chromatography system (11 g, Biotage 50 μm KP-amino-d column), eluting with a gradient of 0 to 100% ethyl acetate in hexanes. The solid was dried under vacuum at 25° C. for 16 hours to give title compound (23 mg, 47% yield) as a white solid. ¹H NMR (400 MHz, CDCl₃) δ=7.89 (s, 1H), 7.52 (d, J=7.7 Hz, 1H), 7.32 (d, J=8.3 Hz, 1H), 7.20 (dt, J=1.3, 7.5 Hz, 1H), 7.16-7.10 (m, 1H), 6.73 (s, 1H), 5.31-5.25 (m, 1H), 5.12 (s, 1H), 4.62-4.55 (m, 1H), 4.50-4.43 (m, 1H), 4.00-3.85 (m, 2H), 3.58-3.47 (m, 1H), 3.27-3.08 (m, 2H), 3.08-2.84 (m, 3H), 2.76 (dd, J=4.3, 16.1 Hz, 1H), 2.60-2.32 (m, 3H), 2.21-2.03 (m, 2H), 2.01-1.80 (m, 3H), 1.26-1.14 (m, 6H); LCMS: m/z=523.2 [M+H]⁺.

### Example 127: tert-Butyl (S)-4-((5-((1S,3R)-9-(tert-butoxycarbonyl)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-yl)oxy)isoxazolidine-2-carboxylate

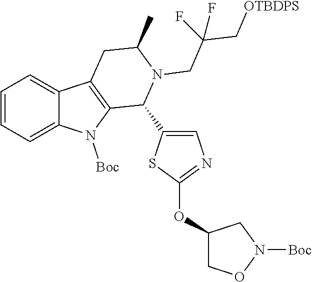

A mixture of tert-butyl (1S,3R)-1-(2-bromothiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate (0.781 g, 1.0 mmol, 1 equiv), tert-butyl (S)-4-hydroxyisoxazolidine-2-carboxylate (0.632 g, 3.34 mmol, 3.34 equiv) and 1,4-diazabicyclo[2.2.2]octane (0.337 g, 3.0 mmol, 3 equiv) in acetonitrile (15 mL) was sparged with nitrogen for 20 minutes. Nickel(II) chloride (glyme) complex (0.110 g, 0.50 mmol, 0.5 equiv), 4,4′-di-tert-butyl-2,2′-dipyridyl (0.134 g, 0.50 mmol, 0.5 equiv) and (Ir[dF(CF₃)ppy]₂(dtbpy))PF₆ (0.112 g, 0.10 mmol, 0.1 equiv) were sequentially added at room temperature, The mixture was sparged with nitrogen for an additional 5 minutes. The reaction vessel was sealed and the reaction was irradiated with Blue LED light for 18 hours. The mixture was dry-loaded onto Celite® and purified on an Interchim automated chromatography system (Sorbtech 40 g silica gel column), eluting with a gradient of 0 to 60% ethyl acetate in heptanes, to give the title compound (0.686 g, 77% yield) as a white foam. LCMS: m/z=889 [M+H]⁺.

### Example 128: (S)-4-((5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)thiazol-2-yl)oxy)isoxazolidine

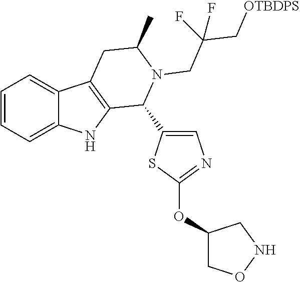

Trifluoroacetic acid (3.50 mL, 45.88 mmol, 60 equiv) was added to a solution of tert-butyl (S)-4-((5-((1S,3R)-9-(tert-butoxycarbonyl)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-yl)oxy)isoxazolidine-2-carboxylate (0.680 g, 0.765 mmol, 1 equiv) in dichloromethane (10 mL) at 0° C. After stirring at 0° C. for 29 hours, LC analysis indicated that the reaction was complete. The pH was adjusted to 10 with saturated sodium carbonate at 0° C. The mixture was extracted with dichloromethane (3×50 mL). The combined organic layers were dried over anhydrous sodium sulfate, filtered and concentrated under reduced pressure to give the title compound (0.482 g, 91% yield) as a yellow foam. LCMS: m/z=689 [M+H]⁺.

### Example 129: (S)-4-((5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)thiazol-2-yl)oxy)-2-(3-fluoropropyl)isoxazolidine

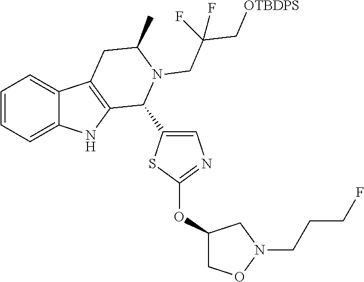

N,N-Diisopropylethylamine (0.60 mL, 3.425 mmol, 5 equiv) was added to a solution of(S)-4-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-yl)oxy)isoxazolidine (0.472 g, 0.685 mmol, 1 equiv) in N,N-dimethylacetamide (5 mL). After stirring at room temperature for 10 minutes, 1-bromo-3-fluoropropane (116 mg, 0.822 mmol, 1.2 equiv) was added and the mixture was stirred at room temperature for 6 days at which time LC analysis indicated ˜25% of product. The mixture was diluted with ethyl acetate (50 mL) and washed with water (20 mL). The aqueous layer was extracted with ethyl acetate (3×20 mL). The combined organic layers were washed with saturated brine (50 mL), dried over anhydrous sodium sulfate, filtered and concentrated under reduced pressure. The residue was purified on an Interchim automated chromatography system (Sorbtech 40 g silica gel column), eluting with a gradient of 0 to 100% ethyl acetate in heptanes, to give the title compound (62.4 mg, 12% yield) as a brown oil. LCMS: m/z=749 [M+H]⁺.

### Example 130: 2,2-Difluoro-3-((1S,3R)-1-(2-(((S)-2-(3-fluoropropyl)isoxazolidin-4-yl)oxy)thiazol-5-yl)-3-methyl-1,3,4,9-tetrahydro-2H-pyrido[3,4-b]indol-2-yl)propan-1-ol

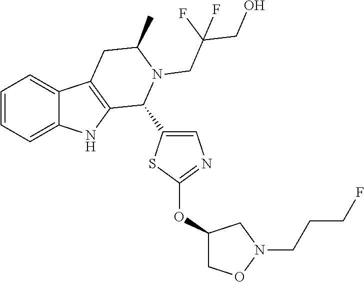 [SMILES: C[C@@H]1Cc2c([nH]c3ccccc23)[C@@H](c2cnc(O[C@@H]3CON(CCCF)C3)s2)N1CC(F)(F)CO]

1 M Tetra-n-butylammonium fluoride in THF (0.091 mL, 0.091 mmol, 1.1 equiv) was added dropwise at room temperature to a solution of (S)-4-((5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)thiazol-2-yl)oxy)-2-(3-fluoropropyl)isoxazolidine (62 mg, 0.0828 mmol, 1 equiv) in THF (2 mL). After stirring for 2 hours, LC analysis indicated >99% conversion. The residue was diluted with saturated ammonium chloride (10 mL). The suspension was extracted with ethyl acetate (3×20 mL). The combined organic layers were dried over sodium sulfate, filtered and concentrated under reduced pressure. The residue was purified on an Interchim automated chromatography system (Biotage 11 g KPNH column), eluting with a gradient of 0 to 100% ethyl acetate in heptanes, to give the title compound (27 mg, 64% yield) as a white solid. LCMS: m/z=511 [M+H]⁺; ¹H NMR (400 MHz, CDCl₃) δ=7.90 (s, 1H), 7.52 (d, J=7.8 Hz, 1H), 7.32 (d, J=7.6 Hz, 1H), 7.20 (t, J=7.2 Hz, 1H), 7.16-7.11 (m, 1H), 6.71 (s, 1H), 5.65-5.58 (m, 1H), 5.13 (s, 1H), 4.62 (br s, 1H), 4.50 (br s, 1H), 4.31 (br s, 1H), 4.08-3.84 (m, 3H), 3.77-3.58 (m, 1H), 3.58-3.34 (m, 2H), 3.29-3.02 (m, 2H), 3.02-2.81 (m, 4H), 2.74 (dd, J=4.3, 16.1 Hz, 1H), 2.56 (dd, J=9.7, 16.1 Hz, 1H), 2.09-2.08-1.92 (m, 2H), 1.69-1.51 (m, 1H), 1.33 (br s, 1H), 1.24 (d, J=6.8 Hz, 3H), 1.01-0.74 (m, 1H).

### Example 131: tert-Butyl (1S,3R)-1-(2-(((R)-1-(tert-butoxycarbonyl)piperidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

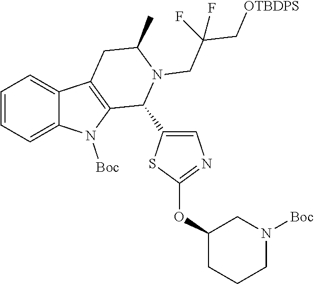

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.2 g, 0.26 mmol, 1 equiv) and tert-butyl (R)-3-hydroxypiperidine-1-carboxylate (0.172 g, 0.86 mmol, 3.34 equiv) in anhydrous acetonitrile (15 mL) was sparged with nitrogen for 20 minutes. 1,4-Diazabicyclo[2.2.2]octane (0.086 g, 0.77 mmol, 3.0 equiv), nickel (II) chloride ethylene glycol dimethylene ether complex (0.028 g, 0.13 mmol, 0.5 equiv), 4,4′-bis(di-t-butyl)-2,2′-bipyridine (0.034 g, 0.13 mmol, 0.5 equiv) and Ir[dF(CF₃)PPy]₂dtbbpy]PF₆ catalyst (0.029 g, 0.026 mmol, 0.1 equiv) were sequentially added and the mixture was sparged with nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure, and the residue was purified on a Büchi automated chromatography system (40 g, Sorbtech 40-75 μm silica gel column), eluting with a gradient of 0 to 100% ethyl acetate in heptanes, to give title compound (0.100 g, 43% yield) as a white solid. LCMS: m/z=902.2 [M+H]⁺.

### Example 132: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-piperidin-3-yl)oxy)thiazole

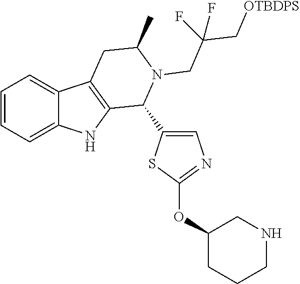

Trifluoroacetic acid (0.51 mL, 6.7 mmol, 60 equiv) was added dropwise over ˜2 minutes to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-piperidin-3-yl)oxy)thiazole (0.100 g, 0.11 mmol, 1.0 equiv) in dichloromethane (0.51 mL) in a 20 mL vial at 0° C. After stirring at 0° C. for 4 days, the pH was adjusted to 10 with cold saturated sodium. The solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.077 g, 100% yield) as a yellow foam, which was used subsequently. LCMS: m/z=702.2 [M+H]⁺.

### Example 133: 5-((1S,3R)-2-(3-((tert-Butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)piperidin-3-yl)oxy)thiazole

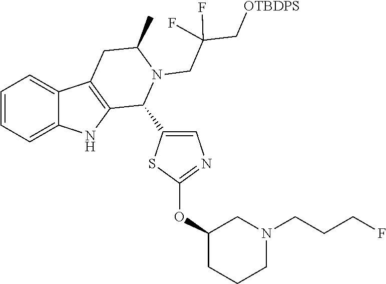

1-Bromo-3-fluoropropane (12 μL, 0.13 mmol, 1.2 equiv) and N,N-diisopropylethylamine (0.096 mL, 0.55 mmol, 5.0 equiv) were added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-piperidin-3-yl)oxy)thiazole (0.077 g, 0.11 mmol, 1.0 equiv) in N,N-dimethylacetamide (2.0 mL) in a 40 mL vial and stirred at room temperature for 2 days. The reaction mixture was diluted with ethyl acetate (20 mL) and washed with saturated brine (2×15 mL). The organic layer was dried over sodium sulfate, filtered and concentrated under reduced pressure. The residue was dried under vacuum at 25° C. for 1.5 hours to give the title compound (0.062 g, 74% yield) as an orange oil, which was used subsequently. LCMS: m/z=762.2 [M+H]⁺.

### Example 134: 2,2-Difluoro-3-((1S,3R)-1-(2-(((R)-1-(3-fluoropropyl)piperidin-3-yl)oxy)thiazol-5-yl)-3-methyl-1,3,4,9-tetrahydro-2H-pyrido[3,4-b]indol-2-yl)propan-1-ol

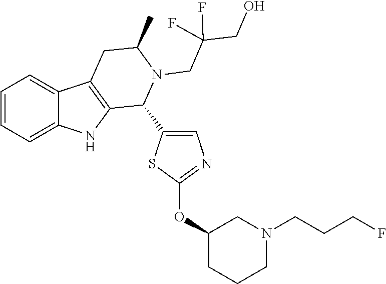 [SMILES: C[C@@H]1Cc2c([nH]c3ccccc23)[C@@H](c2cnc(O[C@@H]3CCCN(CCCF)C3)s2)N1CC(F)(F)CO]

1M Tetra-n-butyl ammonium fluoride in tetrahydrofuran (0.09 mL, 0.091 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((R)-1-(3-fluoropropyl)piperidin-3-yl)oxy)thiazole (0.062 g, 0.081 mmol, 1.0 equiv) in tetrahydrofuran (1.5 mL) in a 40 mL vial and stirred at room temperature for 1.5 hours. The solvent was concentrated under reduced pressure and the residue was diluted with saturated ammonium chloride (5 mL). The solution was extracted with ethyl acetate (3×10 mL). The combined organic layers were washed with saturated brine (1×20 mL, dried over sodium sulfate, filtered and concentrated under reduced pressure onto silica gel (12 g). The material was purified on a Büchi automated chromatography system (11 g, Biotage 50 μm KP-amino-d column), eluting with a gradient of 0 to 100% ethyl acetate in hexanes. The solid was dried under vacuum at 25° C. for 16 hours to give title compound (15 mg, 36% yield) as a white solid. ¹H NMR (400 MHz, CDCl₃) δ=7.86 (s, 1H), 7.52 (d, J=7.8 Hz, 1H), 7.32 (d, J=7.9 Hz, 1H), 7.22-7.10 (m, 2H), 6.76 (s, 1H), 5.11 (s, 1H), 4.95 (tt, J=3.9, 7.9 Hz, 1H), 4.53 (t, J=6.1 Hz, 1H), 4.41 (t, J=6.0 Hz, 1H), 4.00-3.84 (m, 2H), 3.66-3.45 (m, 1H), 3.26-3.07 (m, 1H), 2.99-2.85 (m, 2H), 2.77 (dd, J=4.3, 16.1 Hz, 1H), 2.61-2.38 (m, 5H), 2.24 (br t, J=8.3 Hz, 1H), 2.08-1.96 (m, 1H), 1.92-1.75 (m, 3H), 1.69-1.49 (m, 3H), 1.23 (d, J=6.7 Hz, 3H); LCMS: m/z=523.3 [M+H]⁺.

### Example 135: tert-butyl (1S,3R)-1-(2-(((S)-1-(tert-butoxycarbonyl)piperidin-3-yl)oxy)thiazol-5-yl)-2-(3-((tert-butyldiphenylsilyl)oxy)-2,2-difluoropropyl)-3-methyl-1,2,3,4-tetrahydro-9H-pyrido[3,4-b]indole-9-carboxylate

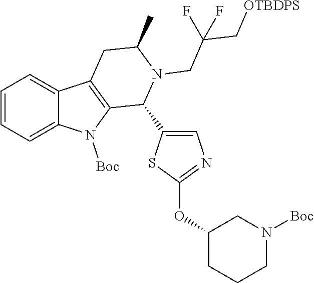

A solution of tert-butyl (1S,3R)-1-(2-bromothiazol-5-yl)-2-[3-[tert-butyl(diphenyl)silyl]oxy-2,2-difluoro-propyl]-3-methyl-3,4-dihydro-1H-pyrido[3,4-b]indole-9-carboxylate (0.2 g, 0.26 mmol, 1 equiv) and tert-butyl (S)-3-hydroxypiperidine-1-carboxylate (0.172 g, 0.86 mmol, 3.34 equiv) in anhydrous acetonitrile (15 mL) was sparged with nitrogen for 20 minutes. 1,4-Diazabicyclo[2.2.2]octane) (0.086 g, 0.77 mmol, 3.0 equiv), nickel (II) chloride ethylene glycol dimethylene ether complex (0.028 g, 0.13 mmol, 0.5 equiv), 4,4′-bis(di-t-butyl)-2,2′-bipyridine (0.034 g, 0.13 mmol, 0.5 equiv) and Ir[dF(CF₃)PPy]₂dtbbpy]PF₆ catalyst (0.029 g, 0.026 mmol, 0.1 equiv) were sequentially added and the mixture was sparged with nitrogen for 20 additional minutes. The resulting clear yellow solution was stirred at room temperature under irradiation with blue LED light for 24 hours. The mixture was filtered through Celite®, which was washed with ethyl acetate (3×20 mL). The filtrate was concentrated under reduced pressure and the residue was purified on a Büchi automated chromatography system (40 g, Sorbtech 40-75 μm silica gel column), eluting with a gradient of 0 to 100% ethyl acetate in heptanes, to give the title compound (0.140 g, 60% yield) as a white solid. LCMS: m/z=902.2 [M+H]⁺.

### Example 136: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-piperidin-3-yl)oxy)thiazole

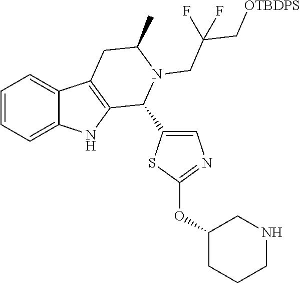

Trifluoroacetic acid (0.71 mL, 9.3 mmol, 60 equiv) was added dropwise over ˜2 minutes to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-piperidin-3-yl)oxy)thiazole (0.140 g, 0.16 mmol, 1.0 equiv) in dichloromethane (0.51 mL) in a 20 mL vial at 0° C. After stirring at 0° C. for 4 days, the pH was adjusted to 10 with cold saturated sodium carbonate. The solution was extracted with dichloromethane (3×15 mL). The combined organic layers were washed with saturated brine (1×20 mL), dried over sodium sulfate, filtered and concentrated under reduced pressure. The product was dried under vacuum at 25° C. for 1.5 hours to give the title compound (0.112 g, 100% yield) as a yellow foam, which was used subsequently. LCMS: m/z=702.2 [M+H]⁺.

### Example 137: 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-1-(3-fluoropropyl)piperidin-3-yl)oxy)thiazole

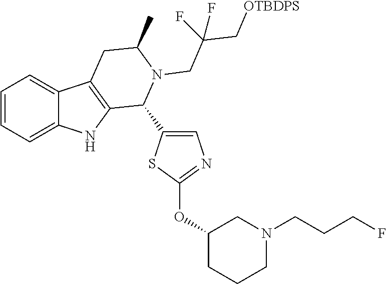

1-Bromo-3-fluoropropane (18 μL, 0.19 mmol, 1.2 equiv) and N,N-diisopropylethylamine (0.14 mL, 0.80 mmol, 5.0 equiv) was added to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-piperidin-3-yl)oxy)thiazole (0.112 g, 0.16 mmol, 1.0 equiv) in N,N-dimethylacetamide (2.0 mL) in a 40 mL vial and stirred at room temperature for 2 days. The reaction mixture was diluted with ethyl acetate (20 mL) and washed with saturated brine (2×15 mL). The organic layer was dried over sodium sulfate, filtered and concentrated under reduced pressure. The residue was dried under vacuum at 25° C. for 1.5 hours to give the title compound (0.073 g, 60% yield) as an orange oil, which was used. LCMS: m/z=762.2 [M+H]⁺.

### Example 138: 2,2-Difluoro-3-((1S,3R)-1-(2-(((S)-1-(3-fluoropropyl)piperidin-3-yl)oxy)thiazol-5-yl)-3-methyl-1,3,4,9-tetrahydro-2H-pyrido[3,4-b]indol-2-yl)propan-1-ol

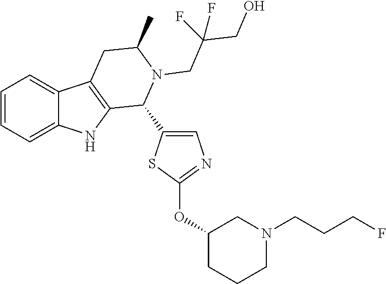 [SMILES: C[C@@H]1Cc2c([nH]c3ccccc23)[C@@H](c2cnc(O[C@H]3CCCN(CCCF)C3)s2)N1CC(F)(F)CO]

1M Tetra-n-butyl ammonium fluoride (in tetrahydrofuran (0.11 mL, 0.11 mmol, 1.10 equiv) was added dropwise to 5-((1S,3R)-2-(3-((tert-butyldiphenylsilyl)oxy)-2,2-difluoropropyl)-3-methyl-2,3,4,9-tetrahydro-1H-pyrido[3,4-b]indol-1-yl)-2-(((S)-1-(3-fluoropropyl)piperidin-3-yl)oxy)thiazole (0.073 g, 0.096 mmol, 1.0 equiv) in tetrahydrofuran (1.5 mL) in a 40 mL vial and stirred at room temperature for 1.5 hours. The solvent was concentrated under reduced pressure and the residue was diluted with saturated ammonium chloride (5 mL). The solution was extracted with ethyl acetate (3×10 mL). The combined organic layers were washed with saturated brine (1×20 mL), dried over sodium sulfate, filtered and concentrated under reduced pressure onto silica gel (12 g). The material was purified on a Büchi automated chromatography system (11 g, Biotage 50 μm KP-amino-d column), eluting with a gradient of 0 to 100% ethyl acetate in hexanes. The solid was dried under vacuum at 25° C. for 16 hours to give the title compound (18 mg, 36% yield) as a white solid. ¹H NMR (400 MHz, CDCl₃) δ=7.52 (d, J=7.8 Hz, 1H), 7.32 (d, J=7.8 Hz, 1H), 7.20 (dt, J=1.2, 7.6 Hz, 1H), 7.15-7.10 (m, 1H), 6.74 (s, 1H), 5.12 (s, 1H), 4.95 (tt, J=4.0, 8.0 Hz, 1H), 4.54 (t, J=6.0 Hz, 1H), 4.42 (t, J=6.0 Hz, 1H), 4.01-3.84 (m, 2H), 3.64-3.48 (m, 1H), 3.25-3.03 (m, 2H), 3.03-2.85 (m, 2H), 2.76 (dd, J=4.3, 16.1 Hz, 1H), 2.60-2.45 (m, 4H), 2.39 (br dd, J=8.1, 10.5 Hz, 1H), 2.24 (br t, J=8.1 Hz, 1H), 2.09-1.96 (m, 1H), 1.93-1.74 (m, 3H), 1.64-1.53 (m, 2H), 1.29-1.17 (m, 3H); LCMS: m/z=523.2 [M+H]⁺.

### Example 139: Biological Evaluation

ECC-1 cells were trypsinized and resuspended in hormone-depleted media and plated at a density of 15 k cells per well into a 96-well plate for at least 4 hours. Cells were treated with test compounds in the absence of E2 (for agonist mode) or in the presence of 500 pM E2 (for antagonist mode) for 3 days and plates were subsequently frozen at −80° C. Thawed plates were incubated with a chromogenic substrate of AP, p-nitrophenyl phosphate (Thermo Fisher Scientific), for 40 minutes at 42° C., and absorbances were read at 405 nm. AP activity was normalized to the activity of 500 pM E2 alone. This assay was shown to correlate with the in vivo studies comparing uterine wet weight in ovariectomized rats following treatment with a number of antiestrogens.

Table 6 shows estrogen receptor modulation (e.g., agonism and antagonism) of certain compounds of the present disclosure. The compound numbers correspond to the compound numbers of Table 1 and 2. Compounds having an activity designated as “+” provided estrogen receptor agonism having (i) at least 80% increase in the E2-normalized signal in the AP assay (agonist mode) and (ii) no more than 80% reduction in the E2-normalized signal in the AP assay (antagonist mode). Compounds having an activity designated as “++” provided estrogen receptor antagonism having (i) between 10% and 80% increase in the E2-normalized signal in the AP assay (agonist mode) and (ii) between 10% and 80% reduction in the E2-normalized signal in the AP assay (antagonist mode). Compounds having an activity designated as “+++” provided estrogen receptor antagonism (i) with pIC₅₀ greater than 7.5 and at least a 10% reduction in the E2-normalized signal in the AP assay (antagonist mode); and no more than 10% increase in E2-normalized signal in the AP assay (agonist mode).

In some embodiments, particularly useful modulators of the estrogen receptor are compounds having greater than “+” activity in Table 6. In some embodiments, particularly useful modulators of the estrogen receptor are compounds having greater than “++” activity in Table 6. In some embodiments, particularly useful modulators of the estrogen receptor are compounds having “+++” activity in Table 6.

TABLE 6

| Compound No. | Activity |
|---|---|
| I-1 | ++ |
| I-2 | ++ |
| I-3 | ++ |
| I-4 | +++ |
| I-5 | + |
| I-6 | ++ |
| I-8 | ++ |
| I-9 | ++ |
| I-10 | + |
| I-11 | ++ |
| I-12 | +++ |
| I-13 | +++ |
| I-14 | + |
| I-15 | + |
| I-16 | +++ |
| I-17 | + |
| I-18 | ++ |
| I-19 | +++ |
| I-20 | ++ |
| I-21 | ++ |
| I-22 | ++ |
| I-23 | +++ |
| I-24 | ++ |
| I-25 | + |
| I-26 | +++ |
| I-27 | +++ |
| I-28 | +++ |
| I-29 | + |
| I-30 | + |
| I-31 | ++ |
| I-32 | + |
| I-33 | +++ |
| I-34 | + |
| I-35 | + |
| I-36 | + |
| I-37 | +++ |
| I-38 | + |

The embodiments of the disclosure described above are intended to be merely exemplary, numerous variations and modifications will be apparent to those skilled in the art.

All such variations and modifications are intended to be within the scope of the present invention as defined in any appended claims.

1. A compound of Formula I: 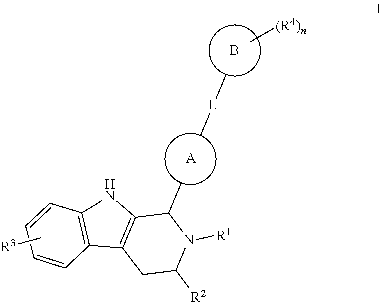
or a pharmaceutically acceptable salt thereof, wherein:
A is selected from optionally substituted 5-membered heteroaryl comprising 1-3 heteroatoms selected from N, O, and S, wherein at least 1 heteroatom is S or O, optionally substituted 7- to 10-membered bicyclic heteroaryl comprising 1-4 heteroatoms selected from N, O, and S, and optionally substituted 3- to 6-membered heterocyclyl comprising 1-3 heteroatoms selected from N, O, and S;
L is a covalent bond or an optionally substituted bivalent group selected from —O—C₀-C₅ aliphatic- and —C₁-C₅ aliphatic-O—;
B is selected from 3- to 12-membered heterocyclyl comprising 1-3 heteroatoms selected from N, O, and S and C₃-C₆ cycloaliphatic;
R¹ is selected from hydrogen and optionally substituted C₁-C₆ aliphatic;
R² is selected from hydrogen and optionally substituted C₁-C₆ aliphatic;
R³ is selected from hydrogen, halogen, —CN, —OR^{a}, —C(O)R^{a}, —C(O)OR^{a}, —OC(O)R^{a}, —C(O)N(R^{a})₂, —OC(O)N(R^{a})₂, —NO₂, —N(R^{a})₂, —N(R^{a})C(O)R^{a}, —N(R^{a})C(O)OR^{a}, —N(R^{a})S(O)₂R^{a}, —SR^{a}, —S(O)₂R^{a}, —S(O)N(R^{a})₂, —S(O)₂N(R^{a})₂, and an optionally substituted C₁₋₆ aliphatic group;
each R⁴ is independently halogen, —CN, —OR^{a}, —N(R^{a})₂, —C(O)R^{a}, —OC(O)R^{a}, —C(O)OR^{a}, —C(O)N(R^{a})₂, —N(R^{a})C(O)R^{a}, or an optionally substituted group selected from C₁-C₆ aliphatic and 3- to 12-membered heterocyclyl comprising 1 to 3 heteroatoms selected from N, O, and S;
each R^{a} is independently selected from hydrogen and optionally substituted C₁-C₆ aliphatic; and
n is 0 to 5.

2. The compound of claim 1, wherein R¹ is optionally substituted C₁-C₆ aliphatic.

3. The compound of claim 1 or 2, wherein R¹ is C₁-C₆ aliphatic optionally substituted with one or more halogen or —OH.

4. The compound of claim 3, wherein R¹ is C₁-C₆ aliphatic optionally substituted with one or more fluoro or —OH.

5. The compound of any one of claims 1-4, wherein R¹ is selected from:

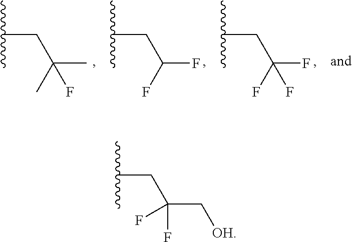

6. The compound of any one of claims 1-5, wherein R² is optionally substituted C₁-C₆ aliphatic.

7. The compound of any one of claims 1-6, wherein R² is methyl.

8. The compound of any one of claims 1-7, wherein R³ is hydrogen.

9. The compound of any one of claims 1-8, wherein A is optionally substituted 5-membered heteroaryl comprising 1-3 heteroatoms selected from N, O, and S, wherein at least 1 heteroatom is S or O, or optionally substituted 7- to 10-membered bicyclic heteroaryl comprising 1-4 heteroatoms selected from N, O, and S.

10. The compound of any one of claims 1-9, wherein A is optionally substituted 5-membered heteroaryl comprising 1-3 heteroatoms selected from N and S, and wherein at least 1 heteroatom is S.

11. The compound of any one of claims 1-10, wherein A is optionally substituted thiophenyl or optionally substituted thiazolyl.

12. The compound of any one of claims 1-11, wherein A is optionally substituted with halogen or C₁-C₆ aliphatic.

13. The compound of any one of claims 1-11, wherein A is selected from: 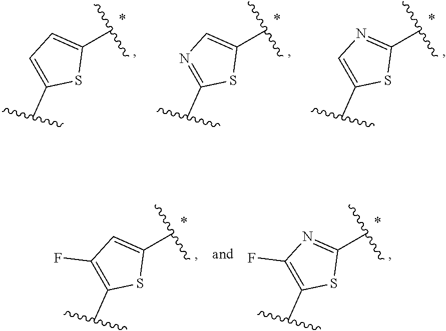 [SMILES: *c1ccc(*)s1 ; *c1cnc(*)s1 ; *c1cnc(*)s1 ; *c1cc(F)c(*)s1 ; *c1nc(F)c(*)s1]
wherein * represents a point of attachment to moiety L.

14. The compound of any one of claims 1-9, wherein A is optionally substituted 7- to 10-membered bicyclic heteroaryl comprising 1-4 heteroatoms selected from N, O, and S.

15. The compound of any one of claims 1-8, wherein A is 3- to 6-membered heterocyclyl comprising 1-3 heteroatoms selected from N, O, and S.

16. The compound of any one of claims 1-15, wherein B is 3- to 7-membered heterocyclyl comprising 1-3 heteroatoms selected from N, O, and S.

17. The compound of any one of claims 1-16, wherein B is 4- to 6-membered heterocyclyl comprising 1-3 heteroatoms selected from N, O, and S.

18. The compound of any one of claims 1-17, wherein B is azetidinyl, pyrrolidinyl, or piperidinyl.

19. The compound of any one of claims 1-18,
wherein: 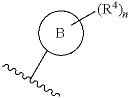
is a moiety selected from:

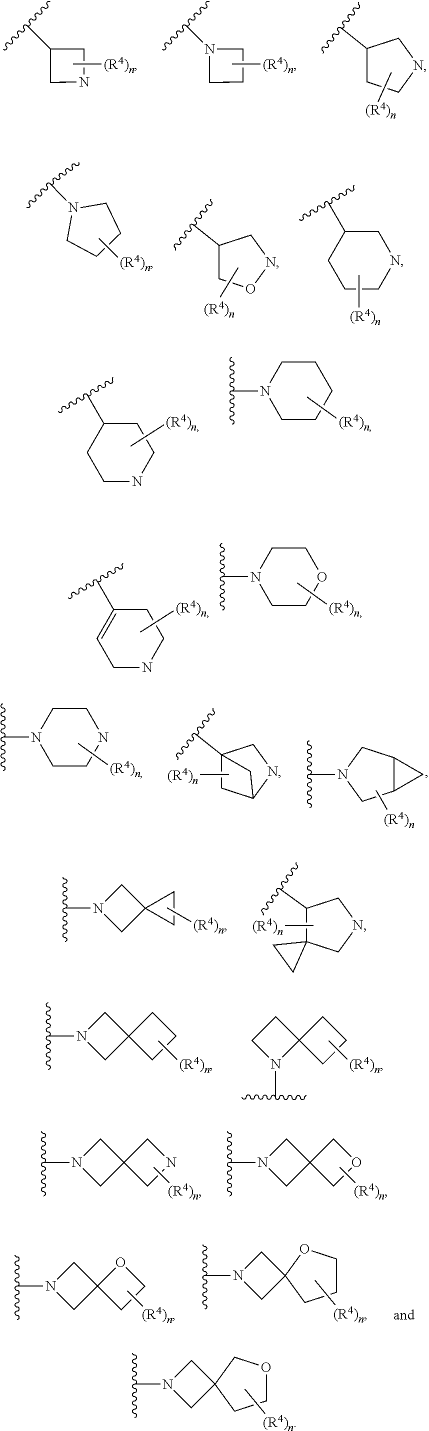

20. The compound of any one of claims 1-19, wherein 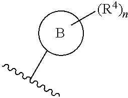
is a moiety selected from:

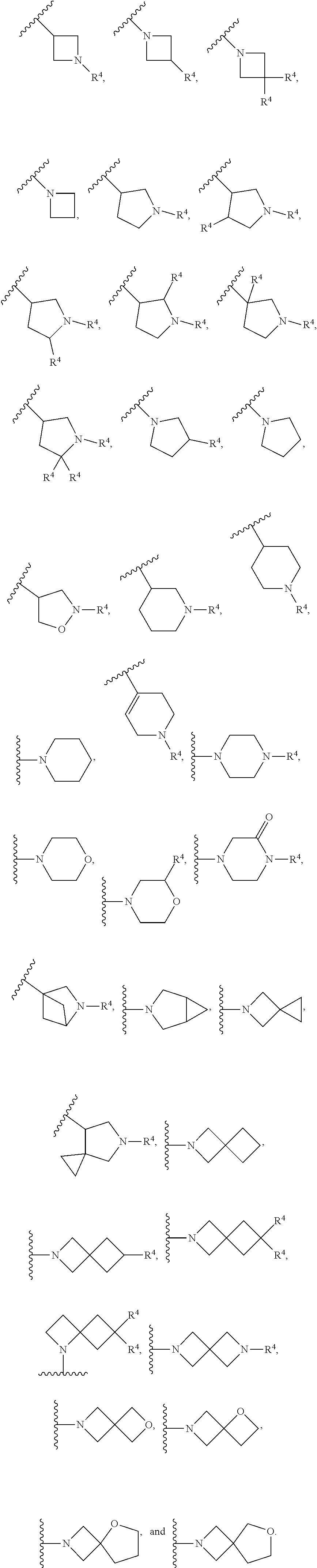

21. The compound of any one of claims 1-15, wherein B is C₃-C₆ cycloaliphatic.

22. The compound of claim 21, wherein B is selected from cyclopropyl, cyclobutyl, cyclopentyl and cyclohexyl.

23. The compound of any one of claims 1-22, wherein L is optionally substituted —O—C₀-C₅ aliphatic.

24. The compound of any one of claims 1-23, wherein L is —O—.

25. The compound of any one of claim 1-19 or 21-24, wherein n is 1.

26. The compound of any one of claim 1-19 or 21-24, wherein n is 0.

27. The compound of any one of claims 1-25, wherein each R⁴ is independently selected from halogen and optionally substituted C₁-C₆ aliphatic group.

28. The compound of any one of claim 1-25 or 27, wherein R⁴ is —(CH₂)₂CH₃.

29. The compound of any one of claim 1-25 or 27, wherein R⁴ is —(CH₂)₃F.

30. The compound of any one of claim 1-25 or 27, wherein each R⁴ is independently selected from fluoro, —CH₃, —CH₂CH₃, —(CH₂)₂CH₃, —(CH₂)₃F, —(CH₂)₂F, and —(CH₂)₂CHF₂.

31. The compound of any one of claims 1-30, wherein the compound is of Formula II: 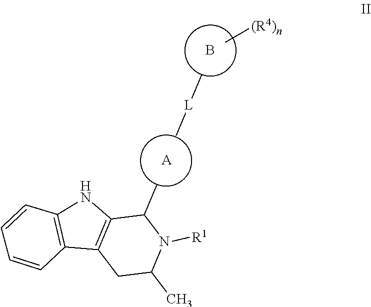
or a pharmaceutically acceptable salt thereof.

32. The compound of claim 31, wherein the compound is of Formula II-a: 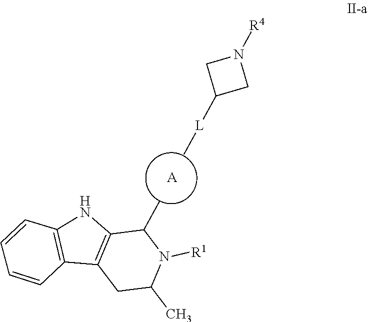
or a pharmaceutically acceptable salt thereof.

33. The compound of claim 31, wherein the compound is of Formula II-b: 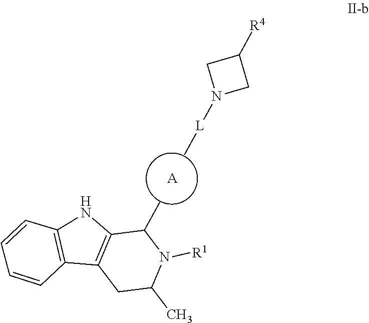
or a pharmaceutically acceptable salt thereof.

34. The compound of claim 31, wherein the compound is of Formula II-c: 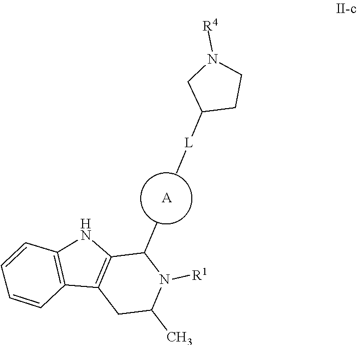
or a pharmaceutically acceptable salt thereof.

35. The compound of claim 31, wherein the compound is of Formula II-d: 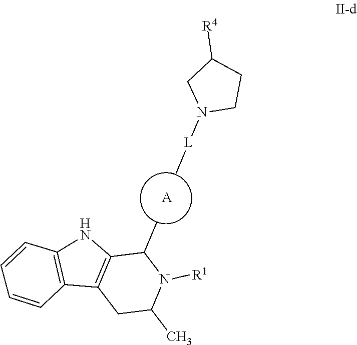
or a pharmaceutically acceptable salt thereof.

36. The compound of any one of claims 1-30, wherein the compound is of Formula II-e: 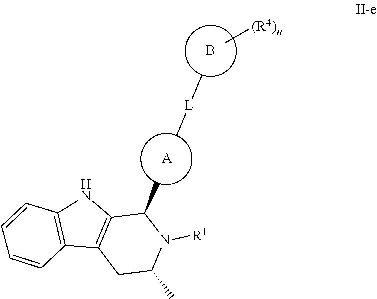
or a pharmaceutically acceptable salt thereof.

37. The compound of any one of claims 1-30, wherein the compound is of Formula II-f: 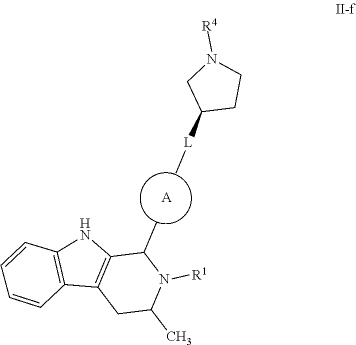
or a pharmaceutically acceptable salt thereof.

38. The compound of any one of claims 1-30, wherein the compound is of Formula II-g: 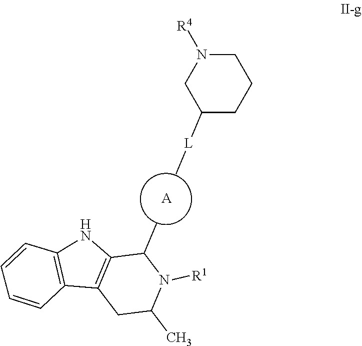
or a pharmaceutically acceptable salt thereof.

39. The compound of any one of claims 1-30, wherein the compound is of Formula II-h: 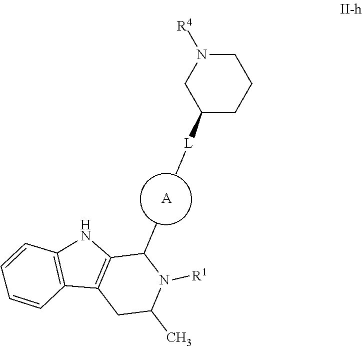
or a pharmaceutically acceptable salt thereof.

40. The compound of any one of claims 1-30, wherein the compound is of Formula III: 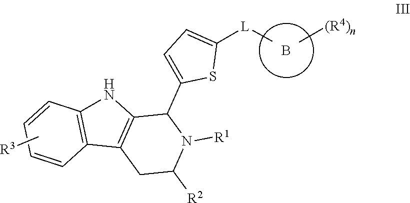
or a pharmaceutically acceptable salt thereof.

41. The compound of any one of claims 1-30, wherein the compound is of Formula IV: 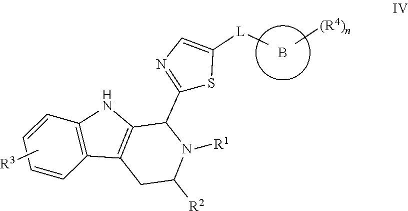
or a pharmaceutically acceptable salt thereof.

42. The compound of any one of claims 1-30, wherein the compound is of Formula V: 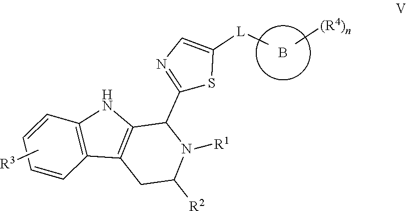
or a pharmaceutically acceptable salt thereof.

43. The compound of any one of claims 1-30, wherein the compound is of Formula VI: 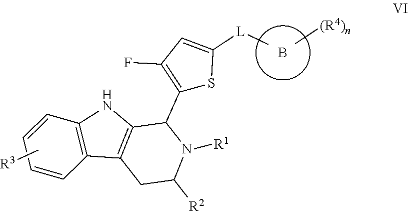
or a pharmaceutically acceptable salt thereof.

44. The compound of any one of claims 1-30, wherein the compound is of Formula VII: 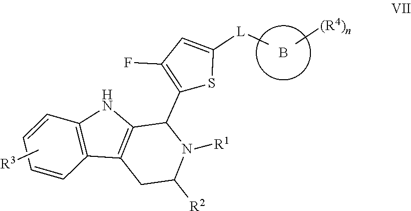
or a pharmaceutically acceptable salt thereof.

45. The compound of any one of claims 1-30, wherein the compound is of Formula VIII: 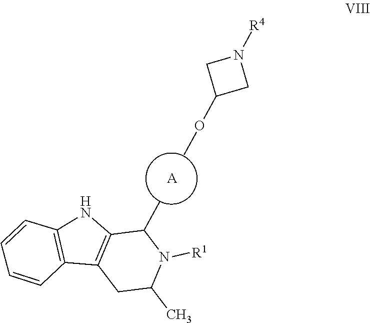
or a pharmaceutically acceptable salt thereof.

46. The compound of any one of claims 1-30, wherein the compound is of Formula IX: 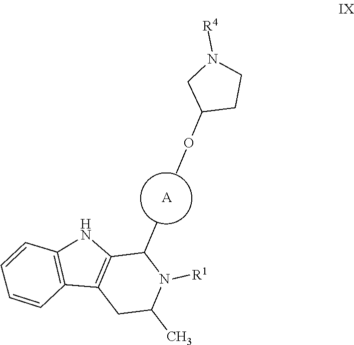
or a pharmaceutically acceptable salt thereof.

47. The compound of any one of claims 1-30, wherein the compound is of Formula IX-a: 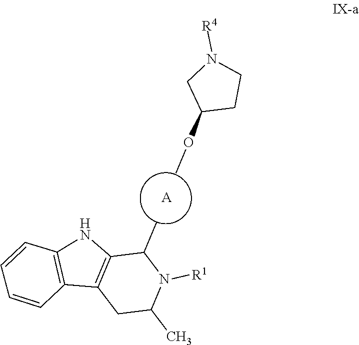
or a pharmaceutically acceptable salt thereof.

48. The compound of any one of claims 1-30, wherein the compound is of Formula X: 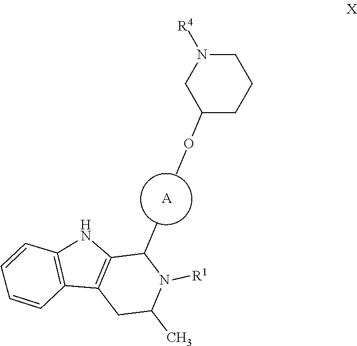
or a pharmaceutically acceptable salt thereof.

49. The compound of any one of claims 1-30, wherein the compound is of Formula X-a: 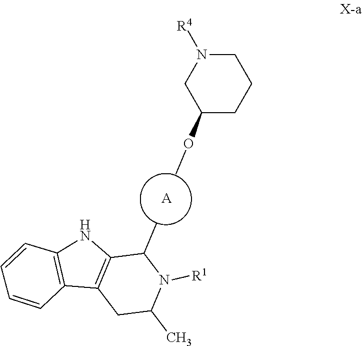
or a pharmaceutically acceptable salt thereof.

50. A compound selected from Table 1, or a pharmaceutically acceptable salt thereof.

51. A compound selected from Table 3, or a pharmaceutically acceptable salt thereof.

52. A compound selected from Table 4, or a pharmaceutically acceptable salt thereof.

53. A compound selected from Table 5, or a pharmaceutically acceptable salt thereof.

54. A pharmaceutical composition comprising a compound of any one of claims 1-53, or a pharmaceutically acceptable salt thereof, and a pharmaceutically acceptable carrier or excipient.

55. A method for treating a disorder mediated by an estrogen receptor in a subject, comprising administering to the subject a therapeutically effective amount of the compound of any one of claims 1-53, or a pharmaceutically acceptable salt thereof, or the composition of claim 54.

56. The method of claim 55, wherein the disorder is selected from the group consisting of breast cancer, ovarian cancer, endometrial cancer, vaginal cancer, lung cancer, bone cancer, uterine cancer, and endometriosis.

57. The method of claim 55 or 56, further comprising administering the compound or composition in combination or alternation with an anti-cancer agent.

58. The method of claim 57, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

59. The method of claim 55 or 56, further comprising administering the compound or composition in combination or alternation with an estrogen receptor antagonist or a partial estrogen receptor antagonist.

60. The method of any one of claims 55-59, wherein the disorder is breast cancer.

61. A method of treating a subject suffering from a cancer comprising administering the compound of any one of claims 1-53, or a pharmaceutically acceptable salt thereof, or the composition of claim 54, in combination with an anti-cancer agent.

62. The method of claim 61, wherein the anti-cancer agent is selected from an mTOR inhibitor, a CDK4/6 inhibitor, a PI3 kinase inhibitor, an aromatase inhibitor, an antibody to or inhibitor of PD-1, PD-L1 or CTLA-4, or an antibody to or inhibitor of EGFR, PGFR, or IGFR.

63. The method of claim 61, wherein the anti-cancer agent is an mTOR inhibitor.

64. The method of claim 63, wherein the mTOR inhibitor is selected from everolimus, sirolimus, temsirolimus, and LY3023414.

65. The method of claim 61, wherein the anti-cancer agent is a CDK4/6 inhibitor.

66. The method of claim 65, wherein the CDK4/6 inhibitor is selected from palbociclib, abemaciclib, ribociclib, lerociclib, trilaciclib, and SHR6390.

67. The method of claim 61, wherein the anti-cancer agent is an antibody to or inhibitor of PD-1, PD-L1 or CTLA-4.

68. The method of claim 61, wherein the anti-cancer agent is an antibody to or inhibitor of EGFR, PGFR, or IGFR.

69. The method of claim 61, wherein the anti-cancer agent is a HER2 inhibitor.

70. The method of claim 69, wherein the HER2 inhibitor is selected from tucatinib, trastuzumab, pertuzumab, ado-trastuzumab, trastuzumab emtansine, ado-trastuzumab emtansine, trastuzumab deruxtecan pertuzumab, lapatinib, and neratinib.

71. The method of claim 61, wherein the anti-cancer agent is a PI3 kinase inhibitor.

72. The method of claim 71, wherein the PI3 kinase inhibitor is selected from perifosine, CAL101, BEZ235, XL147, XL765, GDC-0941, and IPI-145.

73. The method of claim 61, wherein the anti-cancer agent is a PIK3CA inhibitor.

74. The method of claim 73, wherein the PIK3CA inhibitor is selected from alpelisib, taselisib, and LY3023414.

75. The method of claim 61, wherein the anti-cancer agent is an aromatase inhibitor.

76. The method of claim 75, wherein the aromatase inhibitor is selected from aminoglutethimide, testolactone, anastrozole, letrozole, exemestane, vorozole, formestane, fadrozole, 4-hydroxyandrostenedione, 1,4,6-androstatrien-3,17-dione, and 4-androstene-3,6,17-trione.

77. The method of claim 75, wherein the aromatase inhibitor is selected from anastrozole, letrozole, and exemestane.

78. A method of preventing recurrence of a cancer in a subject comprising administering to the subject the compound of any one of claims 1-53, or a pharmaceutically acceptable salt thereof, or the composition of claim 54.

79. The method of claim 78, wherein the cancer is selected from breast cancer, ovarian cancer, endometrial cancer, vaginal cancer, lung cancer, bone cancer, and uterine cancer.

80. The method of claim 78 or 79, wherein the compound or composition is administered as an adjunctive therapy after or instead of chemotherapy, radiation, or surgery.

81. The method of any one of claims 78-80, wherein the compound or composition is administered after surgery.

82. The method of claim 78 or 79, wherein the compound or composition is administered prior to surgery.

83. The method of any one of claims 78-82, wherein the cancer is breast cancer.

84. The method of claim 83, wherein the breast cancer has progressed in the presence of endocrine or aromatase therapy.
